SEQUENCE LISTING (740 sequences)

SEQ ID NO: 1 - protein, 120 aa, synthetic construct
  REGION 1..120: note = Heavy Chain of samrotamab IgG
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 2 - protein, 107 aa, synthetic construct
  REGION 1..107: note = Light Chain of samrotamab IgG
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 3 - protein, 10 aa, synthetic construct
  REGION 1..10: note = Heavy Chain CDR1 of samrotamab
GYKFSSYWIE

SEQ ID NO: 4 - protein, 17 aa, synthetic construct
  REGION 1..17: note = Heavy Chain CDR2 of samrotamab
EILPGSDTTNYNEKFKD

SEQ ID NO: 5 - protein, 13 aa, synthetic construct
  REGION 1..13: note = Heavy Chain CDR3 of samrotamab
ARDRGNYRAWFGY

SEQ ID NO: 6 - protein, 11 aa, synthetic construct
  REGION 1..11: note = Light Chain CDR1 of samrotamab
RASQDISNYLN

SEQ ID NO: 7 - protein, 7 aa, synthetic construct
  REGION 1..7: note = Light Chain CDR2 of samrotamab
YTSRLHS

SEQ ID NO: 8 - protein, 9 aa, synthetic construct
  REGION 1..9: note = Light Chain CDR3 of samrotamab
QQGEALPWT

SEQ ID NO: 9 - protein, 581 aa, synthetic construct
  REGION 1..581: note = Mature Human LRRC15
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 10 - DNA, 1743 bp, synthetic construct
  misc_feature 1..1743: note = cDNA Encoding Mature Human LRRC15
  source 1..1743: mol_type = other DNA
atgccactgaagcattatctccttttgctggtgggctgccaagcctggggtgcagggttggcctaccatggctgccctagcgagtgtacctgctccagggcctcccaggtggagtgcaccggggcacgcattgtggcagtgcccacccctctgccctggaacgccatgagcctgcagatcctcaacacgcacatcactgaactcaatgagtccccgttcctcaatatctcagccctcatcgccctgaggattgagaagaatgagctgtcgcgcatcacgcctggggccttccgaaacctgggctcgctgcgctatctcagcctcgccaacaacaagctgcaggttctgcccatcggcctcttccagggcctggacagcctcgagtctctccttctgtccagtaaccagctgttgcagatccagccggcccacttctcccagtgcagcaacctcaaggagctgcagttgcacggcaaccacctggaatacatccctgacggagccttcgaccacctggtaggactcacgaagctcaatctgggcaagaatagcctcacccacatctcacccagggtcttccagcacctgggcaacctccaggtcctccggctgtatgagaacaggctcacggatatccccatgggcacttttgatgggcttgttaacctgcaggaactggctctgcagcagaaccagattggactgctctcccctggtctcttccacaacaaccacaacctccagagactctacctgtccaacaaccacatctcccagctgccacccagcgtcttcatgcagctgccccagctcaaccgtcttactctctttgggaattccctgaaggagctctctccggggatcttcgggcccatgcccaacctgcgggagctttggctctatgacaaccacatctcttctctacccgacaatgtcttcagcaacctccgccagttgcaggtcctgattcttagccgcaatcagatcagcttcatctccccgggtgccttcaacgggctaacggagcttcgggagctgtccctccacaccaacgcactgcaggacctggacgggaacgtcttccgcatgttggccaacctgcagaacatctccctgcagaacaaccgcctcagacagctcccagggaatatcttcgccaacgtcaatggcctcatggccatccagctgcagaacaaccagctggagaacttgcccctcggcatcttcgatcacctggggaaactgtgtgagctgcggctgtatgacaatccctggaggtgtgactcagacatccttccgctccgcaactggctcctgctcaaccagcctaggttagggacggacactgtacctgtgtgtttcagcccagccaatgtccgaggccagtccctcattatcatcaatgtcaacgttgctgttccaagcgtccatgtccccgaggtgcctagttacccagaaacaccatggtacccagacacacccagttaccctgacaccacatccgtctcttctaccactgagctaaccagccctgtggaagactacactgatctgactaccattcaggtcactgatgaccgcagcgtttggggcatgacccaggcccagagcgggctggccattgccgccattgtaattggcattgtcgccctggcctgctccctggctgcctgcgtcggctgttgctgctgcaagaagaggagccaagctgtcctgatgcagatgaaggcacccaatgagtgt

SEQ ID NO: 11 - protein, 517 aa, synthetic construct
  REGION 1..517: note = Extracellular Domain of LRRC15
YHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSG

SEQ ID NO: 12 - DNA, 1551 bp, synthetic construct
  misc_feature 1..1551: note = cDNA Encoding Extracellular Domain of LRRC15
  source 1..1551: mol_type = other DNA
taccatggctgccctagcgagtgtacctgctccagggcctcccaggtggagtgcaccggggcacgcattgtggcagtgcccacccctctgccctggaacgccatgagcctgcagatcctcaacacgcacatcactgaactcaatgagtccccgttcctcaatatctcagccctcatcgccctgaggattgagaagaatgagctgtcgcgcatcacgcctggggccttccgaaacctgggctcgctgcgctatctcagcctcgccaacaacaagctgcaggttctgcccatcggcctcttccagggcctggacagcctcgagtctctccttctgtccagtaaccagctgttgcagatccagccggcccacttctcccagtgcagcaacctcaaggagctgcagttgcacggcaaccacctggaatacatccctgacggagccttcgaccacctggtaggactcacgaagctcaatctgggcaagaatagcctcacccacatctcacccagggtcttccagcacctgggcaacctccaggtcctccggctgtatgagaacaggctcacggatatccccatgggcacttttgatgggcttgttaacctgcaggaactggctctgcagcagaaccagattggactgctctcccctggtctcttccacaacaaccacaacctccagagactctacctgtccaacaaccacatctcccagctgccacccagcgtcttcatgcagctgccccagctcaaccgtcttactctctttgggaattccctgaaggagctctctccggggatcttcgggcccatgcccaacctgcgggagctttggctctatgacaaccacatctcttctctacccgacaatgtcttcagcaacctccgccagttgcaggtcctgattcttagccgcaatcagatcagcttcatctccccgggtgccttcaacgggctaacggagcttcgggagctgtccctccacaccaacgcactgcaggacctggacgggaacgtcttccgcatgttggccaacctgcagaacatctccctgcagaacaaccgcctcagacagctcccagggaatatcttcgccaacgtcaatggcctcatggccatccagctgcagaacaaccagctggagaacttgcccctcggcatcttcgatcacctggggaaactgtgtgagctgcggctgtatgacaatccctggaggtgtgactcagacatccttccgctccgcaactggctcctgctcaaccagcctaggttagggacggacactgtacctgtgtgtttcagcccagccaatgtccgaggccagtccctcattatcatcaatgtcaacgttgctgttccaagcgtccatgtccccgaggtgcctagttacccagaaacaccatggtacccagacacacccagttaccctgacaccacatccgtctcttctaccactgagctaaccagccctgtggaagactacactgatctgactaccattcaggtcactgatgaccgcagcgtttggggcatgacccaggcccagagcggg

SEQ ID NO: 13 - protein, 120 aa, synthetic construct
  REGION 1..120: note = Heavy Chain of samrotamab IgG histidine scanning variant #1
EVQLVQSGAEVKKPGASVKVSCKASHYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 14 - protein, 120 aa, synthetic construct
  REGION 1..120: note = Heavy Chain of samrotamab IgG histidine scanning variant #2
EVQLVQSGAEVKKPGASVKVSCKASGHKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 15 - protein, 120 aa, synthetic construct
  REGION 1..120: note = Heavy Chain of samrotamab IgG histidine scanning variant #3
EVQLVQSGAEVKKPGASVKVSCKASGYHFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 16 - protein, 120 aa, synthetic construct
  REGION 1..120: note = Heavy Chain of samrotamab IgG histidine scanning variant #4
EVQLVQSGAEVKKPGASVKVSCKASGYKHSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 17 - protein, 120 aa, synthetic construct
  REGION 1..120: note = Heavy Chain of samrotamab IgG histidine scanning variant #5
EVQLVQSGAEVKKPGASVKVSCKASGYKFHSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 18 - protein, 120 aa, synthetic construct
  REGION 1..120: note = Heavy Chain of samrotamab IgG histidine scanning variant #6
EVQLVQSGAEVKKPGASVKVSCKASGYKFSHYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 19 - protein, 120 aa, synthetic construct
  REGION 1..120: note = Heavy Chain of samrotamab IgG histidine scanning variant #7
EVQLVQSGAEVKKPGASVKVSCKASGYKFSSHWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 20 - protein, 120 aa, synthetic construct
  REGION 1..120: note = Heavy Chain of samrotamab IgG histidine scanning variant #8
EVQLVQSGAEVKKPGASVKVSCKASGYKFSSYHIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 21 - protein, 120 aa, synthetic construct
  REGION 1..120: note = Heavy Chain of samrotamab IgG histidine scanning variant #9
EVQLVQSGAEVKKPGASVKVSCKASGYKFSSYWHEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 22 - protein, 120 aa, synthetic construct
  REGION 1..120: note = Heavy Chain of samrotamab IgG histidine scanning variant #10
EVQLVQSGAEVKKPGASVKVSCKASGYKFSSYWIHWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 23 - protein, 120 aa, synthetic construct
  REGION 1..120: note = Heavy Chain of samrotamab IgG histidine scanning variant #11
EVQLVQSGAEVKKPGASVKVSCKASGYKFSSYWIEWVKQAPGQGLEWIGHILPGSDTTNYNEKFKDRATFTSDTSINTAYMELSRLRSDDTAVYYCARDRGNYRAWFGYWGQGTLVTVSS

SEQ ID NO: 24 - protein, 120 aa, synthetic construct
  REGION 1..120: note = Heavy Chain of samrotamab IgG histidine scanning variant #12
EVQLVQSGAEVKKPGASVKVSCKASGYKFSSYWIEWVKQAPGQGLEWIGEHLPGSDTTNYNEKFKDRATFTSDTSINTAYMELSRLRSDDTAVYYCARDRGNYRAWFGYWGQGTLVTVSS

SEQ ID NO: 25 - protein, 120 aa, synthetic construct
  REGION 1..120: note = Heavy Chain of samrotamab IgG histidine scanning variant #13
EVQLVQSGAEVKKPGASVKVSCKASGYKFSSYWIEWVKQAPGQGLEWIGEIHPGSDTTNYNEKFKDRATFTSDTSINTAYMELSRLRSDDTAVYYCARDRGNYRAWFGYWGQGTLVTVSS

SEQ ID NO: 26 - protein, 120 aa, synthetic construct
  REGION 1..120: note = Heavy Chain of samrotamab IgG histidine scanning variant #14
EVQLVQSGAEVKKPGASVKVSCKASGYKFSSYWIEWVKQAPGQGLEWIGEILHGSDTTNYNEKFKDRATFTSDTSINTAYMELSRLRSDDTAVYYCARDRGNYRAWFGYWGQGTLVTVSS

SEQ ID NO: 27 - protein, 120 aa, synthetic construct
  REGION 1..120: note = Heavy Chain of samrotamab IgG histidine scanning variant #15
EVQLVQSGAEVKKPGASVKVSCKASGYKFSSYWIEWVKQAPGQGLEWIGEILPHSDTTNYNEKFKDRATFTSDTSINTAYMELSRLRSDDTAVYYCARDRGNYRAWFGYWGQGTLVTVSS

SEQ ID NO: 28 - protein, 120 aa, synthetic construct
  REGION 1..120: note = Heavy Chain of samrotamab IgG histidine scanning variant #16
EVQLVQSGAEVKKPGASVKVSCKASGYKFSSYWIEWVKQAPGQGLEWIGEILPGHDTTNYNEKFKDRATFTSDTSINTAYMELSRLRSDDTAVYYCARDRGNYRAWFGYWGQGTLVTVSS

SEQ ID NO: 29 - protein, 120 aa, synthetic construct
  REGION 1..120: note = Heavy Chain of samrotamab IgG histidine scanning variant #17
EVQLVQSGAEVKKPGASVKVSCKASGYKFSSYWIEWVKQAPGQGLEWIGEILPGSHTTNYNEKFKDRATFTSDTSINTAYMELSRLRSDDTAVYYCARDRGNYRAWFGYWGQGTLVTVSS

SEQ ID NO: 30 - protein, 120 aa, synthetic construct
  REGION 1..120: note = Heavy Chain of samrotamab IgG histidine scanning variant #18
EVQLVQSGAEVKKPGASVKVSCKASGYKFSSYWIEWVKQAPGQGLEWIGEILPGSDHTNYNEKFKDRATFTSDTSINTAYMELSRLRSDDTAVYYCARDRGNYRAWFGYWGQGTLVTVSS

SEQ ID NO: 31 - protein, 120 aa, synthetic construct
  REGION 1..120: note = Heavy Chain of samrotamab IgG histidine scanning variant #19
EVQLVQSGAEVKKPGASVKVSCKASGYKFSSYWIEWVKQAPGQGLEWIGEILPGSDTHNYNEKFKDRATFTSDTSINTAYMELSRLRSDDTAVYYCARDRGNYRAWFGYWGQGTLVTVSS

SEQ ID NO: 32 - protein, 120 aa, synthetic construct
  REGION 1..120: note = Heavy Chain of samrotamab IgG histidine scanning variant #20
EVQLVQSGAEVKKPGASVKVSCKASGYKFSSYWIEWVKQAPGQGLEWIGEILPGSDTTHYNEKFKDRATFTSDTSINTAYMELSRLRSDDTAVYYCARDRGNYRAWFGYWGQGTLVTVSS

SEQ ID NO: 33 - protein, 120 aa, synthetic construct
  REGION 1..120: note = Heavy Chain of samrotamab IgG histidine scanning variant #21
EVQLVQSGAEVKKPGASVKVSCKASGYKFSSYWIEWVKQAPGQGLEWIGEILPGSDTTNHNEKFKDRATFTSDTSINTAYMELSRLRSDDTAVYYCARDRGNYRAWFGYWGQGTLVTVSS

SEQ ID NO: 34 - protein, 120 aa, synthetic construct
  REGION 1..120: note = Heavy Chain of samrotamab IgG histidine scanning variant #22
EVQLVQSGAEVKKPGASVKVSCKASGYKFSSYWIEWVKQAPGQGLEWIGEILPGSDTTNYHEKFKDRATFTSDTSINTAYMELSRLRSDDTAVYYCARDRGNYRAWFGYWGQGTLVTVSS

SEQ ID NO: 35 - protein, 120 aa, synthetic construct
  REGION 1..120: note = Heavy Chain of samrotamab IgG histidine scanning variant #23
EVQLVQSGAEVKKPGASVKVSCKASGYKFSSYWIEWVKQAPGQGLEWIGEILPGSDTTNYNHKFKDRATFTSDTSINTAYMELSRLRSDDTAVYYCARDRGNYRAWFGYWGQGTLVTVSS

SEQ ID NO: 36 - protein, 120 aa, synthetic construct
  REGION 1..120: note = Heavy Chain of samrotamab IgG histidine scanning variant #24
EVQLVQSGAEVKKPGASVKVSCKASGYKFSSYWIEWVKQAPGQGLEWIGEILPGSDTTNYNEHFKDRATFTSDTSINTAYMELSRLRSDDTAVYYCARDRGNYRAWFGYWGQGTLVTVSS

SEQ ID NO: 37 - protein, 120 aa, synthetic construct
  REGION 1..120: note = Heavy Chain of samrotamab IgG histidine scanning variant #25
EVQLVQSGAEVKKPGASVKVSCKASGYKFSSYWIEWVKQAPGQGLEWIGEILPGSDTTNYNEKHKDRATFTSDTSINTAYMELSRLRSDDTAVYYCARDRGNYRAWFGYWGQGTLVTVSS

SEQ ID NO: 38 - protein, 120 aa, synthetic construct
  REGION 1..120: note = Heavy Chain of samrotamab IgG histidine scanning variant #26
EVQLVQSGAEVKKPGASVKVSCKASGYKFSSYWIEWVKQAPGQGLEWIGEILPGSDTTNYNEKFHDRATFTSDTSINTAYMELSRLRSDDTAVYYCARDRGNYRAWFGYWGQGTLVTVSS

SEQ ID NO: 39 - protein, 120 aa, synthetic construct
  REGION 1..120: note = Heavy Chain of samrotamab IgG histidine scanning variant #27
EVQLVQSGAEVKKPGASVKVSCKASGYKFSSYWIEWVKQAPGQGLEWIGEILPGSDTTNYNEKFKHRATFTSDTSINTAYMELSRLRSDDTAVYYCARDRGNYRAWFGYWGQGTLVTVSS

SEQ ID NO: 40 - protein, 120 aa, synthetic construct
  REGION 1..120: note = Heavy Chain of samrotamab IgG histidine scanning variant #28
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCHRDRGNYRAWFGYWGQGTLVTVSS

SEQ ID NO: 41 - protein, 120 aa, synthetic construct
  REGION 1..120: note = Heavy Chain of samrotamab IgG histidine scanning variant #29
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCAHDRGNYRAWFGYWGQGTLVTVSS

SEQ ID NO: 42 - protein, 120 aa, synthetic construct
  REGION 1..120: note = Heavy Chain of samrotamab IgG histidine scanning variant #30
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARHRGNYRAWFGYWGQGTLVTVSS

SEQ ID NO: 43 - protein, 120 aa, synthetic construct
  REGION 1..120: note = Heavy Chain of samrotamab IgG histidine scanning variant #31
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDHGNYRAWFGYWGQGTLVTVSS

SEQ ID NO: 44 - protein, 120 aa, synthetic construct
  REGION 1..120: note = Heavy Chain of samrotamab IgG histidine scanning variant #32
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRHNYRAWFGYWGQGTLVTVSS

SEQ ID NO: 45 - protein, 120 aa, synthetic construct
  REGION 1..120: note = Heavy Chain of samrotamab IgG histidine scanning variant #33
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGHYRAWFGYWGQGTLVTVSS

SEQ ID NO: 46 - protein, 120 aa, synthetic construct
  REGION 1..120: note = Heavy Chain of samrotamab IgG histidine scanning variant #34
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNHRAWFGYWGQGTLVTVSS

SEQ ID NO: 47 - protein, 120 aa, synthetic construct
  REGION 1..120: note = Heavy Chain of samrotamab IgG histidine scanning variant #35
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYHAWFGYWGQGTLVTVSS

SEQ ID NO: 48 - protein, 120 aa, synthetic construct
  REGION 1..120: note = Heavy Chain of samrotamab IgG histidine scanning variant #36
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRHWFGYWGQGTLVTVSS

SEQ ID NO: 49 - protein, 120 aa, synthetic construct
  REGION 1..120: note = Heavy Chain of samrotamab IgG histidine scanning variant #37
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAHFGYWGQGTLVTVSS

SEQ ID NO: 50 - protein, 120 aa, synthetic construct
  REGION 1..120: note = Heavy Chain of samrotamab IgG histidine scanning variant #38
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWHGYWGQGTLVTVSS

SEQ ID NO: 51 - protein, 120 aa, synthetic construct
  REGION 1..120: note = Heavy Chain of samrotamab IgG histidine scanning variant #39
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFHYWGQGTLVTVSS

SEQ ID NO: 52 - protein, 120 aa, synthetic construct
  REGION 1..120: note = Heavy Chain of samrotamab IgG histidine scanning variant #40
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGHWGQGTLVTVSS

SEQ ID NO: 53 - protein, 107 aa, synthetic construct
  REGION 1..107: note = Light Chain of samrotamab IgG histidine scanning variant #1?
DIQMTQSPSSLSASVGDRVTITCHASQDISNYLNWYQQKPGGAVKFLIYYTSRLHSGVPSRFSGSGSGTDYTLTISSLQPEDFATYFCQQGEALPWTFGGGTKVEIK

SEQ ID NO: 54 - protein, 107 aa, synthetic construct
  REGION 1..107: note = Light Chain of samrotamab IgG histidine scanning variant #2
DIQMTQSPSSLSASVGDRVTITCRHSQDISNYLNWYQQKPGGAVKFLIYYTSRLHSGVPSRFSGSGSGTDYTLTISSLQPEDFATYFCQQGEALPWTFGGGTKVEIK

SEQ ID NO: 55 - protein, 107 aa, synthetic construct
  REGION 1..107: note = Light Chain of samrotamab IgG histidine scanning variant #3
DIQMTQSPSSLSASVGDRVTITCRAHQDISNYLNWYQQKPGGAVKFLIYYTSRLHSGVPSRFSGSGSGTDYTLTISSLQPEDFATYFCQQGEALPWTFGGGTKVEIK

SEQ ID NO: 56 - protein, 107 aa, synthetic construct
  REGION 1..107: note = Light Chain of samrotamab IgG histidine scanning variant #4
DIQMTQSPSSLSASVGDRVTITCRASHDISNYLNWYQQKPGGAVKFLIYYTSRLHSGVPSRFSGSGSGTDYTLTISSLQPEDFATYFCQQGEALPWTFGGGTKVEIK

SEQ ID NO: 57 - protein, 107 aa, synthetic construct
  REGION 1..107: note = Light Chain of samrotamab IgG histidine scanning variant #5
DIQMTQSPSSLSASVGDRVTITCRASQHISNYLNWYQQKPGGAVKFLIYYTSRLHSGVPSRFSGSGSGTDYTLTISSLQPEDFATYFCQQGEALPWTFGGGTKVEIK

SEQ ID NO: 58 - protein, 107 aa, synthetic construct
  REGION 1..107: note = Light Chain of samrotamab IgG histidine scanning variant #6
DIQMTQSPSSLSASVGDRVTITCRASQDHSNYLNWYQQKPGGAVKFLIYYTSRLHSGVPSRFSGSGSGTDYTLTISSLQPEDFATYFCQQGEALPWTFGGGTKVEIK

SEQ ID NO: 59 - protein, 107 aa, synthetic construct
  REGION 1..107: note = Light Chain of samrotamab IgG histidine scanning variant #7
DIQMTQSPSSLSASVGDRVTITCRASQDIHNYLNWYQQKPGGAVKFLIYYTSRLHSGVPSRFSGSGSGTDYTLTISSLQPEDFATYFCQQGEALPWTFGGGTKVEIK

SEQ ID NO: 60 - protein, 107 aa, synthetic construct
  REGION 1..107: note = Light Chain of samrotamab IgG histidine scanning variant #8
DIQMTQSPSSLSASVGDRVTITCRASQDISHYLNWYQQKPGGAVKFLIYYTSRLHSGVPSRFSGSGSGTDYTLTISSLQPEDFATYFCQQGEALPWTFGGGTKVEIK

SEQ ID NO: 61 - protein, 107 aa, synthetic construct
  REGION 1..107: note = Light Chain of samrotamab IgG histidine scanning variant #9
DIQMTQSPSSLSASVGDRVTITCRASQDISNHLNWYQQKPGGAVKFLIYYTSRLHSGVPSRFSGSGSGTDYTLTISSLQPEDFATYFCQQGEALPWTFGGGTKVEIK

SEQ ID NO: 62 - protein, 107 aa, synthetic construct
  REGION 1..107: note = Light Chain of samrotamab IgG histidine scanning variant #10
DIQMTQSPSSLSASVGDRVTITCRASQDISNYHNWYQQKPGGAVKFLIYYTSRLHSGVPSRFSGSGSGTDYTLTISSLQPEDFATYFCQQGEALPWTFGGGTKVEIK

SEQ ID NO: 63 - protein, 107 aa, synthetic construct
  REGION 1..107: note = Light Chain of samrotamab IgG histidine scanning variant #11
DIQMTQSPSSLSASVGDRVTITCRASQDISNYLHWYQQKPGGAVKFLIYYTSRLHSGVPSRFSGSGSGTDYTLTISSLQPEDFATYFCQQGEALPWTFGGGTKVEIK

SEQ ID NO: 64 - protein, 107 aa, synthetic construct
  REGION 1..107: note = Light Chain of samrotamab IgG histidine scanning variant #12
DIQMTQSPSSLSASVGDRVTITCRASQDISNYLNWYQQKPGGAVKFLIYHTSRLHSGVPSRFSGSGSGTDYTLTISSLQPEDFATYFCQQGEALPWTFGGGTKVEIK

SEQ ID NO: 65 - protein, 107 aa, synthetic construct
  REGION 1..107: note = Light Chain of samrotamab IgG histidine scanning variant #13
DIQMTQSPSSLSASVGDRVTITCRASQDISNYLNWYQQKPGGAVKFLIYYHSRLHSGVPSRFSGSGSGTDYTLTISSLQPEDFATYFCQQGEALPWTFGGGTKVEIK

SEQ ID NO: 66 - protein, 107 aa, synthetic construct
  REGION 1..107: note = Light Chain of samrotamab IgG histidine scanning variant #14
DIQMTQSPSSLSASVGDRVTITCRASQDISNYLNWYQQKPGGAVKFLIYYTHRLHSGVPSRFSGSGSGTDYTLTISSLQPEDFATYFCQQGEALPWTFGGGTKVEIK

SEQ ID NO: 67 - protein, 107 aa, synthetic construct
  REGION 1..107: note = Light Chain of samrotamab IgG histidine scanning variant #15
DIQMTQSPSSLSASVGDRVTITCRASQDISNYLNWYQQKPGGAVKFLIYYTSHLHSGVPSRFSGSGSGTDYTLTISSLQPEDFATYFCQQGEALPWTFGGGTKVEIK

SEQ ID NO: 68 - protein, 107 aa, synthetic construct
  REGION 1..107: note = Light Chain of samrotamab IgG histidine scanning variant #16
DIQMTQSPSSLSASVGDRVTITCRASQDISNYLNWYQQKPGGAVKFLIYYTSRHHSGVPSRFSGSGSGTDYTLTISSLQPEDFATYFCQQGEALPWTFGGGTKVEIK

SEQ ID NO: 69 - protein, 107 aa, synthetic construct
  REGION 1..107: note = Light Chain of samrotamab IgG histidine scanning variant #17
DIQMTQSPSSLSASVGDRVTITCRASQDISNYLNWYQQKPGGAVKFLIYYTSRLASGVPSRFSGSGSGTDYTLTISSLQPEDFATYFCQQGEALPWTFGGGTKVEIK

SEQ ID NO: 70 - protein, 107 aa, synthetic construct
  REGION 1..107: note = Light Chain of samrotamab IgG histidine scanning variant #18
DIQMTQSPSSLSASVGDRVTITCRASQDISNYLNWYQQKPGGAVKFLIYYTSRLHHGVPSRFSGSGSGTDYTLTISSLQPEDFATYFCQQGEALPWTFGGGTKVEIK

SEQ ID NO: 71 - protein, 107 aa, synthetic construct
  REGION 1..107: note = Light Chain of samrotamab IgG histidine scanning variant #19
DIQMTQSPSSLSASVGDRVTITCRASQDISNYLNWYQQKPGGAVKFLIYYTSRLHSGVPSRFSGSGSGTDYTLTISSLQPEDFATYFCHQGEALPWTFGGGTKVEIK

SEQ ID NO: 72 - protein, 107 aa, synthetic construct
  REGION 1..107: note = Light Chain of samrotamab IgG histidine scanning variant #20
DIQMTQSPSSLSASVGDRVTITCRASQDISNYLNWYQQKPGGAVKFLIYYTSRLHSGVPSRFSGSGSGTDYTLTISSLQPEDFATYFCQHGEALPWTFGGGTKVEIK

SEQ ID NO: 73 - protein, 107 aa, synthetic construct
  REGION 1..107: note = Light Chain of samrotamab IgG histidine scanning variant #21
DIQMTQSPSSLSASVGDRVTITCRASQDISNYLNWYQQKPGGAVKFLIYYTSRLHSGVPSRFSGSGSGTDYTLTISSLQPEDFATYFCQQHEALPWTFGGGTKVEIK

SEQ ID NO: 74 - protein, 107 aa, synthetic construct
  REGION 1..107: note = Light Chain of samrotamab IgG histidine scanning variant #22
DIQMTQSPSSLSASVGDRVTITCRASQDISNYLNWYQQKPGGAVKFLIYYTSRLHSGVPSRFSGSGSGTDYTLTISSLQPEDFATYFCQQGHALPWTFGGGTKVEIK

SEQ ID NO: 75 - protein, 107 aa, synthetic construct
  REGION 1..107: note = Light Chain of samrotamab IgG histidine scanning variant #23
DIQMTQSPSSLSASVGDRVTITCRASQDISNYLNWYQQKPGGAVKFLIYYTSRLHSGVPSRFSGSGSGTDYTLTISSLQPEDFATYFCQQGEHLPWTFGGGTKVEIK

SEQ ID NO: 76 - protein, 107 aa, synthetic construct
  REGION 1..107: note = Light Chain of samrotamab IgG histidine scanning variant #24
DIQMTQSPSSLSASVGDRVTITCRASQDISNYLNWYQQKPGGAVKFLIYYTSRLHSGVPSRFSGSGSGTDYTLTISSLQPEDFATYFCQQGEAHPWTFGGGTKVEIK

SEQ ID NO: 77 - protein, 107 aa, synthetic construct
  REGION 1..107: note = Light Chain of samrotamab IgG histidine scanning variant #25
DIQMTQSPSSLSASVGDRVTITCRASQDISNYLNWYQQKPGGAVKFLIYYTSRLHSGVPSRFSGSGSGTDYTLTISSLQPEDFATYFCQQGEALHWTFGGGTKVEIK

SEQ ID NO: 78 - protein, 107 aa, synthetic construct
  REGION 1..107: note = Light Chain of samrotamab IgG histidine scanning variant #26
DIQMTQSPSSLSASVGDRVTITCRASQDISNYLNWYQQKPGGAVKFLIYYTSRLHSGVPSRFSGSGSGTDYTLTISSLQPEDFATYFCQQGEALPHTFGGGTKVEIK

SEQ ID NO: 79 - protein, 107 aa, synthetic construct
  REGION 1..107: note = Light Chain of samrotamab IgG histidine scanning variant #27
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWHFGGGTKVEIK

SEQ ID NO: 80 - protein, 120 aa, synthetic construct
  REGION 1..120: note = Heavy Chain Combinations of samrotamab IgG histidine scanning variant #1
EVQLVQSGAEVKKPGASVKVSCKASGYKFSSYWHEWVKQAPGQGLEWIGEILPGSDTTHYNEKFKDRATFTSDTSINTAYMELSRLRSDDTAVYYCARDRGNYRAWFGYWGQGTLVTVSS

SEQ ID NO: 81 - protein, 120 aa, synthetic construct
  REGION 1..120: note = Heavy Chain Combinations of samrotamab IgG histidine scanning variant #2
EVQLVQSGAEVKKPGASVKVSCKASGYKFSSYWHEWVKQAPGQGLEWIGEILPGSDTTNYNEKFKDRATFTSDTSINTAYMELSRLRSDDTAVYYCARDRGNHRAWFGYWGQGTLVTVSS

SEQ ID NO: 82 - protein, 120 aa, synthetic construct
  REGION 1..120: note = Heavy Chain Combinations of samrotamab IgG histidine scanning variant #3
EVQLVQSGAEVKKPGASVKVSCKASGYKFSSYWIEWVKQAPGQGLEWIGEILPGSDTTHYNEKFKDRATFTSDTSINTAYMELSRLRSDDTAVYYCARDRGNHRAWFGYWGQGTLVTVSS

SEQ ID NO: 83 - protein, 120 aa, synthetic construct
  REGION 1..120: note = Heavy Chain Combinations of samrotamab IgG histidine scanning variant #4
EVQLVQSGAEVKKPGASVKVSCKASGYKFSSYWHEWVKQAPGQGLEWIGEILPGSDTTHYNEKFKDRATFTSDTSINTAYMELSRLRSDDTAVYYCARDRGNHRAWFGYWGQGTLVTVSS

SEQ ID NO: 84 - protein, 120 aa, synthetic construct
  REGION 1..120: note = Heavy Chain of hu139.10 IgG
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 85 - protein, 112 aa, synthetic construct
  REGION 1..112: note = Light Chain of hu139.10 IgG
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 86 - protein, 10 aa, synthetic construct
  REGION 1..10: note = Heavy Chain CDR1 of hu139.10
GFSLTSYGVH

SEQ ID NO: 87 - protein, 16 aa, synthetic construct
  REGION 1..16: note = Heavy Chain CDR2 of hu139.10
VIWAGGSTNYNSALMS

SEQ ID NO: 88 - protein, 14 aa, synthetic construct
  REGION 1..14: note = Heavy Chain CDR3 of hu139.10
ATHMITEDYYGMDY

SEQ ID NO: 89 - protein, 17 aa, synthetic construct
  REGION 1..17: note = Light Chain CDR1 of hu139.10
KSSQSLLNSRTRKNYLA

SEQ ID NO: 90 - protein, 7 aa, synthetic construct
  REGION 1..7: note = Light Chain CDR2 of hu139.10
WASTRES

SEQ ID NO: 91 - protein, 8 aa, synthetic construct
  REGION 1..8: note = Light Chain CDR3 of hu139.10
KQSYNLPT

SEQ ID NO: 92 - protein, 120 aa, synthetic construct
  REGION 1..120: note = Heavy Chain of hu139.10 IgG histidine scanning variant #1
EVQLVESGGGLVQPGGSLRLSCAVSHFSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 93 - protein, 120 aa, synthetic construct
  REGION 1..120: note = Heavy Chain of hu139.10 IgG histidine scanning variant #2
EVQLVESGGGLVQPGGSLRLSCAVSGHSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 94 - protein, 120 aa, synthetic construct
  REGION 1..120: note = Heavy Chain of hu139.10 IgG histidine scanning variant #3
EVQLVESGGGLVQPGGSLRLSCAVSGFHLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 95 - protein, 120 aa, synthetic construct
  REGION 1..120: note = Heavy Chain of hu139.10 IgG histidine scanning variant #4
EVQLVESGGGLVQPGGSLRLSCAVSGFSHTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 96 - protein, 120 aa, synthetic construct
  REGION 1..120: note = Heavy Chain of hu139.10 IgG histidine scanning variant #5
EVQLVESGGGLVQPGGSLRLSCAVSGFSLHSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 97 - protein, 120 aa, synthetic construct
  REGION 1..120: note = Heavy Chain of hu139.10 IgG histidine scanning variant #6
EVQLVESGGGLVQPGGSLRLSCAVSGFSLTHYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 98 - protein, 120 aa, synthetic construct
  REGION 1..120: note = Heavy Chain of hu139.10 IgG histidine scanning variant #7
EVQLVESGGGLVQPGGSLRLSCAVSGFSLTSHGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 99 - protein, 120 aa, synthetic construct
  REGION 1..120: note = Heavy Chain of hu139.10 IgG histidine scanning variant #8
EVQLVESGGGLVQPGGSLRLSCAVSGFSLTSYHVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 100 - protein, 120 aa, synthetic construct
  REGION 1..120: note = Heavy Chain of hu139.10 IgG histidine scanning variant #9
EVQLVESGGGLVQPGGSLRLSCAVSGFSLTSYGHHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 101 - protein, 120 aa, synthetic construct
  REGION 1..120: note = Heavy Chain of hu139.10 IgG histidine scanning variant #10
EVQLVESGGGLVQPGGSLRLSCAVSGFSLTSYGVAWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 102 - protein, 120 aa, synthetic construct
  REGION 1..120: note = Heavy Chain of hu139.10 IgG histidine scanning variant #11
EVQLVESGGGLVQPGGSLRLSCAVSGFSLTSYGVHWVRQATGKGLEWLGHIWAGGSTNYNSALMSRLTISKENAKSSVYLQMNSLRAGDTAMYYCATHMITEDYYGMDYWGQGTTVTVSS

SEQ ID NO: 103 - protein, 120 aa, synthetic construct
  REGION 1..120: note = Heavy Chain of hu139.10 IgG histidine scanning variant #12
EVQLVESGGGLVQPGGSLRLSCAVSGFSLTSYGVHWVRQATGKGLEWLGVHWAGGSTNYNSALMSRLTISKENAKSSVYLQMNSLRAGDTAMYYCATHMITEDYYGMDYWGQGTTVTVSS

SEQ ID NO: 104 - protein, 120 aa, synthetic construct
  REGION 1..120: note = Heavy Chain of hu139.10 IgG histidine scanning variant #13
EVQLVESGGGLVQPGGSLRLSCAVSGFSLTSYGVHWVRQATGKGLEWLGVIHAGGSTNYNSALMSRLTISKENAKSSVYLQMNSLRAGDTAMYYCATHMITEDYYGMDYWGQGTTVTVSS

SEQ ID NO: 105 - protein, 120 aa, synthetic construct
  REGION 1..120: note = Heavy Chain of hu139.10 IgG histidine scanning variant #14
EVQLVESGGGLVQPGGSLRLSCAVSGFSLTSYGVHWVRQATGKGLEWLGVIWHGGSTNYNSALMSRLTISKENAKSSVYLQMNSLRAGDTAMYYCATHMITEDYYGMDYWGQGTTVTVSS

SEQ ID NO: 106 - protein, 120 aa, synthetic construct
  REGION 1..120: note = Heavy Chain of hu139.10 IgG histidine scanning variant #15
EVQLVESGGGLVQPGGSLRLSCAVSGFSLTSYGVHWVRQATGKGLEWLGVIWAHGSTNYNSALMSRLTISKENAKSSVYLQMNSLRAGDTAMYYCATHMITEDYYGMDYWGQGTTVTVSS

SEQ ID NO: 107 - protein, 120 aa, synthetic construct
  REGION 1..120: note = Heavy Chain of hu139.10 IgG histidine scanning variant #16
EVQLVESGGGLVQPGGSLRLSCAVSGFSLTSYGVHWVRQATGKGLEWLGVIWAGHSTNYNSALMSRLTISKENAKSSVYLQMNSLRAGDTAMYYCATHMITEDYYGMDYWGQGTTVTVSS

SEQ ID NO: 108 - protein, 120 aa, synthetic construct
  REGION 1..120: note = Heavy Chain of hu139.10 IgG histidine scanning variant #17
EVQLVESGGGLVQPGGSLRLSCAVSGFSLTSYGVHWVRQATGKGLEWLGVIWAGGHTNYNSALMSRLTISKENAKSSVYLQMNSLRAGDTAMYYCATHMITEDYYGMDYWGQGTTVTVSS

SEQ ID NO: 109 - protein, 120 aa, synthetic construct
  REGION 1..120: note = Heavy Chain of hu139.10 IgG histidine scanning variant #18
EVQLVESGGGLVQPGGSLRLSCAVSGFSLTSYGVHWVRQATGKGLEWLGVIWAGGSHNYNSALMSRLTISKENAKSSVYLQMNSLRAGDTAMYYCATHMITEDYYGMDYWGQGTTVTVSS

SEQ ID NO: 110 - protein, 120 aa, synthetic construct
  REGION 1..120: note = Heavy Chain of hu139.10 IgG histidine scanning variant #19
EVQLVESGGGLVQPGGSLRLSCAVSGFSLTSYGVHWVRQATGKGLEWLGVIWAGGSTHYNSALMSRLTISKENAKSSVYLQMNSLRAGDTAMYYCATHMITEDYYGMDYWGQGTTVTVSS

SEQ ID NO: 111 - protein, 120 aa, synthetic construct
  REGION 1..120: note = Heavy Chain of hu139.10 IgG histidine scanning variant #20
EVQLVESGGGLVQPGGSLRLSCAVSGFSLTSYGVHWVRQATGKGLEWLGVIWAGGSTNHNSALMSRLTISKENAKSSVYLQMNSLRAGDTAMYYCATHMITEDYYGMDYWGQGTTVTVSS

SEQ ID NO: 112 - protein, 120 aa, synthetic construct
  REGION 1..120: note = Heavy Chain of hu139.10 IgG histidine scanning variant #21
EVQLVESGGGLVQPGGSLRLSCAVSGFSLTSYGVHWVRQATGKGLEWLGVIWAGGSTNYHSALMSRLTISKENAKSSVYLQMNSLRAGDTAMYYCATHMITEDYYGMDYWGQGTTVTVSS

SEQ ID NO: 113 - protein, 120 aa, synthetic construct
  REGION 1..120: note = Heavy Chain of hu139.10 IgG histidine scanning variant #22
EVQLVESGGGLVQPGGSLRLSCAVSGFSLTSYGVHWVRQATGKGLEWLGVIWAGGSTNYNHALMSRLTISKENAKSSVYLQMNSLRAGDTAMYYCATHMITEDYYGMDYWGQGTTVTVSS

SEQ ID NO: 114 - protein, 120 aa, synthetic construct
  REGION 1..120: note = Heavy Chain of hu139.10 IgG histidine scanning variant #23
EVQLVESGGGLVQPGGSLRLSCAVSGFSLTSYGVHWVRQATGKGLEWLGVIWAGGSTNYNSHLMSRLTISKENAKSSVYLQMNSLRAGDTAMYYCATHMITEDYYGMDYWGQGTTVTVSS

SEQ ID NO: 115 - protein, 120 aa, synthetic construct
  REGION 1..120: note = Heavy Chain of hu139.10 IgG histidine scanning variant #24
EVQLVESGGGLVQPGGSLRLSCAVSGFSLTSYGVHWVRQATGKGLEWLGVIWAGGSTNYNSAHMSRLTISKENAKSSVYLQMNSLRAGDTAMYYCATHMITEDYYGMDYWGQGTTVTVSS

SEQ ID NO: 116 - protein, 120 aa, synthetic construct
  REGION 1..120: note = Heavy Chain of hu139.10 IgG histidine scanning variant #25
EVQLVESGGGLVQPGGSLRLSCAVSGFSLTSYGVHWVRQATGKGLEWLGVIWAGGSTNYNSALHSRLTISKENAKSSVYLQMNSLRAGDTAMYYCATHMITEDYYGMDYWGQGTTVTVSS

SEQ ID NO: 117 - protein, 120 aa, synthetic construct
  REGION 1..120: note = Heavy Chain of hu139.10 IgG histidine scanning variant #26
EVQLVESGGGLVQPGGSLRLSCAVSGFSLTSYGVHWVRQATGKGLEWLGVIWAGGSTNYNSALMHRLTISKENAKSSVYLQMNSLRAGDTAMYYCATHMITEDYYGMDYWGQGTTVTVSS

SEQ ID NO: 118 - protein, 120 aa, synthetic construct
  REGION 1..120: note = Heavy Chain of hu139.10 IgG histidine scanning variant #27
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCHTHMITEDYYGMDYWGQGTTVTVSS

SEQ ID NO: 119 - protein, 120 aa, synthetic construct
  REGION 1..120: note = Heavy Chain of hu139.10 IgG histidine scanning variant #28
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCAHHMITEDYYGMDYWGQGTTVTVSS

SEQ ID NO: 120 - protein, 120 aa, synthetic construct
  REGION 1..120: note = Heavy Chain of hu139.10 IgG histidine scanning variant #29
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATAMITEDYYGMDYWGQGTTVTVSS

SEQ ID NO: 121 - protein, 120 aa, synthetic construct
  REGION 1..120: note = Heavy Chain of hu139.10 IgG histidine scanning variant #30
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHHITEDYYGMDYWGQGTTVTVSS

SEQ ID NO: 122 - protein, 120 aa, synthetic construct
  REGION 1..120: note = Heavy Chain of hu139.10 IgG histidine scanning variant #31
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMHTEDYYGMDYWGQGTTVTVSS

SEQ ID NO: 123 - protein, 120 aa, synthetic construct
  REGION 1..120: note = Heavy Chain of hu139.10 IgG histidine scanning variant #32
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMIHEDYYGMDYWGQGTTVTVSS

SEQ ID NO: 124 - protein, 120 aa, synthetic construct
  REGION 1..120: note = Heavy Chain of hu139.10 IgG histidine scanning variant #33
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITHDYYGMDYWGQGTTVTVSS

SEQ ID NO: 125 - protein, 120 aa, synthetic construct
  REGION 1..120: note = Heavy Chain of hu139.10 IgG histidine scanning variant #34
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEHYYGMDYWGQGTTVTVSS

SEQ ID NO: 126 - protein, 120 aa, synthetic construct
  REGION 1..120: note = Heavy Chain of hu139.10 IgG histidine scanning variant #35
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDHYGMDYWGQGTTVTVSS

SEQ ID NO: 127 - protein, 120 aa, synthetic construct
  REGION 1..120: note = Heavy Chain of hu139.10 IgG histidine scanning variant #36
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYHGMDYWGQGTTVTVSS

SEQ ID NO: 128 - protein, 120 aa, synthetic construct
  REGION 1..120: note = Heavy Chain of hu139.10 IgG histidine scanning variant #37
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYHMDYWGQGTTVTVSS

SEQ ID NO: 129 - protein, 120 aa, synthetic construct
  REGION 1..120: note = Heavy Chain of hu139.10 IgG histidine scanning variant #38
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGHDYWGQGTTVTVSS

SEQ ID NO: 130 - protein, 120 aa, synthetic construct
  REGION 1..120: note = Heavy Chain of hu139.10 IgG histidine scanning variant #39
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMHYWGQGTTVTVSS

SEQ ID NO: 131 - protein, 120 aa, synthetic construct
  REGION 1..120: note = Heavy Chain of hu139.10 IgG histidine scanning variant #40
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDHWGQGTTVTVSS

SEQ ID NO: 132 - protein, 112 aa, synthetic construct
  REGION 1..112: note = Light Chain of hu139.10 IgG histidine scanning variant #1
DIVMTQSPDSLAVSLGERATINCHSSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 133 - protein, 112 aa, synthetic construct
  REGION 1..112: note = Light Chain of hu139.10 IgG histidine scanning variant #2
DIVMTQSPDSLAVSLGERATINCKHSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 134 - protein, 112 aa, synthetic construct
  REGION 1..112: note = Light Chain of hu139.10 IgG histidine scanning variant #3
DIVMTQSPDSLAVSLGERATINCKSHQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 135 - protein, 112 aa, synthetic construct
  REGION 1..112: note = Light Chain of hu139.10 IgG histidine scanning variant #4
DIVMTQSPDSLAVSLGERATINCKSSHSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 136 - protein, 112 aa, synthetic construct
  REGION 1..112: note = Light Chain of hu139.10 IgG histidine scanning variant #5
DIVMTQSPDSLAVSLGERATINCKSSQHLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 137 - protein, 112 aa, synthetic construct
  REGION 1..112: note = Light Chain of hu139.10 IgG histidine scanning variant #6
DIVMTQSPDSLAVSLGERATINCKSSQSHLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 138 - protein, 112 aa, synthetic construct
  REGION 1..112: note = Light Chain of hu139.10 IgG histidine scanning variant #7
DIVMTQSPDSLAVSLGERATINCKSSQSLHNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 139 - protein, 112 aa, synthetic construct
  REGION 1..112: note = Light Chain of hu139.10 IgG histidine scanning variant #8
DIVMTQSPDSLAVSLGERATINCKSSQSLLHSRTRKNYLAWYQQKPGQSPKLLIYWASTRESGVPDRFSGSGSGTDFTLTISSLQAEDVAVYYCKQSYNLPTFGGGTKVEIK

SEQ ID NO: 140 - protein, 112 aa, synthetic construct
  REGION 1..112: note = Light Chain of hu139.10 IgG histidine scanning variant #9
DIVMTQSPDSLAVSLGERATINCKSSQSLLNHRTRKNYLAWYQQKPGQSPKLLIYWASTRESGVPDRFSGSGSGTDFTLTISSLQAEDVAVYYCKQSYNLPTFGGGTKVEIK

SEQ ID NO: 141 - protein, 112 aa, synthetic construct
  REGION 1..112: note = Light Chain of hu139.10 IgG histidine scanning variant #10
DIVMTQSPDSLAVSLGERATINCKSSQSLLNSHTRKNYLAWYQQKPGQSPKLLIYWASTRESGVPDRFSGSGSGTDFTLTISSLQAEDVAVYYCKQSYNLPTFGGGTKVEIK

SEQ ID NO: 142 - protein, 112 aa, synthetic construct
  REGION 1..112: note = Light Chain of hu139.10 IgG histidine scanning variant #11
DIVMTQSPDSLAVSLGERATINCKSSQSLLNSRHRKNYLAWYQQKPGQSPKLLIYWASTRESGVPDRFSGSGSGTDFTLTISSLQAEDVAVYYCKQSYNLPTFGGGTKVEIK

SEQ ID NO: 143 - protein, 112 aa, synthetic construct
  REGION 1..112: note = Light Chain of hu139.10 IgG histidine scanning variant #12
DIVMTQSPDSLAVSLGERATINCKSSQSLLNSRTHKNYLAWYQQKPGQSPKLLIYWASTRESGVPDRFSGSGSGTDFTLTISSLQAEDVAVYYCKQSYNLPTFGGGTKVEIK

SEQ ID NO: 144 - protein, 112 aa, synthetic construct
  REGION 1..112: note = Light Chain of hu139.10 IgG histidine scanning variant #13
DIVMTQSPDSLAVSLGERATINCKSSQSLLNSRTRHNYLAWYQQKPGQSPKLLIYWASTRESGVPDRFSGSGSGTDFTLTISSLQAEDVAVYYCKQSYNLPTFGGGTKVEIK

SEQ ID NO: 145 - protein, 112 aa, synthetic construct
  REGION 1..112: note = Light Chain of hu139.10 IgG histidine scanning variant #14
DIVMTQSPDSLAVSLGERATINCKSSQSLLNSRTRKHYLAWYQQKPGQSPKLLIYWASTRESGVPDRFSGSGSGTDFTLTISSLQAEDVAVYYCKQSYNLPTFGGGTKVEIK

SEQ ID NO: 146 - protein, 112 aa, synthetic construct
  REGION 1..112: note = Light Chain of hu139.10 IgG histidine scanning variant #15
DIVMTQSPDSLAVSLGERATINCKSSQSLLNSRTRKNHLAWYQQKPGQSPKLLIYWASTRESGVPDRFSGSGSGTDFTLTISSLQAEDVAVYYCKQSYNLPTFGGGTKVEIK

SEQ ID NO: 147 - protein, 112 aa, synthetic construct
  REGION 1..112: note = Light Chain of hu139.10 IgG histidine scanning variant #16
DIVMTQSPDSLAVSLGERATINCKSSQSLLNSRTRKNYHAWYQQKPGQSPKLLIYWASTRESGVPDRFSGSGSGTDFTLTISSLQAEDVAVYYCKQSYNLPTFGGGTKVEIK

SEQ ID NO: 148 - protein, 112 aa, synthetic construct
  REGION 1..112: note = Light Chain of hu139.10 IgG histidine scanning variant #17
DIVMTQSPDSLAVSLGERATINCKSSQSLLNSRTRKNYLHWYQQKPGQSPKLLIYWASTRESGVPDRFSGSGSGTDFTLTISSLQAEDVAVYYCKQSYNLPTFGGGTKVEIK

SEQ ID NO: 149 - protein, 112 aa, synthetic construct
  REGION 1..112: note = Light Chain of hu139.10 IgG histidine scanning variant #18
DIVMTQSPDSLAVSLGERATINCKSSQSLLNSRTRKNYLAWYQQKPGQSPKLLIYHASTRESGVPDRFSGSGSGTDFTLTISSLQAEDVAVYYCKQSYNLPTFGGGTKVEIK

SEQ ID NO: 150 - protein, 112 aa, synthetic construct
  REGION 1..112: note = Light Chain of hu139.10 IgG histidine scanning variant #19
DIVMTQSPDSLAVSLGERATINCKSSQSLLNSRTRKNYLAWYQQKPGQSPKLLIYWHSTRESGVPDRFSGSGSGTDFTLTISSLQAEDVAVYYCKQSYNLPTFGGGTKVEIK

SEQ ID NO: 151 - protein, 112 aa, synthetic construct
  REGION 1..112: note = Light Chain of hu139.10 IgG histidine scanning variant #20
DIVMTQSPDSLAVSLGERATINCKSSQSLLNSRTRKNYLAWYQQKPGQSPKLLIYWAHTRESGVPDRFSGSGSGTDFTLTISSLQAEDVAVYYCKQSYNLPTFGGGTKVEIK

SEQ ID NO: 152 - protein, 112 aa, synthetic construct
  REGION 1..112: note = Light Chain of hu139.10 IgG histidine scanning variant #21
DIVMTQSPDSLAVSLGERATINCKSSQSLLNSRTRKNYLAWYQQKPGQSPKLLIYWASHRESGVPDRFSGSGSGTDFTLTISSLQAEDVAVYYCKQSYNLPTFGGGTKVEIK

SEQ ID NO: 153 - protein, 112 aa, synthetic construct
  REGION 1..112: note = Light Chain of hu139.10 IgG histidine scanning variant #22
DIVMTQSPDSLAVSLGERATINCKSSQSLLNSRTRKNYLAWYQQKPGQSPKLLIYWASTHESGVPDRFSGSGSGTDFTLTISSLQAEDVAVYYCKQSYNLPTFGGGTKVEIK

SEQ ID NO: 154 - protein, 112 aa, synthetic construct
  REGION 1..112: note = Light Chain of hu139.10 IgG histidine scanning variant #23
DIVMTQSPDSLAVSLGERATINCKSSQSLLNSRTRKNYLAWYQQKPGQSPKLLIYWASTRHSGVPDRFSGSGSGTDFTLTISSLQAEDVAVYYCKQSYNLPTFGGGTKVEIK

SEQ ID NO: 155 - protein, 112 aa, synthetic construct
  REGION 1..112: note = Light Chain of hu139.10 IgG histidine scanning variant #24
DIVMTQSPDSLAVSLGERATINCKSSQSLLNSRTRKNYLAWYQQKPGQSPKLLIYWASTREHGVPDRFSGSGSGTDFTLTISSLQAEDVAVYYCKQSYNLPTFGGGTKVEIK

SEQ ID NO: 156 - protein, 112 aa, synthetic construct
  REGION 1..112: note = Light Chain of hu139.10 IgG histidine scanning variant #25
DIVMTQSPDSLAVSLGERATINCKSSQSLLNSRTRKNYLAWYQQKPGQSPKLLIYWASTRESGVPDRFSGSGSGTDFTLTISSLQAEDVAVYYCHQSYNLPTFGGGTKVEIK

SEQ ID NO: 157 - protein, 112 aa, synthetic construct
  REGION 1..112: note = Light Chain of hu139.10 IgG histidine scanning variant #26
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKHSYNLPTFGGGTKVEIK

SEQ ID NO: 158 - protein, 112 aa, synthetic construct
  REGION 1..112: note = Light Chain of hu139.10 IgG histidine scanning variant #27
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQHYNLPTFGGGTKVEIK

SEQ ID NO: 159 - protein, 112 aa, synthetic construct
  REGION 1..112: note = Light Chain of hu139.10 IgG histidine scanning variant #28
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSHNLPTFGGGTKVEIK

SEQ ID NO: 160 - protein, 112 aa, synthetic construct
  REGION 1..112: note = Light Chain of hu139.10 IgG histidine scanning variant #29
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYHLPTFGGGTKVEIK

SEQ ID NO: 161 - protein, 112 aa, synthetic construct
  REGION 1..112: note = Light Chain of hu139.10 IgG histidine scanning variant #30
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNHPTFGGGTKVEIK

SEQ ID NO: 162 - protein, 112 aa, synthetic construct
  REGION 1..112: note = Light Chain of hu139.10 IgG histidine scanning variant #31
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLHTFGGGTKVEIK

SEQ ID NO: 163 - protein, 112 aa, synthetic construct
  REGION 1..112: note = Light Chain of hu139.10 IgG histidine scanning variant #32
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPHFGGGTKVEIK

SEQ ID NO: 164 - protein, 120 aa, synthetic construct
  REGION 1..120: note = Heavy Chain Combinations of hu139.10 IgG histidine scanning variant #1
EVQLVESGGGLVQPGGSLRLSCAVSGFSLTSYGVHWVRQATGKGLEWLGVIWAGGHTNYNSALMSRLTISKENAKSSVYLQMNSLRAGDTAMYYCATAMITEDYYGMDYWGQGTTVTVSS

SEQ ID NO: 165 - protein, 120 aa, synthetic construct
  REGION 1..120: note = Heavy Chain Combinations of hu139.10 IgG histidine scanning variant #2
EVQLVESGGGLVQPGGSLRLSCAVSGFSLTSYGVHWVRQATGKGLEWLGVIWAGGHTNYNSALMSRLTISKENAKSSVYLQMNSLRAGDTAMYYCATHMITEDHYGMDYWGQGTTVTVSS

SEQ ID NO: 166 - protein, 120 aa, synthetic construct
  REGION 1..120: note = Heavy Chain Combinations of hu139.10 IgG histidine scanning variant #3
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATAMITEDHYGMDYWGQGTTVTVSS

SEQ ID NO: 167 - protein, 120 aa, synthetic construct
  REGION 1..120: note = Heavy Chain Combinations of hu139.10 IgG histidine scanning variant #4
EVQLVESGGGLVQPGGSLRLSCAVSGFSLTSYGVHWVRQATGKGLEWLGVIWAGGHTNYNSALMSRLTISKENAKSSVYLQMNSLRAGDTAMYYCATAMITEDHYGMDYWGQGTTVTVSS

SEQ ID NO: 168 - protein, 112 aa, synthetic construct
  REGION 1..112: note = Light Chain Combinations of hu139.10 IgG histidine scanning variant #1
DIVMTQSPDSLAVSLGERATINCKSSQSLLNSRTRKNYLHWYQQKPGQSPKLLIYHASTRESGVPDRFSGSGSGTDFTLTISSLQAEDVAVYYCKQSYNLPTFGGGTKVEIK

SEQ ID NO: 169 - protein, 112 aa, synthetic construct
  REGION 1..112: note = Light Chain Combinations of hu139.10 IgG histidine scanning variant #2
DIVMTQSPDSLAVSLGERATINCKSSQSLLNSRTRKNYLHWYQQKPGQSPKLLIYWASTRHSGVPDRFSGSGSGTDFTLTISSLQAEDVAVYYCKQSYNLPTFGGGTKVEIK

SEQ ID NO: 170 - protein, 112 aa, synthetic construct
  REGION 1..112: note = Light Chain Combinations of hu139.10 IgG histidine scanning variant #3
DIVMTQSPDSLAVSLGERATINCKSSQSLLNSRTRKNYLHWYQQKPGQSPKLLIYWASTRESGVPDRFSGSGSGTDFTLTISSLQAEDVAVYYCKQSYNHPTFGGGTKVEIK

SEQ ID NO: 171 - protein, 112 aa, synthetic construct
  REGION 1..112: note = Light Chain Combinations of hu139.10 IgG histidine scanning variant #4
DIVMTQSPDSLAVSLGERATINCKSSQSLLNSRTRKNYLAWYQQKPGQSPKLLIYHASTRHSGVPDRFSGSGSGTDFTLTISSLQAEDVAVYYCKQSYNLPTFGGGTKVEIK

SEQ ID NO: 172 - protein, 112 aa, synthetic construct
  REGION 1..112: note = Light Chain Combinations of hu139.10 IgG histidine scanning variant #5
DIVMTQSPDSLAVSLGERATINCKSSQSLLNSRTRKNYLAWYQQKPGQSPKLLIYHASTRESGVPDRFSGSGSGTDFTLTISSLQAEDVAVYYCKQSYNHPTFGGGTKVEIK

SEQ ID NO: 173 - protein, 112 aa, synthetic construct
  REGION 1..112: note = Light Chain Combinations of hu139.10 IgG histidine scanning variant #6
DIVMTQSPDSLAVSLGERATINCKSSQSLLNSRTRKNYLAWYQQKPGQSPKLLIYWASTRHSGVPDRFSGSGSGTDFTLTISSLQAEDVAVYYCKQSYNHPTFGGGTKVEIK

SEQ ID NO: 174 - protein, 112 aa, synthetic construct
  REGION 1..112: note = Light Chain Combinations of hu139.10 IgG histidine scanning variant #7
DIVMTQSPDSLAVSLGERATINCKSSQSLLNSRTRKNYLHWYQQKPGQSPKLLIYHASTRHSGVPDRFSGSGSGTDFTLTISSLQAEDVAVYYCKQSYNLPTFGGGTKVEIK

SEQ ID NO: 175 - protein, 112 aa, synthetic construct
  REGION 1..112: note = Light Chain Combinations of hu139.10 IgG histidine scanning variant #8
DIVMTQSPDSLAVSLGERATINCKSSQSLLNSRTRKNYLHWYQQKPGQSPKLLIYHASTRESGVPDRFSGSGSGTDFTLTISSLQAEDVAVYYCKQSYNHPTFGGGTKVEIK

SEQ ID NO: 176 - protein, 112 aa, synthetic construct
  REGION 1..112: note = Light Chain Combinations of hu139.10 IgG histidine scanning variant #9
DIVMTQSPDSLAVSLGERATINCKSSQSLLNSRTRKNYLHWYQQKPGQSPKLLIYWASTRHSGVPDRFSGSGSGTDFTLTISSLQAEDVAVYYCKQSYNHPTFGGGTKVEIK

SEQ ID NO: 177 - protein, 112 aa, synthetic construct
  REGION 1..112: note = Light Chain Combinations of hu139.10 IgG histidine scanning variant #10
DIVMTQSPDSLAVSLGERATINCKSSQSLLNSRTRKNYLAWYQQKPGQSPKLLIYHASTRHSGVPDRFSGSGSGTDFTLTISSLQAEDVAVYYCKQSYNHPTFGGGTKVEIK

SEQ ID NO: 178 - protein, 119 aa, synthetic construct
  REGION 1..119: note = Heavy Chain of huAD208.4.1 IgG
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 179 - protein, 110 aa, synthetic construct
  REGION 1..110: note = Light Chain of huAD208.4.1 IgG
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 180 - protein, 13 aa, synthetic construct
  REGION 1..13: note = Heavy Chain CDR1 of huAD208.4.1
KASGFTFTDYYIH

SEQ ID NO: 181 - protein, 17 aa, synthetic construct
  REGION 1..17: note = Heavy Chain CDR2 of huAD208.4.1
LVYPYIGGTNYNQKFKG

SEQ ID NO: 182 - protein, 12 aa, synthetic construct
  REGION 1..12: note = Heavy Chain CDR3 of huAD208.4.1
ARGDNKYDAMDY

SEQ ID NO: 183 - protein, 15 aa, synthetic construct
  REGION 1..15: note = Light Chain CDR1 of huAD208.4.1
RASQSVSTSSYSYMH

SEQ ID NO: 184 - protein, 7 aa, synthetic construct
  REGION 1..7: note = Light Chain CDR2 of huAD208.4.1
YASSLES

SEQ ID NO: 185 - protein, 8 aa, synthetic construct
  REGION 1..8: note = Light Chain CDR3 of huAD208.4.1
EQSWEIRT

SEQ ID NO: 186 - protein, 119 aa, synthetic construct
  REGION 1..119: note = Heavy Chain of huAD208.4.1 IgG histidine scanning variant #1
EVQLVQSGAEVKKPGSSVKVSCHASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 187 - protein, 119 aa, synthetic construct
  REGION 1..119: note = Heavy Chain of huAD208.4.1 IgG histidine scanning variant #2
EVQLVQSGAEVKKPGSSVKVSCKHSGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 188 - protein, 119 aa, synthetic construct
  REGION 1..119: note = Heavy Chain of huAD208.4.1 IgG histidine scanning variant #3
EVQLVQSGAEVKKPGSSVKVSCKAHGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 189 - protein, 119 aa, synthetic construct
  REGION 1..119: note = Heavy Chain of huAD208.4.1 IgG histidine scanning variant #4
EVQLVQSGAEVKKPGSSVKVSCKASHFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 190 - protein, 119 aa, synthetic construct
  REGION 1..119: note = Heavy Chain of huAD208.4.1 IgG histidine scanning variant #5
EVQLVQSGAEVKKPGSSVKVSCKASGHTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 191 - protein, 119 aa, synthetic construct
  REGION 1..119: note = Heavy Chain of huAD208.4.1 IgG histidine scanning variant #6
EVQLVQSGAEVKKPGSSVKVSCKASGFHFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 192 - protein, 119 aa, synthetic construct
  REGION 1..119: note = Heavy Chain of huAD208.4.1 IgG histidine scanning variant #7
EVQLVQSGAEVKKPGSSVKVSCKASGFTHTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 193 - protein, 119 aa, synthetic construct
  REGION 1..119: note = Heavy Chain of huAD208.4.1 IgG histidine scanning variant #8
EVQLVQSGAEVKKPGSSVKVSCKASGFTFHDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 194 - protein, 119 aa, synthetic construct
  REGION 1..119: note = Heavy Chain of huAD208.4.1 IgG histidine scanning variant #9
EVQLVQSGAEVKKPGSSVKVSCKASGFTFTHYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 195 - protein, 119 aa, synthetic construct
  REGION 1..119: note = Heavy Chain of huAD208.4.1 IgG histidine scanning variant #10
EVQLVQSGAEVKKPGSSVKVSCKASGFTFTDHYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 196 - protein, 119 aa, synthetic construct
  REGION 1..119: note = Heavy Chain of huAD208.4.1 IgG histidine scanning variant #11
EVQLVQSGAEVKKPGSSVKVSCKASGFTFTDYHIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 197 - protein, 119 aa, synthetic construct
  REGION 1..119: note = Heavy Chain of huAD208.4.1 IgG histidine scanning variant #12
EVQLVQSGAEVKKPGSSVKVSCKASGFTFTDYYHHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 198 - protein, 119 aa, synthetic construct
  REGION 1..119: note = Heavy Chain of huAD208.4.1 IgG histidine scanning variant #13
EVQLVQSGAEVKKPGSSVKVSCKASGFTFTDYYIAWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 199 - protein, 119 aa, synthetic construct
  REGION 1..119: note = Heavy Chain of huAD208.4.1 IgG histidine scanning variant #14
EVQLVQSGAEVKKPGSSVKVSCKASGFTFTDYYIHWVKQAPGQGLEWIGHVYPYIGGTNYNQKFKGKATLTVDTSTTTAYMEMSSLRSEDTAVYYCARGDNKYDAMDYWGQGTTVTVSS

SEQ ID NO: 200 - protein, 119 aa, synthetic construct
  REGION 1..119: note = Heavy Chain of huAD208.4.1 IgG histidine scanning variant #15
EVQLVQSGAEVKKPGSSVKVSCKASGFTFTDYYIHWVKQAPGQGLEWIGLHYPYIGGTNYNQKFKGKATLTVDTSTTTAYMEMSSLRSEDTAVYYCARGDNKYDAMDYWGQGTTVTVSS

SEQ ID NO: 201 - protein, 119 aa, synthetic construct
  REGION 1..119: note = Heavy Chain of huAD208.4.1 IgG histidine scanning variant #16
EVQLVQSGAEVKKPGSSVKVSCKASGFTFTDYYIHWVKQAPGQGLEWIGLVHPYIGGTNYNQKFKGKATLTVDTSTTTAYMEMSSLRSEDTAVYYCARGDNKYDAMDYWGQGTTVTVSS

SEQ ID NO: 202 - protein, 119 aa, synthetic construct
  REGION 1..119: note = Heavy Chain of huAD208.4.1 IgG histidine scanning variant #17
EVQLVQSGAEVKKPGSSVKVSCKASGFTFTDYYIHWVKQAPGQGLEWIGLVYHYIGGTNYNQKFKGKATLTVDTSTTTAYMEMSSLRSEDTAVYYCARGDNKYDAMDYWGQGTTVTVSS

SEQ ID NO: 203 - protein, 119 aa, synthetic construct
  REGION 1..119: note = Heavy Chain of huAD208.4.1 IgG histidine scanning variant #18
EVQLVQSGAEVKKPGSSVKVSCKASGFTFTDYYIHWVKQAPGQGLEWIGLVYPHIGGTNYNQKFKGKATLTVDTSTTTAYMEMSSLRSEDTAVYYCARGDNKYDAMDYWGQGTTVTVSS

SEQ ID NO: 204 - protein, 119 aa, synthetic construct
  REGION 1..119: note = Heavy Chain of huAD208.4.1 IgG histidine scanning variant #19
EVQLVQSGAEVKKPGSSVKVSCKASGFTFTDYYIHWVKQAPGQGLEWIGLVYPYHGGTNYNQKFKGKATLTVDTSTTTAYMEMSSLRSEDTAVYYCARGDNKYDAMDYWGQGTTVTVSS

SEQ ID NO: 205 - protein, 119 aa, synthetic construct
  REGION 1..119: note = Heavy Chain of huAD208.4.1 IgG histidine scanning variant #20
EVQLVQSGAEVKKPGSSVKVSCKASGFTFTDYYIHWVKQAPGQGLEWIGLVYPYIHGTNYNQKFKGKATLTVDTSTTTAYMEMSSLRSEDTAVYYCARGDNKYDAMDYWGQGTTVTVSS

SEQ ID NO: 206 - protein, 119 aa, synthetic construct
  REGION 1..119: note = Heavy Chain of huAD208.4.1 IgG histidine scanning variant #21
EVQLVQSGAEVKKPGSSVKVSCKASGFTFTDYYIHWVKQAPGQGLEWIGLVYPYIGHTNYNQKFKGKATLTVDTSTTTAYMEMSSLRSEDTAVYYCARGDNKYDAMDYWGQGTTVTVSS

SEQ ID NO: 207 - protein, 119 aa, synthetic construct
  REGION 1..119: note = Heavy Chain of huAD208.4.1 IgG histidine scanning variant #22
EVQLVQSGAEVKKPGSSVKVSCKASGFTFTDYYIHWVKQAPGQGLEWIGLVYPYIGGHNYNQKFKGKATLTVDTSTTTAYMEMSSLRSEDTAVYYCARGDNKYDAMDYWGQGTTVTVSS

SEQ ID NO: 208 - protein, 119 aa, synthetic construct
  REGION 1..119: note = Heavy Chain of huAD208.4.1 IgG histidine scanning variant #23
EVQLVQSGAEVKKPGSSVKVSCKASGFTFTDYYIHWVKQAPGQGLEWIGLVYPYIGGTHYNQKFKGKATLTVDTSTTTAYMEMSSLRSEDTAVYYCARGDNKYDAMDYWGQGTTVTVSS

SEQ ID NO: 209 - protein, 119 aa, synthetic construct
  REGION 1..119: note = Heavy Chain of huAD208.4.1 IgG histidine scanning variant #24
EVQLVQSGAEVKKPGSSVKVSCKASGFTFTDYYIHWVKQAPGQGLEWIGLVYPYIGGTNHNQKFKGKATLTVDTSTTTAYMEMSSLRSEDTAVYYCARGDNKYDAMDYWGQGTTVTVSS

SEQ ID NO: 210 - protein, 119 aa, synthetic construct
  REGION 1..119: note = Heavy Chain of huAD208.4.1 IgG histidine scanning variant #25
EVQLVQSGAEVKKPGSSVKVSCKASGFTFTDYYIHWVKQAPGQGLEWIGLVYPYIGGTNYHQKFKGKATLTVDTSTTTAYMEMSSLRSEDTAVYYCARGDNKYDAMDYWGQGTTVTVSS

SEQ ID NO: 211 - protein, 119 aa, synthetic construct
  REGION 1..119: note = Heavy Chain of huAD208.4.1 IgG histidine scanning variant #26
EVQLVQSGAEVKKPGSSVKVSCKASGFTFTDYYIHWVKQAPGQGLEWIGLVYPYIGGTNYNHKFKGKATLTVDTSTTTAYMEMSSLRSEDTAVYYCARGDNKYDAMDYWGQGTTVTVSS

SEQ ID NO: 212 - protein, 119 aa, synthetic construct
  REGION 1..119: note = Heavy Chain of huAD208.4.1 IgG histidine scanning variant #27
EVQLVQSGAEVKKPGSSVKVSCKASGFTFTDYYIHWVKQAPGQGLEWIGLVYPYIGGTNYNQHFKGKATLTVDTSTTTAYMEMSSLRSEDTAVYYCARGDNKYDAMDYWGQGTTVTVSS

SEQ ID NO: 213 - protein, 119 aa, synthetic construct
  REGION 1..119: note = Heavy Chain of huAD208.4.1 IgG histidine scanning variant #28
EVQLVQSGAEVKKPGSSVKVSCKASGFTFTDYYIHWVKQAPGQGLEWIGLVYPYIGGTNYNQKHKGKATLTVDTSTTTAYMEMSSLRSEDTAVYYCARGDNKYDAMDYWGQGTTVTVSS

SEQ ID NO: 214 - protein, 119 aa, synthetic construct
  REGION 1..119: note = Heavy Chain of huAD208.4.1 IgG histidine scanning variant #29
EVQLVQSGAEVKKPGSSVKVSCKASGFTFTDYYIHWVKQAPGQGLEWIGLVYPYIGGTNYNQKFHGKATLTVDTSTTTAYMEMSSLRSEDTAVYYCARGDNKYDAMDYWGQGTTVTVSS

SEQ ID NO: 215 - protein, 119 aa, synthetic construct
  REGION 1..119: note = Heavy Chain of huAD208.4.1 IgG histidine scanning variant #30
EVQLVQSGAEVKKPGSSVKVSCKASGFTFTDYYIHWVKQAPGQGLEWIGLVYPYIGGTNYNQKFKHKATLTVDTSTTTAYMEMSSLRSEDTAVYYCARGDNKYDAMDYWGQGTTVTVSS

SEQ ID NO: 216 - protein, 119 aa, synthetic construct
  REGION 1..119: note = Heavy Chain of huAD208.4.1 IgG histidine scanning variant #31
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCHRGDNKYDAMDYWGQGTTVTVSS

SEQ ID NO: 217 - protein, 119 aa, synthetic construct
  REGION 1..119: note = Heavy Chain of huAD208.4.1 IgG histidine scanning variant #32
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCAHGDNKYDAMDYWGQGTTVTVSS

SEQ ID NO: 218 - protein, 119 aa, synthetic construct
  REGION 1..119: note = Heavy Chain of huAD208.4.1 IgG histidine scanning variant #33
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARHDNKYDAMDYWGQGTTVTVSS

SEQ ID NO: 219 - protein, 119 aa, synthetic construct
  REGION 1..119: note = Heavy Chain of huAD208.4.1 IgG histidine scanning variant #34
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGHNKYDAMDYWGQGTTVTVSS

SEQ ID NO: 220 - protein, 119 aa, synthetic construct
  REGION 1..119: note = Heavy Chain of huAD208.4.1 IgG histidine scanning variant #35
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDHKYDAMDYWGQGTTVTVSS

SEQ ID NO: 221 - protein, 119 aa, synthetic construct
  REGION 1..119: note = Heavy Chain of huAD208.4.1 IgG histidine scanning variant #36
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNHYDAMDYWGQGTTVTVSS

SEQ ID NO: 222 - protein, 119 aa, synthetic construct
  REGION 1..119: note = Heavy Chain of huAD208.4.1 IgG histidine scanning variant #37
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKHDAMDYWGQGTTVTVSS

SEQ ID NO: 223 - protein, 119 aa, synthetic construct
  REGION 1..119: note = Heavy Chain of huAD208.4.1 IgG histidine scanning variant #38
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYHAMDYWGQGTTVTVSS

SEQ ID NO: 224 - protein, 119 aa, synthetic construct
  REGION 1..119: note = Heavy Chain of huAD208.4.1 IgG histidine scanning variant #39
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDHMDYWGQGTTVTVSS

SEQ ID NO: 225 - protein, 119 aa, synthetic construct
  REGION 1..119: note = Heavy Chain of huAD208.4.1 IgG histidine scanning variant #40
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAHDYWGQGTTVTVSS

SEQ ID NO: 226 - protein, 119 aa, synthetic construct
  REGION 1..119: note = Heavy Chain of huAD208.4.1 IgG histidine scanning variant #41
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMHYWGQGTTVTVSS

SEQ ID NO: 227 - protein, 119 aa, synthetic construct
  REGION 1..119: note = Heavy Chain of huAD208.4.1 IgG histidine scanning variant #42
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDHWGQGTTVTVSS

SEQ ID NO: 228 - protein, 110 aa, synthetic construct
  REGION 1..110: note = Light Chain of huAD208.4.1 IgG histidine scanning variant #1
DIVLTQSPDSLAVSLGERATINCHASQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 229 - protein, 110 aa, synthetic construct
  REGION 1..110: note = Light Chain of huAD208.4.1 IgG histidine scanning variant #2
DIVLTQSPDSLAVSLGERATINCRHSQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 230 - protein, 110 aa, synthetic construct
  REGION 1..110: note = Light Chain of huAD208.4.1 IgG histidine scanning variant #3
DIVLTQSPDSLAVSLGERATINCRAHQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 231 - protein, 110 aa, synthetic construct
  REGION 1..110: note = Light Chain of huAD208.4.1 IgG histidine scanning variant #4
DIVLTQSPDSLAVSLGERATINCRASHSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 232 - protein, 110 aa, synthetic construct
  REGION 1..110: note = Light Chain of huAD208.4.1 IgG histidine scanning variant #5
DIVLTQSPDSLAVSLGERATINCRASQHVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 233 - protein, 110 aa, synthetic construct
  REGION 1..110: note = Light Chain of huAD208.4.1 IgG histidine scanning variant #6
DIVLTQSPDSLAVSLGERATINCRASQSHSTSSYSYMHWYQQKPGQPPKLLIKYASSLESGVPDRFSGSGSGTDFTLTISSLQAEDVAVYYCEQSWEIRTFGGGTKVEIK

SEQ ID NO: 234 - protein, 110 aa, synthetic construct
  REGION 1..110: note = Light Chain of huAD208.4.1 IgG histidine scanning variant #7
DIVLTQSPDSLAVSLGERATINCRASQSVHTSSYSYMHWYQQKPGQPPKLLIKYASSLESGVPDRFSGSGSGTDFTLTISSLQAEDVAVYYCEQSWEIRTFGGGTKVEIK

SEQ ID NO: 235 - protein, 110 aa, synthetic construct
  REGION 1..110: note = Light Chain of huAD208.4.1 IgG histidine scanning variant #8
DIVLTQSPDSLAVSLGERATINCRASQSVSHSSYSYMHWYQQKPGQPPKLLIKYASSLESGVPDRFSGSGSGTDFTLTISSLQAEDVAVYYCEQSWEIRTFGGGTKVEIK

SEQ ID NO: 236 - protein, 110 aa, synthetic construct
  REGION 1..110: note = Light Chain of huAD208.4.1 IgG histidine scanning variant #9
DIVLTQSPDSLAVSLGERATINCRASQSVSTHSYSYMHWYQQKPGQPPKLLIKYASSLESGVPDRFSGSGSGTDFTLTISSLQAEDVAVYYCEQSWEIRTFGGGTKVEIK

SEQ ID NO: 237 - protein, 110 aa, synthetic construct
  REGION 1..110: note = Light Chain of huAD208.4.1 IgG histidine scanning variant #10
DIVLTQSPDSLAVSLGERATINCRASQSVSTSHYSYMHWYQQKPGQPPKLLIKYASSLESGVPDRFSGSGSGTDFTLTISSLQAEDVAVYYCEQSWEIRTFGGGTKVEIK

SEQ ID NO: 238 - protein, 110 aa, synthetic construct
  REGION 1..110: note = Light Chain of huAD208.4.1 IgG histidine scanning variant #11
DIVLTQSPDSLAVSLGERATINCRASQSVSTSSHSYMHWYQQKPGQPPKLLIKYASSLESGVPDRFSGSGSGTDFTLTISSLQAEDVAVYYCEQSWEIRTFGGGTKVEIK

SEQ ID NO: 239 - protein, 110 aa, synthetic construct
  REGION 1..110: note = Light Chain of huAD208.4.1 IgG histidine scanning variant #12
DIVLTQSPDSLAVSLGERATINCRASQSVSTSSYHYMHWYQQKPGQPPKLLIKYASSLESGVPDRFSGSGSGTDFTLTISSLQAEDVAVYYCEQSWEIRTFGGGTKVEIK

SEQ ID NO: 240 - protein, 110 aa, synthetic construct
  REGION 1..110: note = Light Chain of huAD208.4.1 IgG histidine scanning variant #13
DIVLTQSPDSLAVSLGERATINCRASQSVSTSSYSHMHWYQQKPGQPPKLLIKYASSLESGVPDRFSGSGSGTDFTLTISSLQAEDVAVYYCEQSWEIRTFGGGTKVEIK

SEQ ID NO: 241 - protein, 110 aa, synthetic construct
  REGION 1..110: note = Light Chain of huAD208.4.1 IgG histidine scanning variant #14
DIVLTQSPDSLAVSLGERATINCRASQSVSTSSYSYHHWYQQKPGQPPKLLIKYASSLESGVPDRFSGSGSGTDFTLTISSLQAEDVAVYYCEQSWEIRTFGGGTKVEIK

SEQ ID NO: 242 - protein, 110 aa, synthetic construct
  REGION 1..110: note = Light Chain of huAD208.4.1 IgG histidine scanning variant #15
DIVLTQSPDSLAVSLGERATINCRASQSVSTSSYSYMAWYQQKPGQPPKLLIKYASSLESGVPDRFSGSGSGTDFTLTISSLQAEDVAVYYCEQSWEIRTFGGGTKVEIK

SEQ ID NO: 243 - protein, 110 aa, synthetic construct
  REGION 1..110: note = Light Chain of huAD208.4.1 IgG histidine scanning variant #16
DIVLTQSPDSLAVSLGERATINCRASQSVSTSSYSYMHWYQQKPGQPPKLLIKHASSLESGVPDRFSGSGSGTDFTLTISSLQAEDVAVYYCEQSWEIRTFGGGTKVEIK

SEQ ID NO: 244 - protein, 110 aa, synthetic construct
  REGION 1..110: note = Light Chain of huAD208.4.1 IgG histidine scanning variant #17
DIVLTQSPDSLAVSLGERATINCRASQSVSTSSYSYMHWYQQKPGQPPKLLIKYHSSLESGVPDRFSGSGSGTDFTLTISSLQAEDVAVYYCEQSWEIRTFGGGTKVEIK

SEQ ID NO: 245 - protein, 110 aa, synthetic construct
  REGION 1..110: note = Light Chain of huAD208.4.1 IgG histidine scanning variant #18
DIVLTQSPDSLAVSLGERATINCRASQSVSTSSYSYMHWYQQKPGQPPKLLIKYAHSLESGVPDRFSGSGSGTDFTLTISSLQAEDVAVYYCEQSWEIRTFGGGTKVEIK

SEQ ID NO: 246 - protein, 110 aa, synthetic construct
  REGION 1..110: note = Light Chain of huAD208.4.1 IgG histidine scanning variant #19
DIVLTQSPDSLAVSLGERATINCRASQSVSTSSYSYMHWYQQKPGQPPKLLIKYASHLESGVPDRFSGSGSGTDFTLTISSLQAEDVAVYYCEQSWEIRTFGGGTKVEIK

SEQ ID NO: 247 - protein, 110 aa, synthetic construct
  REGION 1..110: note = Light Chain of huAD208.4.1 IgG histidine scanning variant #20
DIVLTQSPDSLAVSLGERATINCRASQSVSTSSYSYMHWYQQKPGQPPKLLIKYASSHESGVPDRFSGSGSGTDFTLTISSLQAEDVAVYYCEQSWEIRTFGGGTKVEIK

SEQ ID NO: 248 - protein, 110 aa, synthetic construct
  REGION 1..110: note = Light Chain of huAD208.4.1 IgG histidine scanning variant #21
DIVLTQSPDSLAVSLGERATINCRASQSVSTSSYSYMHWYQQKPGQPPKLLIKYASSLHSGVPDRFSGSGSGTDFTLTISSLQAEDVAVYYCEQSWEIRTFGGGTKVEIK

SEQ ID NO: 249 - protein, 110 aa, synthetic construct
  REGION 1..110: note = Light Chain of huAD208.4.1 IgG histidine scanning variant #22
DIVLTQSPDSLAVSLGERATINCRASQSVSTSSYSYMHWYQQKPGQPPKLLIKYASSLEHGVPDRFSGSGSGTDFTLTISSLQAEDVAVYYCEQSWEIRTFGGGTKVEIK

SEQ ID NO: 250 - protein, 110 aa, synthetic construct
  REGION 1..110: note = Light Chain of huAD208.4.1 IgG histidine scanning variant #23
DIVLTQSPDSLAVSLGERATINCRASQSVSTSSYSYMHWYQQKPGQPPKLLIKYASSLESGVPDRFSGSGSGTDFTLTISSLQAEDVAVYYCHQSWEIRTFGGGTKVEIK

SEQ ID NO: 251 - protein, 110 aa, synthetic construct
  REGION 1..110: note = Light Chain of huAD208.4.1 IgG histidine scanning variant #24
DIVLTQSPDSLAVSLGERATINCRASQSVSTSSYSYMHWYQQKPGQPPKLLIKYASSLESGVPDRFSGSGSGTDFTLTISSLQAEDVAVYYCEHSWEIRTFGGGTKVEIK

SEQ ID NO: 252 - protein, 110 aa, synthetic construct
  REGION 1..110: note = Light Chain of huAD208.4.1 IgG histidine scanning variant #25
DIVLTQSPDSLAVSLGERATINCRASQSVSTSSYSYMHWYQQKPGQPPKLLIKYASSLESGVPDRFSGSGSGTDFTLTISSLQAEDVAVYYCEQHWEIRTFGGGTKVEIK

SEQ ID NO: 253 - protein, 110 aa, synthetic construct
  REGION 1..110: note = Light Chain of huAD208.4.1 IgG histidine scanning variant #26
DIVLTQSPDSLAVSLGERATINCRASQSVSTSSYSYMHWYQQKPGQPPKLLIKYASSLESGVPDRFSGSGSGTDFTLTISSLQAEDVAVYYCEQSHEIRTFGGGTKVEIK

SEQ ID NO: 254 - protein, 110 aa, synthetic construct
  REGION 1..110: note = Light Chain of huAD208.4.1 IgG histidine scanning variant #27
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWHIRTFGGGTKVEIK

SEQ ID NO: 255 - protein, 110 aa, synthetic construct
  REGION 1..110: note = Light Chain of huAD208.4.1 IgG histidine scanning variant #28
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEHRTFGGGTKVEIK

SEQ ID NO: 256 - protein, 110 aa, synthetic construct
  REGION 1..110: note = Light Chain of huAD208.4.1 IgG histidine scanning variant #29
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIHTFGGGTKVEIK

SEQ ID NO: 257 - protein, 110 aa, synthetic construct
  REGION 1..110: note = Light Chain of huAD208.4.1 IgG histidine scanning variant #30
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRHFGGGTKVEIK

SEQ ID NO: 258 - protein, 119 aa, synthetic construct
  REGION 1..119: note = Heavy Chain Combinations of huAD208.4.1 IgG histidine scanning variant #1
EVQLVQSGAEVKKPGSSVKVSCKASGFTFTDYHIHWVKQAPGQGLEWIGLVYPYIGGTNYNQKFKGKATLTVDTSTTTAYMEMSSLRSEDTAVYYCARGDNKHDAMDYWGQGTTVTVSS

SEQ ID NO: 259 - protein, 119 aa, synthetic construct
  REGION 1..119: note = Heavy Chain Combinations of huAD208.4.1 IgG histidine scanning variant #2
EVQLVQSGAEVKKPGSSVKVSCKASGFTFTDYHIHWVKQAPGQGLEWIGLVYPYIGGTNYNQKFKGKATLTVDTSTTTAYMEMSSLRSEDTAVYYCARGDNKYDAHDYWGQGTTVTVSS

SEQ ID NO: 260 - protein, 119 aa, synthetic construct
  REGION 1..119: note = Heavy Chain Combinations of huAD208.4.1 IgG histidine scanning variant #3
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKHDAHDYWGQGTTVTVSS

SEQ ID NO: 261 - protein, 119 aa, synthetic construct
  REGION 1..119: note = Heavy Chain Combinations of huAD208.4.1 IgG histidine scanning variant #4
EVQLVQSGAEVKKPGSSVKVSCKASGFTFTDYHIHWVKQAPGQGLEWIGLVYPYIGGTNYNQKFKGKATLTVDTSTTTAYMEMSSLRSEDTAVYYCARGDNKHDAHDYWGQGTTVTVSS

SEQ ID NO: 262 - protein, 110 aa, synthetic construct
  REGION 1..110: note = Light Chain Combinations of huAD208.4.1 IgG histidine scanning variant #1
DIVLTQSPDSLAVSLGERATINCRHHQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 263 - protein, 110 aa, synthetic construct
  REGION 1..110: note = Light Chain Combinations of huAD208.4.1 IgG histidine scanning variant #2
DIVLTQSPDSLAVSLGERATINCRHSQSVSHSSYSYMHWYQQKPGQPPKLLIKYASSLESGVPDRFSGSGSGTDFTLTISSLQAEDVAVYYCEQSWEIRTFGGGTKVEIK

SEQ ID NO: 264 - protein, 110 aa, synthetic construct
  REGION 1..110: note = Light Chain Combinations of huAD208.4.1 IgG histidine scanning variant #3
DIVLTQSPDSLAVSLGERATINCRHSQSVSTSSYSHMHWYQQKPGQPPKLLIKYASSLESGVPDRFSGSGSGTDFTLTISSLQAEDVAVYYCEQSWEIRTFGGGTKVEIK

SEQ ID NO: 265 - protein, 110 aa, synthetic construct
  REGION 1..110: note = Light Chain Combinations of huAD208.4.1 IgG histidine scanning variant #4
DIVLTQSPDSLAVSLGERATINCRAHQSVSHSSYSYMHWYQQKPGQPPKLLIKYASSLESGVPDRFSGSGSGTDFTLTISSLQAEDVAVYYCEQSWEIRTFGGGTKVEIK

SEQ ID NO: 266 - protein, 110 aa, synthetic construct
  REGION 1..110: note = Light Chain Combinations of huAD208.4.1 IgG histidine scanning variant #5
DIVLTQSPDSLAVSLGERATINCRAHQSVSTSSYSHMHWYQQKPGQPPKLLIKYASSLESGVPDRFSGSGSGTDFTLTISSLQAEDVAVYYCEQSWEIRTFGGGTKVEIK

SEQ ID NO: 267 - protein, 110 aa, synthetic construct
  REGION 1..110: note = Light Chain Combinations of huAD208.4.1 IgG histidine scanning variant #6
DIVLTQSPDSLAVSLGERATINCRASQSVSHSSYSHMHWYQQKPGQPPKLLIKYASSLESGVPDRFSGSGSGTDFTLTISSLQAEDVAVYYCEQSWEIRTFGGGTKVEIK

SEQ ID NO: 268 - protein, 110 aa, synthetic construct
  REGION 1..110: note = Light Chain Combinations of huAD208.4.1 IgG histidine scanning variant #7
DIVLTQSPDSLAVSLGERATINCRHHQSVSHSSYSYMHWYQQKPGQPPKLLIKYASSLESGVPDRFSGSGSGTDFTLTISSLQAEDVAVYYCEQSWEIRTFGGGTKVEIK

SEQ ID NO: 269 - protein, 110 aa, synthetic construct
  REGION 1..110: note = Light Chain Combinations of huAD208.4.1 IgG histidine scanning variant #8
DIVLTQSPDSLAVSLGERATINCRHHQSVSTSSYSHMHWYQQKPGQPPKLLIKYASSLESGVPDRFSGSGSGTDFTLTISSLQAEDVAVYYCEQSWEIRTFGGGTKVEIK

SEQ ID NO: 270 - protein, 110 aa, synthetic construct
  REGION 1..110: note = Light Chain Combinations of huAD208.4.1 IgG histidine scanning variant #9
DIVLTQSPDSLAVSLGERATINCRHSQSVSHSSYSHMHWYQQKPGQPPKLLIKYASSLESGVPDRFSGSGSGTDFTLTISSLQAEDVAVYYCEQSWEIRTFGGGTKVEIK

SEQ ID NO: 271 - protein, 110 aa, synthetic construct
  REGION 1..110: note = Light Chain Combinations of huAD208.4.1 IgG histidine scanning variant #10
DIVLTQSPDSLAVSLGERATINCRAHQSVSHSSYSHMHWYQQKPGQPPKLLIKYASSLESGVPDRFSGSGSGTDFTLTISSLQAEDVAVYYCEQSWEIRTFGGGTKVEIK

SEQ ID NO: 272 - protein, 121 aa, synthetic construct
  REGION 1..121: note = Heavy Chain of huAD208.12.1 IgG
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 273 - protein, 107 aa, synthetic construct
  REGION 1..107: note = Light Chain of huAD208.12.1 IgG
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFTFGQGTKLEIK

SEQ ID NO: 274 - protein, 13 aa, synthetic construct
  REGION 1..13: note = Heavy Chain CDR1 of huAD208.12.1
KASGYTFTNYWMH

SEQ ID NO: 275 - protein, 17 aa, synthetic construct
  REGION 1..17: note = Heavy Chain CDR2 of huAD208.12.1
MIHPNSGSTKHNEKFRG

SEQ ID NO: 276 - protein, 14 aa, synthetic construct
  REGION 1..14: note = Heavy Chain CDR3 of huAD208.12.1
ARSDFGNYRWYFDV

SEQ ID NO: 277 - protein, 11 aa, synthetic construct
  REGION 1..11: note = Light Chain CDR1 of huAD208.12.1
RASQSSSNNLH

SEQ ID NO: 278 - protein, 7 aa, synthetic construct
  REGION 1..7: note = Light Chain CDR2 of huAD208.12.1
YVSQSIS

SEQ ID NO: 279 - protein, 9 aa, synthetic construct
  REGION 1..9: note = Light Chain CDR3 of huAD208.12.1
QQSNSWPFT

SEQ ID NO: 280 - protein, 121 aa, synthetic construct
  REGION 1..121: note = Heavy Chain of huAD208.12.1 IgG histidine scanning variant #1
EVQLVQSGAEVKKPGSSVKVSCHASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 281 - protein, 121 aa, synthetic construct
  REGION 1..121: note = Heavy Chain of huAD208.12.1 IgG histidine scanning variant #2
EVQLVQSGAEVKKPGSSVKVSCKHSGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 282 - protein, 121 aa, synthetic construct
  REGION 1..121: note = Heavy Chain of huAD208.12.1 IgG histidine scanning variant #3
EVQLVQSGAEVKKPGSSVKVSCKAHGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 283 - protein, 121 aa, synthetic construct
  REGION 1..121: note = Heavy Chain of huAD208.12.1 IgG histidine scanning variant #4
EVQLVQSGAEVKKPGSSVKVSCKASHYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 284 - protein, 121 aa, synthetic construct
  REGION 1..121: note = Heavy Chain of huAD208.12.1 IgG histidine scanning variant #5
EVQLVQSGAEVKKPGSSVKVSCKASGHTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 285 - protein, 121 aa, synthetic construct
  REGION 1..121: note = Heavy Chain of huAD208.12.1 IgG histidine scanning variant #6
EVQLVQSGAEVKKPGSSVKVSCKASGYHFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 286 - protein, 121 aa, synthetic construct
  REGION 1..121: note = Heavy Chain of huAD208.12.1 IgG histidine scanning variant #7
EVQLVQSGAEVKKPGSSVKVSCKASGYTHTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 287 - protein, 121 aa, synthetic construct
  REGION 1..121: note = Heavy Chain of huAD208.12.1 IgG histidine scanning variant #8
EVQLVQSGAEVKKPGSSVKVSCKASGYTFHNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 288 - protein, 121 aa, synthetic construct
  REGION 1..121: note = Heavy Chain of huAD208.12.1 IgG histidine scanning variant #9
EVQLVQSGAEVKKPGSSVKVSCKASGYTFTHYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 289 - protein, 121 aa, synthetic construct
  REGION 1..121: note = Heavy Chain of huAD208.12.1 IgG histidine scanning variant #10
EVQLVQSGAEVKKPGSSVKVSCKASGYTFTNHWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 290 - protein, 121 aa, synthetic construct
  REGION 1..121: note = Heavy Chain of huAD208.12.1 IgG histidine scanning variant #11
EVQLVQSGAEVKKPGSSVKVSCKASGYTFTNYHMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 291 - protein, 121 aa, synthetic construct
  REGION 1..121: note = Heavy Chain of huAD208.12.1 IgG histidine scanning variant #12
EVQLVQSGAEVKKPGSSVKVSCKASGYTFTNYWHHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 292 - protein, 121 aa, synthetic construct
  REGION 1..121: note = Heavy Chain of huAD208.12.1 IgG histidine scanning variant #13
EVQLVQSGAEVKKPGSSVKVSCKASGYTFTNYWMAWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 293 - protein, 121 aa, synthetic construct
  REGION 1..121: note = Heavy Chain of huAD208.12.1 IgG histidine scanning variant #14?
EVQLVQSGAEVKKPGSSVKVSCKASGYTFTNYWMHWVKQAPGQGLEWIGHIHPNSGSTKHNEKFRGKATLTVDESTTTAYMELSSLRSEDTAVYYCARSDFGNYRWYFDVWGQGTTVTVSS

SEQ ID NO: 294 - protein, 121 aa, synthetic construct
  REGION 1..121: note = Heavy Chain of huAD208.12.1 IgG histidine scanning variant #15
EVQLVQSGAEVKKPGSSVKVSCKASGYTFTNYWMHWVKQAPGQGLEWIGMHHPNSGSTKHNEKFRGKATLTVDESTTTAYMELSSLRSEDTAVYYCARSDFGNYRWYFDVWGQGTTVTVSS

SEQ ID NO: 295 - protein, 121 aa, synthetic construct
  REGION 1..121: note = Heavy Chain of huAD208.12.1 IgG histidine scanning variant #16
EVQLVQSGAEVKKPGSSVKVSCKASGYTFTNYWMHWVKQAPGQGLEWIGMIAPNSGSTKHNEKFRGKATLTVDESTTTAYMELSSLRSEDTAVYYCARSDFGNYRWYFDVWGQGTTVTVSS

SEQ ID NO: 296 - protein, 121 aa, synthetic construct
  REGION 1..121: note = Heavy Chain of huAD208.12.1 IgG histidine scanning variant #17
EVQLVQSGAEVKKPGSSVKVSCKASGYTFTNYWMHWVKQAPGQGLEWIGMIHHNSGSTKHNEKFRGKATLTVDESTTTAYMELSSLRSEDTAVYYCARSDFGNYRWYFDVWGQGTTVTVSS

SEQ ID NO: 297 - protein, 121 aa, synthetic construct
  REGION 1..121: note = Heavy Chain of huAD208.12.1 IgG histidine scanning variant #18
EVQLVQSGAEVKKPGSSVKVSCKASGYTFTNYWMHWVKQAPGQGLEWIGMIHPHSGSTKHNEKFRGKATLTVDESTTTAYMELSSLRSEDTAVYYCARSDFGNYRWYFDVWGQGTTVTVSS

SEQ ID NO: 298 - protein, 121 aa, synthetic construct
  REGION 1..121: note = Heavy Chain of huAD208.12.1 IgG histidine scanning variant #19
EVQLVQSGAEVKKPGSSVKVSCKASGYTFTNYWMHWVKQAPGQGLEWIGMIHPNHGSTKHNEKFRGKATLTVDESTTTAYMELSSLRSEDTAVYYCARSDFGNYRWYFDVWGQGTTVTVSS

SEQ ID NO: 299 - protein, 121 aa, synthetic construct
  REGION 1..121: note = Heavy Chain of huAD208.12.1 IgG histidine scanning variant #20
EVQLVQSGAEVKKPGSSVKVSCKASGYTFTNYWMHWVKQAPGQGLEWIGMIHPNSHSTKHNEKFRGKATLTVDESTTTAYMELSSLRSEDTAVYYCARSDFGNYRWYFDVWGQGTTVTVSS

SEQ ID NO: 300 - protein, 121 aa, synthetic construct
  REGION 1..121: note = Heavy Chain of huAD208.12.1 IgG histidine scanning variant #21
EVQLVQSGAEVKKPGSSVKVSCKASGYTFTNYWMHWVKQAPGQGLEWIGMIHPNSGHTKHNEKFRGKATLTVDESTTTAYMELSSLRSEDTAVYYCARSDFGNYRWYFDVWGQGTTVTVSS

SEQ ID NO: 301 - protein, 121 aa, synthetic construct
  REGION 1..121: note = Heavy Chain of huAD208.12.1 IgG histidine scanning variant #22
EVQLVQSGAEVKKPGSSVKVSCKASGYTFTNYWMHWVKQAPGQGLEWIGMIHPNSGSHKHNEKFRGKATLTVDESTTTAYMELSSLRSEDTAVYYCARSDFGNYRWYFDVWGQGTTVTVSS

SEQ ID NO: 302 - protein, 121 aa, synthetic construct
  REGION 1..121: note = Heavy Chain of huAD208.12.1 IgG histidine scanning variant #23
EVQLVQSGAEVKKPGSSVKVSCKASGYTFTNYWMHWVKQAPGQGLEWIGMIHPNSGSTHHNEKFRGKATLTVDESTTTAYMELSSLRSEDTAVYYCARSDFGNYRWYFDVWGQGTTVTVSS

SEQ ID NO: 303 - protein, 121 aa, synthetic construct
  REGION 1..121: note = Heavy Chain of huAD208.12.1 IgG histidine scanning variant #24
EVQLVQSGAEVKKPGSSVKVSCKASGYTFTNYWMHWVKQAPGQGLEWIGMIHPNSGSTKANEKFRGKATLTVDESTTTAYMELSSLRSEDTAVYYCARSDFGNYRWYFDVWGQGTTVTVSS

SEQ ID NO: 304 - protein, 121 aa, synthetic construct
  REGION 1..121: note = Heavy Chain of huAD208.12.1 IgG histidine scanning variant #25
EVQLVQSGAEVKKPGSSVKVSCKASGYTFTNYWMHWVKQAPGQGLEWIGMIHPNSGSTKHHEKFRGKATLTVDESTTTAYMELSSLRSEDTAVYYCARSDFGNYRWYFDVWGQGTTVTVSS

SEQ ID NO: 305 - protein, 121 aa, synthetic construct
  REGION 1..121: note = Heavy Chain of huAD208.12.1 IgG histidine scanning variant #26
EVQLVQSGAEVKKPGSSVKVSCKASGYTFTNYWMHWVKQAPGQGLEWIGMIHPNSGSTKHNHKFRGKATLTVDESTTTAYMELSSLRSEDTAVYYCARSDFGNYRWYFDVWGQGTTVTVSS

SEQ ID NO: 306 - protein, 121 aa, synthetic construct
  REGION 1..121: note = Heavy Chain of huAD208.12.1 IgG histidine scanning variant #27
EVQLVQSGAEVKKPGSSVKVSCKASGYTFTNYWMHWVKQAPGQGLEWIGMIHPNSGSTKHNEHFRGKATLTVDESTTTAYMELSSLRSEDTAVYYCARSDFGNYRWYFDVWGQGTTVTVSS

SEQ ID NO: 307 - protein, 121 aa, synthetic construct
  REGION 1..121: note = Heavy Chain of huAD208.12.1 IgG histidine scanning variant #28
EVQLVQSGAEVKKPGSSVKVSCKASGYTFTNYWMHWVKQAPGQGLEWIGMIHPNSGSTKHNEKHRGKATLTVDESTTTAYMELSSLRSEDTAVYYCARSDFGNYRWYFDVWGQGTTVTVSS

SEQ ID NO: 308 - protein, 121 aa, synthetic construct
  REGION 1..121: note = Heavy Chain of huAD208.12.1 IgG histidine scanning variant #29
EVQLVQSGAEVKKPGSSVKVSCKASGYTFTNYWMHWVKQAPGQGLEWIGMIHPNSGSTKHNEKFHGKATLTVDESTTTAYMELSSLRSEDTAVYYCARSDFGNYRWYFDVWGQGTTVTVSS

SEQ ID NO: 309 - protein, 121 aa, synthetic construct
  REGION 1..121: note = Heavy Chain of huAD208.12.1 IgG histidine scanning variant #30
EVQLVQSGAEVKKPGSSVKVSCKASGYTFTNYWMHWVKQAPGQGLEWIGMIHPNSGSTKHNEKFRHKATLTVDESTTTAYMELSSLRSEDTAVYYCARSDFGNYRWYFDVWGQGTTVTVSS

SEQ ID NO: 310 - protein, 121 aa, synthetic construct
  REGION 1..121: note = Heavy Chain of huAD208.12.1 IgG histidine scanning variant #31
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCHRSDFGNYRWYFDVWGQGTTVTVSS

SEQ ID NO: 311 - protein, 121 aa, synthetic construct
  REGION 1..121: note = Heavy Chain of huAD208.12.1 IgG histidine scanning variant #32
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCAHSDFGNYRWYFDVWGQGTTVTVSS

SEQ ID NO: 312 - protein, 121 aa, synthetic construct
  REGION 1..121: note = Heavy Chain of huAD208.12.1 IgG histidine scanning variant #33
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARHDFGNYRWYFDVWGQGTTVTVSS

SEQ ID NO: 313 - protein, 121 aa, synthetic construct
  REGION 1..121: note = Heavy Chain of huAD208.12.1 IgG histidine scanning variant #34
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSHFGNYRWYFDVWGQGTTVTVSS

SEQ ID NO: 314 - protein, 121 aa, synthetic construct
  REGION 1..121: note = Heavy Chain of huAD208.12.1 IgG histidine scanning variant #35
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDHGNYRWYFDVWGQGTTVTVSS

SEQ ID NO: 315 - protein, 121 aa, synthetic construct
  REGION 1..121: note = Heavy Chain of huAD208.12.1 IgG histidine scanning variant #36
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFHNYRWYFDVWGQGTTVTVSS

SEQ ID NO: 316 - protein, 121 aa, synthetic construct
  REGION 1..121: note = Heavy Chain of huAD208.12.1 IgG histidine scanning variant #37
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGHYRWYFDVWGQGTTVTVSS

SEQ ID NO: 317 - protein, 121 aa, synthetic construct
  REGION 1..121: note = Heavy Chain of huAD208.12.1 IgG histidine scanning variant #38
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNHRWYFDVWGQGTTVTVSS

SEQ ID NO: 318 - protein, 121 aa, synthetic construct
  REGION 1..121: note = Heavy Chain of huAD208.12.1 IgG histidine scanning variant #39
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYHWYFDVWGQGTTVTVSS

SEQ ID NO: 319 - protein, 121 aa, synthetic construct
  REGION 1..121: note = Heavy Chain of huAD208.12.1 IgG histidine scanning variant #40
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRHYFDVWGQGTTVTVSS

SEQ ID NO: 320 - protein, 121 aa, synthetic construct
  REGION 1..121: note = Heavy Chain of huAD208.12.1 IgG histidine scanning variant #41
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWHFDVWGQGTTVTVSS

SEQ ID NO: 321 - protein, 121 aa, synthetic construct
  REGION 1..121: note = Heavy Chain of huAD208.12.1 IgG histidine scanning variant #42
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYHDVWGQGTTVTVSS

SEQ ID NO: 322 - protein, 121 aa, synthetic construct
  REGION 1..121: note = Heavy Chain of huAD208.12.1 IgG histidine scanning variant #43
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFHVWGQGTTVTVSS

SEQ ID NO: 323 - protein, 121 aa, synthetic construct
  REGION 1..121: note = Heavy Chain of huAD208.12.1 IgG histidine scanning variant #44
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDHWGQGTTVTVSS

SEQ ID NO: 324 - protein, 107 aa, synthetic construct
  REGION 1..107: note = Light Chain of huAD208.12.1 IgG histidine scanning variant #1
EIVLTQSPATLSLSPGERATLSCHASQSSSNNLHWYQQKPGQAPRVLIKYVSQSISGIPARFSGSGSGTDFTLTISSLEPEDFAVYFCQQSNSWPFTFGQGTKLEIK

SEQ ID NO: 325 - protein, 107 aa, synthetic construct
  REGION 1..107: note = Light Chain of huAD208.12.1 IgG histidine scanning variant #2
EIVLTQSPATLSLSPGERATLSCRHSQSSSNNLHWYQQKPGQAPRVLIKYVSQSISGIPARFSGSGSGTDFTLTISSLEPEDFAVYFCQQSNSWPFTFGQGTKLEIK

SEQ ID NO: 326 - protein, 107 aa, synthetic construct
  REGION 1..107: note = Light Chain of huAD208.12.1 IgG histidine scanning variant #3
EIVLTQSPATLSLSPGERATLSCRAHQSSSNNLHWYQQKPGQAPRVLIKYVSQSISGIPARFSGSGSGTDFTLTISSLEPEDFAVYFCQQSNSWPFTFGQGTKLEIK

SEQ ID NO: 327 - protein, 107 aa, synthetic construct
  REGION 1..107: note = Light Chain of huAD208.12.1 IgG histidine scanning variant #4
EIVLTQSPATLSLSPGERATLSCRASHSSSNNLHWYQQKPGQAPRVLIKYVSQSISGIPARFSGSGSGTDFTLTISSLEPEDFAVYFCQQSNSWPFTFGQGTKLEIK

SEQ ID NO: 328 - protein, 107 aa, synthetic construct
  REGION 1..107: note = Light Chain of huAD208.12.1 IgG histidine scanning variant #5
EIVLTQSPATLSLSPGERATLSCRASQHSSNNLHWYQQKPGQAPRVLIKYVSQSISGIPARFSGSGSGTDFTLTISSLEPEDFAVYFCQQSNSWPFTFGQGTKLEIK

SEQ ID NO: 329 - protein, 107 aa, synthetic construct
  REGION 1..107: note = Light Chain of huAD208.12.1 IgG histidine scanning variant #6
EIVLTQSPATLSLSPGERATLSCRASQSHSNNLHWYQQKPGQAPRVLIKYVSQSISGIPARFSGSGSGTDFTLTISSLEPEDFAVYFCQQSNSWPFTFGQGTKLEIK

SEQ ID NO: 330 - protein, 107 aa, synthetic construct
  REGION 1..107: note = Light Chain of huAD208.12.1 IgG histidine scanning variant #7
EIVLTQSPATLSLSPGERATLSCRASQSSHNNLHWYQQKPGQAPRVLIKYVSQSISGIPARFSGSGSGTDFTLTISSLEPEDFAVYFCQQSNSWPFTFGQGTKLEIK

SEQ ID NO: 331 - protein, 107 aa, synthetic construct
  REGION 1..107: note = Light Chain of huAD208.12.1 IgG histidine scanning variant #8
EIVLTQSPATLSLSPGERATLSCRASQSSSHNLHWYQQKPGQAPRVLIKYVSQSISGIPARFSGSGSGTDFTLTISSLEPEDFAVYFCQQSNSWPFTFGQGTKLEIK

SEQ ID NO: 332 - protein, 107 aa, synthetic construct
  REGION 1..107: note = Light Chain of huAD208.12.1 IgG histidine scanning variant #9
EIVLTQSPATLSLSPGERATLSCRASQSSSNHLHWYQQKPGQAPRVLIKYVSQSISGIPARFSGSGSGTDFTLTISSLEPEDFAVYFCQQSNSWPFTFGQGTKLEIK

SEQ ID NO: 333 - protein, 107 aa, synthetic construct
  REGION 1..107: note = Light Chain of huAD208.12.1 IgG histidine scanning variant #10
EIVLTQSPATLSLSPGERATLSCRASQSSSNNHHWYQQKPGQAPRVLIKYVSQSISGIPARFSGSGSGTDFTLTISSLEPEDFAVYFCQQSNSWPFTFGQGTKLEIK

SEQ ID NO: 334 - protein, 107 aa, synthetic construct
  REGION 1..107: note = Light Chain of huAD208.12.1 IgG histidine scanning variant #11
EIVLTQSPATLSLSPGERATLSCRASQSSSNNLAWYQQKPGQAPRVLIKYVSQSISGIPARFSGSGSGTDFTLTISSLEPEDFAVYFCQQSNSWPFTFGQGTKLEIK

SEQ ID NO: 335 - protein, 107 aa, synthetic construct
  REGION 1..107: note = Light Chain of huAD208.12.1 IgG histidine scanning variant #12
EIVLTQSPATLSLSPGERATLSCRASQSSSNNLHWYQQKPGQAPRVLIKHVSQSISGIPARFSGSGSGTDFTLTISSLEPEDFAVYFCQQSNSWPFTFGQGTKLEIK

SEQ ID NO: 336 - protein, 107 aa, synthetic construct
  REGION 1..107: note = Light Chain of huAD208.12.1 IgG histidine scanning variant #13
EIVLTQSPATLSLSPGERATLSCRASQSSSNNLHWYQQKPGQAPRVLIKYHSQSISGIPARFSGSGSGTDFTLTISSLEPEDFAVYFCQQSNSWPFTFGQGTKLEIK

SEQ ID NO: 337 - protein, 107 aa, synthetic construct
  REGION 1..107: note = Light Chain of huAD208.12.1 IgG histidine scanning variant #14
EIVLTQSPATLSLSPGERATLSCRASQSSSNNLHWYQQKPGQAPRVLIKYVHQSISGIPARFSGSGSGTDFTLTISSLEPEDFAVYFCQQSNSWPFTFGQGTKLEIK

SEQ ID NO: 338 - protein, 107 aa, synthetic construct
  REGION 1..107: note = Light Chain of huAD208.12.1 IgG histidine scanning variant #15
EIVLTQSPATLSLSPGERATLSCRASQSSSNNLHWYQQKPGQAPRVLIKYVSHSISGIPARFSGSGSGTDFTLTISSLEPEDFAVYFCQQSNSWPFTFGQGTKLEIK

SEQ ID NO: 339 - protein, 107 aa, synthetic construct
  REGION 1..107: note = Light Chain of huAD208.12.1 IgG histidine scanning variant #16
EIVLTQSPATLSLSPGERATLSCRASQSSSNNLHWYQQKPGQAPRVLIKYVSQHISGIPARFSGSGSGTDFTLTISSLEPEDFAVYFCQQSNSWPFTFGQGTKLEIK

SEQ ID NO: 340 - protein, 107 aa, synthetic construct
  REGION 1..107: note = Light Chain of huAD208.12.1 IgG histidine scanning variant #17
EIVLTQSPATLSLSPGERATLSCRASQSSSNNLHWYQQKPGQAPRVLIKYVSQSHSGIPARFSGSGSGTDFTLTISSLEPEDFAVYFCQQSNSWPFTFGQGTKLEIK

SEQ ID NO: 341 - protein, 107 aa, synthetic construct
  REGION 1..107: note = Light Chain of huAD208.12.1 IgG histidine scanning variant #18
EIVLTQSPATLSLSPGERATLSCRASQSSSNNLHWYQQKPGQAPRVLIKYVSQSIHGIPARFSGSGSGTDFTLTISSLEPEDFAVYFCQQSNSWPFTFGQGTKLEIK

SEQ ID NO: 342 - protein, 107 aa, synthetic construct
  REGION 1..107: note = Light Chain of huAD208.12.1 IgG histidine scanning variant #19
EIVLTQSPATLSLSPGERATLSCRASQSSSNNLHWYQQKPGQAPRVLIKYVSQSISGIPARFSGSGSGTDFTLTISSLEPEDFAVYFCHQSNSWPFTFGQGTKLEIK

SEQ ID NO: 343 - protein, 107 aa, synthetic construct
  REGION 1..107: note = Light Chain of huAD208.12.1 IgG histidine scanning variant #20
EIVLTQSPATLSLSPGERATLSCRASQSSSNNLHWYQQKPGQAPRVLIKYVSQSISGIPARFSGSGSGTDFTLTISSLEPEDFAVYFCQHSNSWPFTFGQGTKLEIK

SEQ ID NO: 344 - protein, 107 aa, synthetic construct
  REGION 1..107: note = Light Chain of huAD208.12.1 IgG histidine scanning variant #21
EIVLTQSPATLSLSPGERATLSCRASQSSSNNLHWYQQKPGQAPRVLIKYVSQSISGIPARFSGSGSGTDFTLTISSLEPEDFAVYFCQQHNSWPFTFGQGTKLEIK

SEQ ID NO: 345 - protein, 107 aa, synthetic construct
  REGION 1..107: note = Light Chain of huAD208.12.1 IgG histidine scanning variant #22
EIVLTQSPATLSLSPGERATLSCRASQSSSNNLHWYQQKPGQAPRVLIKYVSQSISGIPARFSGSGSGTDFTLTISSLEPEDFAVYFCQQSHSWPFTFGQGTKLEIK

SEQ ID NO: 346 - protein, 107 aa, synthetic construct
  REGION 1..107: note = Light Chain of huAD208.12.1 IgG histidine scanning variant #23
EIVLTQSPATLSLSPGERATLSCRASQSSSNNLHWYQQKPGQAPRVLIKYVSQSISGIPARFSGSGSGTDFTLTISSLEPEDFAVYFCQQSNHWPFTFGQGTKLEIK

SEQ ID NO: 347 - protein, 107 aa, synthetic construct
  REGION 1..107: note = Light Chain of huAD208.12.1 IgG histidine scanning variant #24
EIVLTQSPATLSLSPGERATLSCRASQSSSNNLHWYQQKPGQAPRVLIKYVSQSISGIPARFSGSGSGTDFTLTISSLEPEDFAVYFCQQSNSHPFTFGQGTKLEIK

SEQ ID NO: 348 - protein, 107 aa, synthetic construct
  REGION 1..107: note = Light Chain of huAD208.12.1 IgG histidine scanning variant #25
EIVLTQSPATLSLSPGERATLSCRASQSSSNNLHWYQQKPGQAPRVLIKYVSQSISGIPARFSGSGSGTDFTLTISSLEPEDFAVYFCQQSNSWHFTFGQGTKLEIK

SEQ ID NO: 349 - protein, 107 aa, synthetic construct
  REGION 1..107: note = Light Chain of huAD208.12.1 IgG histidine scanning variant #26
EIVLTQSPATLSLSPGERATLSCRASQSSSNNLHWYQQKPGQAPRVLIKYVSQSISGIPARFSGSGSGTDFTLTISSLEPEDFAVYFCQQSNSWPHTFGQGTKLEIK

SEQ ID NO: 350 - protein, 107 aa, synthetic construct
  REGION 1..107: note = Light Chain of huAD208.12.1 IgG histidine scanning variant #27
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFHFGQGTKLEIK

SEQ ID NO: 351 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 352 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 353 - protein, 107 aa, Homo sapiens [sequence omitted]

SEQ ID NO: 354 - protein, 449 aa, synthetic construct
  REGION 1..449: note = Heavy Chain of samrotamab IgG + LS mutation
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 355 - protein, 449 aa, synthetic construct
  REGION 1..449: note = Heavy Chain of samrotamab IgG + YTE mutation
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 356 - protein, 449 aa, synthetic construct
  REGION 1..449: note = Heavy Chain of samrotamab IgG + A118C
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 357 - protein, 449 aa, synthetic construct
  REGION 1..449: note = Heavy Chain of samrotamab IgG + A118C + LS mutation
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 358 - protein, 449 aa, synthetic construct
  REGION 1..449: note = Heavy Chain of samrotamab IgG + A118C + YTE
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 359 - protein, 214 aa, synthetic construct
  REGION 1..214: note = Light Chain of samrotamab IgG + V205C
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 360 - protein, 449 aa, synthetic construct
  REGION 1..449: note = Heavy Chain of hu139.10 IgG + LS Mutation
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 361 - protein, 449 aa, synthetic construct
  REGION 1..449: note = Heavy Chain of hu139.10 IgG + YTE Mutation
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 362 - protein, 449 aa, synthetic construct
  REGION 1..449: note = Heavy Chain of hu139.10 IgG + A118C Mutation
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 363 - protein, 449 aa, synthetic construct
  REGION 1..449: note = Heavy Chain of hu139.10 IgG + A118C + LS Mutation
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 364 - protein, 449 aa, synthetic construct
  REGION 1..449: note = Heavy Chain of hu139.10 IgG + A118C + YTE Mutation
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 365 - protein, 219 aa, synthetic construct
  REGION 1..219: note = Light Chain of hy139.10 IgG + V205
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 366 - protein, 448 aa, synthetic construct
  REGION 1..448: note = Heavy Chain of huAD208.4.1 IgG + LS Mutation
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 367 - protein, 448 aa, synthetic construct
  REGION 1..448: note = Heavy Chain of huAD208.4.1 IgG + YTE Mutation
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 368 - protein, 448 aa, synthetic construct
  REGION 1..448: note = Heavy Chain of huAD208.4.1 IgG + A118C Mutation
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 369 - protein, 448 aa, synthetic construct; identical to SEQ ID NO: 366
  REGION 1..448: note = Heavy Chain of huAD208.4.1 IgG + A118C + LS Mutation

SEQ ID NO: 370 - protein, 448 aa, synthetic construct
  REGION 1..448: note = Heavy Chain of huAD208.4.1 IgG + A118C + YTE Mutation
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 371 - protein, 217 aa, synthetic construct
  REGION 1..217: note = Light Chain of huAD208.4.1 IgG + V205C
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 372 - protein, 450 aa, synthetic construct
  REGION 1..450: note = Heavy Chain of huAD208.12.1 IgG + LS Mutation
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 373 - protein, 450 aa, synthetic construct
  REGION 1..450: note = Heavy Chain of huAD208.12.1 IgG + YTE Mutation
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 374 - protein, 450 aa, synthetic construct
  REGION 1..450: note = Heavy Chain of huAD208.12.1 IgG + A118C Mutation
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 375 - protein, 450 aa, synthetic construct
  REGION 1..450: note = Heavy Chain variable domain of huAD208.12.1 IgG + A118C Mutation + LS Mutation
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 376 - protein, 450 aa, synthetic construct
  REGION 1..450: note = Heavy Chain variable domain of huAD208.12.1 IgG + A118C Mutation + YTE Mutation
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 377 - protein, 214 aa, synthetic construct
  REGION 1..214: note = Light Chain of huAD208.12.1 IgG + V205C
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 378 - protein, 121 aa, synthetic construct
  REGION 1..121: note = 15G7 rabbit VH
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 379 - protein, 11 aa, synthetic construct
  REGION 1..11: note = 15G7 HCDR1
GLDFRRTNYIC

SEQ ID NO: 380 - protein, 17 aa, synthetic construct
  REGION 1..17: note = 15G7 HCDR2
CIYIADVVHYYASWAKG

SEQ ID NO: 381 - protein, 14 aa, synthetic construct
  REGION 1..14: note = 15G7 HCDR3
ARGADGGFVYGMDL

SEQ ID NO: 382 - protein, 122 aa, synthetic construct
  REGION 1..122: note = 15G7 humanized (hu15G7) VH
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGGFVYGMDLWGQGTLVTVSS

SEQ ID NO: 383 - protein, 121 aa, synthetic construct
  REGION 1..121: note = 15G7 HC His variants in rabbit VH 1
QEQLEESGGDLVKPGASLTLTCTASHLDFRRTNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 384 - protein, 121 aa, synthetic construct
  REGION 1..121: note = 15G7 HC His variants in rabbit VH 2
QEQLEESGGDLVKPGASLTLTCTASGHDFRRTNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 385 - protein, 121 aa, synthetic construct
  REGION 1..121: note = 15G7 HC His variants in rabbit VH 3
QEQLEESGGDLVKPGASLTLTCTASGLHFRRTNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 386 - protein, 121 aa, synthetic construct
  REGION 1..121: note = 15G7 HC His variants in rabbit VH 4
QEQLEESGGDLVKPGASLTLTCTASGLDHRRTNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 387 - protein, 121 aa, synthetic construct
  REGION 1..121: note = 15G7 HC His variants in rabbit VH 5
QEQLEESGGDLVKPGASLTLTCTASGLDFHRTNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 388 - protein, 121 aa, synthetic construct
  REGION 1..121: note = 15G7 HC His variants in rabbit VH 6
QEQLEESGGDLVKPGASLTLTCTASGLDFRHTNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 389 - protein, 121 aa, synthetic construct
  REGION 1..121: note = 15G7 HC His variants in rabbit VH 7
QEQLEESGGDLVKPGASLTLTCTASGLDFRRHNYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 390 - protein, 121 aa, synthetic construct
  REGION 1..121: note = 15G7 HC His variants in rabbit VH 8
QEQLEESGGDLVKPGASLTLTCTASGLDFRRTHYICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 391 - protein, 121 aa, synthetic construct
  REGION 1..121: note = 15G7 HC His variants in rabbit VH 9
QEQLEESGGDLVKPGASLTLTCTASGLDFRRTNHICWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 392 - protein, 121 aa, synthetic construct
  REGION 1..121: note = 15G7 HC His variants in rabbit VH 10
QEQLEESGGDLVKPGASLTLTCTASGLDFRRTNYHCWVRQAPGKGLEWIGCIYIADVVHYYASWAKGRFTFSKTSSTTVTLQMTSLTAADTATYFCARGADGGFVYGMDLWGPGTLVTVSS

SEQ ID NO: 393 - protein, 121 aa, synthetic construct
  REGION 1..121: note = 15G7 HC His variants in rabbit VH 11
QEQLEESGGDLVKPGASLTLTCTASGLDFRRTNYIHWVRQAPGKGLEWIGSIYIADVVHYYASWAKGRFTFSKTSSTTVTLQMTSLTAADTATYFCARGADGGFVYGMDLWGPGTLVTVSS

SEQ ID NO: 394 - protein, 121 aa, synthetic construct
  REGION 1..121: note = 15G7 HC His variants in rabbit VH 12
QEQLEESGGDLVKPGASLTLTCTASGLDFRRTNYISWVRQAPGKGLEWIGHIYIADVVHYYASWAKGRFTFSKTSSTTVTLQMTSLTAADTATYFCARGADGGFVYGMDLWGPGTLVTVSS

SEQ ID NO: 395 - protein, 121 aa, synthetic construct
  REGION 1..121: note = 15G7 HC His variants in rabbit VH 13
QEQLEESGGDLVKPGASLTLTCTASGLDFRRTNYICWVRQAPGKGLEWIGCHYIADVVHYYASWAKGRFTFSKTSSTTVTLQMTSLTAADTATYFCARGADGGFVYGMDLWGPGTLVTVSS

SEQ ID NO: 396 - protein, 121 aa, synthetic construct
  REGION 1..121: note = 15G7 HC His variants in rabbit VH 14
QEQLEESGGDLVKPGASLTLTCTASGLDFRRTNYICWVRQAPGKGLEWIGCIHIADVVHYYASWAKGRFTFSKTSSTTVTLQMTSLTAADTATYFCARGADGGFVYGMDLWGPGTLVTVSS

SEQ ID NO: 397 - protein, 121 aa, synthetic construct
  REGION 1..121: note = 15G7 HC His variants in rabbit VH 15
QEQLEESGGDLVKPGASLTLTCTASGLDFRRTNYICWVRQAPGKGLEWIGCIYHADVVHYYASWAKGRFTFSKTSSTTVTLQMTSLTAADTATYFCARGADGGFVYGMDLWGPGTLVTVSS

SEQ ID NO: 398 - protein, 121 aa, synthetic construct
  REGION 1..121: note = 15G7 HC His variants in rabbit VH 16
QEQLEESGGDLVKPGASLTLTCTASGLDFRRTNYICWVRQAPGKGLEWIGCIYIHDVVHYYASWAKGRFTFSKTSSTTVTLQMTSLTAADTATYFCARGADGGFVYGMDLWGPGTLVTVSS

SEQ ID NO: 399 - protein, 121 aa, synthetic construct
  REGION 1..121: note = 15G7 HC His variants in rabbit VH 17
QEQLEESGGDLVKPGASLTLTCTASGLDFRRTNYICWVRQAPGKGLEWIGCIYIAHVVHYYASWAKGRFTFSKTSSTTVTLQMTSLTAADTATYFCARGADGGFVYGMDLWGPGTLVTVSS

SEQ ID NO: 400 - protein, 121 aa, synthetic construct
  REGION 1..121: note = 15G7 HC His variants in rabbit VH 18
QEQLEESGGDLVKPGASLTLTCTASGLDFRRTNYICWVRQAPGKGLEWIGCIYIADHVHYYASWAKGRFTFSKTSSTTVTLQMTSLTAADTATYFCARGADGGFVYGMDLWGPGTLVTVSS

SEQ ID NO: 401 - protein, 121 aa, synthetic construct
  REGION 1..121: note = 15G7 HC His variants in rabbit VH 19
QEQLEESGGDLVKPGASLTLTCTASGLDFRRTNYICWVRQAPGKGLEWIGCIYIADVHHYYASWAKGRFTFSKTSSTTVTLQMTSLTAADTATYFCARGADGGFVYGMDLWGPGTLVTVSS

SEQ ID NO: 402 - protein, 121 aa, synthetic construct
  REGION 1..121: note = 15G7 HC His variants in rabbit VH 20
QEQLEESGGDLVKPGASLTLTCTASGLDFRRTNYICWVRQAPGKGLEWIGCIYIADVVAYYASWAKGRFTFSKTSSTTVTLQMTSLTAADTATYFCARGADGGFVYGMDLWGPGTLVTVSS

SEQ ID NO: 403 - protein, 121 aa, synthetic construct
  REGION 1..121: note = 15G7 HC His variants in rabbit VH 21
QEQLEESGGDLVKPGASLTLTCTASGLDFRRTNYICWVRQAPGKGLEWIGCIYIADVVHHYASWAKGRFTFSKTSSTTVTLQMTSLTAADTATYFCARGADGGFVYGMDLWGPGTLVTVSS

SEQ ID NO: 404 - protein, 121 aa, synthetic construct
  REGION 1..121: note = 15G7 HC His variants in rabbit VH 22
QEQLEESGGDLVKPGASLTLTCTASGLDFRRTNYICWVRQAPGKGLEWIGCIYIADVVHYHASWAKGRFTFSKTSSTTVTLQMTSLTAADTATYFCARGADGGFVYGMDLWGPGTLVTVSS

SEQ ID NO: 405 - protein, 121 aa, synthetic construct
  REGION 1..121: note = 15G7 HC His variants in rabbit VH 23
QEQLEESGGDLVKPGASLTLTCTASGLDFRRTNYICWVRQAPGKGLEWIGCIYIADVVHYYHSWAKGRFTFSKTSSTTVTLQMTSLTAADTATYFCARGADGGFVYGMDLWGPGTLVTVSS

SEQ ID NO: 406 - protein, 121 aa, synthetic construct
  REGION 1..121: note = 15G7 HC His variants in rabbit VH 24
QEQLEESGGDLVKPGASLTLTCTASGLDFRRTNYICWVRQAPGKGLEWIGCIYIADVVHYYAHWAKGRFTFSKTSSTTVTLQMTSLTAADTATYFCARGADGGFVYGMDLWGPGTLVTVSS

SEQ ID NO: 407 - protein, 121 aa, synthetic construct
  REGION 1..121: note = 15G7 HC His variants in rabbit VH 25
QEQLEESGGDLVKPGASLTLTCTASGLDFRRTNYICWVRQAPGKGLEWIGCIYIADVVHYYASHAKGRFTFSKTSSTTVTLQMTSLTAADTATYFCARGADGGFVYGMDLWGPGTLVTVSS

SEQ ID NO: 408 - protein, 121 aa, synthetic construct
  REGION 1..121: note = 15G7 HC His variants in rabbit VH 26
QEQLEESGGDLVKPGASLTLTCTASGLDFRRTNYICWVRQAPGKGLEWIGCIYIADVVHYYASWHKGRFTFSKTSSTTVTLQMTSLTAADTATYFCARGADGGFVYGMDLWGPGTLVTVSS

SEQ ID NO: 409 - protein, 121 aa, synthetic construct
  REGION 1..121: note = 15G7 HC His variants in rabbit VH 27
QEQLEESGGDLVKPGASLTLTCTASGLDFRRTNYICWVRQAPGKGLEWIGCIYIADVVHYYASWAHGRFTFSKTSSTTVTLQMTSLTAADTATYFCARGADGGFVYGMDLWGPGTLVTVSS

SEQ ID NO: 410 - protein, 121 aa, synthetic construct
  REGION 1..121: note = 15G7 HC His variants in rabbit VH 28
QEQLEESGGDLVKPGASLTLTCTASGLDFRRTNYICWVRQAPGKGLEWIGCIYIADVVHYYASWAKHRFTFSKTSSTTVTLQMTSLTAADTATYFCARGADGGFVYGMDLWGPGTLVTVSS

SEQ ID NO: 411 - protein, 121 aa, synthetic construct
  REGION 1..121: note = 15G7 HC His variants in rabbit VH 29
QEQLEESGGDLVKPGASLTLTCTASGLDFRRTNYICWVRQAPGKGLEWIGCIYIADVVHYYASWAKGRFTFSKTSSTTVTLQMTSLTAADTATYFCHRGADGGFVYGMDLWGPGTLVTVSS

SEQ ID NO: 412 - protein, 121 aa, synthetic construct
  REGION 1..121: note = 15G7 HC His variants in rabbit VH 30
QEQLEESGGDLVKPGASLTLTCTASGLDFRRTNYICWVRQAPGKGLEWIGCIYIADVVHYYASWAKGRFTFSKTSSTTVTLQMTSLTAADTATYFCAHGADGGFVYGMDLWGPGTLVTVSS

SEQ ID NO: 413 - protein, 121 aa, synthetic construct
  REGION 1..121: note = 15G7 HC His variants in rabbit VH 31
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARHADGGFVYGMDLWGPGTLVTVSS

SEQ ID NO: 414 - protein, 121 aa, synthetic construct
  REGION 1..121: note = 15G7 HC His variants in rabbit VH 32
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGHDGGFVYGMDLWGPGTLVTVSS

SEQ ID NO: 415 - protein, 121 aa, synthetic construct
  REGION 1..121: note = 15G7 HC His variants in rabbit VH 33
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGAHGGFVYGMDLWGPGTLVTVSS

SEQ ID NO: 416 - protein, 121 aa, synthetic construct
  REGION 1..121: note = 15G7 HC His variants in rabbit VH 34
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADHGFVYGMDLWGPGTLVTVSS

SEQ ID NO: 417 - protein, 121 aa, synthetic construct
  REGION 1..121: note = 15G7 HC His variants in rabbit VH 35
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGHFVYGMDLWGPGTLVTVSS

SEQ ID NO: 418 - protein, 121 aa, synthetic construct
  REGION 1..121: note = 15G7 HC His variants in rabbit VH 36
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGHVYGMDLWGPGTLVTVSS

SEQ ID NO: 419 - protein, 121 aa, synthetic construct
  REGION 1..121: note = 15G7 HC His variants in rabbit VH 37
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGFHYGMDLWGPGTLVTVSS

SEQ ID NO: 420 - protein, 121 aa, synthetic construct
  REGION 1..121: note = 15G7 HC His variants in rabbit VH 38
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGFVHGMDLWGPGTLVTVSS

SEQ ID NO: 421 - protein, 121 aa, synthetic construct
  REGION 1..121: note = 15G7 HC His variants in rabbit VH 39
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGFVYHMDLWGPGTLVTVSS

SEQ ID NO: 422 - protein, 121 aa, synthetic construct
  REGION 1..121: note = 15G7 HC His variants in rabbit VH 40
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGFVYGHDLWGPGTLVTVSS

SEQ ID NO: 423 - protein, 121 aa, synthetic construct
  REGION 1..121: note = 15G7 HC His variants in rabbit VH 41
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGFVYGMHLWGPGTLVTVSS

SEQ ID NO: 424 - protein, 121 aa, synthetic construct
  REGION 1..121: note = 15G7 HC His variants in rabbit VH 42
QEQLEESGGDLVKPGASLTLTCTASGLDFRRTNYICWVRQAPGKGLEWIGCIYIADVVHYYASWAKGRFTFSKTSSTTVTLQMTSLTAADTATYFCARGADGGFVYGMDHWGPGTLVTVSS

SEQ ID NO: 425 - protein, 122 aa, synthetic construct
  REGION 1..122: note = hu15G7 HC his variant in humanized scaffold 1
EVQLVESGGGLVQPGGSLRLSCTASGLDFRRTNYHCWVRQAPGKGLEWVGCIYIADVVHYYASWAKGRFTFSRDTSKNTVYLQMNSLRAEDTAVYYCARGADGGFVYGMDLWGQGTLVTVSS

SEQ ID NO: 426 - protein, 122 aa, synthetic construct
  REGION 1..122: note = hu15G7 HC his variant in humanized scaffold 2
EVQLVESGGGLVQPGGSLRLSCTASGLDFRRTNHICWVRQAPGKGLEWVGCIYIADVVHYYASWAKGRFTFSRDTSKNTVYLQMNSLRAEDTAVYYCARGADGGFVYGMDLWGQGTLVTVSS

SEQ ID NO: 427 - protein, 122 aa, synthetic construct
  REGION 1..122: note = hu15G7 HC his variant in humanized scaffold 3
EVQLVESGGGLVQPGGSLRLSCTASGLDFRRTNYICWVRQAPGKGLEWVGCIHIADVVHYYASWAKGRFTFSRDTSKNTVYLQMNSLRAEDTAVYYCARGADGGFVYGMDLWGQGTLVTVSS

SEQ ID NO: 428 - protein, 122 aa, synthetic construct
  REGION 1..122: note = hu15G7 HC his variant in humanized scaffold 4
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGHFVYGMDLWGQGTLVTVSS

SEQ ID NO: 429 - protein, 122 aa, synthetic construct
  REGION 1..122: note = hu15G7 HC his variant in humanized scaffold 5
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGGHVYGMDLWGQGTLVTVSS

SEQ ID NO: 430 - protein, 122 aa, synthetic construct
  REGION 1..122: note = hu15G7 HC his variant in humanized scaffold 6
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGGFHYGMDLWGQGTLVTVSS

SEQ ID NO: 431 - protein, 122 aa, synthetic construct
  REGION 1..122: note = hu15G7 HC his combo in humanized scaffold 7
EVQLVESGGGLVQPGGSLRLSCTASGLDFRRTNHICWVRQAPGKGLEWVGCIHIADVVHYYASWAKGRFTFSRDTSKNTVYLQMNSLRAEDTAVYYCARGADGGFVYGMDLWGQGTLVTVSS

SEQ ID NO: 432 - protein, 122 aa, synthetic construct
  REGION 1..122: note = hu15G7 HC his combo in humanized scaffold 8
EVQLVESGGGLVQPGGSLRLSCTASGLDFRRTNHICWVRQAPGKGLEWVGCIYIADVVHYYASWAKGRFTFSRDTSKNTVYLQMNSLRAEDTAVYYCARGADGHFVYGMDLWGQGTLVTVSS

SEQ ID NO: 433 - protein, 122 aa, synthetic construct
  REGION 1..122: note = hu15G7 HC his combo in humanized scaffold 9
EVQLVESGGGLVQPGGSLRLSCTASGLDFRRTNHICWVRQAPGKGLEWVGCIYIADVVHYYASWAKGRFTFSRDTSKNTVYLQMNSLRAEDTAVYYCARGADGGHVYGMDLWGQGTLVTVSS

SEQ ID NO: 434 - protein, 122 aa, synthetic construct
  REGION 1..122: note = hu15G7 HC his combo in humanized scaffold 10
EVQLVESGGGLVQPGGSLRLSCTASGLDFRRTNHICWVRQAPGKGLEWVGCIYIADVVHYYASWAKGRFTFSRDTSKNTVYLQMNSLRAEDTAVYYCARGADGGFHYGMDLWGQGTLVTVSS

SEQ ID NO: 435 - protein, 122 aa, synthetic construct
  REGION 1..122: note = hu15G7 HC his combo in humanized scaffold 11
EVQLVESGGGLVQPGGSLRLSCTASGLDFRRTNYICWVRQAPGKGLEWVGCIHIADVVHYYASWAKGRFTFSRDTSKNTVYLQMNSLRAEDTAVYYCARGADGHFVYGMDLWGQGTLVTVSS

SEQ ID NO: 436 - protein, 122 aa, synthetic construct
  REGION 1..122: note = hu15G7 HC his combo in humanized scaffold 12
EVQLVESGGGLVQPGGSLRLSCTASGLDFRRTNYICWVRQAPGKGLEWVGCIHIADVVHYYASWAKGRFTFSRDTSKNTVYLQMNSLRAEDTAVYYCARGADGGHVYGMDLWGQGTLVTVSS

SEQ ID NO: 437 - protein, 122 aa, synthetic construct
  REGION 1..122: note = hu15G7 HC his combo in humanized scaffold 13
EVQLVESGGGLVQPGGSLRLSCTASGLDFRRTNYICWVRQAPGKGLEWVGCIHIADVVHYYASWAKGRFTFSRDTSKNTVYLQMNSLRAEDTAVYYCARGADGGFHYGMDLWGQGTLVTVSS

SEQ ID NO: 438 - protein, 122 aa, synthetic construct
  REGION 1..122: note = hu15G7 HC his combo in humanized scaffold 14
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGHHVYGMDLWGQGTLVTVSS

SEQ ID NO: 439 - protein, 122 aa, synthetic construct
  REGION 1..122: note = hu15G7 HC his combo in humanized scaffold 15
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGHFHYGMDLWGQGTLVTVSS

SEQ ID NO: 440 - protein, 122 aa, synthetic construct
  REGION 1..122: note = hu15G7 HC his combo in humanized scaffold 16
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGGHHYGMDLWGQGTLVTVSS

SEQ ID NO: 441 - protein, 122 aa, synthetic construct
  REGION 1..122: note = hu15G7 HC his combo in humanized scaffold 17
EVQLVESGGGLVQPGGSLRLSCTASGLDFRRTNHICWVRQAPGKGLEWVGCIHIADVVHYYASWAKGRFTFSRDTSKNTVYLQMNSLRAEDTAVYYCARGADGHFVYGMDLWGQGTLVTVSS

SEQ ID NO: 442 - protein, 122 aa, synthetic construct
  REGION 1..122: note = hu15G7 HC his combo in humanized scaffold 18
EVQLVESGGGLVQPGGSLRLSCTASGLDFRRTNHICWVRQAPGKGLEWVGCIHIADVVHYYASWAKGRFTFSRDTSKNTVYLQMNSLRAEDTAVYYCARGADGGHVYGMDLWGQGTLVTVSS

SEQ ID NO: 443 - protein, 122 aa, synthetic construct
  REGION 1..122: note = hu15G7 HC his combo in humanized scaffold 19
EVQLVESGGGLVQPGGSLRLSCTASGLDFRRTNHICWVRQAPGKGLEWVGCIHIADVVHYYASWAKGRFTFSRDTSKNTVYLQMNSLRAEDTAVYYCARGADGGFHYGMDLWGQGTLVTVSS

SEQ ID NO: 444 - protein, 122 aa, synthetic construct
  REGION 1..122: note = hu15G7 HC his combo in humanized scaffold 20
EVQLVESGGGLVQPGGSLRLSCTASGLDFRRTNHICWVRQAPGKGLEWVGCIYIADVVHYYASWAKGRFTFSRDTSKNTVYLQMNSLRAEDTAVYYCARGADGHHVYGMDLWGQGTLVTVSS

SEQ ID NO: 445 - protein, 122 aa, synthetic construct
  REGION 1..122: note = hu15G7 HC his combo in humanized scaffold 21
EVQLVESGGGLVQPGGSLRLSCTASGLDFRRTNHICWVRQAPGKGLEWVGCIYIADVVHYYASWAKGRFTFSRDTSKNTVYLQMNSLRAEDTAVYYCARGADGHFHYGMDLWGQGTLVTVSS

SEQ ID NO: 446 - protein, 122 aa, synthetic construct
  REGION 1..122: note = hu15G7 HC his combo in humanized scaffold 22
EVQLVESGGGLVQPGGSLRLSCTASGLDFRRTNHICWVRQAPGKGLEWVGCIYIADVVHYYASWAKGRFTFSRDTSKNTVYLQMNSLRAEDTAVYYCARGADGGHHYGMDLWGQGTLVTVSS

SEQ ID NO: 447 - protein, 122 aa, synthetic construct
  REGION 1..122: note = hu15G7 HC his combo in humanized scaffold 23
EVQLVESGGGLVQPGGSLRLSCTASGLDFRRTNYICWVRQAPGKGLEWVGCIHIADVVHYYASWAKGRFTFSRDTSKNTVYLQMNSLRAEDTAVYYCARGADGHHVYGMDLWGQGTLVTVSS

SEQ ID NO: 448 - protein, 122 aa, synthetic construct
  REGION 1..122: note = hu15G7 HC his combo in humanized scaffold 24
EVQLVESGGGLVQPGGSLRLSCTASGLDFRRTNYICWVRQAPGKGLEWVGCIHIADVVHYYASWAKGRFTFSRDTSKNTVYLQMNSLRAEDTAVYYCARGADGHFHYGMDLWGQGTLVTVSS

SEQ ID NO: 449 - protein, 122 aa, synthetic construct
  REGION 1..122: note = hu15G7 HC his combo in humanized scaffold 25
EVQLVESGGGLVQPGGSLRLSCTASGLDFRRTNYICWVRQAPGKGLEWVGCIHIADVVHYYASWAKGRFTFSRDTSKNTVYLQMNSLRAEDTAVYYCARGADGGHHYGMDLWGQGTLVTVSS

SEQ ID NO: 450 - protein, 122 aa, synthetic construct
  REGION 1..122: note = hu15G7 HC his combo in humanized scaffold 26
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGHHHYGMDLWGQGTLVTVSS

SEQ ID NO: 451 - protein, 110 aa, synthetic construct
  REGION 1..110: note = rabbit 15G7 VL
DVVMTQTPASVSEPVGGTVTIKCQASQSIGNYLSWYQQKPGQRPKRLIYSASTLPSGVPSRFKGAGSGTEFTLTISDLESADAATYYCQNNYWLAGYGHPFGGGTEVVVK

SEQ ID NO: 452 - protein, 11 aa, synthetic construct
  REGION 1..11: note = 15G7 LCDR1
QASQSIGNYLS

SEQ ID NO: 453 - protein, 7 aa, synthetic construct
  REGION 1..7: note = 15G7 LCDR2
SASTLPS

SEQ ID NO: 454 - protein, 12 aa, synthetic construct
  REGION 1..12: note = 15G7 LCDR3
QNNYWLAGYGHP

SEQ ID NO: 455 - protein, 110 aa, synthetic construct
  REGION 1..110: note = 15G7 humanized (hu15G7) VL
EIVMTQSPSTLSASVGDRVIITCQASQSIGNYLSWYQQKPGKAPKRLIYSASTLPSGVPSRFSGSGSGAEFTLTISSLQPDDFATYYCQNNYWLAGYGHPFGQGTKLTVL

SEQ ID NO: 456 - protein, 110 aa, synthetic construct
  REGION 1..110: note = 15G7 LC histidine variants in rabbit VL 1
DVVMTQTPASVSEPVGGTVTIKCHASQSIGNYLSWYQQKPGQRPKRLIYSASTLPSGVPSRFKGAGSGTEFTLTISDLESADAATYYCQNNYWLAGYGHPFGGGTEVVVK

SEQ ID NO: 457 - protein, 110 aa, synthetic construct
  REGION 1..110: note = 15G7 LC histidine variants in rabbit VL 2
DVVMTQTPASVSEPVGGTVTIKCQHSQSIGNYLSWYQQKPGQRPKRLIYSASTLPSGVPSRFKGAGSGTEFTLTISDLESADAATYYCQNNYWLAGYGHPFGGGTEVVVK

SEQ ID NO: 458 - protein, 110 aa, synthetic construct
  REGION 1..110: note = 15G7 LC histidine variants in rabbit VL 3
DVVMTQTPASVSEPVGGTVTIKCQAHQSIGNYLSWYQQKPGQRPKRLIYSASTLPSGVPSRFKGAGSGTEFTLTISDLESADAATYYCQNNYWLAGYGHPFGGGTEVVVK

SEQ ID NO: 459 - protein, 110 aa, synthetic construct
  REGION 1..110: note = 15G7 LC histidine variants in rabbit VL 4
DVVMTQTPASVSEPVGGTVTIKCQASHSIGNYLSWYQQKPGQRPKRLIYSASTLPSGVPSRFKGAGSGTEFTLTISDLESADAATYYCQNNYWLAGYGHPFGGGTEVVVK

SEQ ID NO: 460 - protein, 110 aa, synthetic construct
  REGION 1..110: note = 15G7 LC histidine variants in rabbit VL 5
DVVMTQTPASVSEPVGGTVTIKCQASQHIGNYLSWYQQKPGQRPKRLIYSASTLPSGVPSRFKGAGSGTEFTLTISDLESADAATYYCQNNYWLAGYGHPFGGGTEVVVK

SEQ ID NO: 461 - protein, 110 aa, synthetic construct
  REGION 1..110: note = 15G7 LC histidine variants in rabbit VL 6
DVVMTQTPASVSEPVGGTVTIKCQASQSHGNYLSWYQQKPGQRPKRLIYSASTLPSGVPSRFKGAGSGTEFTLTISDLESADAATYYCQNNYWLAGYGHPFGGGTEVVVK

SEQ ID NO: 462 - protein, 110 aa, synthetic construct
  REGION 1..110: note = 15G7 LC histidine variants in rabbit VL 7
DVVMTQTPASVSEPVGGTVTIKCQASQSIHNYLSWYQQKPGQRPKRLIYSASTLPSGVPSRFKGAGSGTEFTLTISDLESADAATYYCQNNYWLAGYGHPFGGGTEVVVK

SEQ ID NO: 463 - protein, 110 aa, synthetic construct
  REGION 1..110: note = 15G7 LC histidine variants in rabbit VL 8
DVVMTQTPASVSEPVGGTVTIKCQASQSIGHYLSWYQQKPGQRPKRLIYSASTLPSGVPSRFKGAGSGTEFTLTISDLESADAATYYCQNNYWLAGYGHPFGGGTEVVVK

SEQ ID NO: 464 - protein, 110 aa, synthetic construct
  REGION 1..110: note = 15G7 LC histidine variants in rabbit VL 9
DVVMTQTPASVSEPVGGTVTIKCQASQSIGNHLSWYQQKPGQRPKRLIYSASTLPSGVPSRFKGAGSGTEFTLTISDLESADAATYYCQNNYWLAGYGHPFGGGTEVVVK

SEQ ID NO: 465 - protein, 110 aa, synthetic construct
  REGION 1..110: note = 15G7 LC histidine variants in rabbit VL 10
DVVMTQTPASVSEPVGGTVTIKCQASQSIGNYHSWYQQKPGQRPKRLIYSASTLPSGVPSRFKGAGSGTEFTLTISDLESADAATYYCQNNYWLAGYGHPFGGGTEVVVK

SEQ ID NO: 466 - protein, 110 aa, synthetic construct
  REGION 1..110: note = 15G7 LC histidine variants in rabbit VL 11
DVVMTQTPASVSEPVGGTVTIKCQASQSIGNYLHWYQQKPGQRPKRLIYSASTLPSGVPSRFKGAGSGTEFTLTISDLESADAATYYCQNNYWLAGYGHPFGGGTEVVVK

SEQ ID NO: 467 - protein, 110 aa, synthetic construct
  REGION 1..110: note = 15G7 LC histidine variants in rabbit VL 12
DVVMTQTPASVSEPVGGTVTIKCQASQSIGNYLSWYQQKPGQRPKRLIYHASTLPSGVPSRFKGAGSGTEFTLTISDLESADAATYYCQNNYWLAGYGHPFGGGTEVVVK

SEQ ID NO: 468 - protein, 110 aa, synthetic construct
  REGION 1..110: note = 15G7 LC histidine variants in rabbit VL 13
DVVMTQTPASVSEPVGGTVTIKCQASQSIGNYLSWYQQKPGQRPKRLIYSHSTLPSGVPSRFKGAGSGTEFTLTISDLESADAATYYCQNNYWLAGYGHPFGGGTEVVVK

SEQ ID NO: 469 - protein, 110 aa, synthetic construct
  REGION 1..110: note = 15G7 LC histidine variants in rabbit VL 14
DVVMTQTPASVSEPVGGTVTIKCQASQSIGNYLSWYQQKPGQRPKRLIYSAHTLPSGVPSRFKGAGSGTEFTLTISDLESADAATYYCQNNYWLAGYGHPFGGGTEVVVK

SEQ ID NO: 470 - protein, 110 aa, synthetic construct
  REGION 1..110: note = 15G7 LC histidine variants in rabbit VL 15
DVVMTQTPASVSEPVGGTVTIKCQASQSIGNYLSWYQQKPGQRPKRLIYSASHLPSGVPSRFKGAGSGTEFTLTISDLESADAATYYCQNNYWLAGYGHPFGGGTEVVVK

SEQ ID NO: 471 - protein, 110 aa, synthetic construct
  REGION 1..110: note = 15G7 LC histidine variants in rabbit VL 16
DVVMTQTPASVSEPVGGTVTIKCQASQSIGNYLSWYQQKPGQRPKRLIYSASTHPSGVPSRFKGAGSGTEFTLTISDLESADAATYYCQNNYWLAGYGHPFGGGTEVVVK

SEQ ID NO: 472 - protein, 110 aa, synthetic construct
  REGION 1..110: note = 15G7 LC histidine variants in rabbit VL 17
DVVMTQTPASVSEPVGGTVTIKCQASQSIGNYLSWYQQKPGQRPKRLIYSASTLHSGVPSRFKGAGSGTEFTLTISDLESADAATYYCQNNYWLAGYGHPFGGGTEVVVK

SEQ ID NO: 473 - protein, 110 aa, synthetic construct
  REGION 1..110: note = 15G7 LC histidine variants in rabbit VL 18
DVVMTQTPASVSEPVGGTVTIKCQASQSIGNYLSWYQQKPGQRPKRLIYSASTLPHGVPSRFKGAGSGTEFTLTISDLESADAATYYCQNNYWLAGYGHPFGGGTEVVVK

SEQ ID NO: 474 - protein, 110 aa, synthetic construct
  REGION 1..110: note = 15G7 LC histidine variants in rabbit VL 19
DVVMTQTPASVSEPVGGTVTIKCQASQSIGNYLSWYQQKPGQRPKRLIYSASTLPSGVPSRFKGAGSGTEFTLTISDLESADAATYYCHNNYWLAGYGHPFGGGTEVVVK

SEQ ID NO: 475 - protein, 110 aa, synthetic construct
  REGION 1..110: note = 15G7 LC histidine variants in rabbit VL 20
DVVMTQTPASVSEPVGGTVTIKCQASQSIGNYLSWYQQKPGQRPKRLIYSASTLPSGVPSRFKGAGSGTEFTLTISDLESADAATYYCQHNYWLAGYGHPFGGGTEVVVK

SEQ ID NO: 476 - protein, 110 aa, synthetic construct
  REGION 1..110: note = 15G7 LC histidine variants in rabbit VL 21
DVVMTQTPASVSEPVGGTVTIKCQASQSIGNYLSWYQQKPGQRPKRLIYSASTLPSGVPSRFKGAGSGTEFTLTISDLESADAATYYCQNHYWLAGYGHPFGGGTEVVVK

SEQ ID NO: 477 - protein, 110 aa, synthetic construct
  REGION 1..110: note = 15G7 LC histidine variants in rabbit VL 22
DVVMTQTPASVSEPVGGTVTIKCQASQSIGNYLSWYQQKPGQRPKRLIYSASTLPSGVPSRFKGAGSGTEFTLTISDLESADAATYYCQNNHWLAGYGHPFGGGTEVVVK

SEQ ID NO: 478 - protein, 110 aa, synthetic construct
  REGION 1..110: note = 15G7 LC histidine variants in rabbit VL 23
DVVMTQTPASVSEPVGGTVTIKCQASQSIGNYLSWYQQKPGQRPKRLIYSASTLPSGVPSRFKGAGSGTEFTLTISDLESADAATYYCQNNYHLAGYGHPFGGGTEVVVK

SEQ ID NO: 479 - protein, 110 aa, synthetic construct
  REGION 1..110: note = 15G7 LC histidine variants in rabbit VL 24
DVVMTQTPASVSEPVGGTVTIKCQASQSIGNYLSWYQQKPGQRPKRLIYSASTLPSGVPSRFKGAGSGTEFTLTISDLESADAATYYCQNNYWHAGYGHPFGGGTEVVVK

SEQ ID NO: 480 - protein, 110 aa, synthetic construct
  REGION 1..110: note = 15G7 LC histidine variants in rabbit VL 25
DVVMTQTPASVSEPVGGTVTIKCQASQSIGNYLSWYQQKPGQRPKRLIYSASTLPSGVPSRFKGAGSGTEFTLTISDLESADAATYYCQNNYWLHGYGHPFGGGTEVVVK

SEQ ID NO: 481 - protein, 110 aa, synthetic construct
  REGION 1..110: note = 15G7 LC histidine variants in rabbit VL 26
DVVMTQTPASVSEPVGGTVTIKCQASQSIGNYLSWYQQKPGQRPKRLIYSASTLPSGVPSRFKGAGSGTEFTLTISDLESADAATYYCQNNYWLAHYGHPFGGGTEVVVK

SEQ ID NO: 482 - protein, 110 aa, synthetic construct
  REGION 1..110: note = 15G7 LC histidine variants in rabbit VL 27
DVVMTQTPASVSEPVGGTVTIKCQASQSIGNYLSWYQQKPGQRPKRLIYSASTLPSGVPSRFKGAGSGTEFTLTISDLESADAATYYCQNNYWLAGHGHPFGGGTEVVVK

SEQ ID NO: 483 - protein, 110 aa, synthetic construct
  REGION 1..110: note = 15G7 LC histidine variants in rabbit VL 28
DVVMTQTPASVSEPVGGTVTIKCQASQSIGNYLSWYQQKPGQRPKRLIYSASTLPSGVPSRFKGAGSGTEFTLTISDLESADAATYYCQNNYWLAGYHHPFGGGTEVVVK

SEQ ID NO: 484 - protein, 110 aa, synthetic construct
  REGION 1..110: note = 15G7 LC histidine variants in rabbit VL 29
DVVMTQTPASVSEPVGGTVTIKCQASQSIGNYLSWYQQKPGQRPKRLIYSASTLPSGVPSRFKGAGSGTEFTLTISDLESADAATYYCQNNYWLAGYGAPFGGGTEVVVK

SEQ ID NO: 485 - protein, 110 aa, synthetic construct
  REGION 1..110: note = 15G7 LC histidine variants in rabbit VL 30
DVVMTQTPASVSEPVGGTVTIKCQASQSIGNYLSWYQQKPGQRPKRLIYSASTLPSGVPSRFKGAGSGTEFTLTISDLESADAATYYCQNNYWLAGYGHHFGGGTEVVVK

SEQ ID NO: 486 - protein, 110 aa, synthetic construct
  REGION 1..110: note = hu15G7 LC his variant in humanized scaffold 1
EIVMTQSPSTLSASVGDRVIITCQASQSIGNYLSWYQQKPGKAPKRLIYSASTLPSGVPSRFSGSGSGAEFTLTISSLQPDDFATYYCQNNYWLAGYGHHFGQGTKLTVL

SEQ ID NO: 487 - protein, 110 aa, synthetic construct
  REGION 1..110: note = hu15G7 LC his variant in humanized scaffold 2
EIVMTQSPSTLSASVGDRVIITCQASQSIHNYLSWYQQKPGKAPKRLIYSASTLPSGVPSRFSGSGSGAEFTLTISSLQPDDFATYYCQNNYWLAGYGHPFGQGTKLTVL

SEQ ID NO: 488 - protein, 110 aa, synthetic construct
  REGION 1..110: note = hu15G7 LC his variant in humanized scaffold 3
EIVMTQSPSTLSASVGDRVIITCQASQSIGNHLSWYQQKPGKAPKRLIYSASTLPSGVPSRFSGSGSGAEFTLTISSLQPDDFATYYCQNNYWLAGYGHPFGQGTKLTVL

SEQ ID NO: 489 - protein, 110 aa, synthetic construct
  REGION 1..110: note = hu15G7 LC his variant in humanized scaffold 4
EIVMTQSPSTLSASVGDRVIITCQASQSIGNYLSWYQQKPGKAPKRLIYSASTLPSGVPSRFSGSGSGAEFTLTISSLQPDDFATYYCQNNHWLAGYGHPFGQGTKLTVL

SEQ ID NO: 490 - protein, 110 aa, synthetic construct
  REGION 1..110: note = hu15G7 LC his variant in humanized scaffold 5
EIVMTQSPSTLSASVGDRVIITCQASQSIGNYLSWYQQKPGKAPKRLIYSASTLPSGVPSRFSGSGSGAEFTLTISSLQPDDFATYYCQNNYHLAGYGHPFGQGTKLTVL

SEQ ID NO: 491 - protein, 110 aa, synthetic construct
  REGION 1..110: note = hu15G7 LC his variant in humanized scaffold 6
EIVMTQSPSTLSASVGDRVIITCQASQSIGNYLSWYQQKPGKAPKRLIYSASTLPSGVPSRFSGSGSGAEFTLTISSLQPDDFATYYCQNNYWLAHYGHPFGQGTKLTVL

SEQ ID NO: 492 - protein, 110 aa, synthetic construct
  REGION 1..110: note = hu15G7 LC his combo in humanized scaffold 7
EIVMTQSPSTLSASVGDRVIITCQASQSIHNHLSWYQQKPGKAPKRLIYSASTLPSGVPSRFSGSGSGAEFTLTISSLQPDDFATYYCQNNYWLAGYGHPFGQGTKLTVL

SEQ ID NO: 493 - protein, 110 aa, synthetic construct
  REGION 1..110: note = hu15G7 LC his combo in humanized scaffold 8
EIVMTQSPSTLSASVGDRVIITCQASQSIHNYLSWYQQKPGKAPKRLIYSASTLPSGVPSRFSGSGSGAEFTLTISSLQPDDFATYYCQNNHWLAGYGHPFGQGTKLTVL

SEQ ID NO: 494 - protein, 110 aa, synthetic construct
  REGION 1..110: note = hu15G7 LC his combo in humanized scaffold 9
EIVMTQSPSTLSASVGDRVIITCQASQSIHNYLSWYQQKPGKAPKRLIYSASTLPSGVPSRFSGSGSGAEFTLTISSLQPDDFATYYCQNNYHLAGYGHPFGQGTKLTVL

SEQ ID NO: 495 - protein, 110 aa, synthetic construct
  REGION 1..110: note = hu15G7 LC his combo in humanized scaffold 10
EIVMTQSPSTLSASVGDRVIITCQASQSIHNYLSWYQQKPGKAPKRLIYSASTLPSGVPSRFSGSGSGAEFTLTISSLQPDDFATYYCQNNYWLAHYGHPFGQGTKLTVL

SEQ ID NO: 496 - protein, 110 aa, synthetic construct
  REGION 1..110: note = hu15G7 LC his combo in humanized scaffold 11
EIVMTQSPSTLSASVGDRVIITCQASQSIGNHLSWYQQKPGKAPKRLIYSASTLPSGVPSRFSGSGSGAEFTLTISSLQPDDFATYYCQNNHWLAGYGHPFGQGTKLTVL

SEQ ID NO: 497 - protein, 110 aa, synthetic construct
  REGION 1..110: note = hu15G7 LC his combo in humanized scaffold 12
EIVMTQSPSTLSASVGDRVIITCQASQSIGNHLSWYQQKPGKAPKRLIYSASTLPSGVPSRFSGSGSGAEFTLTISSLQPDDFATYYCQNNYHLAGYGHPFGQGTKLTVL

SEQ ID NO: 498 - protein, 110 aa, synthetic construct
  REGION 1..110: note = hu15G7 LC his combo in humanized scaffold 13
EIVMTQSPSTLSASVGDRVIITCQASQSIGNHLSWYQQKPGKAPKRLIYSASTLPSGVPSRFSGSGSGAEFTLTISSLQPDDFATYYCQNNYWLAHYGHPFGQGTKLTVL

SEQ ID NO: 499 - protein, 110 aa, synthetic construct
  REGION 1..110: note = hu15G7 LC his combo in humanized scaffold 14
EIVMTQSPSTLSASVGDRVIITCQASQSIGNYLSWYQQKPGKAPKRLIYSASTLPSGVPSRFSGSGSGAEFTLTISSLQPDDFATYYCQNNHHLAGYGHPFGQGTKLTVL

SEQ ID NO: 500 - protein, 110 aa, synthetic construct
  REGION 1..110: note = hu15G7 LC his combo in humanized scaffold 15
EIVMTQSPSTLSASVGDRVIITCQASQSIGNYLSWYQQKPGKAPKRLIYSASTLPSGVPSRFSGSGSGAEFTLTISSLQPDDFATYYCQNNHWLAHYGHPFGQGTKLTVL

SEQ ID NO: 501 - protein, 110 aa, synthetic construct
  REGION 1..110: note = hu15G7 LC his combo in humanized scaffold 16
EIVMTQSPSTLSASVGDRVIITCQASQSIGNYLSWYQQKPGKAPKRLIYSASTLPSGVPSRFSGSGSGAEFTLTISSLQPDDFATYYCQNNYHLAHYGHPFGQGTKLTVL

SEQ ID NO: 502 - protein, 110 aa, synthetic construct
  REGION 1..110: note = hu15G7 LC his combo in humanized scaffold 17
EIVMTQSPSTLSASVGDRVIITCQASQSIHNHLSWYQQKPGKAPKRLIYSASTLPSGVPSRFSGSGSGAEFTLTISSLQPDDFATYYCQNNHWLAGYGHPFGQGTKLTVL

SEQ ID NO: 503 - protein, 110 aa, synthetic construct
  REGION 1..110: note = hu15G7 LC his combo in humanized scaffold 18
EIVMTQSPSTLSASVGDRVIITCQASQSIHNHLSWYQQKPGKAPKRLIYSASTLPSGVPSRFSGSGSGAEFTLTISSLQPDDFATYYCQNNYHLAGYGHPFGQGTKLTVL

SEQ ID NO: 504 - protein, 110 aa, synthetic construct
  REGION 1..110: note = hu15G7 LC his combo in humanized scaffold 19
EIVMTQSPSTLSASVGDRVIITCQASQSIHNHLSWYQQKPGKAPKRLIYSASTLPSGVPSRFSGSGSGAEFTLTISSLQPDDFATYYCQNNYWLAHYGHPFGQGTKLTVL

SEQ ID NO: 505 - protein, 110 aa, synthetic construct
  REGION 1..110: note = hu15G7 LC his combo in humanized scaffold 20
EIVMTQSPSTLSASVGDRVIITCQASQSIHNYLSWYQQKPGKAPKRLIYSASTLPSGVPSRFSGSGSGAEFTLTISSLQPDDFATYYCQNNHHLAGYGHPFGQGTKLTVL

SEQ ID NO: 506 - protein, 110 aa, synthetic construct
  REGION 1..110: note = hu15G7 LC his combo in humanized scaffold 21
EIVMTQSPSTLSASVGDRVIITCQASQSIHNYLSWYQQKPGKAPKRLIYSASTLPSGVPSRFSGSGSGAEFTLTISSLQPDDFATYYCQNNHWLAHYGHPFGQGTKLTVL

SEQ ID NO: 507 - protein, 110 aa, synthetic construct
  REGION 1..110: note = hu15G7 LC his combo in humanized scaffold 22
EIVMTQSPSTLSASVGDRVIITCQASQSIHNYLSWYQQKPGKAPKRLIYSASTLPSGVPSRFSGSGSGAEFTLTISSLQPDDFATYYCQNNYHLAHYGHPFGQGTKLTVL

SEQ ID NO: 508 - protein, 110 aa, synthetic construct
  REGION 1..110: note = hu15G7 LC his combo in humanized scaffold 23
EIVMTQSPSTLSASVGDRVIITCQASQSIGNHLSWYQQKPGKAPKRLIYSASTLPSGVPSRFSGSGSGAEFTLTISSLQPDDFATYYCQNNHHLAGYGHPFGQGTKLTVL

SEQ ID NO: 509 - protein, 110 aa, synthetic construct
  REGION 1..110: note = hu15G7 LC his combo in humanized scaffold 24
EIVMTQSPSTLSASVGDRVIITCQASQSIGNHLSWYQQKPGKAPKRLIYSASTLPSGVPSRFSGSGSGAEFTLTISSLQPDDFATYYCQNNHWLAHYGHPFGQGTKLTVL

SEQ ID NO: 510 - protein, 110 aa, synthetic construct
  REGION 1..110: note = hu15G7 LC his combo in humanized scaffold 25
EIVMTQSPSTLSASVGDRVIITCQASQSIGNHLSWYQQKPGKAPKRLIYSASTLPSGVPSRFSGSGSGAEFTLTISSLQPDDFATYYCQNNYHLAHYGHPFGQGTKLTVL

SEQ ID NO: 511 - protein, 110 aa, synthetic construct
  REGION 1..110: note = hu15G7 LC his combo in humanized scaffold 26
EIVMTQSPSTLSASVGDRVIITCQASQSIGNYLSWYQQKPGKAPKRLIYSASTLPSGVPSRFSGSGSGAEFTLTISSLQPDDFATYYCQNNHHLAHYGHPFGQGTKLTVL

SEQ ID NO: 512 - protein, 124 aa, synthetic construct
  REGION 1..124: note = 24D9 rabbit VH
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 513 - protein, 11 aa, synthetic construct
  REGION 1..11: note = 24D9 HCDR1
GFSFSGSYWIC

SEQ ID NO: 514 - protein, 18 aa, synthetic construct
  REGION 1..18: note = 24D9 HCDR2
CINGGSLDITYYASWAKG

SEQ ID NO: 515 - protein, 16 aa, synthetic construct
  REGION 1..16: note = 24D9 HCDR3
ASDTSYTAYGNFYFNL

SEQ ID NO: 516 - protein, 125 aa, synthetic construct
  REGION 1..125: note = 24D9 humanized (hu24D9) VH
EVQLVESGGGLVQPGGSLRLSCTASGFSFSGSYWICWVRQAPGKGLEWVGCINGGSLDITYYASWAKGRFTISRDTSKNTVYLQKTSLRAEDTAVYYCASDTSYTAYGNFYFNLWGQGTLVTVSS

SEQ ID NO: 517 - protein, 113 aa, synthetic construct
  REGION 1..113: note = hu24D9 LC "EIK" humanization variant
EIVMTQSPSPLSASVGDRVIITCQSSQSVLNNNWLAWYQQKPGKAPKLLIYDASSLASGVPSRFSGSGSGAEFTLTISSLQPDDFATYYCQGEFSCGSADCNAFGQGTKLEIK

SEQ ID NO: 518 - protein, 122 aa, synthetic construct
  REGION 1..122: note = 24D9 rabbit VL
AQVLTQTASPVSAAVGGTVTINCQSSQSVLNNNWLAWYQQKPGQPPKLLIYDASSLASGVPSRFKGSGSGTQFTLTISDLESDDAATYYCQGEFSCGSADCNAFGGGTEVVVKGDPVAPTVL

SEQ ID NO: 519 - protein, 13 aa, synthetic construct
  REGION 1..13: note = 24D9 LCDR1
QSSQSVLNNNWLA

SEQ ID NO: 520 - protein, 7 aa, synthetic construct
  REGION 1..7: note = 24D9 LCDR2
DASSLAS

SEQ ID NO: 521 - protein, 13 aa, synthetic construct
  REGION 1..13: note = 24D9 LCDR3
QGEFSCGSADCNA

SEQ ID NO: 522 - protein, 113 aa, synthetic construct
  REGION 1..113: note = 24D9 humanized (hu24D9) VL
EIVMTQSPSPLSASVGDRVIITCQSSQSVLNNNWLAWYQQKPGKAPKLLIYDASSLASGVPSRFSGSGSGAEFTLTISSLQPDDFATYYCQGEFSCGSADCNAFGQGTKLTVL

SEQ ID NO: 523 - protein, 124 aa, synthetic construct
  REGION 1..124: note = 24D9 HC His variants in rabbit VH 1
QQQLVESGGGLVKPGASLTLTCKASHFSFSGSYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 524 - protein, 124 aa, synthetic construct
  REGION 1..124: note = 24D9 HC His variants in rabbit VH 2
QQQLVESGGGLVKPGASLTLTCKASGHSFSGSYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 525 - protein, 124 aa, synthetic construct
  REGION 1..124: note = 24D9 HC His variants in rabbit VH 3
QQQLVESGGGLVKPGASLTLTCKASGFHFSGSYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 526 - protein, 124 aa, synthetic construct
  REGION 1..124: note = 24D9 HC His variants in rabbit VH 4
QQQLVESGGGLVKPGASLTLTCKASGFSHSGSYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 527 - protein, 124 aa, synthetic construct
  REGION 1..124: note = 24D9 HC His variants in rabbit VH 5
QQQLVESGGGLVKPGASLTLTCKASGFSFHGSYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 528 - protein, 124 aa, synthetic construct
  REGION 1..124: note = 24D9 HC His variants in rabbit VH 6
QQQLVESGGGLVKPGASLTLTCKASGFSFSHSYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 529 - protein, 124 aa, synthetic construct
  REGION 1..124: note = 24D9 HC His variants in rabbit VH 7
QQQLVESGGGLVKPGASLTLTCKASGFSFSGHYWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 530 - protein, 124 aa, synthetic construct
  REGION 1..124: note = 24D9 HC His variants in rabbit VH 8
QQQLVESGGGLVKPGASLTLTCKASGFSFSGSHWICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 531 - protein, 124 aa, synthetic construct
  REGION 1..124: note = 24D9 HC His variants in rabbit VH 9
QQQLVESGGGLVKPGASLTLTCKASGFSFSGSYHICWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 532 - protein, 124 aa, synthetic construct
  REGION 1..124: note = 24D9 HC His variants in rabbit VH 10
QQQLVESGGGLVKPGASLTLTCKASGFSFSGSYWHCWVRQAPGKGLEWIGCINGGSLDITYYASWAKGRFTISKASSTTVTLQKTSLTAADTATYFCASDTSYTAYGNFYFNLWGPGTLVTVSS

SEQ ID NO: 533 - protein, 124 aa, synthetic construct
  REGION 1..124: note = 24D9 HC His variants in rabbit VH 11
QQQLVESGGGLVKPGASLTLTCKASGFSFSGSYWIHWVRQAPGKGLEWIGSINGGSLDITYYASWAKGRFTISKASSTTVTLQKTSLTAADTATYFCASDTSYTAYGNFYFNLWGPGTLVTVSS

SEQ ID NO: 534 - protein, 124 aa, synthetic construct
  REGION 1..124: note = 24D9 HC His variants in rabbit VH 12
QQQLVESGGGLVKPGASLTLTCKASGFSFSGSYWISWVRQAPGKGLEWIGHINGGSLDITYYASWAKGRFTISKASSTTVTLQKTSLTAADTATYFCASDTSYTAYGNFYFNLWGPGTLVTVSS

SEQ ID NO: 535 - protein, 124 aa, synthetic construct
  REGION 1..124: note = 24D9 HC His variants in rabbit VH 13
QQQLVESGGGLVKPGASLTLTCKASGFSFSGSYWICWVRQAPGKGLEWIGCHNGGSLDITYYASWAKGRFTISKASSTTVTLQKTSLTAADTATYFCASDTSYTAYGNFYFNLWGPGTLVTVSS

SEQ ID NO: 536 - protein, 124 aa, synthetic construct
  REGION 1..124: note = 24D9 HC His variants in rabbit VH 14
QQQLVESGGGLVKPGASLTLTCKASGFSFSGSYWICWVRQAPGKGLEWIGCIHGGSLDITYYASWAKGRFTISKASSTTVTLQKTSLTAADTATYFCASDTSYTAYGNFYFNLWGPGTLVTVSS

SEQ ID NO: 537 - protein, 124 aa, synthetic construct
  REGION 1..124: note = 24D9 HC His variants in rabbit VH 15
QQQLVESGGGLVKPGASLTLTCKASGFSFSGSYWICWVRQAPGKGLEWIGCINHGSLDITYYASWAKGRFTISKASSTTVTLQKTSLTAADTATYFCASDTSYTAYGNFYFNLWGPGTLVTVSS

SEQ ID NO: 538 - protein, 124 aa, synthetic construct
  REGION 1..124: note = 24D9 HC His variants in rabbit VH 16
QQQLVESGGGLVKPGASLTLTCKASGFSFSGSYWICWVRQAPGKGLEWIGCINGHSLDITYYASWAKGRFTISKASSTTVTLQKTSLTAADTATYFCASDTSYTAYGNFYFNLWGPGTLVTVSS

SEQ ID NO: 539 - protein, 124 aa, synthetic construct
  REGION 1..124: note = 24D9 HC His variants in rabbit VH 17
QQQLVESGGGLVKPGASLTLTCKASGFSFSGSYWICWVRQAPGKGLEWIGCINGGHLDITYYASWAKGRFTISKASSTTVTLQKTSLTAADTATYFCASDTSYTAYGNFYFNLWGPGTLVTVSS

SEQ ID NO: 540 - protein, 124 aa, synthetic construct
  REGION 1..124: note = 24D9 HC His variants in rabbit VH 18
QQQLVESGGGLVKPGASLTLTCKASGFSFSGSYWICWVRQAPGKGLEWIGCINGGSHDITYYASWAKGRFTISKASSTTVTLQKTSLTAADTATYFCASDTSYTAYGNFYFNLWGPGTLVTVSS

SEQ ID NO: 541 - protein, 124 aa, synthetic construct
  REGION 1..124: note = 24D9 HC His variants in rabbit VH 19
QQQLVESGGGLVKPGASLTLTCKASGFSFSGSYWICWVRQAPGKGLEWIGCINGGSLHITYYASWAKGRFTISKASSTTVTLQKTSLTAADTATYFCASDTSYTAYGNFYFNLWGPGTLVTVSS

SEQ ID NO: 542 - protein, 124 aa, synthetic construct
  REGION 1..124: note = 24D9 HC His variants in rabbit VH 20
QQQLVESGGGLVKPGASLTLTCKASGFSFSGSYWICWVRQAPGKGLEWIGCINGGSLDHTYYASWAKGRFTISKASSTTVTLQKTSLTAADTATYFCASDTSYTAYGNFYFNLWGPGTLVTVSS

SEQ ID NO: 543 - protein, 124 aa, synthetic construct
  REGION 1..124: note = 24D9 HC His variants in rabbit VH 21
QQQLVESGGGLVKPGASLTLTCKASGFSFSGSYWICWVRQAPGKGLEWIGCINGGSLDIHYYASWAKGRFTISKASSTTVTLQKTSLTAADTATYFCASDTSYTAYGNFYFNLWGPGTLVTVSS

SEQ ID NO: 544 - protein, 124 aa, synthetic construct
  REGION 1..124: note = 24D9 HC His variants in rabbit VH 22
QQQLVESGGGLVKPGASLTLTCKASGFSFSGSYWICWVRQAPGKGLEWIGCINGGSLDITHYASWAKGRFTISKASSTTVTLQKTSLTAADTATYFCASDTSYTAYGNFYFNLWGPGTLVTVSS

SEQ ID NO: 545 - protein, 124 aa, synthetic construct
  REGION 1..124: note = 24D9 HC His variants in rabbit VH 23
QQQLVESGGGLVKPGASLTLTCKASGFSFSGSYWICWVRQAPGKGLEWIGCINGGSLDITYHASWAKGRFTISKASSTTVTLQKTSLTAADTATYFCASDTSYTAYGNFYFNLWGPGTLVTVSS

SEQ ID NO: 546 - protein, 124 aa, synthetic construct
  REGION 1..124: note = 24D9 HC His variants in rabbit VH 24
QQQLVESGGGLVKPGASLTLTCKASGFSFSGSYWICWVRQAPGKGLEWIGCINGGSLDITYYHSWAKGRFTISKASSTTVTLQKTSLTAADTATYFCASDTSYTAYGNFYFNLWGPGTLVTVSS

SEQ ID NO: 547 - protein, 124 aa, synthetic construct
  REGION 1..124: note = 24D9 HC His variants in rabbit VH 25
QQQLVESGGGLVKPGASLTLTCKASGFSFSGSYWICWVRQAPGKGLEWIGCINGGSLDITYYAHWAKGRFTISKASSTTVTLQKTSLTAADTATYFCASDTSYTAYGNFYFNLWGPGTLVTVSS

SEQ ID NO: 548 - protein, 124 aa, synthetic construct
  REGION 1..124: note = 24D9 HC His variants in rabbit VH 26
QQQLVESGGGLVKPGASLTLTCKASGFSFSGSYWICWVRQAPGKGLEWIGCINGGSLDITYYASHAKGRFTISKASSTTVTLQKTSLTAADTATYFCASDTSYTAYGNFYFNLWGPGTLVTVSS

SEQ ID NO: 549 - protein, 124 aa, synthetic construct
  REGION 1..124: note = 24D9 HC His variants in rabbit VH 27
QQQLVESGGGLVKPGASLTLTCKASGFSFSGSYWICWVRQAPGKGLEWIGCINGGSLDITYYASWHKGRFTISKASSTTVTLQKTSLTAADTATYFCASDTSYTAYGNFYFNLWGPGTLVTVSS

SEQ ID NO: 550 - protein, 124 aa, synthetic construct
  REGION 1..124: note = 24D9 HC His variants in rabbit VH 28
QQQLVESGGGLVKPGASLTLTCKASGFSFSGSYWICWVRQAPGKGLEWIGCINGGSLDITYYASWAHGRFTISKASSTTVTLQKTSLTAADTATYFCASDTSYTAYGNFYFNLWGPGTLVTVSS

SEQ ID NO: 551 - protein, 124 aa, synthetic construct
  REGION 1..124: note = 24D9 HC His variants in rabbit VH 29
QQQLVESGGGLVKPGASLTLTCKASGFSFSGSYWICWVRQAPGKGLEWIGCINGGSLDITYYASWAKHRFTISKASSTTVTLQKTSLTAADTATYFCASDTSYTAYGNFYFNLWGPGTLVTVSS

SEQ ID NO: 552 - protein, 124 aa, synthetic construct
  REGION 1..124: note = 24D9 HC His variants in rabbit VH 30
QQQLVESGGGLVKPGASLTLTCKASGFSFSGSYWICWVRQAPGKGLEWIGCINGGSLDITYYASWAKGRFTISKASSTTVTLQKTSLTAADTATYFCHSDTSYTAYGNFYFNLWGPGTLVTVSS

SEQ ID NO: 553 - protein, 124 aa, synthetic construct
  REGION 1..124: note = 24D9 HC His variants in rabbit VH 31
QQQLVESGGGLVKPGASLTLTCKASGFSFSGSYWICWVRQAPGKGLEWIGCINGGSLDITYYASWAKGRFTISKASSTTVTLQKTSLTAADTATYFCAHDTSYTAYGNFYFNLWGPGTLVTVSS

SEQ ID NO: 554 - protein, 124 aa, synthetic construct
  REGION 1..124: note = 24D9 HC His variants in rabbit VH 32
QQQLVESGGGLVKPGASLTLTCKASGFSFSGSYWICWVRQAPGKGLEWIGCINGGSLDITYYASWAKGRFTISKASSTTVTLQKTSLTAADTATYFCASHTSYTAYGNFYFNLWGPGTLVTVSS

SEQ ID NO: 555 - protein, 124 aa, synthetic construct
  REGION 1..124: note = 24D9 HC His variants in rabbit VH 33
QQQLVESGGGLVKPGASLTLTCKASGFSFSGSYWICWVRQAPGKGLEWIGCINGGSLDITYYASWAKGRFTISKASSTTVTLQKTSLTAADTATYFCASDHSYTAYGNFYFNLWGPGTLVTVSS

SEQ ID NO: 556 - protein, 124 aa, synthetic construct
  REGION 1..124: note = 24D9 HC His variants in rabbit VH 34
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTHYTAYGNFYFNLWGPGTLVTVSS

SEQ ID NO: 557 - protein, 124 aa, synthetic construct
  REGION 1..124: note = 24D9 HC His variants in rabbit VH 35
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSHTAYGNFYFNLWGPGTLVTVSS

SEQ ID NO: 558 - protein, 124 aa, synthetic construct
  REGION 1..124: note = 24D9 HC His variants in rabbit VH 36
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYHAYGNFYFNLWGPGTLVTVSS

SEQ ID NO: 559 - protein, 124 aa, synthetic construct
  REGION 1..124: note = 24D9 HC His variants in rabbit VH 37
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTHYGNFYFNLWGPGTLVTVSS

SEQ ID NO: 560 - protein, 124 aa, synthetic construct
  REGION 1..124: note = 24D9 HC His variants in rabbit VH 38
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAHGNFYFNLWGPGTLVTVSS

SEQ ID NO: 561 - protein, 124 aa, synthetic construct
  REGION 1..124: note = 24D9 HC His variants in rabbit VH 39
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYHNFYFNLWGPGTLVTVSS

SEQ ID NO: 562 - protein, 124 aa, synthetic construct
  REGION 1..124: note = 24D9 HC His variants in rabbit VH 40
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYGHFYFNLWGPGTLVTVSS

SEQ ID NO: 563 - protein, 124 aa, synthetic construct
  REGION 1..124: note = 24D9 HC His variants in rabbit VH 41
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYGNHYFNLWGPGTLVTVSS

SEQ ID NO: 564 - protein, 124 aa, synthetic construct
  REGION 1..124: note = 24D9 HC His variants in rabbit VH 42
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYGNFHFNLWGPGTLVTVSS

SEQ ID NO: 565 - protein, 124 aa, synthetic construct
  REGION 1..124: note = 24D9 HC His variants in rabbit VH 43
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYGNFYHNLWGPGTLVTVSS

SEQ ID NO: 566 - protein, 124 aa, synthetic construct
  REGION 1..124: note = 24D9 HC His variants in rabbit VH 44
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYGNFYFHLWGPGTLVTVSS

SEQ ID NO: 567 - protein, 124 aa, synthetic construct
  REGION 1..124: note = 24D9 HC His variants in rabbit VH 45
QQQLVESGGGLVKPGASLTLTCKASGFSFSGSYWICWVRQAPGKGLEWIGCINGGSLDITYYASWAKGRFTISKASSTTVTLQKTSLTAADTATYFCASDTSYTAYGNFYFNHWGPGTLVTVSS

SEQ ID NO: 568 - protein, 125 aa, synthetic construct
  REGION 1..125: note = hu24D9 HC his variant in humanized scaffold 1
EVQLVESGGGLVQPGGSLRLSCTASGFSFSGSYWICWVRQAPGKGLEWVGCIHGGSLDITYYASWAKGRFTISRDTSKNTVYLQKTSLRAEDTAVYYCASDTSYTAYGNFYFNLWGQGTLVTVSS

SEQ ID NO: 569 - protein, 125 aa, synthetic construct
  REGION 1..125: note = hu24D9 HC his variant in humanized scaffold 2
EVQLVESGGGLVQPGGSLRLSCTASGFSFSGHYWICWVRQAPGKGLEWVGCINGGSLDITYYASWAKGRFTISRDTSKNTVYLQKTSLRAEDTAVYYCASDTSYTAYGNFYFNLWGQGTLVTVSS

SEQ ID NO: 570 - protein, 125 aa, synthetic construct
  REGION 1..125: note = hu24D9 HC his variant in humanized scaffold 3
EVQLVESGGGLVQPGGSLRLSCTASGFSFSGSYWICWVRQAPGKGLEWVGCINGGSHDITYYASWAKGRFTISRDTSKNTVYLQKTSLRAEDTAVYYCASDTSYTAYGNFYFNLWGQGTLVTVSS

SEQ ID NO: 571 - protein, 125 aa, synthetic construct
  REGION 1..125: note = hu24D9 HC his combo in humanized scaffold
EVQLVESGGGLVQPGGSLRLSCTASGFSFSGHYWICWVRQAPGKGLEWVGCIHGGSHDITYYASWAKGRFTISRDTSKNTVYLQKTSLRAEDTAVYYCASDTSYTAYGNFYFNLWGQGTLVTVSS

SEQ ID NO: 572 - protein, 123 aa, synthetic construct
  REGION 1..123: note = 29F1 rabbit VH
QEQLEESGGDLVQPEGSLTLTCTASGFSFSSSHWICWVRQAPGKGLEWIACIYAGSIGSTYYASWAKGRFTITRSTSLNTVDLKMTSLTAADTATYFCARAPNDIYNYYFNLWGPGTLVTVSS

SEQ ID NO: 573 - protein, 123 aa, synthetic construct; identical to SEQ ID NO: 572
  REGION 1..123: note = 29F1 HCDR1

SEQ ID NO: 574 - protein, 123 aa, synthetic construct; identical to SEQ ID NO: 572
  REGION 1..123: note = 29F1 HCDR2

SEQ ID NO: 575 - protein, 123 aa, synthetic construct; identical to SEQ ID NO: 572
  REGION 1..123: note = 29F1 HCDR3

SEQ ID NO: 576 - protein, 123 aa, synthetic construct
  REGION 1..123: note = 29F1 humanized (hu29F1) VH
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 577 - protein, 117 aa, synthetic construct
  REGION 1..117: note = rabbit 29F1 VL
DVVMTQTPSSVSAAVGGTVTIKCQASENIYNYLAWYQQKPGQPPNLLIYYASTLASGVPSRFSGSGSGTEYTLTISGVQSADAAIYYCQSGYVSKSSTFGGGTEVVVKGDPVAPTVL

SEQ ID NO: 578 - protein, 117 aa, synthetic construct; identical to SEQ ID NO: 577
  REGION 1..117: note = 29F1 LCDR1

SEQ ID NO: 579 - protein, 117 aa, synthetic construct; identical to SEQ ID NO: 577
  REGION 1..117: note = 29F1 LCDR2

SEQ ID NO: 580 - protein, 117 aa, synthetic construct; identical to SEQ ID NO: 577
  REGION 1..117: note = 29F1 LCDR3

SEQ ID NO: 581 - protein, 108 aa, synthetic construct
  REGION 1..108: note = 29F1 humanized (hu29F1) VL
EIVMTQSPSTLSASVGDRVIITCQASENIYNYLAWYQQKPGKAPKLLIYYASTLASGVPSRFSGSGSGAEFTLTISSLQPDDFATYYCQSGYVSKSSTFGQGTKLTVL

SEQ ID NO: 582 - protein, 123 aa, synthetic construct
  REGION 1..123: note = hu29F1 VH aspartate variant in hu29F1 VH 1
EVQLVESGGGLVQPGGSLRLSCTASDFSFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 583 - protein, 123 aa, synthetic construct
  REGION 1..123: note = hu29F1 VH aspartate variant in hu29F1 VH 2
EVQLVESGGGLVQPGGSLRLSCTASGDSFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 584 - protein, 123 aa, synthetic construct
  REGION 1..123: note = hu29F1 VH aspartate variant in hu29F1 VH 3
EVQLVESGGGLVQPGGSLRLSCTASGFDFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 585 - protein, 123 aa, synthetic construct
  REGION 1..123: note = hu29F1 VH aspartate variant in hu29F1 VH 4
EVQLVESGGGLVQPGGSLRLSCTASGFSDSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 586 - protein, 123 aa, synthetic construct
  REGION 1..123: note = hu29F1 VH aspartate variant in hu29F1 VH 5
EVQLVESGGGLVQPGGSLRLSCTASGFSFDSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 587 - protein, 123 aa, synthetic construct
  REGION 1..123: note = hu29F1 VH aspartate variant in hu29F1 VH 6
EVQLVESGGGLVQPGGSLRLSCTASGFSFSDSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 588 - protein, 123 aa, synthetic construct
  REGION 1..123: note = hu29F1 VH aspartate variant in hu29F1 VH 7
EVQLVESGGGLVQPGGSLRLSCTASGFSFSSDHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 589 - protein, 123 aa, synthetic construct
  REGION 1..123: note = hu29F1 VH aspartate variant in hu29F1 VH 8
EVQLVESGGGLVQPGGSLRLSCTASGFSFSSSDWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 590 - protein, 123 aa, synthetic construct
  REGION 1..123: note = hu29F1 VH aspartate variant in hu29F1 VH 9
EVQLVESGGGLVQPGGSLRLSCTASGFSFSSSHDICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 591 - protein, 123 aa, synthetic construct
  REGION 1..123: note = hu29F1 VH aspartate variant in hu29F1 VH 10
EVQLVESGGGLVQPGGSLRLSCTASGFSFSSSHWDCWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 592 - protein, 123 aa, synthetic construct
  REGION 1..123: note = hu29F1 VH aspartate variant in hu29F1 VH 11
EVQLVESGGGLVQPGGSLRLSCTASGFSFSSSHWIDWVRQAPGKGLEWVGSIYAGSIGSTYYASWAKGRFTISRDTSKNTVYLQMNSLRAEDTAVYYCARAPNDIYNYYFNLWGQGTLVTVSS

SEQ ID NO: 593 - protein, 123 aa, synthetic construct
  REGION 1..123: note = hu29F1 VH aspartate variant in hu29F1 VH 12
EVQLVESGGGLVQPGGSLRLSCTASGFSFSSSHWISWVRQAPGKGLEWVGDIYAGSIGSTYYASWAKGRFTISRDTSKNTVYLQMNSLRAEDTAVYYCARAPNDIYNYYFNLWGQGTLVTVSS

SEQ ID NO: 594 - protein, 123 aa, synthetic construct
  REGION 1..123: note = hu29F1 VH aspartate variant in hu29F1 VH 13
EVQLVESGGGLVQPGGSLRLSCTASGFSFSSSHWICWVRQAPGKGLEWVGCDYAGSIGSTYYASWAKGRFTISRDTSKNTVYLQMNSLRAEDTAVYYCARAPNDIYNYYFNLWGQGTLVTVSS

SEQ ID NO: 595 - protein, 123 aa, synthetic construct
  REGION 1..123: note = hu29F1 VH aspartate variant in hu29F1 VH 14
EVQLVESGGGLVQPGGSLRLSCTASGFSFSSSHWICWVRQAPGKGLEWVGCIDAGSIGSTYYASWAKGRFTISRDTSKNTVYLQMNSLRAEDTAVYYCARAPNDIYNYYFNLWGQGTLVTVSS

SEQ ID NO: 596 - protein, 123 aa, synthetic construct
  REGION 1..123: note = hu29F1 VH aspartate variant in hu29F1 VH 15
EVQLVESGGGLVQPGGSLRLSCTASGFSFSSSHWICWVRQAPGKGLEWVGCIYDGSIGSTYYASWAKGRFTISRDTSKNTVYLQMNSLRAEDTAVYYCARAPNDIYNYYFNLWGQGTLVTVSS

SEQ ID NO: 597 - protein, 123 aa, synthetic construct
  REGION 1..123: note = hu29F1 VH aspartate variant in hu29F1 VH 16
EVQLVESGGGLVQPGGSLRLSCTASGFSFSSSHWICWVRQAPGKGLEWVGCIYADSIGSTYYASWAKGRFTISRDTSKNTVYLQMNSLRAEDTAVYYCARAPNDIYNYYFNLWGQGTLVTVSS

SEQ ID NO: 598 - protein, 123 aa, synthetic construct
  REGION 1..123: note = hu29F1 VH aspartate variant in hu29F1 VH 17
EVQLVESGGGLVQPGGSLRLSCTASGFSFSSSHWICWVRQAPGKGLEWVGCIYAGDIGSTYYASWAKGRFTISRDTSKNTVYLQMNSLRAEDTAVYYCARAPNDIYNYYFNLWGQGTLVTVSS

SEQ ID NO: 599 - protein, 123 aa, synthetic construct
  REGION 1..123: note = hu29F1 VH aspartate variant in hu29F1 VH 18
EVQLVESGGGLVQPGGSLRLSCTASGFSFSSSHWICWVRQAPGKGLEWVGCIYAGSDGSTYYASWAKGRFTISRDTSKNTVYLQMNSLRAEDTAVYYCARAPNDIYNYYFNLWGQGTLVTVSS

SEQ ID NO: 600 - protein, 123 aa, synthetic construct
  REGION 1..123: note = hu29F1 VH aspartate variant in hu29F1 VH 19
EVQLVESGGGLVQPGGSLRLSCTASGFSFSSSHWICWVRQAPGKGLEWVGCIYAGSIDSTYYASWAKGRFTISRDTSKNTVYLQMNSLRAEDTAVYYCARAPNDIYNYYFNLWGQGTLVTVSS

SEQ ID NO: 601 - protein, 123 aa, synthetic construct
  REGION 1..123: note = hu29F1 VH aspartate variant in hu29F1 VH 20
EVQLVESGGGLVQPGGSLRLSCTASGFSFSSSHWICWVRQAPGKGLEWVGCIYAGSIGDTYYASWAKGRFTISRDTSKNTVYLQMNSLRAEDTAVYYCARAPNDIYNYYFNLWGQGTLVTVSS

SEQ ID NO: 602 - protein, 123 aa, synthetic construct
  REGION 1..123: note = hu29F1 VH aspartate variant in hu29F1 VH 21
EVQLVESGGGLVQPGGSLRLSCTASGFSFSSSHWICWVRQAPGKGLEWVGCIYAGSIGSDYYASWAKGRFTISRDTSKNTVYLQMNSLRAEDTAVYYCARAPNDIYNYYFNLWGQGTLVTVSS

SEQ ID NO: 603 - protein, 123 aa, synthetic construct
  REGION 1..123: note = hu29F1 VH aspartate variant in hu29F1 VH 22
EVQLVESGGGLVQPGGSLRLSCTASGFSFSSSHWICWVRQAPGKGLEWVGCIYAGSIGSTDYASWAKGRFTISRDTSKNTVYLQMNSLRAEDTAVYYCARAPNDIYNYYFNLWGQGTLVTVSS

SEQ ID NO: 604 - protein, 123 aa, synthetic construct
  REGION 1..123: note = hu29F1 VH aspartate variant in hu29F1 VH 23
EVQLVESGGGLVQPGGSLRLSCTASGFSFSSSHWICWVRQAPGKGLEWVGCIYAGSIGSTYDASWAKGRFTISRDTSKNTVYLQMNSLRAEDTAVYYCARAPNDIYNYYFNLWGQGTLVTVSS

SEQ ID NO: 605 - protein, 123 aa, synthetic construct
  REGION 1..123: note = hu29F1 VH aspartate variant in hu29F1 VH 24
EVQLVESGGGLVQPGGSLRLSCTASGFSFSSSHWICWVRQAPGKGLEWVGCIYAGSIGSTYYDSWAKGRFTISRDTSKNTVYLQMNSLRAEDTAVYYCARAPNDIYNYYFNLWGQGTLVTVSS

SEQ ID NO: 606 - protein, 123 aa, synthetic construct
  REGION 1..123: note = hu29F1 VH aspartate variant in hu29F1 VH 25
EVQLVESGGGLVQPGGSLRLSCTASGFSFSSSHWICWVRQAPGKGLEWVGCIYAGSIGSTYYADWAKGRFTISRDTSKNTVYLQMNSLRAEDTAVYYCARAPNDIYNYYFNLWGQGTLVTVSS

SEQ ID NO: 607 - protein, 123 aa, synthetic construct
  REGION 1..123: note = hu29F1 VH aspartate variant in hu29F1 VH 26
EVQLVESGGGLVQPGGSLRLSCTASGFSFSSSHWICWVRQAPGKGLEWVGCIYAGSIGSTYYASDAKGRFTISRDTSKNTVYLQMNSLRAEDTAVYYCARAPNDIYNYYFNLWGQGTLVTVSS

SEQ ID NO: 608 - protein, 123 aa, synthetic construct
  REGION 1..123: note = hu29F1 VH aspartate variant in hu29F1 VH 27
EVQLVESGGGLVQPGGSLRLSCTASGFSFSSSHWICWVRQAPGKGLEWVGCIYAGSIGSTYYASWDKGRFTISRDTSKNTVYLQMNSLRAEDTAVYYCARAPNDIYNYYFNLWGQGTLVTVSS

SEQ ID NO: 609 - protein, 123 aa, synthetic construct
  REGION 1..123: note = hu29F1 VH aspartate variant in hu29F1 VH 28
EVQLVESGGGLVQPGGSLRLSCTASGFSFSSSHWICWVRQAPGKGLEWVGCIYAGSIGSTYYASWADGRFTISRDTSKNTVYLQMNSLRAEDTAVYYCARAPNDIYNYYFNLWGQGTLVTVSS

SEQ ID NO: 610 - protein, 123 aa, synthetic construct
  REGION 1..123: note = hu29F1 VH aspartate variant in hu29F1 VH 29
EVQLVESGGGLVQPGGSLRLSCTASGFSFSSSHWICWVRQAPGKGLEWVGCIYAGSIGSTYYASWAKDRFTISRDTSKNTVYLQMNSLRAEDTAVYYCARAPNDIYNYYFNLWGQGTLVTVSS

SEQ ID NO: 611 - protein, 123 aa, synthetic construct
  REGION 1..123: note = hu29F1 VH aspartate variant in hu29F1 VH 30
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCDRAPNDIYNYYFNLWGQGTLVTVSS

SEQ ID NO: 612 - protein, 123 aa, synthetic construct
  REGION 1..123: note = hu29F1 VH aspartate variant in hu29F1 VH 31
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCADAPNDIYNYYFNLWGQGTLVTVSS

SEQ ID NO: 613 - protein, 123 aa, synthetic construct
  REGION 1..123: note = hu29F1 VH aspartate variant in hu29F1 VH 32
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARDPNDIYNYYFNLWGQGTLVTVSS

SEQ ID NO: 614 - protein, 123 aa, synthetic construct
  REGION 1..123: note = hu29F1 VH aspartate variant in hu29F1 VH 33
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARADNDIYNYYFNLWGQGTLVTVSS

SEQ ID NO: 615 - protein, 123 aa, synthetic construct
  REGION 1..123: note = hu29F1 VH aspartate variant in hu29F1 VH 34
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPDDIYNYYFNLWGQGTLVTVSS

SEQ ID NO: 616 - protein, 123 aa, synthetic construct
  REGION 1..123: note = hu29F1 VH aspartate variant in hu29F1 VH 35
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNAIYNYYFNLWGQGTLVTVSS

SEQ ID NO: 617 - protein, 123 aa, synthetic construct
  REGION 1..123: note = hu29F1 VH aspartate variant in hu29F1 VH 36
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDDYNYYFNLWGQGTLVTVSS

SEQ ID NO: 618 - protein, 123 aa, synthetic construct
  REGION 1..123: note = hu29F1 VH aspartate variant in hu29F1 VH 37
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIDNYYFNLWGQGTLVTVSS

SEQ ID NO: 619 - protein, 123 aa, synthetic construct
  REGION 1..123: note = hu29F1 VH aspartate variant in hu29F1 VH 38
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYDYYFNLWGQGTLVTVSS

SEQ ID NO: 620 - protein, 123 aa, synthetic construct
  REGION 1..123: note = hu29F1 VH aspartate variant in hu29F1 VH 39
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNDYFNLWGQGTLVTVSS

SEQ ID NO: 621 - protein, 123 aa, synthetic construct
  REGION 1..123: note = hu29F1 VH aspartate variant in hu29F1 VH 40
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYDFNLWGQGTLVTVSS

SEQ ID NO: 622 - protein, 123 aa, synthetic construct
  REGION 1..123: note = hu29F1 VH aspartate variant in hu29F1 VH 41
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYDNLWGQGTLVTVSS

SEQ ID NO: 623 - protein, 123 aa, synthetic construct
  REGION 1..123: note = hu29F1 VH aspartate variant in hu29F1 VH 42
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYFDLWGQGTLVTVSS

SEQ ID NO: 624 - protein, 123 aa, synthetic construct
  REGION 1..123: note = hu29F1 VH aspartate variant in hu29F1 VH 43
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYFNDWGQGTLVTVSS

SEQ ID NO: 625 - protein, 108 aa, synthetic construct
  REGION 1..108: note = hu29F1 LC aspartate variants in hu29F1 VL 1
EIVMTQSPSTLSASVGDRVIITCDASENIYNYLAWYQQKPGKAPKLLIYYASTLASGVPSRFSGSGSGAEFTLTISSLQPDDFATYYCQSGYVSKSSTFGQGTKLTVL

SEQ ID NO: 626 - protein, 108 aa, synthetic construct
  REGION 1..108: note = hu29F1 LC aspartate variants in hu29F1 VL 2
EIVMTQSPSTLSASVGDRVIITCQDSENIYNYLAWYQQKPGKAPKLLIYYASTLASGVPSRFSGSGSGAEFTLTISSLQPDDFATYYCQSGYVSKSSTFGQGTKLTVL

SEQ ID NO: 627 - protein, 108 aa, synthetic construct
  REGION 1..108: note = hu29F1 LC aspartate variants in hu29F1 VL 3
EIVMTQSPSTLSASVGDRVIITCQADENIYNYLAWYQQKPGKAPKLLIYYASTLASGVPSRFSGSGSGAEFTLTISSLQPDDFATYYCQSGYVSKSSTFGQGTKLTVL

SEQ ID NO: 628 - protein, 108 aa, synthetic construct
  REGION 1..108: note = hu29F1 LC aspartate variants in hu29F1 VL 4
EIVMTQSPSTLSASVGDRVIITCQASDNIYNYLAWYQQKPGKAPKLLIYYASTLASGVPSRFSGSGSGAEFTLTISSLQPDDFATYYCQSGYVSKSSTFGQGTKLTVL

SEQ ID NO: 629 - protein, 108 aa, synthetic construct
  REGION 1..108: note = hu29F1 LC aspartate variants in hu29F1 VL 5
EIVMTQSPSTLSASVGDRVIITCQASEDIYNYLAWYQQKPGKAPKLLIYYASTLASGVPSRFSGSGSGAEFTLTISSLQPDDFATYYCQSGYVSKSSTFGQGTKLTVL

SEQ ID NO: 630 - protein, 108 aa, synthetic construct
  REGION 1..108: note = hu29F1 LC aspartate variants in hu29F1 VL 6
EIVMTQSPSTLSASVGDRVIITCQASENDYNYLAWYQQKPGKAPKLLIYYASTLASGVPSRFSGSGSGAEFTLTISSLQPDDFATYYCQSGYVSKSSTFGQGTKLTVL

SEQ ID NO: 631 - protein, 108 aa, synthetic construct
  REGION 1..108: note = hu29F1 LC aspartate variants in hu29F1 VL 7
EIVMTQSPSTLSASVGDRVIITCQASENIDNYLAWYQQKPGKAPKLLIYYASTLASGVPSRFSGSGSGAEFTLTISSLQPDDFATYYCQSGYVSKSSTFGQGTKLTVL

SEQ ID NO: 632 - protein, 108 aa, synthetic construct
  REGION 1..108: note = hu29F1 LC aspartate variants in hu29F1 VL 8
EIVMTQSPSTLSASVGDRVIITCQASENIYDYLAWYQQKPGKAPKLLIYYASTLASGVPSRFSGSGSGAEFTLTISSLQPDDFATYYCQSGYVSKSSTFGQGTKLTVL

SEQ ID NO: 633 - protein, 108 aa, synthetic construct
  REGION 1..108: note = hu29F1 LC aspartate variants in hu29F1 VL 9
EIVMTQSPSTLSASVGDRVIITCQASENIYNDLAWYQQKPGKAPKLLIYYASTLASGVPSRFSGSGSGAEFTLTISSLQPDDFATYYCQSGYVSKSSTFGQGTKLTVL

SEQ ID NO: 634 - protein, 108 aa, synthetic construct
  REGION 1..108: note = hu29F1 LC aspartate variants in hu29F1 VL 10
EIVMTQSPSTLSASVGDRVIITCQASENIYNYDAWYQQKPGKAPKLLIYYASTLASGVPSRFSGSGSGAEFTLTISSLQPDDFATYYCQSGYVSKSSTFGQGTKLTVL

SEQ ID NO: 635 - protein, 108 aa, synthetic construct
  REGION 1..108: note = hu29F1 LC aspartate variants in hu29F1 VL 11
EIVMTQSPSTLSASVGDRVIITCQASENIYNYLDWYQQKPGKAPKLLIYYASTLASGVPSRFSGSGSGAEFTLTISSLQPDDFATYYCQSGYVSKSSTFGQGTKLTVL

SEQ ID NO: 636 - protein, 108 aa, synthetic construct
  REGION 1..108: note = hu29F1 LC aspartate variants in hu29F1 VL 12
EIVMTQSPSTLSASVGDRVIITCQASENIYNYLAWYQQKPGKAPKLLIYDASTLASGVPSRFSGSGSGAEFTLTISSLQPDDFATYYCQSGYVSKSSTFGQGTKLTVL

SEQ ID NO: 637 - protein, 108 aa, synthetic construct
  REGION 1..108: note = hu29F1 LC aspartate variants in hu29F1 VL 13
EIVMTQSPSTLSASVGDRVIITCQASENIYNYLAWYQQKPGKAPKLLIYYDSTLASGVPSRFSGSGSGAEFTLTISSLQPDDFATYYCQSGYVSKSSTFGQGTKLTVL

SEQ ID NO: 638 - protein, 108 aa, synthetic construct
  REGION 1..108: note = hu29F1 LC aspartate variants in hu29F1 VL 14
EIVMTQSPSTLSASVGDRVIITCQASENIYNYLAWYQQKPGKAPKLLIYYADTLASGVPSRFSGSGSGAEFTLTISSLQPDDFATYYCQSGYVSKSSTFGQGTKLTVL

SEQ ID NO: 639 - protein, 108 aa, synthetic construct
  REGION 1..108: note = hu29F1 LC aspartate variants in hu29F1 VL 15
EIVMTQSPSTLSASVGDRVIITCQASENIYNYLAWYQQKPGKAPKLLIYYASDLASGVPSRFSGSGSGAEFTLTISSLQPDDFATYYCQSGYVSKSSTFGQGTKLTVL

SEQ ID NO: 640 - protein, 108 aa, synthetic construct
  REGION 1..108: note = hu29F1 LC aspartate variants in hu29F1 VL 16
EIVMTQSPSTLSASVGDRVIITCQASENIYNYLAWYQQKPGKAPKLLIYYASTDASGVPSRFSGSGSGAEFTLTISSLQPDDFATYYCQSGYVSKSSTFGQGTKLTVL

SEQ ID NO: 641 - protein, 108 aa, synthetic construct
  REGION 1..108: note = hu29F1 LC aspartate variants in hu29F1 VL 17
EIVMTQSPSTLSASVGDRVIITCQASENIYNYLAWYQQKPGKAPKLLIYYASTLDSGVPSRFSGSGSGAEFTLTISSLQPDDFATYYCQSGYVSKSSTFGQGTKLTVL

SEQ ID NO: 642 - protein, 108 aa, synthetic construct
  REGION 1..108: note = hu29F1 LC aspartate variants in hu29F1 VL 18
EIVMTQSPSTLSASVGDRVIITCQASENIYNYLAWYQQKPGKAPKLLIYYASTLADGVPSRFSGSGSGAEFTLTISSLQPDDFATYYCQSGYVSKSSTFGQGTKLTVL

SEQ ID NO: 643 - protein, 108 aa, synthetic construct
  REGION 1..108: note = hu29F1 LC aspartate variants in hu29F1 VL 19
EIVMTQSPSTLSASVGDRVIITCQASENIYNYLAWYQQKPGKAPKLLIYYASTLASGVPSRFSGSGSGAEFTLTISSLQPDDFATYYCDSGYVSKSSTFGQGTKLTVL

SEQ ID NO: 644 - protein, 108 aa, synthetic construct
  REGION 1..108: note = hu29F1 LC aspartate variants in hu29F1 VL 20
EIVMTQSPSTLSASVGDRVIITCQASENIYNYLAWYQQKPGKAPKLLIYYASTLASGVPSRFSGSGSGAEFTLTISSLQPDDFATYYCQDGYVSKSSTFGQGTKLTVL

SEQ ID NO: 645 - protein, 108 aa, synthetic construct
  REGION 1..108: note = hu29F1 LC aspartate variants in hu29F1 VL 21
EIVMTQSPSTLSASVGDRVIITCQASENIYNYLAWYQQKPGKAPKLLIYYASTLASGVPSRFSGSGSGAEFTLTISSLQPDDFATYYCQSDYVSKSSTFGQGTKLTVL

SEQ ID NO: 646 - protein, 108 aa, synthetic construct
  REGION 1..108: note = hu29F1 LC aspartate variants in hu29F1 VL 22
EIVMTQSPSTLSASVGDRVIITCQASENIYNYLAWYQQKPGKAPKLLIYYASTLASGVPSRFSGSGSGAEFTLTISSLQPDDFATYYCQSGDVSKSSTFGQGTKLTVL

SEQ ID NO: 647 - protein, 108 aa, synthetic construct
  REGION 1..108: note = hu29F1 LC aspartate variants in hu29F1 VL 23
EIVMTQSPSTLSASVGDRVIITCQASENIYNYLAWYQQKPGKAPKLLIYYASTLASGVPSRFSGSGSGAEFTLTISSLQPDDFATYYCQSGYDSKSSTFGQGTKLTVL

SEQ ID NO: 648 - protein, 108 aa, synthetic construct
  REGION 1..108: note = hu29F1 LC aspartate variants in hu29F1 VL 24
EIVMTQSPSTLSASVGDRVIITCQASENIYNYLAWYQQKPGKAPKLLIYYASTLASGVPSRFSGSGSGAEFTLTISSLQPDDFATYYCQSGYVDKSSTFGQGTKLTVL

SEQ ID NO: 649 - protein, 108 aa, synthetic construct
  REGION 1..108: note = hu29F1 LC aspartate variants in hu29F1 VL 25
EIVMTQSPSTLSASVGDRVIITCQASENIYNYLAWYQQKPGKAPKLLIYYASTLASGVPSRFSGSGSGAEFTLTISSLQPDDFATYYCQSGYVSDSSTFGQGTKLTVL

SEQ ID NO: 650 - protein, 108 aa, synthetic construct
  REGION 1..108: note = hu29F1 LC aspartate variants in hu29F1 VL 26
EIVMTQSPSTLSASVGDRVIITCQASENIYNYLAWYQQKPGKAPKLLIYYASTLASGVPSRFSGSGSGAEFTLTISSLQPDDFATYYCQSGYVSKDSTFGQGTKLTVL

SEQ ID NO: 651 - protein, 108 aa, synthetic construct
  REGION 1..108: note = hu29F1 LC aspartate variants in hu29F1 VL 27
EIVMTQSPSTLSASVGDRVIITCQASENIYNYLAWYQQKPGKAPKLLIYYASTLASGVPSRFSGSGSGAEFTLTISSLQPDDFATYYCQSGYVSKSDTFGQGTKLTVL

SEQ ID NO: 652 - protein, 108 aa, synthetic construct
  REGION 1..108: note = hu29F1 LC aspartate variants in hu29F1 VL 28
EIVMTQSPSTLSASVGDRVIITCQASENIYNYLAWYQQKPGKAPKLLIYYASTLASGVPSRFSGSGSGAEFTLTISSLQPDDFATYYCQSGYVSKSSDFGQGTKLTVL

SEQ ID NO: 653 - protein, 123 aa, synthetic construct
  REGION 1..123: note = hu29F1 VH glutamate variant in hu29F1 VH 1
EVQLVESGGGLVQPGGSLRLSCTASEFSFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 654 - protein, 123 aa, synthetic construct
  REGION 1..123: note = hu29F1 VH glutamate variant in hu29F1 VH 2
EVQLVESGGGLVQPGGSLRLSCTASGESFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 655 - protein, 123 aa, synthetic construct
  REGION 1..123: note = hu29F1 VH glutamate variant in hu29F1 VH 3
EVQLVESGGGLVQPGGSLRLSCTASGFEFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 656 - protein, 123 aa, synthetic construct
  REGION 1..123: note = hu29F1 VH glutamate variant in hu29F1 VH 4
EVQLVESGGGLVQPGGSLRLSCTASGFSESSSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 657 - protein, 123 aa, synthetic construct
  REGION 1..123: note = hu29F1 VH glutamate variant in hu29F1 VH 5
EVQLVESGGGLVQPGGSLRLSCTASGFSFESSHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 658 - protein, 123 aa, synthetic construct
  REGION 1..123: note = hu29F1 VH glutamate variant in hu29F1 VH 6
EVQLVESGGGLVQPGGSLRLSCTASGFSFSESHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 659 - protein, 123 aa, synthetic construct
  REGION 1..123: note = hu29F1 VH glutamate variant in hu29F1 VH 7
EVQLVESGGGLVQPGGSLRLSCTASGFSFSSEHWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 660 - protein, 123 aa, synthetic construct
  REGION 1..123: note = hu29F1 VH glutamate variant in hu29F1 VH 8
EVQLVESGGGLVQPGGSLRLSCTASGFSFSSSEWICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 661 - protein, 123 aa, synthetic construct
  REGION 1..123: note = hu29F1 VH glutamate variant in hu29F1 VH 9
EVQLVESGGGLVQPGGSLRLSCTASGFSFSSSHEICWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 662 - protein, 123 aa, synthetic construct
  REGION 1..123: note = hu29F1 VH glutamate variant in hu29F1 VH 10
EVQLVESGGGLVQPGGSLRLSCTASGFSFSSSHWECWVRQAPGKGLEWVGCIYAGSIGSTYYASWAKGRFTISRDTSKNTVYLQMNSLRAEDTAVYYCARAPNDIYNYYFNLWGQGTLVTVSS

SEQ ID NO: 663 - protein, 123 aa, synthetic construct
  REGION 1..123: note = hu29F1 VH glutamate variant in hu29F1 VH 11
EVQLVESGGGLVQPGGSLRLSCTASGFSFSSSHWIEWVRQAPGKGLEWVGSIYAGSIGSTYYASWAKGRFTISRDTSKNTVYLQMNSLRAEDTAVYYCARAPNDIYNYYFNLWGQGTLVTVSS

SEQ ID NO: 664 - protein, 123 aa, synthetic construct
  REGION 1..123: note = hu29F1 VH glutamate variant in hu29F1 VH 12
EVQLVESGGGLVQPGGSLRLSCTASGFSFSSSHWISWVRQAPGKGLEWVGEIYAGSIGSTYYASWAKGRFTISRDTSKNTVYLQMNSLRAEDTAVYYCARAPNDIYNYYFNLWGQGTLVTVSS

SEQ ID NO: 665 - protein, 123 aa, synthetic construct
  REGION 1..123: note = hu29F1 VH glutamate variant in hu29F1 VH 13
EVQLVESGGGLVQPGGSLRLSCTASGFSFSSSHWICWVRQAPGKGLEWVGCEYAGSIGSTYYASWAKGRFTISRDTSKNTVYLQMNSLRAEDTAVYYCARAPNDIYNYYFNLWGQGTLVTVSS

SEQ ID NO: 666 - protein, 123 aa, synthetic construct
  REGION 1..123: note = hu29F1 VH glutamate variant in hu29F1 VH 14
EVQLVESGGGLVQPGGSLRLSCTASGFSFSSSHWICWVRQAPGKGLEWVGCIEAGSIGSTYYASWAKGRFTISRDTSKNTVYLQMNSLRAEDTAVYYCARAPNDIYNYYFNLWGQGTLVTVSS

SEQ ID NO: 667 - protein, 123 aa, synthetic construct
  REGION 1..123: note = hu29F1 VH glutamate variant in hu29F1 VH 15
EVQLVESGGGLVQPGGSLRLSCTASGFSFSSSHWICWVRQAPGKGLEWVGCIYEGSIGSTYYASWAKGRFTISRDTSKNTVYLQMNSLRAEDTAVYYCARAPNDIYNYYFNLWGQGTLVTVSS

SEQ ID NO: 668 - protein, 123 aa, synthetic construct
  REGION 1..123: note = hu29F1 VH glutamate variant in hu29F1 VH 16
EVQLVESGGGLVQPGGSLRLSCTASGFSFSSSHWICWVRQAPGKGLEWVGCIYAESIGSTYYASWAKGRFTISRDTSKNTVYLQMNSLRAEDTAVYYCARAPNDIYNYYFNLWGQGTLVTVSS

SEQ ID NO: 669 - protein, 123 aa, synthetic construct
  REGION 1..123: note = hu29F1 VH glutamate variant in hu29F1 VH 17
EVQLVESGGGLVQPGGSLRLSCTASGFSFSSSHWICWVRQAPGKGLEWVGCIYAGEIGSTYYASWAKGRFTISRDTSKNTVYLQMNSLRAEDTAVYYCARAPNDIYNYYFNLWGQGTLVTVSS

SEQ ID NO: 670 - protein, 123 aa, synthetic construct
  REGION 1..123: note = hu29F1 VH glutamate variant in hu29F1 VH 18
EVQLVESGGGLVQPGGSLRLSCTASGFSFSSSHWICWVRQAPGKGLEWVGCIYAGSEGSTYYASWAKGRFTISRDTSKNTVYLQMNSLRAEDTAVYYCARAPNDIYNYYFNLWGQGTLVTVSS

SEQ ID NO: 671 - protein, 123 aa, synthetic construct
  REGION 1..123: note = hu29F1 VH glutamate variant in hu29F1 VH 19
EVQLVESGGGLVQPGGSLRLSCTASGFSFSSSHWICWVRQAPGKGLEWVGCIYAGSIESTYYASWAKGRFTISRDTSKNTVYLQMNSLRAEDTAVYYCARAPNDIYNYYFNLWGQGTLVTVSS

SEQ ID NO: 672 - protein, 123 aa, synthetic construct
  REGION 1..123: note = hu29F1 VH glutamate variant in hu29F1 VH 20
EVQLVESGGGLVQPGGSLRLSCTASGFSFSSSHWICWVRQAPGKGLEWVGCIYAGSIGETYYASWAKGRFTISRDTSKNTVYLQMNSLRAEDTAVYYCARAPNDIYNYYFNLWGQGTLVTVSS

SEQ ID NO: 673 - protein, 123 aa, synthetic construct
  REGION 1..123: note = hu29F1 VH glutamate variant in hu29F1 VH 21
EVQLVESGGGLVQPGGSLRLSCTASGFSFSSSHWICWVRQAPGKGLEWVGCIYAGSIGSEYYASWAKGRFTISRDTSKNTVYLQMNSLRAEDTAVYYCARAPNDIYNYYFNLWGQGTLVTVSS

SEQ ID NO: 674 - protein, 123 aa, synthetic construct
  REGION 1..123: note = hu29F1 VH glutamate variant in hu29F1 VH 22
EVQLVESGGGLVQPGGSLRLSCTASGFSFSSSHWICWVRQAPGKGLEWVGCIYAGSIGSTEYASWAKGRFTISRDTSKNTVYLQMNSLRAEDTAVYYCARAPNDIYNYYFNLWGQGTLVTVSS

SEQ ID NO: 675 - protein, 123 aa, synthetic construct
  REGION 1..123: note = hu29F1 VH glutamate variant in hu29F1 VH 23
EVQLVESGGGLVQPGGSLRLSCTASGFSFSSSHWICWVRQAPGKGLEWVGCIYAGSIGSTYEASWAKGRFTISRDTSKNTVYLQMNSLRAEDTAVYYCARAPNDIYNYYFNLWGQGTLVTVSS

SEQ ID NO: 676 - protein, 123 aa, synthetic construct
  REGION 1..123: note = hu29F1 VH glutamate variant in hu29F1 VH 24
EVQLVESGGGLVQPGGSLRLSCTASGFSFSSSHWICWVRQAPGKGLEWVGCIYAGSIGSTYYESWAKGRFTISRDTSKNTVYLQMNSLRAEDTAVYYCARAPNDIYNYYFNLWGQGTLVTVSS

SEQ ID NO: 677 - protein, 123 aa, synthetic construct
  REGION 1..123: note = hu29F1 VH glutamate variant in hu29F1 VH 25
EVQLVESGGGLVQPGGSLRLSCTASGFSFSSSHWICWVRQAPGKGLEWVGCIYAGSIGSTYYAEWAKGRFTISRDTSKNTVYLQMNSLRAEDTAVYYCARAPNDIYNYYFNLWGQGTLVTVSS

SEQ ID NO: 678 - protein, 123 aa, synthetic construct
  REGION 1..123: note = hu29F1 VH glutamate variant in hu29F1 VH 26
EVQLVESGGGLVQPGGSLRLSCTASGFSFSSSHWICWVRQAPGKGLEWVGCIYAGSIGSTYYASEAKGRFTISRDTSKNTVYLQMNSLRAEDTAVYYCARAPNDIYNYYFNLWGQGTLVTVSS

SEQ ID NO: 679 - protein, 123 aa, synthetic construct
  REGION 1..123: note = hu29F1 VH glutamate variant in hu29F1 VH 27
EVQLVESGGGLVQPGGSLRLSCTASGFSFSSSHWICWVRQAPGKGLEWVGCIYAGSIGSTYYASWEKGRFTISRDTSKNTVYLQMNSLRAEDTAVYYCARAPNDIYNYYFNLWGQGTLVTVSS

SEQ ID NO: 680 - protein, 123 aa, synthetic construct
  REGION 1..123: note = hu29F1 VH glutamate variant in hu29F1 VH 28
EVQLVESGGGLVQPGGSLRLSCTASGFSFSSSHWICWVRQAPGKGLEWVGCIYAGSIGSTYYASWAEGRFTISRDTSKNTVYLQMNSLRAEDTAVYYCARAPNDIYNYYFNLWGQGTLVTVSS

SEQ ID NO: 681 - protein, 123 aa, synthetic construct
  REGION 1..123: note = hu29F1 VH glutamate variant in hu29F1 VH 29
EVQLVESGGGLVQPGGSLRLSCTASGFSFSSSHWICWVRQAPGKGLEWVGCIYAGSIGSTYYASWAKERFTISRDTSKNTVYLQMNSLRAEDTAVYYCARAPNDIYNYYFNLWGQGTLVTVSS

SEQ ID NO: 682 - protein, 123 aa, synthetic construct
  REGION 1..123: note = hu29F1 VH glutamate variant in hu29F1 VH 30
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCERAPNDIYNYYFNLWGQGTLVTVSS

SEQ ID NO: 683 - protein, 123 aa, synthetic construct
  REGION 1..123: note = hu29F1 VH glutamate variant in hu29F1 VH 31
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCAEAPNDIYNYYFNLWGQGTLVTVSS

SEQ ID NO: 684 - protein, 123 aa, synthetic construct
  REGION 1..123: note = hu29F1 VH glutamate variant in hu29F1 VH 32
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCAREPNDIYNYYFNLWGQGTLVTVSS

SEQ ID NO: 685 - protein, 123 aa, synthetic construct
  REGION 1..123: note = hu29F1 VH glutamate variant in hu29F1 VH 33
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAENDIYNYYFNLWGQGTLVTVSS

SEQ ID NO: 686 - protein, 123 aa, synthetic construct
  REGION 1..123: note = hu29F1 VH glutamate variant in hu29F1 VH 34
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPEDIYNYYFNLWGQGTLVTVSS

SEQ ID NO: 687 - protein, 123 aa, synthetic construct
  REGION 1..123: note = hu29F1 VH glutamate variant in hu29F1 VH 35
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNEIYNYYFNLWGQGTLVTVSS

SEQ ID NO: 688 - protein, 123 aa, synthetic construct
  REGION 1..123: note = hu29F1 VH glutamate variant in hu29F1 VH 36
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDEYNYYFNLWGQGTLVTVSS

SEQ ID NO: 689 - protein, 123 aa, synthetic construct
  REGION 1..123: note = hu29F1 VH glutamate variant in hu29F1 VH 37
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIENYYFNLWGQGTLVTVSS

SEQ ID NO: 690 - protein, 123 aa, synthetic construct
  REGION 1..123: note = hu29F1 VH glutamate variant in hu29F1 VH 38
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYEYYFNLWGQGTLVTVSS

SEQ ID NO: 691 - protein, 123 aa, synthetic construct
  REGION 1..123: note = hu29F1 VH glutamate variant in hu29F1 VH 39
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNEYFNLWGQGTLVTVSS

SEQ ID NO: 692 - protein, 123 aa, synthetic construct
  REGION 1..123: note = hu29F1 VH glutamate variant in hu29F1 VH 40
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYEFNLWGQGTLVTVSS

SEQ ID NO: 693 - protein, 123 aa, synthetic construct
  REGION 1..123: note = hu29F1 VH glutamate variant in hu29F1 VH 41
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYENLWGQGTLVTVSS

SEQ ID NO: 694 - protein, 123 aa, synthetic construct
  REGION 1..123: note = hu29F1 VH glutamate variant in hu29F1 VH 42
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYFELWGQGTLVTVSS

SEQ ID NO: 695 - protein, 123 aa, synthetic construct
  REGION 1..123: note = hu29F1 VH glutamate variant in hu29F1 VH 43
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYFNEWGQGTLVTVSS

SEQ ID NO: 696 - protein, 108 aa, synthetic construct
  REGION 1..108: note = hu29F1 LC glutamate variants in hu29F1 VL 1
EIVMTQSPSTLSASVGDRVIITCEASENIYNYLAWYQQKPGKAPKLLIYYASTLASGVPSRFSGSGSGAEFTLTISSLQPDDFATYYCQSGYVSKSSTFGQGTKLTVL

SEQ ID NO: 697 - protein, 108 aa, synthetic construct
  REGION 1..108: note = hu29F1 LC glutamate variants in hu29F1 VL 2
EIVMTQSPSTLSASVGDRVIITCQESENIYNYLAWYQQKPGKAPKLLIYYASTLASGVPSRFSGSGSGAEFTLTISSLQPDDFATYYCQSGYVSKSSTFGQGTKLTVL

SEQ ID NO: 698 - protein, 108 aa, synthetic construct
  REGION 1..108: note = hu29F1 LC glutamate variants in hu29F1 VL 3
EIVMTQSPSTLSASVGDRVIITCQAEENIYNYLAWYQQKPGKAPKLLIYYASTLASGVPSRFSGSGSGAEFTLTISSLQPDDFATYYCQSGYVSKSSTFGQGTKLTVL

SEQ ID NO: 699 - protein, 108 aa, synthetic construct
  REGION 1..108: note = hu29F1 LC glutamate variants in hu29F1 VL 4
EIVMTQSPSTLSASVGDRVIITCQASANIYNYLAWYQQKPGKAPKLLIYYASTLASGVPSRFSGSGSGAEFTLTISSLQPDDFATYYCQSGYVSKSSTFGQGTKLTVL

SEQ ID NO: 700 - protein, 108 aa, synthetic construct
  REGION 1..108: note = hu29F1 LC glutamate variants in hu29F1 VL 5
EIVMTQSPSTLSASVGDRVIITCQASEEIYNYLAWYQQKPGKAPKLLIYYASTLASGVPSRFSGSGSGAEFTLTISSLQPDDFATYYCQSGYVSKSSTFGQGTKLTVL

SEQ ID NO: 701 - protein, 108 aa, synthetic construct
  REGION 1..108: note = hu29F1 LC glutamate variants in hu29F1 VL 6
EIVMTQSPSTLSASVGDRVIITCQASENEYNYLAWYQQKPGKAPKLLIYYASTLASGVPSRFSGSGSGAEFTLTISSLQPDDFATYYCQSGYVSKSSTFGQGTKLTVL

SEQ ID NO: 702 - protein, 108 aa, synthetic construct
  REGION 1..108: note = hu29F1 LC glutamate variants in hu29F1 VL 7
EIVMTQSPSTLSASVGDRVIITCQASENIENYLAWYQQKPGKAPKLLIYYASTLASGVPSRFSGSGSGAEFTLTISSLQPDDFATYYCQSGYVSKSSTFGQGTKLTVL

SEQ ID NO: 703 - protein, 108 aa, synthetic construct
  REGION 1..108: note = hu29F1 LC glutamate variants in hu29F1 VL 8
EIVMTQSPSTLSASVGDRVIITCQASENIYEYLAWYQQKPGKAPKLLIYYASTLASGVPSRFSGSGSGAEFTLTISSLQPDDFATYYCQSGYVSKSSTFGQGTKLTVL

SEQ ID NO: 704 - protein, 108 aa, synthetic construct
  REGION 1..108: note = hu29F1 LC glutamate variants in hu29F1 VL 9
EIVMTQSPSTLSASVGDRVIITCQASENIYNELAWYQQKPGKAPKLLIYYASTLASGVPSRFSGSGSGAEFTLTISSLQPDDFATYYCQSGYVSKSSTFGQGTKLTVL

SEQ ID NO: 705 - protein, 108 aa, synthetic construct
  REGION 1..108: note = hu29F1 LC glutamate variants in hu29F1 VL 10
EIVMTQSPSTLSASVGDRVIITCQASENIYNYEAWYQQKPGKAPKLLIYYASTLASGVPSRFSGSGSGAEFTLTISSLQPDDFATYYCQSGYVSKSSTFGQGTKLTVL

SEQ ID NO: 706 - protein, 108 aa, synthetic construct
  REGION 1..108: note = hu29F1 LC glutamate variants in hu29F1 VL 11
EIVMTQSPSTLSASVGDRVIITCQASENIYNYLEWYQQKPGKAPKLLIYYASTLASGVPSRFSGSGSGAEFTLTISSLQPDDFATYYCQSGYVSKSSTFGQGTKLTVL

SEQ ID NO: 707 - protein, 108 aa, synthetic construct
  REGION 1..108: note = hu29F1 LC glutamate variants in hu29F1 VL 12
EIVMTQSPSTLSASVGDRVIITCQASENIYNYLAWYQQKPGKAPKLLIYEASTLASGVPSRFSGSGSGAEFTLTISSLQPDDFATYYCQSGYVSKSSTFGQGTKLTVL

SEQ ID NO: 708 - protein, 108 aa, synthetic construct
  REGION 1..108: note = hu29F1 LC glutamate variants in hu29F1 VL 13
EIVMTQSPSTLSASVGDRVIITCQASENIYNYLAWYQQKPGKAPKLLIYYESTLASGVPSRFSGSGSGAEFTLTISSLQPDDFATYYCQSGYVSKSSTFGQGTKLTVL

SEQ ID NO: 709 - protein, 108 aa, synthetic construct
  REGION 1..108: note = hu29F1 LC glutamate variants in hu29F1 VL 14
EIVMTQSPSTLSASVGDRVIITCQASENIYNYLAWYQQKPGKAPKLLIYYAETLASGVPSRFSGSGSGAEFTLTISSLQPDDFATYYCQSGYVSKSSTFGQGTKLTVL

SEQ ID NO: 710 - protein, 108 aa, synthetic construct
  REGION 1..108: note = hu29F1 LC glutamate variants in hu29F1 VL 15
EIVMTQSPSTLSASVGDRVIITCQASENIYNYLAWYQQKPGKAPKLLIYYASELASGVPSRFSGSGSGAEFTLTISSLQPDDFATYYCQSGYVSKSSTFGQGTKLTVL

SEQ ID NO: 711 - protein, 108 aa, synthetic construct
  REGION 1..108: note = hu29F1 LC glutamate variants in hu29F1 VL 16
EIVMTQSPSTLSASVGDRVIITCQASENIYNYLAWYQQKPGKAPKLLIYYASTEASGVPSRFSGSGSGAEFTLTISSLQPDDFATYYCQSGYVSKSSTFGQGTKLTVL

SEQ ID NO: 712 - protein, 108 aa, synthetic construct
  REGION 1..108: note = hu29F1 LC glutamate variants in hu29F1 VL 17
EIVMTQSPSTLSASVGDRVIITCQASENIYNYLAWYQQKPGKAPKLLIYYASTLESGVPSRFSGSGSGAEFTLTISSLQPDDFATYYCQSGYVSKSSTFGQGTKLTVL

SEQ ID NO: 713 - protein, 108 aa, synthetic construct
  REGION 1..108: note = hu29F1 LC glutamate variants in hu29F1 VL 18
EIVMTQSPSTLSASVGDRVIITCQASENIYNYLAWYQQKPGKAPKLLIYYASTLAEGVPSRFSGSGSGAEFTLTISSLQPDDFATYYCQSGYVSKSSTFGQGTKLTVL

SEQ ID NO: 714 - protein, 108 aa, synthetic construct
  REGION 1..108: note = hu29F1 LC glutamate variants in hu29F1 VL 19
EIVMTQSPSTLSASVGDRVIITCQASENIYNYLAWYQQKPGKAPKLLIYYASTLASGVPSRFSGSGSGAEFTLTISSLQPDDFATYYCESGYVSKSSTFGQGTKLTVL

SEQ ID NO: 715 - protein, 108 aa, synthetic construct
  REGION 1..108: note = hu29F1 LC glutamate variants in hu29F1 VL 20
EIVMTQSPSTLSASVGDRVIITCQASENIYNYLAWYQQKPGKAPKLLIYYASTLASGVPSRFSGSGSGAEFTLTISSLQPDDFATYYCQEGYVSKSSTFGQGTKLTVL

SEQ ID NO: 716 - protein, 108 aa, synthetic construct
  REGION 1..108: note = hu29F1 LC glutamate variants in hu29F1 VL 21
EIVMTQSPSTLSASVGDRVIITCQASENIYNYLAWYQQKPGKAPKLLIYYASTLASGVPSRFSGSGSGAEFTLTISSLQPDDFATYYCQSEYVSKSSTFGQGTKLTVL

SEQ ID NO: 717 - protein, 108 aa, synthetic construct
  REGION 1..108: note = hu29F1 LC glutamate variants in hu29F1 VL 22
EIVMTQSPSTLSASVGDRVIITCQASENIYNYLAWYQQKPGKAPKLLIYYASTLASGVPSRFSGSGSGAEFTLTISSLQPDDFATYYCQSGEVSKSSTFGQGTKLTVL

SEQ ID NO: 718 - protein, 108 aa, synthetic construct
  REGION 1..108: note = hu29F1 LC glutamate variants in hu29F1 VL 23
EIVMTQSPSTLSASVGDRVIITCQASENIYNYLAWYQQKPGKAPKLLIYYASTLASGVPSRFSGSGSGAEFTLTISSLQPDDFATYYCQSGYESKSSTFGQGTKLTVL

SEQ ID NO: 719 - protein, 108 aa, synthetic construct
  REGION 1..108: note = hu29F1 LC glutamate variants in hu29F1 VL 24
EIVMTQSPSTLSASVGDRVIITCQASENIYNYLAWYQQKPGKAPKLLIYYASTLASGVPSRFSGSGSGAEFTLTISSLQPDDFATYYCQSGYVEKSSTFGQGTKLTVL

SEQ ID NO: 720 - protein, 108 aa, synthetic construct
  REGION 1..108: note = hu29F1 LC glutamate variants in hu29F1 VL 25
EIVMTQSPSTLSASVGDRVIITCQASENIYNYLAWYQQKPGKAPKLLIYYASTLASGVPSRFSGSGSGAEFTLTISSLQPDDFATYYCQSGYVSESSTFGQGTKLTVL

SEQ ID NO: 721 - protein, 108 aa, synthetic construct
  REGION 1..108: note = hu29F1 LC glutamate variants in hu29F1 VL 26
EIVMTQSPSTLSASVGDRVIITCQASENIYNYLAWYQQKPGKAPKLLIYYASTLASGVPSRFSGSGSGAEFTLTISSLQPDDFATYYCQSGYVSKESTFGQGTKLTVL

SEQ ID NO: 722 - protein, 108 aa, synthetic construct
  REGION 1..108: note = hu29F1 LC glutamate variants in hu29F1 VL 27
EIVMTQSPSTLSASVGDRVIITCQASENIYNYLAWYQQKPGKAPKLLIYYASTLASGVPSRFSGSGSGAEFTLTISSLQPDDFATYYCQSGYVSKSETFGQGTKLTVL

SEQ ID NO: 723 - protein, 108 aa, synthetic construct
  REGION 1..108: note = hu29F1 LC glutamate variants in hu29F1 VL 28
EIVMTQSPSTLSASVGDRVIITCQASENIYNYLAWYQQKPGKAPKLLIYYASTLASGVPSRFSGSGSGAEFTLTISSLQPDDFATYYCQSGYVSKSSEFGQGTKLTVL

SEQ ID NO: 724 - protein, 450 aa, synthetic construct
  REGION 1..450: note = pNGH0761
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 725 - protein, 214 aa, synthetic construct
  REGION 1..214: note = pNGL1019
DIQMTQSPSSLSASVGDRVTITCRASQDISNYLNWYQQKPGGAVKFLIYHTSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 726 - protein, 450 aa, synthetic construct
  REGION 1..450: note = pNGH1909
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 727 - protein, 219 aa, synthetic construct
  REGION 1..219: note = pNGL1322
DIVMTQSPDSLAVSLGERATINCKSSQSLLNSRTRKNYLAWYQQKPGQSPKLLIYWASTRHSGVPDRFSGSGSGTDFTLTISSLQAEDVAVYYCKQSYNLPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 728 - protein, 452 aa, synthetic construct
  REGION 1..452: note = pNGH4960
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGGFHYGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 729 - protein, 217 aa, synthetic construct
  REGION 1..217: note = pNGL2802
EIVMTQSPSTLSASVGDRVIITCQASQSIGNYLSWYQQKPGKAPKRLIYSASTLPSGVPSRFSGSGSGAEFTLTISSLQPDDFATYYCQNNYWLAGYGHPFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 730 - protein, 452 aa, synthetic construct
  REGION 1..452: note = pNGH5088
EVQLVESGGGLVQPGGSLRLSCTASGLDFRRTNYICWVRQAPGKGLEWVGCIYIADVVHYYASWAKGRFTFSRDTSKNTVYLQMNSLRAEDTAVYYCARGADGGFVYGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 731 - protein, 217 aa, synthetic construct
  REGION 1..217: note = pNGL3334
EIVMTQSPSTLSASVGDRVIITCQASQSIGNYLSWYQQKPGKAPKRLIYSASTLPSGVPSRFSGSGSGAEFTLTISSLQPDDFATYYCQNNHWLAGYGHPFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 732 - protein, 217 aa, synthetic construct
  REGION 1..217: note = pNGL3336
EIVMTQSPSTLSASVGDRVIITCQASQSIGNYLSWYQQKPGKAPKRLIYSASTLPSGVPSRFSGSGSGAEFTLTISSLQPDDFATYYCQNNYWLAHYGHPFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 733 - protein, 455 aa, synthetic construct
  REGION 1..455: note = pNGH5090
EVQLVESGGGLVQPGGSLRLSCTASGFSFSGSYWICWVRQAPGKGLEWVGCINGGSLDITYYASWAKGRFTISRDTSKNTVYLQKTSLRAEDTAVYYCASDTSYTAYGNFYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 734 - protein, 220 aa, synthetic construct
  REGION 1..220: note = pNGL2828
EIVMTQSPSPLSASVGDRVIITCQSSQSVLNNNWLAWYQQKPGKAPKLLIYDASSLASGVPSRFSGSGSGAEFTLTISSLQPDDFATYYCQGEFSCGSADCNAFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 735 - protein, 455 aa, synthetic construct
  REGION 1..455: note = pNGH6020
EVQLVESGGGLVQPGGSLRLSCTASGFSFSGHYWICWVRQAPGKGLEWVGCIHGGSHDITYYASWAKGRFTISRDTSKNTVYLQKTSLRAEDTAVYYCASDTSYTAYGNFYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 736 - protein, 220 aa, synthetic construct
  REGION 1..220: note = pNGL4035
EIVMTQSPSPLSASVGDRVIITCQSSQSVLNNNWLAWYQQKPGKAPKLLIYDASSLASGVPSRFSGSGSGAEFTLTISSLQPDDFATYYCQGEFSCGSADCNAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 737 - protein, 453 aa, synthetic construct
  REGION 1..453: note = pNGH4765
EVQLVESGGGLVQPGGSLRLSCTASGFSFSSSHWICWVRQAPGKGLEWVGCIYAGSIGSTYYASWAKGRFTISRDTSKNTVYLQMNSLRAEDTAVYYCARAPNDIYNYYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 738 - protein, 215 aa, synthetic construct
  REGION 1..215: note = pNGL3940
EIVMTQSPSTLSASVGDRVIITCQASENIYNYLAWYQQKPGKAPKLLIYYASDLASGVPSRFSGSGSGAEFTLTISSLQPDDFATYYCQSGYVSKSSTFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 739 - protein, 215 aa, synthetic construct
  REGION 1..215: note = pNGL3958
EIVMTQSPSTLSASVGDRVIITCQASEEIYNYLAWYQQKPGKAPKLLIYYASTLASGVPSRFSGSGSGAEFTLTISSLQPDDFATYYCQSGYVSKSSTFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 740 - protein, 215 aa, synthetic construct
  REGION 1..215: note = pNGL3191
EIVMTQSPSTLSASVGDRVIITCQASENIYNYLAWYQQKPGKAPKLLIYYASTLASGVPSRFSGSGSGAEFTLTISSLQPDDFATYYCQSGYVSKSSTFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

(19) United States
(12) Patent Application Publication
(10) Pub. No.: US 2025/0352661 A1
(43) Pub. Date: Nov. 20, 2025

(54) LRRC-15-BINDING PROTEIN CONSTRUCTS AND USES THEREOF

(71) Applicant: Mythic Therapeutics, Inc., Waltham, MA (US)

(72) Inventors: Nimish Gera, Waltham, MA (US); Brian Fiske, Waltham, MA (US); Kyle Michael Fitzgerald, Waltham, MA (US)

(73) Assignee: Mythic Therapeutics, Inc., Waltham, MA (US)

(21) Appl. No.: 19/119,540

(22) PCT Filed: Oct. 11, 2023
(86) PCT No.: PCT/US2023/076593

§ 371 Date: Apr. 9, 2025

Related U.S. Application Data
(60) Provisional application No. 63/415,594, filed on Oct. 12, 2022.

(51) Int. Cl. A61K 47/68; A61P 35/00; C07K 16/28

(52) U.S. Cl. CPC A61K 47/6849; A61K 47/68031; A61K 47/68035; A61P 35/00; C07K 16/28; C07K 2317/33; C07K 2317/565; C07K 2317/77; C07K 2317/92; C07K 2317/94

(57) ABSTRACT

Provided herein are antigen-binding protein constructs (ABPCs), antibodies, and antibody drug conjugates (ADCs) that bind LRRC15, and uses of the same.

### CROSS-REFERENCE TO RELATED APPLICATIONS

This application claims priority to U.S. Provisional patent application Ser. No. 63/415,594, filed Oct. 12, 2022, the entire contents of which are herein incorporated by reference.

### SEQUENCE LISTING

This application contains a Sequence Listing that has been submitted electronically as an XML file named “LRRC15_PRO_SL.XML”. The XML file, created on Sep. 22, 2023, is 1,035,741 bytes in size. The material in the XML file is hereby incorporated by reference in its entirety.

### TECHNICAL FIELD

The present disclosure relates to the field of biotechnology, and more specifically, to antigen-binding molecules.

### SUMMARY

Antibody-drug-conjugates (ADCs) have been designed to combat a variety of diseases. One particular advantage of this approach is the ability for ADCs to have cytostatic or cytotoxic effects. Despite years of development, improved ADCs are desired.

The present invention is based on novel antigen-binding protein constructs (ABPCs) and antibodies (Abs) that display increased internalization into mammalian cells, increased endolysosomal delivery, increased toxin liberation inside such cells, increased efficacy against conditions including cancer, and increased target mammalian cell killing.

Provided herein are pharmaceutical compositions including an effective amount of an ABPC including: a first antigen-binding domain (ABD) that is capable of specifically binding LRRC15 or an epitope of LRRC15 presented on the surface of a target mammalian cell, where: (a) the dissociation rate of the first ABD at a pH of ˜4.0 to ˜6.5 is faster than the dissociation rate at a pH of ˜7.0 to ˜8.0; and/or (b) the dissociation constant (KD) of the first ABD at a pH of ˜4.0 to ˜6.5 is greater than the KD at a pH of ˜7.0 to ˜8.0.

Non-limiting examples of LRRC15-specific ABPCs and antibodies may include those comprising the variable domains and/or CDRs disclosed in WO 2022/216653 A1 (to Mythic Therapeutics, and incorporated herein by reference), including those ABPCs, antibodies, variable domains, and/or CDRs present in MYT2737 and MYT3315.

Encouraged by their initial findings, and reasoning that anti-LRRC15-ADCs could prove useful against both LRRC15+ tumors as well as LRRC15-tumors associated with LRRC15+ stromal cells, Applicant produced the novel anti-LRRC15 antibodies disclosed herein.

As further detailed herein, the following are non-limiting examples of pH-dependent, anti-LRRC15 ABPCs that exhibit strong target-specific binding at physiologic pH, enhanced dissociation at acidic pH, enhanced internalization into LRRC15+ cells, and increased cytotoxicity when complexed or conjugated to toxic payload: MYT2737 (HCV=SEQ ID NO: 1, LCV=SEQ ID NO: 64), MYT3315 (HCV=SEQ ID NO: 84, LCV=SEQ ID NO: 154), MYT8391 (HCV=SEQ ID NO: 430, LCV=SEQ ID NO: 455), MYT8415 (HCV=SEQ ID NO: 382, LCV=SEQ ID NO: 489), MYT8417 (HCV=SEQ ID NO: 382, LCV-SEQ ID NO: 491), MYT8483 (HCV=SEQ ID NO: 516, LCV=SEQ ID NO: 522), MYT9776 (HCV=SEQ ID NO: 571, LCV=SEQ ID NO: 517), MYT8094 (HCV=SEQ ID NO: 576, LCV=SEQ ID NO: 581), MYT9521 (HCV=SEQ ID NO: 576, LCV=SEQ ID NO: 639), MYT9731 (HCV=SEQ ID NO: 576, LCV=SEQ ID NO: 700), MYT8416 (HCV=SEQ ID NO: 382, LCV=SEQ ID NO: 490), MYT8500 (HCV=SEQ ID NO: 570, LCV=SEQ ID NO: 522), MYT9523 (HCV=SEQ ID NO: 576, LCV=SEQ ID NO: 641), MYT4174 (HCV=SEQ ID NO: 84, LCV=SEQ ID NO: 177), and MYT9507 (HCV=SEQ ID NO: 576, LCV=SEQ ID NO: 625).

In some embodiments, the anti-LRRC15 ABPCs have the same heavy chain and light chains, heavy and light chain variable domains, and/or heavy and light chain CDRs (e.g., as determined by, for example, the Kabat, Chothia, and/or IMGT numbering schemes) as MYT2737-c (comprising SEQ ID NOs: 724 & 725); MYT3315-c (comprising SEQ ID NOs: 726 & 727); MYT8391-c (comprising SEQ ID NOs: 728 & 729); MYT8415-c (comprising SEQ ID NOs: 730 & 731); MYT8417-c (comprising SEQ ID NOs: 730 & 732); MYT8483-c (comprising SEQ ID NOs: 733 & 734); MYT9776-c (comprising SEQ ID NOs: 735 & 736); MYT9521-c (comprising SEQ ID NOs: 737 & 738); MYT9731-c (comprising SEQ ID NOs: 737 & 739); or MYT8094-c (comprising 737 & 740). Each of the foregoing are amenable to toxin conjugation at a variety of positions, including position 205 of the respective light chain (i.e., the sequence set forth in one of SEQ ID NOs: 725, 727, 729, 731, 732, 734, 736, 738, 739, and 740). In some embodiments, position 205 of the light chain comprises an amino acid other than a cysteine (e.g., a valine, leucine, isoleucine, or alanine). In some embodiments, toxin conjugation may be made at other positions known to the skilled artisan. In still other embodiments, toxins may be attached, for example, via hinge conjugation, or any other suitable attachment methodology.

In some embodiments, the first ABD (also, “LRRC15 binding domain”) includes a heavy chain variable (HCV) domain of one of: (a) an HCV domain of 15G7, optionally with one or more amino acids substituted with a histidine; (b) an HCV domain of 24D9, optionally with one or more amino acids substituted with a histidine; and (c) an HCV domain of 29F1, optionally with one or more amino acids substituted with a histidine, an aspartate, or a glutamate. The first ABD may also include an HCV domain of MYT2737 (HC=SEQ ID NO: 1, LC-SEQ ID NO: 64) or MYT3315 (HC=SEQ ID NO: 84, LC=SEQ ID NO: 154). In some embodiments, the first ABD may include the three HCDRs of MYT2737 or MYT3315. As used herein, “15G7”, “24D9”, and “29F1” each refers to a newly discovered rabbit monoclonal antibody (RabMAb) clone and any of its corresponding humanized counterparts. When preceded by “hu” (e.g., “hu15G7”), a humanized clone is intended.

In some embodiments, the anti-LRRC15 antibody comprises a heavy chain and a light chain having the polypeptide sequences as set forth in SEQ ID NOs: 724 & 725; 726 & 727; 728 & 729; 730 & 731; 730 & 732; 733 & 734; 735 & 736; 737 & 738; 737 & 739; or 737 & 740. In other embodiments, the anti-LRRC15 antibody comprises a heavy chain and a light chain having the polypeptide sequences as set forth in SEQ ID NOs: 724 & 725; 726 & 727; 728 & 729; 730 & 731; 730 & 732; 733 & 734; 735 & 736; 737 & 738; 737 & 739; or 737 & 740, wherein position 205 of the respective light chain is not a cysteine. In some embodiments, the anti-LRRC15 antibody comprises a light chain having a valine, leucine, or isoleucine at position 205.

In some embodiments, the anti-LRRC15 antibody may comprise a heavy chain and a light chain comprising favorable “developability mutations” as described, for example, in Table 33. As used herein, “developability mutations” means changes in the amino acid sequence of an ABPC, including an antibody, that tend to improve certain biophysical properties (e.g., reduced potential for aggregation). In other embodiments, the heavy chain and light chain may comprise conservative amino acid substitutions with respect to the favorable developability mutations.

In some embodiments, a composition including the ABPC or antibody provides for one or more of the following: an increase in toxin liberation in a target mammalian cell as compared to a composition including the same amount of a control antibody; an increase in target mammalian cell killing as compared to a composition including the same amount of a control antibody; and/or an increase in endolysosomal delivery in the target mammalian cell as compared to a composition including the same amount of a control antibody.

In some embodiments, a composition including the antibody results in a diminished or undetectable reduction in the level of LRRC15 presented on the surface of a target mammalian cell as compared to a composition including the same amount of a control antibody.

In some embodiments, the antibody is degraded in a target mammalian cell following internalization of the antibody by a target mammalian cell. In some embodiments, the target mammalian cell is a cancer cell. In some embodiments, the antibody is cytotoxic or cytostatic to the target mammalian cell. In some embodiments, the antibody has an avidity that results in increased selectivity for cancer cells over non-cancerous cells.

In some embodiments, the antibody is cross-reactive with one or both of a non-human primate LRRC15 and a human LRRC15; or a rat LRRC15 and a mouse LRRC15.

In some embodiments, the half-life of the antibody in vivo is increased as compared to the half-life of a control antibody in vivo.

In some embodiments, the first ABD includes a light chain variable (LCV) domain of one of: (a) an LCV domain of 15G7, optionally with one or more amino acids substituted with a histidine; (b) an LCV domain of 24D9, optionally with one or more amino acids substituted with a histidine; and (c) an LCV domain of 29F1, optionally with one or more amino acids substituted with a histidine, an aspartate, or a glutamate. The first ABD may also include an LCV domain of MYT2737 or MYT3315.

In some embodiments, the first ABD includes one of: (a) an HCV domain of 15G7, optionally with one or more amino acids substituted with a histidine; and/or an LCV domain of 15G7, optionally with one or more amino acids substituted with a histidine; (b) an HCV domain of 24D9, optionally with one or more amino acids substituted with a histidine; and/or an LCV domain of 24D9, optionally with one or more amino acids substituted with a histidine; and (c) an HCV domain of 29F1, optionally with one or more amino acids substituted with a histidine, an aspartate, or a glutamate; and/or an LCV domain of 29F1, optionally with one or more amino acids substituted with a histidine, an aspartate, or a glutamate. In cases where the wildtype amino acid is already histidine, an alanine may be substituted instead.

In some embodiments, the HCV domain includes one of: (a) an HCV domain of 15G7 including SEQ ID NO: 378, or a humanized version thereof (e.g., SEQ ID NO: 382); (b) an HCV domain of 24D9 including SEQ ID NO: 512, or a humanized version thereof (e.g., SEQ ID NO: 516); and (c) an HCV domain of 29F1 including SEQ ID NO: 572, or a humanized version thereof (e.g., SEQ ID NO: 576). In some embodiments, the LCV domain includes one of: (a) an LCV domain of 15G7 including SEQ ID NO: 451, or a humanized version thereof (e.g., SEQ ID NO: 455); (b) an LCV domain of 24D9 including SEQ ID NO: 518, or a humanized version thereof (e.g., SEQ ID NO: 522); and (c) an LCV domain of 29F1 including SEQ ID NO: 577, or a humanized version thereof (e.g., SEQ ID NO: 581).

In some embodiments, the first LRRC15 binding domain includes an HCV domain of one of: (a) an HCV domain including a CDR1, a CDR2, and a CDR3 of SEQ ID NOs: 379-381, respectively, optionally with collectively a total of one or more amino acid positions in SEQ ID NOs: 379-381 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate.

In some embodiments, the first LRRC15-binding domain includes an LCV domain of one of: (a) an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate.

In some embodiments, the first LRRC15-binding domain includes one of: (a) an HCV domain including a CDR1, a CDR2, and a CDR3 of SEQ ID NOs: 379-381, respectively, optionally with collectively a total of one or more amino acid positions in SEQ ID NOs: 379-381 substituted with a histidine; and/or an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and/or an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate; and/or an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate.

In some embodiments, the first ABD includes an HCV domain of one of: (a) an HCV domain that is at least 90% identical to the sequence set forth in SEQ ID NO: 378 or SEQ ID NO: 382, where the HCV domain includes a histidine (or alanine where a histidine is already present) at one or more positions in SEQ ID NO: 378 or SEQ ID NO: 382 selected from the group consisting of: 32, 34, 53, 60, 103, 104, 105, 109, and 110; (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and (c) an HCV domain that is at least 90% identical to the sequence set forth in SEQ ID NO: 572 or SEQ ID NO: 576, where the HCV domain includes a histidine, an aspartate, or a glutamate at one or more positions in SEQ ID NO: 572 or SEQ ID NO: 576 selected from the group consisting of: 31, 56, 59, and 99. In some embodiments, the first ABD includes an LCV domain of one of: (a) an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; (b) an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an LCV domain that is at least 90% identical to the sequence set forth in SEQ ID NO: 577 or SEQ ID NO: 581, where the LCV domain includes a histidine, an aspartate (D), or a glutamate (E) at one or more positions in SEQ ID NO: 577 or SEQ ID NO: 581 selected from the group consisting of: D positions selected from 27, 28, 31, 52, and 56; and E positions selected from 51 and 56.

In some embodiments, the first ABD includes an HCV domain of one of: (a) an HCV domain that is at least 90% identical to the sequence set forth in SEQ ID NO: 378 or SEQ ID NO: 382, where the HCV domain includes a histidine at two or more positions in SEQ ID NO: 378 or SEQ ID NO: 382 including one pair of positions including one pair selected from the group consisting of: 34, 53; 34, 104; 34, 105; 34, 106; 53, 104; 53, 105 and 53,106; and (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516. In some embodiments, the first ABD includes an LCV domain of one of: (a) an LCV domain that is at least 90% identical to the sequence set forth in SEQ ID NO: 451 or SEQ ID NO: 455, where the LCV domain includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96; and 93, 96; and (b) an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522.

In some embodiments, the first ABD includes one of: (a) an HCV domain that is at least 90% identical to the sequence set forth in SEQ ID NO: 378 or SEQ ID NO: 382, where the HCV domain includes a histidine at one or more positions in SEQ ID NO: 378 or SEQ ID NO: 382 selected from the group consisting of: 32, 34, 53, 60, 103, 104, 105, 109, and 110; and/or an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; and (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an HCV domain that is at least 90% identical to the sequence set forth in SEQ ID NO: 572 or SEQ ID NO: 576, where the HCV domain includes an aspartate (D) or a glutamate (E) at one or more positions in SEQ ID NO: 572 or SEQ ID NO: 576 selected from the group consisting of: D positions selected from 31, 56 and 99; or E position 59; and/or an LCV domain that is at least 90% identical to the sequence set forth in SEQ ID NO: 577 or SEQ ID NO: 581, where the LCV domain includes an aspartate (D) or a glutamate (E) at one or more positions in SEQ ID NO: 577 or SEQ ID NO: 581 selected from the group consisting of: D positions selected from 27, 28, 31, 52 and 56; and E positions selected from 51 and 56.

In some embodiments, the first ABD includes an HCV domain of one of: (a) an HCV domain comprising the sequence of one of SEQ ID NO: 378, SEQ ID NO: 382, one of SEQ ID NOs: 383-424, and one of SEQ ID NOs: 425-450; (b) an HCV domain comprising the sequence of one of SEQ ID NO: 512, SEQ ID NO: 516, one of SEQ ID NOs: 523-567, and one of SEQ ID NOs: 568-571; and (c) an HCV domain comprising the sequence of one of SEQ ID NO: 572, SEQ ID NO: 576, one of SEQ ID NOs: 582-624, and one of SEQ ID NOs: 653-695; and/or the first ABD includes an LCV domain of one of: (a) an LCV domain having the sequence of one of SEQ ID NO: 451, SEQ ID NO: 455, one of SEQ ID NOs: 456-485, and one of SEQ ID NOS: 486-511; (b) an LCV domain comprising the sequence of SEQ ID NO: 518, SEQ ID NO: 522, SEQ ID: 518 (with an L35H substitution), or SEQ ID NO: 518 (with a G97H substitution); and (c) an LCV domain comprising the sequence of one of SEQ ID NO: 577, SEQ ID NO: 581, one of SEQ ID NOs: 625-652, and one of SEQ ID NOs: 696-723.

In some embodiments, the first ABD includes one of: (a) an HCV domain comprising the sequence of one of SEQ ID NO: 378, SEQ ID NO: 382, one of SEQ ID NOs: 383-424, and one of SEQ ID NOs: 425-450, and/or an LCV domain having the sequence of one of SEQ ID NO: 451, SEQ ID NO: 455, one of SEQ ID NOs: 456-485, and one of SEQ ID NOs: 486-511, where the first ABD does not include (i) an HCV domain of SEQ ID NO: 378 or SEQ ID NO: 382 and an LCV domain comprising a sequence that is not one of SEQ ID NO: 461, SEQ ID NO: 462, SEQ ID NO: 464, SEQ ID NO: 466, SEQ ID NO: 467, one of SEQ ID NOs: 477-478, and SEQ ID NOs: 480-482; or (ii) an LCV domain comprising the sequence of SEQ ID NO: 451 or SEQ ID NO: 455 and an HCV domain comprising a sequence that is not one of SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, and SEQ ID NO: 450; (b) an HCV domain comprising the sequence of one of SEQ ID NO: 512, SEQ ID NO: 516, one of SEQ ID NOs: 523-567, and one of SEQ ID NOs: 568-571; and/or an LCV domain comprising the sequence of SEQ ID NO: 518, SEQ ID NO: 522, SEQ ID: 518 (with an L35H substitution), or SEQ ID NO: 518 (with a G97H substitution), where the first ABD does not include (i) an HCV domain comprising the sequence of SEQ ID NO: 512 or SEQ ID NO: 516 and an LCV domain comprising a sequence that is not one of SEQ ID NO: 518 (with an L35H substitution) and SEQ ID NO: 518 (with a G97H substitution); or (ii) an LCV domain comprising the sequence of SEQ ID NO: 518, SEQ ID NO: 522, SEQ ID: 518 (with L35H), or SEQ ID NO: 518 (with G97H) and an HCV domain comprising a sequence that does not include one of: one of SEQ ID NOs: 527-529, one of SEQ ID NOs: 535-536, one of SEQ ID NOs: 541-543, SEQ ID NO: 552, one of SEQ ID NOs: 559-560, and SEQ ID NO: 564; and (c) an HCV domain comprising the sequence of SEQ ID NO: 572 or SEQ ID NO: 576 and/or an LCV domain comprising the sequence of one of SEQ ID NO: 577, SEQ ID NO: 581, one of SEQ ID NOs: 625-652, and one of SEQ ID NOs: 696-723, where the first ABD does not include (i) an HCV domain comprising the sequence of SEQ ID NO: 572 or SEQ ID NO: 576 and an LCV domain comprising a sequence that is not one of SEQ ID NO: 628, SEQ ID NO: 629, SEQ ID NO: 632, SEQ ID NO: 638 and SEQ ID NO: 642; or (ii) an LCV domain comprising a sequence of SEQ ID NO: 577 or SEQ ID NO: 581 and an HCV domain comprising a sequence that is not one of SEQ ID NO: 587, SEQ ID NO: 598, SEQ ID NO: 611, and SEQ ID NO: 672.

In some embodiments, the ABPC is degraded in the target mammalian cell following internalization of the ABPC by the target mammalian cell. In some embodiments, the ABPC includes a conjugated toxin, radioisotope, drug, or small molecule.

In some embodiments, the composition provides for an increase in toxin liberation in the target mammalian cell as compared to a composition including the same amount of a control ABPC. In some embodiments, the composition provides for at least a 20% increase in toxin liberation in the target mammalian cell as compared to a composition including the same amount of a control ABPC. In some embodiments, the composition provides for at least a 50% increase in toxin liberation in the target mammalian cell as compared to a composition including the same amount of a control ABPC. In some embodiments, the composition provides for at least a 2-fold increase in toxin liberation in the target mammalian cell as compared to a composition including the same amount of a control ABPC. In some embodiments, the composition provides for at least a 5-fold increase in toxin liberation in the target mammalian cell as compared to a composition including the same amount of a control ABPC.

In some embodiments, the composition provides for an increase in target mammalian cell killing as compared to a composition including the same amount of a control ABPC. In some embodiments, the composition provides for at least a 20% increase in target mammalian cell killing as compared to a composition including the same amount of a control ABPC. In some embodiments, the composition provides for at least a 50% increase in target mammalian cell killing as compared to a composition including the same amount of a control ABPC. In some embodiments, the composition provides for at least a 2-fold increase in target mammalian cell killing as compared to a composition including the same amount of a control ABPC. In some embodiments, the composition provides for at least a 5-fold increase in target mammalian cell killing as compared to a composition including the same amount of a control ABPC.

In some embodiments, the composition provides for an increase in endolysosomal delivery in the target mammalian cell as compared to a composition including the same amount of a control ABPC. In some embodiments, the composition provides for at least a 20% increase in endolysosomal delivery in the target mammalian cell as compared to a composition including the same amount of a control ABPC. In some embodiments, the composition provides for at least a 50% increase in endolysosomal delivery in the target mammalian cell as compared to a composition including the same amount of a control ABPC. In some embodiments, the composition provides for at least a 2-fold increase in endolysosomal delivery in the target mammalian cell as compared to a composition including the same amount of a control ABPC. In some embodiments, the composition provides for at least a 5-fold increase in endolysosomal delivery in the target mammalian cell as compared to a composition including the same amount of a control ABPC.

In some embodiments, the composition results in a less of a reduction in the level of LRRC15 presented on the surface of the target mammalian cell as compared to a composition including the same amount of a control ABPC. In some embodiments, the composition does not result in a detectable reduction in the level of the LRRC15 presented on the surface of the target mammalian cell.

Also provided herein are pharmaceutical compositions including an effective amount of an antigen-binding protein construct (ABPC) including: a first ABD that is capable of specifically binding LRRC15 or an epitope of LRRC15 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; or the dissociation constant (KD) of the first ABD at a pH of about 4.0 to about 6.5 is greater than the KD at a pH of about 7.0 to about 8.0; and (b) the composition provides for one or more of: an increase in toxin liberation in the target mammalian cell as compared to a composition including the same amount of a control ABPC; an increase in target mammalian cell killing as compared to a composition including the same amount of a control ABPC; and an increase in endolysosomal delivery in the target mammalian cell as compared to a composition including the same amount of a control ABPC.

In some embodiments, the first ABD includes an HCV domain of one of: (a) an HCV domain of 15G7, optionally with one or more amino acids substituted with a histidine; (b) an HCV domain of 24D9, optionally with one or more amino acids substituted with a histidine; and (c) an HCV domain of 29F1, optionally with one or more amino acids substituted with a histidine, an aspartate, or a glutamate. In some embodiments, the first ABD includes an LCV domain of one of: (a) an LCV domain of 15G7, optionally with one or more amino acids substituted with a histidine; (b) an LCV domain of 24D9, optionally with one or more amino acids substituted with a histidine; and (c) an LCV domain of 29F1, optionally with one or more amino acids substituted with a histidine, an aspartate, or a glutamate.

In some embodiments, the first ABD includes one of: (a) an HCV domain of 15G7, optionally with one or more amino acids substituted with a histidine; and/or an LCV domain of 15G7, optionally with one or more amino acids substituted with a histidine; (b) an HCV domain of 24D9, optionally with one or more amino acids substituted with a histidine; and/or an LCV domain of 24D9, optionally with one or more amino acids substituted with a histidine; and (c) an HCV domain of 29F1, optionally with one or more amino acids substituted with a histidine, an aspartate, or a glutamate; and/or an LCV domain of 29F1, optionally with one or more amino acids substituted with a histidine, an aspartate, or a glutamate.

In some embodiments, the HCV domain includes one of: (a) an HCV domain of 15G7 including SEQ ID NO: 378, or a humanized version thereof (e.g., SEQ ID NO: 382); (b) an HCV domain of 24D9 including SEQ ID NO: 512, or a humanized version thereof (e.g., SEQ ID NO: 516); and (c) an HCV domain of 29F1 including SEQ ID NO: 572, or a humanized version thereof (e.g., SEQ ID NO: 576); In some embodiments, the LCV domain includes one of: (a) an LCV domain of 15G7 including SEQ ID NO: 451, or a humanized version thereof (e.g., SEQ ID NO: 455); (b) an LCV domain of 24D9 including SEQ ID NO: 518, or a humanized version thereof (e.g., SEQ ID NO: 522); and (c) an LCV domain of 29F1 including SEQ ID NO: 577, or a humanized version thereof (e.g., SEQ ID NO: 581).

In some embodiments, the first ABD includes an HCV domain including one of: (a) an HCV domain including a CDR1, a CDR2, and a CDR3 of SEQ ID NOS: 379-381, respectively, optionally with collectively a total of one or more amino acid positions in SEQ ID NOS: 379-381 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate.

In some embodiments, the first LRRC15-binding domain includes an LCV domain of one of: (a) an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate.

In some embodiments, the first LRRC15-binding domain includes one of: (a) an HCV domain including a CDR1, a CDR2, and a CDR3 of SEQ ID NOs: 379-381, respectively, optionally with collectively a total of one or more amino acid positions in SEQ ID NOs: 379-381 substituted with a histidine; and/or an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and/or an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate; and/or an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate.

In some embodiments, the first ABD includes an HCV domain of one of: (a) an HCV domain that is at least 90% identical to the sequence set forth in SEQ ID NO: 378 or SEQ ID NO: 382, where the HCV domain includes a histidine at one or more positions in SEQ ID NO: 378 or SEQ ID NO: 382 selected from the group consisting of: 32, 34, 53, 60, 103, 104, 105, 109, and 110; (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and (c) an HCV domain that is at least 90% identical to the sequence set forth in SEQ ID NO: 572 or SEQ ID NO: 576, where the HCV domain includes a histidine, an aspartate, or a glutamate at one or more positions in SEQ ID NO: 572 or SEQ ID NO: 576 selected from positions 31, 56, 59, and 99.

In some embodiments, the first ABD includes an LCV domain of one of: (a) an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; (b) an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an LCV domain that is at least 90% identical to the sequence set forth in SEQ ID NO: 577 or SEQ ID NO: 581, where the LCV domain includes a histidine, an aspartate, or a glutamate at one or more positions in SEQ ID NO: 577 or SEQ ID NO: 581 selected from the group consisting of: 27, 28, 31, 51, 52, and 56.

In some embodiments, the first ABD includes an HCV domain of one of: (a) an HCV domain that is at least 90% identical to the sequence set forth in SEQ ID NO: 378 OR 382, where the HCV domain includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53,106; and (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516. In some embodiments, the first ABD includes an LCV domain of one of: (a) an LCV domain that is at least 90% identical to the sequence set forth in SEQ ID NO: 451 or SEQ ID NO: 455, where the LCV domain includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96; 93, 96; and (b) an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522.

In some embodiments, the first ABD includes one of: (a) an HCV domain that is at least 90% identical to the sequence set forth in SEQ ID NO: 378 OR 382, where the HCV domain includes a histidine at one or more positions in SEQ ID NO: 378 or SEQ ID NO: 382 selected from the group consisting of: 32, 34, 53, 60, 103, 104, 105, 109, and 110; and/or an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an HCV domain that is at least 90% identical to the sequence set forth in SEQ ID NO: 572 or SEQ ID NO: 576, where the HCV domain includes a histidine, an aspartate, or a glutamate at one or more positions in SEQ ID NO: 572 or SEQ ID NO: 576 selected from the group consisting of: 31, 56, 59, and 99; and/or an LCV domain that is at least 90% identical to the sequence set forth in SEQ ID NO: 577 or SEQ ID NO: 581, where the LCV domain includes a histidine, an aspartate, or a glutamate at one or more positions in SEQ ID NO: 577 or SEQ ID NO: 581 selected from the group consisting of: 27, 28, 31, 51, 52, and 56.

In some embodiments, the first ABD includes an HCV domain of one of: (a) an HCV domain comprising the sequence of one of SEQ ID NO: 378, SEQ ID NO: 382, one of SEQ ID NOs: 383-424, and one of SEQ ID NOs: 425-450; (b) an HCV domain comprising the sequence of one of SEQ ID NO: 512, SEQ ID NO: 516, one of SEQ ID NOs: 523-567, and one of SEQ ID NOs: 568-571; and (c) an HCV domain comprising the sequence of one of SEQ ID NO: 572, SEQ ID NO: 576, one of SEQ ID NOs: 582-624, and one of SEQ ID NOs: 653-695.

In some embodiments, the first ABD includes an LCV domain of one of: (a) an LCV domain having the sequence of one of SEQ ID NO: 451, SEQ ID NO: 455, one of SEQ ID NOs: 456-485, and one of SEQ ID NOs: 486-511; (b) an LCV domain comprising the sequence of SEQ ID NO: 518, SEQ ID NO: 522, SEQ ID: 518 (with L35H), or SEQ ID NO: 518 (with G97H); and (c) an LCV domain comprising the sequence of one of SEQ ID NO: 577, SEQ ID NO: 581, one of SEQ ID NOs: 625-652, and one of SEQ ID NOs: 696-723.

In some embodiments, the first ABD includes one of: (a) an HCV domain comprising the sequence of any one of SEQ ID NO: 378, SEQ ID NO: 382, one of SEQ ID NOs: 383-424, and one of SEQ ID NOs: 425-450, and/or an LCV domain of any one of SEQ ID NO: 451, SEQ ID NO: 455, one of SEQ ID NOs: 456-485, and one of SEQ ID NOs: 486-511, where the first ABD does not include (i) an HCV domain of SEQ ID NO: 378 or SEQ ID NO: 382 and an LCV domain comprising a sequence that is not one of SEQ ID NO: 461, SEQ ID NO: 462, SEQ ID NO: 464, SEQ ID NO: 466, SEQ ID NO: 467, one of SEQ ID NOs: 477-478, and one of SEQ ID NOs: 480-482; and (ii) an LCV domain comprising the sequence of SEQ ID NO: 451 or SEQ ID NO: 455 and an HCV domain comprising a sequence that does not include one of: one of SEQ ID NOs: 527-529, one of SEQ ID NOs: 535-536, one of SEQ ID NOs: 541-543, SEQ ID NO: 552, one of SEQ ID NOs: 559-560, and SEQ ID NO: 564; (b) an HCV domain comprising the sequence of any one of SEQ ID NO: 512, 516, 523-567, or 568-571; and/or an LCV domain comprising the sequence of SEQ ID NO: 518, SEQ ID NO: 522, SEQ ID: 518 (with L35H), or SEQ ID NO: 518 (with G97H), where the first ABD does not include (i) an HCV domain comprising the sequence of SEQ ID NO: 512 or SEQ ID NO: 516 and an LCV domain comprising a sequence that is not one of SEQ ID NO: 518 (with an L35H substitution) and SEQ ID NO: 518 (with a G97H substitution); and (ii) an LCV domain comprising the sequence of SEQ ID NO: 518, SEQ ID NO: 522, SEQ ID: 518 (with L35H), or SEQ ID NO: 518 (with G97H) and an HCV domain comprising a sequence that does not include one of: one of SEQ ID NOs: 527-529, one of SEQ ID NOs: 535-536, one of SEQ ID NOs: 541-543, SEQ ID NO: 552, one of SEQ ID NOs: 559-560, and SEQ ID NO: 564; and (c) an HCV domain comprising the sequence of any one of SEQ ID NO: 572, SEQ ID NO: 576, one of SEQ ID NOs: 582-624, and one of SEQ ID NOs: 653-695; and/or an LCV domain of any one of SEQ ID NO: 577, 581, 625-652, or 696-723, where the first ABD does not include (i) an HCV domain comprising the sequence of SEQ ID NO: 572 or SEQ ID NO: 576 and an LCV domain comprising a sequence that is not one of SEQ ID NO: 628, SEQ ID NO: 629, SEQ ID NO: 632, SEQ ID NO: 638 and SEQ ID NO: 642; and (ii) an LCV domain comprising the sequence of SEQ ID NO: 577 or SEQ ID NO: 581 and an HCV domain comprising a sequence that is not one of SEQ ID NO: 587, SEQ ID NO: 598, SEQ ID NO: 611, and SEQ ID NO: 672.

In some embodiments, the composition provides for an increase in toxin liberation in the target mammalian cell as compared to a composition including the same amount of a control ABPC. In some embodiments, the composition provides for at least a 20% increase in toxin liberation in the target mammalian cell as compared to a composition including the same amount of a control ABPC. In some embodiments, the composition provides for at least a 50% increase in toxin liberation in the target mammalian cell as compared to a composition including the same amount of a control ABPC. In some embodiments, the composition provides for at least a 2-fold increase in toxin liberation in the target mammalian cell as compared to a composition including the same amount of a control ABPC. In some embodiments, the composition provides for at least a 5-fold increase in toxin liberation in the target mammalian cell as compared to a composition including the same amount of a control ABPC.

In some embodiments, the composition provides for an increase in target mammalian cell killing as compared to a composition including the same amount of a control ABPC. In some embodiments, the composition provides for at least a 20% increase in target mammalian cell killing as compared to a composition including the same amount of a control ABPC. In some embodiments, the composition provides for at least a 50% increase in target mammalian cell killing as compared to a composition including the same amount of a control ABPC. In some embodiments, the composition provides for at least a 2-fold increase in target mammalian cell killing as compared to a composition including the same amount of a control ABPC. In some embodiments, the composition provides for at least a 5-fold increase in target mammalian cell killing as compared to a composition including the same amount of a control ABPC. In some embodiments, the composition provides for an increase in endolysosomal delivery in the target mammalian cell as compared to a composition including the same amount of a control ABPC.

In some embodiments, the composition provides for at least a 20% increase in endolysosomal delivery in the target mammalian cell as compared to a composition including the same amount of a control ABPC. In some embodiments, the composition provides for at least a 50% increase in endolysosomal delivery in the target mammalian cell as compared to a composition including the same amount of a control ABPC. In some embodiments, the composition provides for at least a 2-fold increase in endolysosomal delivery in the target mammalian cell as compared to a composition including the same amount of a control ABPC. In some embodiments, the composition provides for at least a 5-fold increase in endolysosomal delivery in the target mammalian cell as compared to a composition including the same amount of a control ABPC.

In some embodiments, the composition results in a less of a reduction in the level of LRRC15 presented on the surface of the target mammalian cell as compared to a composition including the same amount of a control ABPC. In some embodiments, the composition does not result in a detectable reduction in the level of the LRRC15 presented on the surface of the target mammalian cell. In some embodiments, the target mammalian cell is a cancer cell.

In some embodiments, the dissociation rate of the ABD at a pH of ˜4.0 to ˜6.5 is at least 10% faster than the dissociation rate of the ABD at a pH of ˜7.0 to ˜8.0. In some embodiments, the dissociation rate of the ABD at a pH of ˜4.0 to ˜6.5 is at least 3-fold faster than the dissociation rate of the ABD at a pH of ˜7.0 to ˜8.0. In some embodiments, the dissociation rate of the ABD at a pH of ˜4.0 to ˜6.5 is at least 10-fold faster than the dissociation rate of the ABD at a pH of ˜7.0 to ˜8.0. In some embodiments, the KD of the ABD at a pH of ˜4.0 to ˜6.5 is at least 10% greater than the K_{D} of the ABD at a pH of ˜7.0 to ˜8.0. In some embodiments, the KD of the ABD at a pH of ˜4.0 to ˜6.5 is at least 3-fold greater than the K_{D} of the ABD at a pH of ˜7.0 to ˜8.0. In some embodiments, the KD of the ABD at a pH of ˜4.0 to ˜6.5 is at least 10-fold greater than the KD of the ABD at a pH of ˜7.0 to ˜8.0.

In some embodiments, the ABPC is cytotoxic or cytostatic to the target mammalian cell. In some embodiments, the ABPC is cross-reactive with a non-human primate LRRC15 and human LRRC15. In some embodiments, the ABPC is cross-reactive with a non-human primate LRRC15, a human LRRC15, and one or both of rat LRRC15 and a mouse LRRC15. In some embodiments, the ABPC is cross-reactive with a non-human primate LRRC15, a human LRRC15, a rat LRRC15, and a mouse LRRC15. In some embodiments, the ABD binds to an epitope of LRRC15 that is present on the surface of cells from an Old World Monkey.

In some embodiments, the ABPC includes a single polypeptide. In some embodiments, the ABD is selected from the group consisting of a VH domain, a VHH domain, a VNAR domain, and a scFv. In some embodiments, the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the ABPC includes two or more polypeptides. In some embodiments, the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAB, and a PROTAC.

In some embodiments, the ABPC or ABD includes a proteolysis-targeting antibody (PROTAB) (as described, e.g., in Marei et al, “Antibody targeting of E3 ubiquitin ligases for receptor degradation. Nature, 6 Oct. 2022). In some embodiments, a PROTAB or PROTAC may tether cell-surface E3 ubiquitin ligases to transmembrane proteins, resulting in target degradation both in vitro and in vivo.

In some embodiments, at least one polypeptide of the ABPC is conjugated to the toxin, the radioisotope, the drug, or the small molecule via a cleavable linker. In some embodiments, at least one polypeptide of the ABPC is conjugated to the toxin, the radioisotope, the drug, or the small molecule via a non-cleavable linker.

In some embodiments, the half-life of the ABPC in vivo is increased as compared to the half-life of a control ABPC in vivo. In some embodiments, the half-life of the ABPC in vivo is increased about 5% to about 95% as compared to the half-life of a control ABPC in vivo. In some embodiments, the half-life of the ABPC in vivo is increased about 10% to about 95% as compared to the half-life of a control ABPC in vivo. In some embodiments, the half-life of the ABPC in vivo is increased about 30% to about 95% as compared to the half-life of a control ABPC in vivo. In some embodiments, the half-life of the ABPC in vivo is increased about 50% to about 95% as compared to the half-life of a control ABPC in vivo. In some embodiments, the half-life of the ABPC in vivo is increased about 70% to about 95% as compared to the half-life of a control ABPC in vivo. In other embodiments, the in vivo half-life may be decreased.

In some embodiments, the control ABPC is capable of specifically binding to LRRC15 or an epitope of LRRC15 presented on the surface of a target mammalian cell, where: (a) the control ABPC includes a first ABD: (b) the dissociation rate of the first ABD of the control ABPC at a pH of about 4.0 to about 6.5 is no more than 3-fold faster than the dissociation rate at a pH of about 7.0 to about 8.0; and (c) the dissociation constant (KD) of the first ABD of the control ABPC at a pH of about 4.0 to about 6.5 is no more than 3-fold greater than the K_{D} at a pH of about 7.0 to about 8.0.

In some embodiments, the control ABPC is capable of specifically binding to LRRC15 or an epitope of LRRC15 presented on the surface of a target mammalian cell, where: (a) the control ABPC includes a first ABD; (b) the dissociation rate of the first ABD of the control ABPC at a pH of about 4.0 to about 6.5 is no more than 2-fold faster than the dissociation rate at a pH of about 7.0 to about 8.0; and (c) the dissociation constant (KD) of the first ABD of the control ABPC at a pH of about 4.0 to about 6.5 is no more than 2-fold greater than the K_{D} at a pH of about 7.0 to about 8.0.

In some embodiments, the control ABPC is capable of specifically binding to LRRC15 or an epitope of LRRC15 presented on the surface of a target mammalian cell, where: (a) the control ABPC includes a first ABD; (b) the dissociation rate of the first ABD of the control ABPC at a pH of about 4.0 to about 6.5 is no more than 1-fold faster than the dissociation rate at a pH of about 7.0 to about 8.0; and (c) the dissociation constant (KD) of the first ABD of the control ABPC at a pH of about 4.0 to about 6.5 is no more than 1-fold greater than the K_{D} at a pH of about 7.0 to about 8.0.

In some embodiments, the control ABPC is selected from one of: (a) 15G7; (b) 24D9; and (c) 29F1. In some embodiments, the control ABPC is selected from one of: (a) hu15G7; (b) hu24D9; and (c) hu29F1.

In some embodiments, the ABPC includes a second ABD.

Also provided herein are kits including at least one dose of any of the pharmaceutical compositions described herein.

Further provided herein are antigen-binding protein constructs (ABPCs) including: a first ABD that is capable of specifically binding LRRC15 or an epitope of LRRC15 presented on the surface of a target mammalian cell, where: (a) the dissociation rate of the first ABD at a pH of about 4.0 to about 6.5 is faster than the dissociation rate at a pH of about 7.0 to about 8.0; or (b) the dissociation constant (K_{D}) of the first ABD at a pH of about 4.0 to about 6.5 is greater than the K_{D} at a pH of about 7.0 to about 8.0.

In some embodiments, the first ABD includes an HCV domain of one of: (a) an HCV domain of 15G7, optionally with one or more amino acids substituted with a histidine; (b) an HCV domain of 24D9, optionally with one or more amino acids substituted with a histidine; and (c) an HCV domain of 29F1, optionally with one or more amino acids substituted with a histidine, an aspartate, or a glutamate. In some embodiments, the first ABD includes an LCV domain of one of: (a) an LCV domain of 15G7, optionally with one or more amino acids substituted with a histidine; (b) an LCV domain of 24D9, optionally with one or more amino acids substituted with a histidine; and (c) an LCV domain of 29F1, optionally with one or more amino acids substituted with a histidine, an aspartate, or a glutamate.

In some embodiments, the first ABD includes one of: (a) an HCV domain of 15G7, optionally with one or more amino acids substituted with a histidine; and/or an LCV domain of 15G7, optionally with one or more amino acids substituted with a histidine; (b) an HCV domain of 24D9, optionally with one or more amino acids substituted with a histidine; and/or an LCV domain of 24D9, optionally with one or more amino acids substituted with a histidine; and (c) an HCV domain of 29F1, optionally with one or more amino acids substituted with a histidine, an aspartate, or a glutamate; and/or an LCV domain of 29F1, optionally with one or more amino acids substituted with a histidine, an aspartate, or a glutamate.

In some embodiments, the HCV domain includes one of: (a) an HCV domain of 15G7 including SEQ ID NO: 378, or a humanized version thereof (e.g., SEQ ID NO: 382); (b) an HCV domain of 24D9 including SEQ ID NO: 512, or a humanized version thereof (e.g., SEQ ID NO: 516); and (c) an HCV domain of 29F1 including SEQ ID NO: 572, or a humanized version thereof (e.g., SEQ ID NO: 576). In some embodiments, the LCV domain includes one of: (a) an LCV domain of 15G7 including SEQ ID NO: 451, or a humanized version thereof (e.g., SEQ ID NO: 455); (b) an LCV domain of 24D9 including SEQ ID NO: 518, or a humanized version thereof (e.g., SEQ ID NO: 522); and (c) an LCV domain of 29F1 including SEQ ID NO: 577, or a humanized version thereof (e.g., SEQ ID NO: 581).

In some embodiments, the first ABD includes an HCV domain of one of: (a) an HCV domain including a CDR1, a CDR2, and a CDR3 of SEQ ID NOs: 379-381, respectively, optionally with collectively a total of one or more amino acid positions in SEQ ID NOs: 379-381 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate. In some embodiments, the first LRRC15-binding domain includes an LCV domain of one of: (a) an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate. In some embodiments, the first LRRC15-binding domain includes one of: (a) an HCV domain including a CDR1, a CDR2, and a CDR3 of SEQ ID NOs: 379-381, respectively, optionally with collectively a total of one or more amino acid positions in SEQ ID NOs: 379-381 substituted with a histidine; and/or an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and/or an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate; and/or an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate.

In some embodiments, the first ABD includes an HCV domain of one of: (a) an HCV domain that is at least 90% identical to the sequence set forth in SEQ ID NO: 378 OR 382, where the HCV domain includes a histidine at one or more positions in SEQ ID NO: 378 or SEQ ID NO: 382 selected from the group consisting of: 32, 34, 53, 60, 103, 104, 105, 109, and 110; (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and (c) an HCV domain that is at least 90% identical to the sequence set forth in SEQ ID NO: 572 or SEQ ID NO: 576, where the HCV domain includes an aspartate (D) or a glutamate (E) at one or more positions in SEQ ID NO: 572 or SEQ ID NO: 576 selected from the group consisting of: D positions selected from 31, 56 and 99; and an E position selected from 59. In some embodiments, the first ABD includes an LCV domain that is one of: (a) an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; (b) an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an LCV domain that is at least 90% identical to the sequence set forth in SEQ ID NO: 577 or SEQ ID NO: 581, where the LCV domain includes a histidine, an aspartate, or a glutamate at one or more positions in SEQ ID NO: 577 or SEQ ID NO: 581 selected from the group consisting of: D positions selected from 27, 28, 31, 52 and 56; and E positions selected from 51 and 56.

In some embodiments, the first ABD includes an HCV domain of one of: (a) an HCV domain that is at least 90% identical to the sequence set forth in SEQ ID NO: 378 OR 382, where the HCV domain includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53,106; and (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516.

In some embodiments, the first ABD includes one of: (a) an HCV domain that is at least 90% identical to the sequence set forth in SEQ ID NO: 378 OR 382, where the HCV domain includes a histidine at one or more positions in SEQ ID NO: 378 or SEQ ID NO: 382 selected from the group consisting of: 32, 34, 53, 60, 103, 104, 105, 109, and 110; and/or an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an HCV domain that is at least 90% identical to the sequence set forth in SEQ ID NO: 572 or SEQ ID NO: 576, where the HCV domain includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCV domain that is at least 90% identical to the sequence set forth in SEQ ID NO: 577 or SEQ ID NO: 581, where the LCV domain includes a histidine, an aspartate, or a glutamate at one or more positions in SEQ ID NO: 577 or SEQ ID NO: 581 selected from the group consisting of: D positions selected from 27, 28, 31, 52 and 56; and E positions selected from 51 and 56.

In some embodiments, the first ABD includes an HCV domain of one of: (a) an HCV domain comprising the sequence of one of SEQ ID NO: 378, SEQ ID NO: 382, one of SEQ ID NOs: 383-424, and one of SEQ ID NOs: 425-450; (b) an HCV domain comprising the sequence of one of SEQ ID NO: 512, SEQ ID NO: 516, one of SEQ ID NOs: 523-567, and one of SEQ ID NOs: 568-571; and (c) an HCV domain comprising the sequence of one of SEQ ID NO: 572, SEQ ID NO: 576, one of SEQ ID NOs: 582-624, and one of SEQ ID NOs: 653-695; and/or the first ABD includes an LCV domain of one of: (a) an LCV domain having the sequence of one of SEQ ID NO: 451, SEQ ID NO: 455, one of SEQ ID NOs: 456-485, and one of SEQ ID NOs: 486-511; (b) an LCV domain comprising the sequence of SEQ ID NO: 518, SEQ ID NO: 522, SEQ ID: 518 (with L35H), or SEQ ID NO: 518 (with G97H); and (c) an LCV domain comprising the sequence of one of SEQ ID NO: 577, SEQ ID NO: 581, one of SEQ ID NOs: 625-652, and one of SEQ ID NOs: 696-723.

In some embodiments, the first ABD includes one of: (a) an HCV domain comprising the sequence of one of SEQ ID NO: 378, SEQ ID NO: 382, one of SEQ ID NOs: 383-424, and one of SEQ ID NOs: 425-450, and/or an LCV domain having the sequence of one of SEQ ID NO: 451, SEQ ID NO: 455, one of SEQ ID NOs: 456-485, and one of SEQ ID NOs: 486-511, where the first ABD does not include (i) an HCV domain of SEQ ID NO: 378 or SEQ ID NO: 382 and an LCV domain comprising a sequence that is not one of SEQ ID NO: 461, SEQ ID NO: 462, SEQ ID NO: 464, SEQ ID NO: 466, SEQ ID NO: 467, one of SEQ ID NOs: 477-478, and one of SEQ ID NOs: 480-482; or (ii) an LCV domain comprising the sequence of SEQ ID NO: 451 or SEQ ID NO: 455 and an HCV domain comprising a sequence that does not include one of: one of SEQ ID NOs: 527-529, one of SEQ ID NOs: 535-536, one of SEQ ID NOs: 541-543, SEQ ID NO: 552, one of SEQ ID NOs: 559-560, and SEQ ID NO: 564; (b) an HCV domain comprising the sequence of one of SEQ ID NO: 512, SEQ ID NO: 516, one of SEQ ID NOS: 523-567, and one of SEQ ID NOs: 568-571; and/or an LCV domain comprising the sequence of SEQ ID NO: 518, SEQ ID NO: 522, SEQ ID: 518 (with L35H), or SEQ ID NO: 518 (with G97H), where the first ABD does not include (i) an HCV domain comprising the sequence of SEQ ID NO: 512 or SEQ ID NO: 516 and an LCV domain comprising a sequence that is not one of SEQ ID NO: 518 (with an L35H substitution) and SEQ ID NO: 518 (with a G97H substitution); or (ii) an LCV domain comprising the sequence of SEQ ID NO: 518 or SEQ ID NO: 518 or SEQ ID NO: 522 and an HCV domain comprising a sequence that does not include one of: one of SEQ ID NOs: 527-529, one of SEQ ID NOs: 535-536, one of SEQ ID NOs: 541-543, SEQ ID NO: 552, one of SEQ ID NOs: 559-560, and SEQ ID NO: 564; and (c) an HCV domain comprising the sequence of one of SEQ ID NO: 572, SEQ ID NO: 576, one of SEQ ID NOs: 582-624, and one of SEQ ID NOs: 653-695; and/or an LCV domain comprising the sequence of one of SEQ ID NO: 577, SEQ ID NO: 581, one of SEQ ID NOs: 625-652, and one of SEQ ID NOs: 696-723, where the first ABD does not include (i) an HCV domain comprising the sequence of SEQ ID NO: 572 or SEQ ID NO: 576 and an LCV domain comprising a sequence that is not one of SEQ ID NO: 628, SEQ ID NO: 629, SEQ ID NO: 632, SEQ ID NO: 638 and SEQ ID NO: 642; or (ii) an LCV domain comprising the sequence of SEQ ID NO: 577 or SEQ ID NO: 581 and an HCV domain comprising a sequence that is not one of SEQ ID NO: 587, SEQ ID NO: 598, SEQ ID NO: 611, and SEQ ID NO: 672.

In some embodiments, the ABPC is degraded in the target mammalian cell following internalization of the ABPC by the target mammalian cell. In some embodiments, the ABPC includes a conjugated toxin, radioisotope, drug, or small molecule.

In some embodiments, a composition including the ABPC provides for an increase in toxin liberation in the target mammalian cell as compared to a composition including the same amount of a control ABPC. In some embodiments, a composition including the ABPC provides for at least a 20% increase in toxin liberation in the target mammalian cell as compared to a composition including the same amount of a control ABPC. In some embodiments, a composition including the ABPC provides for at least a 50% increase in toxin liberation in the target mammalian cell as compared to a composition including the same amount of a control ABPC. In some embodiments, a composition including the ABPC provides for at least a 2-fold increase in toxin liberation in the target mammalian cell as compared to a composition including the same amount of a control ABPC. In some embodiments, a composition including the ABPC provides for at least a 5-fold increase in toxin liberation in the target mammalian cell as compared to a composition including the same amount of a control ABPC.

In some embodiments, a composition including the ABPC provides for an increase in target mammalian cell killing as compared to a composition including the same amount of a control ABPC. In some embodiments, a composition including the ABPC provides for at least a 20% increase in target mammalian cell killing as compared to a composition including the same amount of a control ABPC. In some embodiments, a composition including the ABPC provides for at least a 50% increase in target mammalian cell killing as compared to a composition including the same amount of a control ABPC. In some embodiments, a composition including the ABPC provides for at least a 2-fold increase in target mammalian cell killing as compared to a composition including the same amount of a control ABPC. In some embodiments, a composition including the ABPC provides for at least a 5-fold increase in target mammalian cell killing as compared to a composition including the same amount of a control ABPC.

In some embodiments, a composition including the ABPC provides for an increase in endolysosomal delivery in the target mammalian cell as compared to a composition including the same amount of a control ABPC. In some embodiments, the ABPC provides for at least a 20% increase in endolysosomal delivery in the target mammalian cell as compared to a composition including the same amount of a control ABPC. In some embodiments, a composition including the ABPC provides for at least a 50% increase in endolysosomal delivery in the target mammalian cell as compared to a composition including the same amount of a control ABPC. In some embodiments, a composition including the ABPC provides for at least a 2-fold increase in endolysosomal delivery in the target mammalian cell as compared to a composition including the same amount of a control ABPC. In some embodiments, a composition including the ABPC provides for at least a 5-fold increase in endolysosomal delivery in the target mammalian cell as compared to a composition including the same amount of a control ABPC.

In some embodiments, a composition including the ABPC results in a less of a reduction in the level of LRRC15 presented on the surface of the target mammalian cell as compared to a composition including the same amount of a control ABPC. In some embodiments, a composition including the ABPC does not result in a detectable reduction in the level of the LRRC15 presented on the surface of the target mammalian cell.

Also provided herein are antigen-binding protein constructs (ABPCs) including: a first ABD that is capable of specifically binding LRRC15 or an epitope of LRRC15 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; or the dissociation constant (K_{D}) of the first ABD at a pH of about 4.0 to about 6.5 is greater than the K_{D} at a pH of about 7.0 to about 8.0; and (b) the composition provides for one or more of: an increase in toxin liberation in the target mammalian cell as compared to a composition including the same amount of a control ABPC; an increase in target mammalian cell killing as compared to a composition including the same amount of a control ABPC; and an increase in endolysosomal delivery in the target mammalian cell as compared to a composition including the same amount of a control ABPC.

In some embodiments, the first ABD includes an HCV domain of one of: (a) an HCV domain of 15G7, optionally with one or more amino acids substituted with a histidine; (b) an HCV domain of 24D9, optionally with one or more amino acids substituted with a histidine; and (c) an HCV domain of 29F1, optionally with one or more amino acids substituted with a histidine, an aspartate, or a glutamate. In some embodiments, the first ABD includes an LCV domain of one of: (a) an LCV domain of 15G7, optionally with one or more amino acids substituted with a histidine; (b) an LCV domain of 24D9, optionally with one or more amino acids substituted with a histidine; and (c) an LCV domain of 29F1, optionally with one or more amino acids substituted with a histidine, an aspartate, or a glutamate.

In some embodiments, the first ABD includes one of: (a) an HCV domain of 15G7, optionally with one or more amino acids substituted with a histidine; and/or an LCV domain of 15G7, optionally with one or more amino acids substituted with a histidine; (b) an HCV domain of 24D9, optionally with one or more amino acids substituted with a histidine; and/or an LCV domain of 24D9, optionally with one or more amino acids substituted with a histidine; and (c) an HCV domain of 29F1, optionally with one or more amino acids substituted with a histidine, an aspartate, or a glutamate; and/or an LCV domain of 29F1, optionally with one or more amino acids substituted with a histidine, an aspartate, or a glutamate.

In some embodiments, the HCV domain includes one of: (a) an HCV domain of 15G7 including SEQ ID NO: 378, or a humanized version thereof (e.g., SEQ ID NO: 382); (b) an HCV domain of 24D9 including SEQ ID NO: 512, or a humanized version thereof (e.g., SEQ ID NO: 516); and (c) an HCV domain of 29F1 including SEQ ID NO: 572, or a humanized version thereof (e.g., SEQ ID NO: 576). In some embodiments, the LCV domain includes one of: (a) an LCV domain of 15G7 including SEQ ID NO: 451, or a humanized version thereof (e.g., SEQ ID NO: 455); (b) an LCV domain of 24D9 including SEQ ID NO: 518, or a humanized version thereof (e.g., SEQ ID NO: 522); and (c) an LCV domain of 29F1 including SEQ ID NO: 577, or a humanized version thereof (e.g., SEQ ID NO: 581).

In some embodiments, the first ABD includes an HCV domain of one of: (a) an HCV domain including a CDR1, a CDR2, and a CDR3 of SEQ ID NOs: 379-381, respectively, optionally with collectively a total of one or more amino acid positions in SEQ ID NOs: 379-381 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate. In some embodiments, the first LRRC15-binding domain includes an LCV domain of one of: (a) an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate.

In some embodiments, the first LRRC15-binding domain includes one of: (a) an HCV domain including a CDR1, a CDR2, and a CDR3 of SEQ ID NOs: 379-381, respectively, optionally with collectively a total of one or more amino acid positions in SEQ ID NOs: 379-381 substituted with a histidine; and/or an LCV domain including a CDR1, a CDR2, and a CDR3 of SEQ ID NOs: 452-454, respectively, optionally with collectively a total of one or more amino acid positions in SEQ ID NOs: 452-454 substituted with a histidine; (b) an HCV domain including a CDR1, a CDR2, and a CDR3 of SEQ ID NOs: 513-515, respectively, optionally with collectively a total of one or more amino acid positions in SEQ ID NOs: 513-515 substituted with a histidine; and/or an LCV domain including a CDR1, a CDR2, and a CDR3 of SEQ ID NOs: 519-521, respectively, optionally with collectively a total of one or more amino acid positions in SEQ ID NOs: 519-521 substituted with a histidine; and (c) an HCV domain including a CDR1, a CDR2, and a CDR3 of SEQ ID NOs: 573-575, respectively, optionally with collectively a total of one or more amino acid positions in SEQ ID NOs: 573-575 substituted with a histidine, an aspartate, or a glutamate; and/or an LCV domain including a CDR1, a CDR2, and a CDR3 of SEQ ID NOs: 578-580, respectively, optionally with collectively a total of one or more amino acid positions in SEQ ID NOs: 578-580 substituted with a histidine, an aspartate, or a glutamate.

In some embodiments, the first ABD includes an HCV domain of one of: (a) an HCV domain that is at least 90% identical to the sequence set forth in SEQ ID NO: 378 OR 382, where the HCV domain includes a histidine at one or more positions in SEQ ID NO: 378 or SEQ ID NO: 382 selected from the group consisting of: 32, 34, 53, 60, 103, 104, 105, 109, and 110; (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and (c) an HCV domain that is at least 90% identical to the sequence set forth in SEQ ID NO: 572 or SEQ ID NO: 576, where the HCV domain includes an aspartate (D) or a glutamate (E) at one or more positions in SEQ ID NO: 572 or SEQ ID NO: 576 selected from the group consisting of: D positions selected from 31, 56 and 99; and an E position selected from 59. In some embodiments, the first ABD includes an LCV domain of one of: (a) an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; (b) an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an LCV domain that is at least 90% identical to the sequence set forth in SEQ ID NO: 577 or SEQ ID NO: 581, where the LCV domain includes a histidine, an aspartate, or a glutamate at one or more positions in SEQ ID NO: 577 or SEQ ID NO: 581 selected from the group consisting of: D positions selected from 27, 28, 31, 52 and 56; and E positions selected from 51 and 56.

In some embodiments, the first ABD includes an HCV domain of one of: (a) an HCV domain that is at least 90% identical to the sequence set forth in SEQ ID NO: 378 OR 382, where the HCV domain includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53,106; and (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516.

In some embodiments, the first ABD includes an LCV domain of one of: (a) an LCV domain that is at least 90% identical to the sequence set forth in SEQ ID NO: 451 or SEQ ID NO: 455, where the LCV domain includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96 and 93, 96; and (b) an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522.

In some embodiments, the first ABD includes one of: (a) an HCV domain that is at least 90% identical to the sequence set forth in SEQ ID NO: 378 OR 382, where the HCV domain includes a histidine at one or more positions in SEQ ID NO: 378 or SEQ ID NO: 382 selected from the group consisting of: 32, 34, 53, 60, 103, 104, 105, 109, and 110; and/or an LCV domain that is at least 90% identical to the sequence set forth in SEQ ID NO: 451 or SEQ ID NO: 455, where the LCV domain includes a histidine at one or more positions in SEQ ID NO: 451 or SEQ ID NO: 455 selected from the group consisting of: 29, 30, 32, 34, 50, 92, 93, 95, 96, and 97; (b) an HCV domain that is at least 90% identical to the sequence set forth in SEQ ID NO: 512 or SEQ ID NO: 516, where the HCV domain includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCV domain that is at least 90% identical to the sequence set forth in SEQ ID NO: 518 or SEQ ID NO: 522, where the LCV domain includes a histidine at one or more positions in SEQ ID NO: 518 or SEQ ID NO: 522 selected from the group consisting of: 35 and 97; and (c) an HCV domain that is at least 90% identical to the sequence set forth in SEQ ID NO: 572 or SEQ ID NO: 576, where the HCV domain includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCV domain that is at least 90% identical to the sequence set forth in SEQ ID NO: 577 or SEQ ID NO: 581, where the LCV domain includes a histidine, an aspartate, or a glutamate at one or more positions in SEQ ID NO: 577 or SEQ ID NO: 581 selected from the group consisting of: D positions selected from 27, 28, 31, 52 and 56; and E positions selected from 51 and 56.

In some embodiments, the first ABD includes an HCV domain of one of: (a) an HCV domain comprising the sequence of one of SEQ ID NO: 378, one of SEQ ID NOs: 383-424, and one of SEQ ID NOs: 425-450; (b) an HCV domain comprising the sequence of one of SEQ ID NO: 512, SEQ ID NO: 516, one of SEQ ID NOs: 523-567, and one of SEQ ID NOs: 568-571; and (c) an HCV domain comprising the sequence of one of SEQ ID NO: 572, SEQ ID NO: 576, one of SEQ ID NOs: 582-624, and one of SEQ ID NOs: 653-695. In some embodiments, the first ABD includes an LCV domain of one of: (a) an LCV domain having the sequence of one of SEQ ID NO: 451, SEQ ID NO: 455, one of SEQ ID NOs: 456-485, and one of SEQ ID NOs: 486-511; (b) an LCV domain comprising the sequence of SEQ ID NO: 518, SEQ ID NO: 522, SEQ ID: 518 (with L35H), or SEQ ID NO: 518 (with G97H); and (c) an LCV domain comprising the sequence of one of SEQ ID NO: 577, SEQ ID NO: 581, one of SEQ ID NOs: 625-652, and one of SEQ ID NOs: 696-723.

In some embodiments, the first ABD includes one of: (a) an HCV domain comprising the sequence of one of SEQ ID NO: 378, SEQ ID NO: 382, one of SEQ ID NOs: 383-424, and one of SEQ ID NOs: 425-450, and/or an LCV domain having the sequence of one of SEQ ID NO: 451, SEQ ID NO: 455, one of SEQ ID NOs: 456-485, and one of SEQ ID NOs: 486-511, where the first ABD does not include (i) an HCV domain of SEQ ID NO: 378 OR 382 and an LCV domain comprising a sequence that is not one of SEQ ID NO: 461, SEQ ID NO: 462, SEQ ID NO: 464, SEQ ID NO: 466, SEQ ID NO: 467, one of SEQ ID NOs: 477-478, and one of SEQ ID NOs: 480-482; or (ii) an LCV domain comprising the sequence of SEQ ID NO: 451 and an HCV domain comprising a sequence that does not include one of: one of SEQ ID NOs: 527-529, one of SEQ ID NOs: 535-536, one of SEQ ID NOs: 541-543, SEQ ID NO: 552, one of SEQ ID NOs: 559-560, and SEQ ID NO: 564; (b) an HCV domain comprising the sequence of one of SEQ ID NO: 512, SEQ ID NO: 516, one of SEQ ID NOs: 523-567, and one of SEQ ID NOs: 568-571; and/or an LCV domain comprising the sequence of SEQ ID NO: 518, SEQ ID NO: 522, SEQ ID: 518 (with L35H), or SEQ ID NO: 518 (with G97H), where the first ABD does not include (i) an HCV domain comprising the sequence of SEQ ID NO: 512 or SEQ ID NO: 516 and an LCV domain comprising a sequence that is not one of SEQ ID NO: 518 (with an L35H substitution) and SEQ ID NO: 518 (with a G97H substitution); or (ii) an LCV domain comprising the sequence of SEQ ID NO: 518 or SEQ ID NO: 518 or SEQ ID NO: 522 and an HCV domain comprising a sequence that does not include one of: one of SEQ ID NOs: 527-529, one of SEQ ID NOs: 535-536, one of SEQ ID NOs: 541-543, SEQ ID NO: 552, one of SEQ ID NOs: 559-560, and SEQ ID NO: 564; and (c) an HCV domain comprising the sequence of SEQ ID NO: 572, 576, or 582-624, or 653-695; and/or an LCV domain comprising the sequence of one of SEQ ID NO: 577, SEQ ID NO: 581, one of SEQ ID NOs: 625-652, and one of SEQ ID NOs: 696-723, where the first ABD does not include (i) an HCV domain comprising the sequence of SEQ ID NO: 572 or SEQ ID NO: 576 and an LCV domain comprising a sequence that is not one of SEQ ID NO: 628, SEQ ID NO: 629, SEQ ID NO: 632, SEQ ID NO: 638 and SEQ ID NO: 642; or (ii) an LCV domain comprising the sequence of SEQ ID NO: 577 or SEQ ID NO: 581 and an HCV domain comprising a sequence that is not one of SEQ ID NO: 587, SEQ ID NO: 598, SEQ ID NO: 611, and SEQ ID NO: 672.

In some embodiments, a composition including the ABPC provides for an increase in toxin liberation in the target mammalian cell as compared to a composition including the same amount of a control ABPC. In some embodiments, a composition including the ABPC provides for at least a 20% increase in toxin liberation in the target mammalian cell as compared to a composition including the same amount of a control ABPC. In some embodiments, a composition including the ABPC provides for at least a 50% increase in toxin liberation in the target mammalian cell as compared to a composition including the same amount of a control ABPC. In some embodiments, a composition including the ABPC provides for at least a 2-fold increase in toxin liberation in the target mammalian cell as compared to a composition including the same amount of a control ABPC. In some embodiments, a composition including the ABPC provides for at least a 5-fold increase in toxin liberation in the target mammalian cell as compared to a composition including the same amount of a control ABPC.

In some embodiments, a composition including the ABPC provides for an increase in target mammalian cell killing as compared to a composition including the same amount of a control ABPC. In some embodiments, a composition including the ABPC provides for at least a 20% increase in target mammalian cell killing as compared to a composition including the same amount of a control ABPC. In some embodiments, a composition including the ABPC provides for at least a 50% increase in target mammalian cell killing as compared to a composition including the same amount of a control ABPC. In some embodiments, a composition including the ABPC provides for at least a 2-fold increase in target mammalian cell killing as compared to a composition including the same amount of a control ABPC. In some embodiments, a composition including the ABPC provides for at least a 5-fold increase in target mammalian cell killing as compared to a composition including the same amount of a control ABPC.

In some embodiments, a composition including the ABPC provides for an increase in endolysosomal delivery in the target mammalian cell as compared to a composition including the same amount of a control ABPC. In some embodiments, a composition including the ABPC provides for at least a 20% increase in endolysosomal delivery in the target mammalian cell as compared to a composition including the same amount of a control ABPC. In some embodiments, a composition including the ABPC provides for at least a 50% increase in endolysosomal delivery in the target mammalian cell as compared to a composition including the same amount of a control ABPC. In some embodiments, a composition including the ABPC provides for at least a 2-fold increase in endolysosomal delivery in the target mammalian cell as compared to a composition including the same amount of a control ABPC. In some embodiments, a composition including the ABPC provides for at least a 5-fold increase in endolysosomal delivery in the target mammalian cell as compared to a composition including the same amount of a control ABPC.

In some embodiments, a composition including the ABPC results in a less of a reduction in the level of LRRC15 presented on the surface of the target mammalian cell as compared to a composition including the same amount of a control ABPC. In some embodiments, a composition including the ABPC does not result in a detectable reduction in the level of the LRRC15 presented on the surface of the target mammalian cell. In some embodiments, the target mammalian cell is a cancer cell.

In some embodiments, the dissociation rate of the ABD at a pH of ˜4.0 to ˜6.5 is at least 10% faster than the dissociation rate of the ABD at a pH of ˜7.0 to ˜8.0. In some embodiments, the dissociation rate of the ABD at a pH of ˜4.0 to ˜6.5 is at least 3-fold faster than the dissociation rate of the ABD at a pH of ˜7.0 to ˜8.0. In some embodiments, the dissociation rate of the ABD at a pH of ˜4.0 to ˜6.5 is at least 10-fold faster than the dissociation rate of the ABD at a pH of ˜7.0 to ˜8.0. In some embodiments, the K_{D} of the ABD at a pH of ˜4.0 to ˜6.5 is at least 10% greater than the K_{D} of the ABD at a pH of ˜7.0 to ˜8.0. In some embodiments, the K_{D} of the ABD at a pH of ˜4.0 to ˜6.5 is at least 3-fold greater than the K_{D} of the ABD at a pH of ˜7.0 to ˜8.0. In some embodiments, the K_{D} of the ABD at a pH of ˜4.0 to ˜6.5 is at least 10-fold greater than the K_{D} of the ABD at a pH of ˜7.0 to ˜8.0.

In some embodiments, the ABPC is cytotoxic or cytostatic to the target mammalian cell.

In some embodiments, the ABPC is cross-reactive with a non-human primate LRRC15 and human LRRC15. In some embodiments, the ABPC is cross-reactive with a non-human primate LRRC15, a human LRRC15, and one or both of rat LRRC15 and a mouse LRRC15. In some embodiments, the ABPC is cross-reactive with a non-human primate LRRC15, a human LRRC15, a rat LRRC15, and a mouse LRRC15. In some embodiments, the ABD binds to an epitope of LRRC15 that is present on the surface of cells from an Old World Monkey.

In some embodiments, the ABPC includes a single polypeptide. In some embodiments, the ABD is selected from the group consisting of a VH domain, a VHH domain, a VNAR domain, and a scFv. In some embodiments, the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the ABPC includes two or more polypeptides. In some embodiments, the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAB, and a PROTAC.

In some embodiments, the ABPC or ABD includes a proteolysis-targeting antibody (PROTAB) (as described, e.g., in Marei et al, “Antibody targeting of E3 ubiquitin ligases for receptor degradation. Nature, 6 Oct. 2022). In some embodiments, a PROTAB or PROTAC may tether cell-surface E3 ubiquitin ligases to transmembrane proteins, resulting in target degradation both in vitro and in vivo.

In some embodiments, at least one polypeptide of the ABPC is conjugated to the toxin, the radioisotope, the drug, or the small molecule via a cleavable linker. In some embodiments, at least one polypeptide of the ABPC is conjugated to the toxin, the radioisotope, the drug, or the small molecule via a non-cleavable linker.

In some embodiments, the half-life of the ABPC in vivo is increased as compared to the half-life of a control ABPC in vivo. In some embodiments, the ABPC in vivo is increased ˜5% to ˜95% as compared to the half-life of a control ABPC in vivo. In some embodiments, the half-life of the ABPC in vivo is increased ˜10% to ˜95% as compared to the half-life of a control ABPC in vivo. In some embodiments, the half-life of the ABPC in vivo is increased ˜30% to ˜95% as compared to the half-life of a control ABPC in vivo. In some embodiments, the half-life of the ABPC in vivo is increased ˜50% to ˜95% as compared to the half-life of a control ABPC in vivo. In some embodiments, the half-life of the ABPC in vivo is increased ˜70% to ˜95% as compared to the half-life of a control ABPC in vivo.

In some embodiments, the control ABPC is capable of specifically binding to LRRC15 or an epitope of LRRC15 presented on the surface of a target mammalian cell, where: (a) the control ABPC includes a first ABD; (b) the dissociation rate of the first ABD of the control ABPC at a pH of ˜4.0 to ˜6.5 is no more than 3-fold faster than the dissociation rate at a pH of ˜7.0 to ˜8.0; and (c) the dissociation constant (K_{D}) of the first ABD of the control ABPC at a pH of ˜4.0 to ˜6.5 is no more than 3-fold greater than the K_{D} at a pH of ˜7.0 to ˜8.0.

In some embodiments, the control ABPC is capable of specifically binding to LRRC15 or an epitope of LRRC15 presented on the surface of a target mammalian cell, where: (a) the control ABPC includes a first ABD; (b) the dissociation rate of the first ABD of the control ABPC at a pH of ˜4.0 to ˜6.5 is no more than 2-fold faster than the dissociation rate at a pH of ˜7.0 to ˜8.0; and (c) the dissociation constant (K_{D}) of the first ABD of the ABPC at a pH of ˜4.0 to ˜6.5 is no more than 2-fold greater than the K_{D} at a pH of ˜7.0 to ˜8.0.

In some embodiments, the control ABPC is capable of specifically binding to LRRC15 or an epitope of LRRC15 presented on the surface of a target mammalian cell, where: (a) the control ABPC includes a first ABD; (b) the dissociation rate of the first ABD of the control ABPC at a pH of ˜4.0 to ˜6.5 is no more than 1-fold faster than the dissociation rate at a pH of ˜7.0 to ˜8.0; and (c) the dissociation constant (K_{D}) of the first ABD of the control ABPC at a pH of ˜4.0 to ˜6.5 is no more than 1-fold greater than the K_{D} at a pH of ˜7.0 to ˜8.0.

In some embodiments, the control ABPC is selected from one of: (a) 15G7 (comprising SEQ ID NOs: 378 and 451); (b) 24D9 (comprising SEQ ID NOs: 512 and 518); (c) 29F1 (comprising SEQ ID NOs: 572 and 577); (d) the parental antibody of MYT2737; and (e) the parental antibody of MYT3315.

In some embodiments, the ABPC includes a second ABD.

Also provided herein are kits including at least one dose of any of the ABPCs described herein.

Also provided herein are methods of treating a cancer characterized by having a population of cancer cells, cancer associated fibroblasts (CAFs), and/or stromal cells that have LRRC15 or an epitope of LRRC15 presented on their surface, the method including: administering a therapeutically effective amount of any of the pharmaceutical compositions described herein or any of the ABPCs described herein to a subject identified as having a cancer characterized by having the population of cancer cells, CAFs, and/or stromal cells.

Also provided herein are methods of reducing the volume of a tumor in a subject, where the tumor is characterized by having a population of cancer cells, CAFs, and/or stromal cells that have LRRC15 or an epitope of LRRC15 presented on their surface, the method including: administering a therapeutically effective amount of any of the pharmaceutical compositions described herein or any of the ABPCs described herein to a subject identified as having a cancer characterized by having the population of cancer cells, CAFs, and/or stromal cells.

Also provided herein are methods of inducing cell death in a cancer cell, CAF, and/or stromal cell in a subject, where the cancer cell, CAF, and/or stromal cell has LRRC15 or an epitope of LRRC15 presented on its surface, where the method includes: administering a therapeutically effective amount of any of the pharmaceutical compositions described herein or any of the ABPCs described herein to a subject identified as having a cancer characterized by having a population of the cancer cells, CAFs, and/or stromal cells.

In some embodiments, the cancer is a primary tumor or a metastasis. In some embodiments, the cancer is a non-T-cell-infiltrating tumor. In some embodiments, the cancer is a T-cell infiltrating tumor.

Also provided herein are methods of decreasing the risk of developing a metastasis or decreasing the risk of developing an additional metastasis in a subject having a cancer, where the cancer is characterized by having a population of cancer cells, CAFs, and/or stromal cells that have LRRC15 or an epitope of LRRC15 presented on their surface the method including: administering a therapeutically effective amount of any one of the pharmaceutical compositions described herein or any of the ABPCs described herein to a subject identified as having a cancer characterized by having the population of cancer cells, CAFs, and/or stromal cells. In some embodiments, the cancer is a sarcoma, for example, a leiomyosarcoma, an osteosarcoma, or a chondrosarcoma.

As used herein, the term “antigen-binding protein construct” is (i) a single polypeptide that includes at least one ABD or (ii) a complex of two or more polypeptides (e.g., the same or different polypeptides) that together form at least one ABD. Non-limiting examples and aspects of antigen-binding protein constructs are described herein. Additional examples and aspects of antigen-binding protein constructs are known in the art.

A “multi-specific antigen-binding protein construct” is an antigen-binding protein construct that includes two or more different ABDs that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, the antigen is present on the surface of the cell. In some aspects, a multi-specific antigen-binding protein construct binds two different epitopes (i.e., a “bispecific antigen-binding protein construct”). In some aspects, a multi-specific antigen-binding protein construct binds three different epitopes (i.e., a “trispecific antigen-binding protein construct”). In some aspects, a multi-specific antigen-binding protein construct binds four different epitopes (i.e., a “quadspecific antigen-binding protein construct”). In some aspects, a multi-specific antigen-binding protein construct binds five different epitopes (i.e., a “quintspecific antigen-binding protein construct”). Each binding specificity may be present in any suitable valency. Non-limiting examples of multi-specific antigen-binding protein constructs are described herein.

An “ABD” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an ABD can bind to an antigen or epitope with specificity and affinity similar to that of naturally-occurring antibodies. In some embodiments, the ABD can be an antibody or a fragment thereof. In some embodiments, an ABD can include an alternative scaffold. Non-limiting examples of ABDs are described herein. Additional examples of ABDs are known in the art. In some examples, an ABD can bind to a single antigen.

The term “antibody” is used herein in its broadest sense and includes certain types of immunoglobulin molecules that include one or more ABDs that specifically bind to an antigen or epitope. An antibody specifically includes, e.g., intact antibodies (e.g., intact immunoglobulins, e.g., human IgG (e.g., human IgG1, human IgG2, human IgG3, human IgG4)), antibody fragments, and multi-specific antibodies. One example of an ABD is an ABD formed by a VH-VL dimer. Additional examples of an antibody are described herein. Additional examples of an antibody are known in the art.

The phrase “endosomal/lysosomal pathway” refers to a network of endosomes (early endosomes, multi-vesicular bodies, late endosomes, and lysosomes) in the cytoplasm of a mammalian cell, wherein molecules internalized through cell-mediated internalization processes, e.g., pinocytosis, micropinocytosis, receptor-mediated endocytosis, and/or phagocytosis, are sorted.

Once the endosomes in the endosomal/lysosomal pathway are purified or isolated, assays for a target protein (e.g., an antigen-binding protein construct described herein) can be performed using methods known in the art (ELISA, Western blot, immunofluorescence, and immunoprecipitation followed by an assay for protein concentration), and can be used to determine the concentration or relative level of the target protein in the endosomes. Alternatively, endosomes in the endosomal/lysosomal pathway can be imaged using immunofluorescence microscopy using an detectably-labelled antibody (e.g., a fluorophore-labelled, a dye-labelled, or a GFP-labelled antibody, e.g., CellLight™ Early Endosome-GFP) that specifically binds to a characteristic protein present in the endosomes (e.g., EEA1 for early endosomes) and a fluorophore-labelled antibody that specifically binds to the protein of interest (e.g., an antigen-binding protein construct), and the level of the target protein in the endosomes can be determined by quantitation of the overlap in the fluorescence emissions of the two different antibodies.

The phrase “endolysosomal delivery” refers to rate of accumulation over time or the total accumulation at a specific timepoint of an antigen-binding protein construct (e.g., any of the antigen-binding protein constructs described herein) in the endosomal/lysosomal pathway in a mammalian cell (e.g., any of the exemplary target mammalian cells described herein).

An exemplary method to calculate the increase in endolysosomal delivery of a pH-engineered ABPC variant as compared to its corresponding starting ABPC from cellular fluorescence data is to measure the ratio of the variant's mean fluorescence intensity minus the mean fluorescence intensity of a non-binding IgG control, then all divided by the variant's corresponding starting ABPC's mean fluorescence intensity minus the mean fluorescence intensity of the IgG control.

An exemplary assay for measuring endolysosomal delivery of any of the ABPCs described herein include those which involve labeling of an ABPC with a fluorescent dye, followed by incubation of the labeled ABPC with cells and measurement of cellular fluorescence as an indicator of endolysosomal delivery of the ABPC (e.g., as described generally in Wustner, Traffic 7 (6): 699-715, 2006). Alternatively, pH-sensitive dyes which preferentially fluoresce at acidic pH, but not neutral pH can be used to label any of the ABPCs described herein, which can then be incubated with cells and the cellular fluorescence measured as an indicator of delivery of the ABPC into acidic endolysosomal compartments.

The term “population” when used before a noun means two or more of the specific noun. For example, the phrase “a population of cancer cells” means “two or more cancer cells.” Non-limiting examples of cancer cells are described herein.

The phrase “cytostatic to a cell” refers to a direct or indirect decrease in the proliferation (cell division) of the cell (e.g., a cancer cell) in vivo or in vitro. When an agent is cytostatic to a cell, the agent can, e.g., directly or indirectly result in cell cycle arrest of the cell (e.g., a cancer cell). In some examples, an agent that is cytostatic to a cell can reduce the number of cells in a population of the cells that are in S phase (as compared to the number of cells in a population of the cells that are in S phase prior to contact with the agent). In some examples, an agent that is cytostatic to a cell can reduce the percentage of the cells in S phase by at least 20%, at least 40%, at least 60%, or at least 80% (e.g., as compared to the percentage of cells in a population of the cells that are in S phase prior to contact with the agent).

The phrase “cytotoxic to a cell” refers to the inducement, directly or indirectly, in the death (e.g., necrosis or apoptosis) of the cell (e.g., a mammalian cell, e.g., a cancer cell).

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an ABD and an antigen or epitope. The affinity of a molecule X for its partner Y can be represented by the dissociation equilibrium constant (K_{D}). Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity for an ABD and its corresponding antigen or epitope are known in the art.

The term “epitope” means a portion of an antigen that is specifically bound by an ABD through a set of physical interactions between: (i) all monomers (e.g., individual amino acid residues, sugar side chains, and post-translationally modified amino acid residues) on the portion of the ABD that specifically binds the antigen and (ii) all monomers (e.g., individual amino acid residues, sugar side chains, post-translationally modified amino acid residues) on the portion of the antigen that is specifically bound by the ABD. Epitopes can, e.g., consist of surface-accessible amino acid residues, sugar side chains, phosphorylated amino acid residues, methylated amino acid residues, and/or acetylated amino acid residues and may have specific three-dimensional structural characteristics, as well as specific charge characteristics. Conformational and non-conformational epitopes are distinguished in that binding to the former, but not the latter, may be lost in the presence of denaturing solvents. In some embodiments, an epitope is defined by a linear amino acid sequence of at least about 3 to 6 amino acids, or about 10 to 15 amino acids. In some embodiments, an epitope refers to a portion of a full-length protein or a portion thereof that is defined by a three-dimensional structure (e.g., protein folding). In some embodiments, an epitope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the epitope may not be critical to three-dimensional structure of the epitope. A conformational epitope may be determined and screened using assays that compare binding of antigen-binding protein construct to a denatured version of the antigen, such that a linear epitope is generated. An epitope may include amino acid residues that are directly involved in the binding, and other amino acid residues, which are not directly involved in the binding.

Methods for identifying an epitope to which an ABD specifically binds are known in the art, e.g., structure-based analysis (e.g., X-ray crystallography, NMR, and/or electron microscopy) (e.g., on the antigen and/or the antigen-ABD complex) and/or mutagenesis-based analysis (e.g., alanine scanning mutagenesis, glycine scanning mutagenesis, and homology scanning mutagenesis) wherein mutants are measured in a binding assay with a binding partner, many of which are known in the art.

The term “paratope” means a portion of an ABD that specifically binds to an antigen through a set of physical interactions between: (i) all monomers (e.g., individual amino acid residues, sugar side chains, post-translationally modified amino acid residues) on the portion of the ABD that specifically binds the antigen and (ii) all monomers (e.g., individual amino acid residues, sugar side chains, post-translationally modified amino acid residues) on the portion of the antigen that is specifically bound by the ABD. Paratopes can, e.g., consist of surface-accessible amino acid residues and may have specific three-dimensional structural characteristics, as well as specific charge characteristics. In some embodiments, a paratope refers to a portion of a full-length ABD or a portion thereof that is defined by a three-dimensional structure (e.g., protein folding). In some embodiments, a paratope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the paratope may not be critical to three-dimensional structure of the paratope. A paratope may comprise amino acid residues that are directly involved in the binding, and other amino acid residues, which are not directly involved in the binding.

Methods for identifying a paratope to which an ABD specifically binds are known in the art, e.g., structure-based analysis (e.g., X-ray crystallography, NMR, and/or electron microscopy) (e.g., on the ABD, and/or the ABD-antigen complex), and/or mutagenesis-based analysis (e.g., alanine scanning mutagenesis, glycine scanning mutagenesis, and homology scanning mutagenesis) wherein mutants are measured in a binding assay with a binding partner, many of which are known in the art.

The phrase “present on the surface of a mammalian cell” means (1) an antigen that physically attached to or at least partially embedded in the plasma membrane of a mammalian cell (e.g., a transmembrane protein, a peripheral membrane protein, a lipid-anchored protein (e.g., a GPI-anchor), an N-myristoylated protein, or a S-palmitoylated protein) or (2) an antigen that is stably bound to its cognate receptor, where the cognate receptor is physically attached to the plasma membrane of a mammalian cell (e.g., a ligand bound to its cognate receptor, where the cognate receptor is physically attached to the plasma membrane). Non-limiting methods for determining the presence of antigen on the surface of a mammalian cell include fluorescence-activated cell sorting (FACS), immunohistochemistry, cell-fractionation assays and Western blotting.

The phrase “control ABPC” or “control antigen-binding protein construct” means (i) an ABPC that is capable of specifically binding to LRRC15 or an epitope of LRRC15 presented on the surface of a mammalian cell (e.g., a target mammalian cell), where one or both of the following is true: (a) the dissociation rate of the first ABD at a pH of about 4.0 to about 6.5 (e.g., any of the subranges of this range described herein) is ≤3-fold (e.g., ≤2.8-fold, ≤2.6-fold, ≤2.5-fold, ≤2.4-fold, ≤2.2-fold, ≤2.0-fold, ≤1.8-fold, ≤1.6-fold, ≤1.5-fold, ≤1.4-fold, ≤1.2-fold, ≤1.0-fold, ≤0.8-fold, ≤0.6-fold, ≤0.5-fold, ≤0.4-fold, ≤0.3-fold ≤0.2-fold, or ≤0.1-fold) faster than the dissociation rate at a pH of about 7.0 to about 8.0 (e.g., any of the subranges of this range described herein); or (b) the dissociation constant (K_{D}) of the first ABD at a pH of about 4.0 to about 6.5 (e.g., any of the subranges of this range described herein) is ≤3-fold (e.g., ≤2.8, ≤2.6, ≤2.5, ≤2.4, ≤2.2, ≤2.0, ≤1.8, ≤1.6, ≤1.5, ≤1.4, ≤1.2, ≤1.0, ≤0.8, ≤0.6, ≤0.5, ≤0.4, ≤0.3≤0.2, or ≤0.1 fold) greater than the K_{D} at a pH of about 7.0 to about 8.0 (e.g., any of the subranges of the ranges described herein); (i) 15G7; (ii) 24D9; and/or (iii) 29F1.

The term “extracellular space” means the liquid exterior to the plasma membrane of a mammalian cell. When a mammalian cell is in vitro, the extracellular space can be a liquid culture medium. When a mammalian cell is in vivo, the extracellular space can be, e.g., plasma, serum, blood, interstitial fluid, or lymph.

The term “endolysosomal space” means the fluid encapsulated by the vesicles and organelles that make-up the endosomal/lysosomal pathway in a mammalian cell.

The phrase “a reduced level” or “a decreased level” can be a reduction or decrease of at least a 1% (e.g., ≥2%, ≥4%, ≥6%, ≥8%, ≥10%, ≥12%, ≥14%, ≥16%, ≥18%, ≥20%, ≥22%, ≥24%, ≥26%, ≥30%, ≥35%, ≥40%, ≥45%, ≥50%, ≥55%, ≥60%, ≥65%, ≥70%, ≥75%, ≥80%, ≥85%, ≥90%, ≥95%, or ≥99%) reduction as compared to a reference level or value.

The term “cell killing potency” refers to the ability of an agent (e.g., any of the ABPCs described herein) to induce, directly or indirectly, the apoptosis and/or necrosis of a mammalian cell (e.g., a cancer cell), measured as a rate over time or at a relevant timepoint. Methods for determining the cell killing potency of a cell are known in the art (e.g., trypan blue staining, microscopy, fluorescence-assisted cell sorting, and assays to detect markers of apoptosis (e.g., Annexin V)). In non-limiting examples, cell killing potency can be measured, e.g., by cell killing at a single concentration of an agent, by the IC50 of the agent (i.e. the concentration of the agent whereby half the maximal cell killing potency is achieved), or by the ratio of an agent's dissociation constant KD on mammalian cells divided by its IC50. In some non-limiting examples, the IC50s and/or the KD ratios described herein are compared to those of a control ABPC (as defined herein), and, optionally, demonstrate that the ABPCs described herein have a higher cell killing potency as compared to the control ABPC.

The term “toxin liberation” refers to the ability of a mammalian cell (e.g., a non-cancerous mammalian cell or a cancer cell) to internalize (e.g., via pinocytosis and/or receptor-mediated endocytosis) any of the ABPCs described herein (e.g., any of ABPCs or control ABPCs described herein) that are conjugated to a toxin, and subsequently release the toxin conjugated to the ABPC, measured as a rate over time or at a specific timepoint. Toxin liberation can be assessed using a variety of different exemplary assays, e.g., ELISA, immunofluorescence, cell killing assays, cell cycle arrest assays, DNA damage assays, mass spectrometry, HPLC, and/or an isotope-labeled toxin.

The phrase “target cell” or “target mammalian cell” or “mammalian target cell” means a mammalian cell that has at least one LRRC15 present on its surface. In some examples, a mammalian target cell can be a cancer cell. In some embodiments of a target mammalian cell can have a total of about the following (each±about 10%): 1-10E6, 1-9E6, 1-8E6, 1-7E6, 1-6E6, 1-5E6, 1-4E6, 1-3E6, 1-2E6, 1-1E6, 1-800,000, 1-600,000, 1-400,000, 1-200,000, 1-100,000, 1-80,000, 1-80,000, 1-75,000, 1-70,000, 1-65,000, 1-60,000, 1-55,000, 1-50,000, 1-45,000, 1-40,000, 1-35,000, 1-30,000, 1-25,000, 1-20,000, 1-15,000, 1-10,000, 1-7,500, 1-5,000, 1-4,000, 1-3,000, 1-2,000, 1-1,000, 1-500, 1-100, 1-50, or 1-10, or any of the ranges of numbers recited in U.S. Patent Application No. 2022/0281984, which is incorporated by reference herein in its entirety, of the LRRC15 present on the plasma membrane of the target mammalian cell.

The phrase “antigen density” means the number of LRRC15 present on the surface of a target mammalian cell or the average number of LRRC15 on the surface of a population of particular type of target mammalian cells. It can be measured, e.g., using the QuantiBRITE™ bead kit or radiolabel (e.g., BD Biosciences PE Phycoerythrin Fluorescence Quantitation Kit, catalog #340495).

The phrase “amino acid substituted with a histidine” means the substitution of an amino acid residue that is not histidine in a reference polypeptide sequence with a histidine. Non-limiting methods for substituting an amino acid residue in a reference polypeptide with a histidine are described herein. Additional methods for substituting an amino acid residue in a reference polypeptide with a histidine are known in the art.

The phrase “amino acid substituted with an alanine” means the substitution of an amino acid residue that is a histidine in a reference polypeptide sequence with an alanine. Non-limiting methods for substituting a histidine in a reference polypeptide with an alanine are described herein. Additional methods for substituting a histidine in a reference polypeptide with an alanine are known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1A shows the binding of 15G7 and its HC variants to LRRC15 by biolayer interferometry (BLI). For each individual plot, antigen binding proteins were captured on anti-human Fc biosensors and associated with LRRC15 at pH 7.4. Dissociation was at pH 7.4 (black) or pH 5.4 (grey). Major X-axis ticks=100 seconds, with left dashed vertical line=0 seconds. Major Y-axis ticks=0.5 nm and octet plots are shown MYT5712 and MYT6080-MYT6121.

FIG. 1B shows the binding of 15G7 and its LC variants to LRRC15 by BLI. Major Y-axis ticks=0.2 nm and octet plots are shown MYT6122-MYT6151.

FIG. 1C shows the binding of humanized 15G7 and its HC variants to LRRC15 by BLI. Major Y-axis ticks=0.5 nm and octet plots are shown MYT8387-MYT8411.

FIG. 1D shows the binding of humanized 15G7 and its LC variants to LRRC15 by BLI Major Y-axis ticks=0.2 nm and octet plots are shown MYT8413-MYT8437.

FIG. 1E shows the binding data of FIG. 1A plotted to maximize the vertical separation.

FIG. 1F shows the binding data of FIG. 1B plotted to maximize the vertical separation.

FIG. 1G shows the binding data of FIG. 1C plotted to maximize the vertical separation.

FIG. 1H shows the binding data of FIG. 1D plotted to maximize the vertical separation.

FIG. 2A shows the binding of 24D9 and its HC variants to LRRC15 by BLI. Major Y-axis ticks=0.2 nm and octet plots are shown for MYT5742 and MYT7881-MYT7925.

FIG. 2B shows the binding data of FIG. 2A plotted to maximize the vertical separation.

FIG. 3A shows the binding of 24D9 and its LC variants to LRRC15 by BLI. Major Y-axis ticks=0.2 nm and octet plots are shown for MYT7926-MYT7958.

FIG. 3B shows the binding data of FIG. 3A plotted to maximize the vertical separation.

FIG. 4A shows the binding of humanized 29F1 and its HC aspartate variants to LRRC15 by BLI. Major Y-axis ticks=0.1 nm and octet plots are shown for MYT8094 and MYT9464-MYT9506.

FIG. 4B shows the binding data of FIG. 4A plotted to maximize the vertical separation.

FIG. 5A shows the binding of humanized 29F1 and its LC aspartate variants to LRRC15 by BLI. Major Y-axis ticks=0.1 nm and octet plots are shown for MYT9507-MYT9534.

FIG. 5B shows the binding data of FIG. 5A plotted to maximize the vertical separation.

FIG. 6A shows the binding of humanized 29F1 and its HC glutamate variants to LRRC15 by BLI. Major Y-axis ticks=0.2 nm and octet plots are shown for MYT9684-MYT9726.

FIG. 6B shows the binding data of FIG. 6A plotted to maximize the vertical separation.

FIG. 7A shows the binding of humanized 29F1 and its LC glutamate variants to LRRC15 by BLI. Major Y-axis ticks=0.2 nm and octet plots are shown for MYT9727-MYT9754.

FIG. 7B shows the binding data of FIG. 7A plotted to maximize the vertical separation.

FIG. 8 shows a graph showing the median tumor volume (TV) at the indicated days post administration of the indicated test article or control to mice bearing SA4033 tumors.

FIG. 9 presents a graph of median TV at the indicated days post administration of the indicated test article or control in the CTG-0241 model.

FIG. 10A shows a graph of median TV at the indicated days post administration of the indicated test article or control in the SA3851 model.

FIG. 10B shows a graph of median TV at the indicated days post administration of the indicated test article or control in the SA4040 model.

FIG. 10C shows a graph of median TV at the indicated days post administration of the indicated test article or control in the SA4109 model.

### DETAILED DESCRIPTION

Provided herein are antigen-binding protein constructs (ABPCs) that include: a first ABD that is capable of specifically binding LRRC15 or an epitope of LRRC15 presented on the surface of a target mammalian cell, optionally where: (a) the dissociation rate of the first ABD at a pH of about 4.0 to about 6.5 is faster than the dissociation rate at a pH of about 7.0 to about 8.0; and/or (b) the dissociation constant (K_{D}) of the first ABD at a pH of about 4.0 to about 6.5 is greater than the K_{D} at a pH of about 7.0 to about 8.0. In some examples of these ABPCs, the ABPC is degraded in the target mammalian cell following internalization of the ABPC by the target mammalian cell. Some examples of any of the ABPCs described herein can further include a conjugated toxin, radioisotope, drug, or small molecule (e.g., a fluorophore or dye).

Also provided are antigen-binding protein constructs (ABPCs) that include: a first ABD that is capable of specifically binding LRRC15 or an epitope of LRRC15 presented on the surface of a target mammalian cell; and a conjugated toxin, radioisotope, drug, or small molecule, optionally where: (a) the dissociation rate of the first ABD at a pH of about 4.0 to about 6.5 is faster than the dissociation rate at a pH of about 7.0 to about 8.0; and/or the dissociation constant (K_{D}) of the first ABD at a pH of about 4.0 to about 6.5 is greater than the K_{D} at a pH of about 7.0 to about 8.0; and (b) a composition including the ABPC provides for one or more (e.g., two or three) of: an increase (e.g., a detectable increase) in toxin liberation in the target mammalian cell as compared to a composition comprising the same amount of a control ABPC; an increase (e.g., a detectable increase) in target mammalian cell killing as compared to a composition comprising the same amount of a control ABPC; and an increase (e.g., a detectable increase) in endolysosomal delivery in the target mammalian cell as compared to a composition comprising the same amount of a control ABPC.

In some examples, the first ABD includes an HCV domain of 15G7 optionally with one or more (e.g., 1, 2, 3, 4, 5, 6, 7, 8, 9, or 10) amino acids substituted with a histidine. In some examples, the first ABD includes an LCV domain of 15G7 optionally with one or more (e.g., 1, 2, 3, 4, 5, 6, 7, 8, 9, or 10) amino acids substituted with a histidine. In some examples, the first ABD includes an HCV domain of 15G7 optionally with one or more (e.g., 1, 2, 3, 4, 5, 6, 7, 8, 9, or 10) amino acids substituted with a histidine; and an LCV domain of 15G7 optionally with one or more (e.g., 1, 2, 3, 4, 5, 6, 7, 8, 9, or 10) amino acids substituted with a histidine. In some examples, the HCV domain of 15G7 comprises SEQ ID NO: 378 OR 382. In some examples, the LCV domain of 15G7 comprises SEQ ID NO: 451 or SEQ ID NO: 455.

In some examples, the first ABD comprises an HCV domain comprising a CDR1, a CDR2, and a CDR3 of SEQ ID NO: 379, 380, and 381, respectively, optionally with collectively a total of one or more (e.g., 1, 2, 3, 4, 5, 6, 7, 8, 9, or 10) amino acid positions in SEQ ID NOs: 379-381 substituted with a histidine. In some examples, the first ABD comprises an LCV domain comprising a CDR1, a CDR2, and a CDR3 of SEQ ID NO: 452, 453, and 454, respectively, optionally with collectively a total of one or more (e.g., 1, 2, 3, 4, 5, 6, 7, 8, 9, or 10) amino acid positions in SEQ ID NOs: 452-454 substituted with a histidine. In some examples, the first ABD includes: an HCV domain comprising a CDR1, a CDR2, and a CDR3 of SEQ ID NO: 379, 380, and 381, respectively, optionally with collectively a total of one or more (e.g., 1, 2, 3, 4, 5, 6, 7, 8, 9, or 10) amino acid positions in NOs: 379-381 substituted with a histidine; and an LCV domain comprising a CDR1, a CDR2, and a CDR3 of SEQ ID NO: 452, 453, and 454, respectively, optionally with collectively a total of one or more (e.g., 1, 2, 3, 4, 5, 6, 7, 8, 9, or 10) amino acid positions in SEQ ID NOs: 452-454 substituted with a histidine.

In some examples, the first ABD includes an HCV domain that is at least 90% (e.g., ≥92%, ≥94%, ≥96%, ≥98%, ≥99%, or 100%) identical to the sequence set forth in SEQ ID NO: 378 OR 382, optionally where the HCV domain includes a histidine at one or more (e.g., 1, 2, 3, 4, 5, 6, 7, 8, 9, or 10) amino acid positions in SEQ ID NO: 378 OR 382 selected from the group consisting of: 32, 34, 53, 60, 103, 104, 105, 109, and 110. In some examples, the first ABD includes an LCV domain that is ≥90% (e.g., ≥92%, ≥94%, ≥96%, ≥98%, ≥99%, or 100%) identical to the sequence set forth in SEQ ID NO: 451 or SEQ ID NO: 455, optionally where the LCV domain includes a histidine at one or more (e.g., 1, 2, 3, 4, 5, 6, 7, 8, 9, or 10) amino acid positions in SEQ ID NO: 451 or SEQ ID NO: 455 selected from the group consisting of: 29, 30, 32, 34, 50, 92, 93, 95, 96, and 97. In some examples the first ABD includes: an HCV domain that is ≥90% (e.g., ≥92%, ≥94%, ≥96%, ≥98%, ≥99%, or 100%) identical to the sequence set forth in SEQ ID NO: 378 OR 382, optionally where the HCV domain includes a histidine at one or more (e.g., 1, 2, 3, 4, 5, 6, 7, 8, 9, or 10) amino acid positions in SEQ ID NO: 378 OR 382 selected from the group consisting of: 32, 34, 53, 60, 103, 104, 105, 109, and 110; and an LCV domain that is ≥90% (e.g., ≥92%, ≥94%, ≥96%, ≥98%, ≥99%, or 100%) identical to the sequence set forth in SEQ ID NO: 451 or SEQ ID NO: 455, optionally where the LCV domain includes a histidine at one or more (e.g., 1, 2, 3, 4, 5, 6, 7, 8, 9, or 10) amino acid positions in SEQ ID NO: 451 or SEQ ID NO: 455 selected from the group consisting of: 29, 30, 32, 34, 50, 92, 93, 95, 96, and 97.

In some examples, an HCV domain includes an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

TABLE 1
Exemplary Combinations of Amino Acid Positions in SEQ ID NO: 378 OR SEQ ID NO: 382 that can be Substituted with Histidine

| Column | 1 | 2 | 3 | 4 | 5 | 6 | 7 | 8 | 9 |
|---|---|---|---|---|---|---|---|---|---|
| SEQ ID NO: 378 or SEQ ID | 32 | 34 | 53 | 60 | 103 | 104 | 105 | 109 | 110 |
| NO: 382 Position | | | | | | | | | |

| | |
|---|---|
| A | Each and every unique 2-position combination of X and Y, wherein X and are each |
| | selected from a SEQ ID NO: 378 or SEQ ID NO: 382 amino acid position recited in |
| | one of columns 1 to 9, without replacement (i.e., X + Y). Expressed another way, the |
| | combinations of positions recited in the following combinations of columns: 1 + 2, |
| | 1 + 3, 1 + 4, 1 + 5, 1 + 6, 1 + 7, 1 + 8, 1 + 9, 2 + 3, 2 + 4, 2 + 5, 2 + 6, 2 + 7, 2 + 8, 2 + 9, 3 + 4, 3 + 5, |
| | 3 + 6, 3 + 7, 3 + 8, 3 + 9, 4 + 5, 4 + 6, 4 + 7, 4 + 8, 4 + 9, 5 + 6, 5 + 7, 5 + 8, 5 + 9, 6 + 7, 6 + 8, 6 + 9, |
| | 7 + 8, 7 + 9, and 8 + 9. |
| B | Each and every unique 3-position combination of X, Y, and Z, wherein X and Y are |
| | together one of the 36 2-position combinations recited in “A” (above), and Z is |
| | selected from a SEQ ID NO: 378 or SEQ ID NO: 382 amino acid position recited in |
| | one of columns 1 to 9, without replacement (i.e. X + Y#Z). In other words, 1 + 2 + 3, |
| | 1 + 2 + 4, 1 + 2 + 5, 1 + 2 + 6, 1 + 2 + 7, 1 + 2 + 8, 1 + 2 + 9, 1 + 3 + 4, 1 + 3 + 5, 1 + 3 + 6, 1 + 3 + 7, 1 + 3 + 8, |
| | 1 + 3 + 9, 1 + 4 + 5, 1 + 4 + 6, 1 + 4 + 7, 1 + 4 + 8, 1 + 4 + 9, 1 + 5 + 6, 1 + 5 + 7, 1 + 5 + 8, 1 + 5 + 9, 1 + 6 + 7, |
| | 1 + 6 + 8, 1 + 6 + 9, 1 + 7 + 8, 1 + 7 + 9, 1 + 8 + 9, 2 + 3 + 4, 2 + 3 + 5, 2 + 3 + 6, 2 + 3 + 7, 2 + 3 + 8, 2 + 3 + 9, |
| | 2 + 4 + 5, 2 + 4 + 6, 2 + 4 + 7, 2 + 4 + 8, 2 + 4 + 9, 2 + 5 + 6, 2 + 5 + 7, 2 + 5 + 8, 2 + 5 + 9, 2 + 6 + 7, 2 + 6 + 8, |
| | 2 + 6 + 9, 2 + 7 + 8, 2 + 7 + 9, 2 + 8 + 9, 3 + 4 + 5, 3 + 4 + 6, 3 + 4 + 7, 3 + 4 + 8, 3 + 4 + 9, 3 + 5 + 6, 3 + 5 + 7, |
| | 3 + 5 + 8, 3 + 5 + 9, 3 + 6 + 7, 3 + 6 + 8, 3 + 6 + 9, 3 + 7 + 8, 3 + 7 + 9, 3 + 8 + 9, 4 + 5 + 6, 4 + 5 + 7, 4 + 5 + 8, |
| | 4 + 5 + 9, 4 + 6 + 7, 4 + 6 + 8, 4 + 6 + 9, 4 + 7 + 8, 4 + 7 + 9, 4 + 8 + 9, 5 + 6 + 7, 5 + 6 + 8, 5 + 6 + 9, 5 + 7 + 8, |
| | 5 + 7 + 9, 5 + 8 + 9, 6 + 7 + 8, 6 + 7 + 9, 6 + 8 + 9, and 7 + 8 + 9. |

In some examples, an LCV domain includes an LCV domain that is ≥90% (e.g., ≥92%, ≥94%, ≥96%, ≥98%, ≥99%, or 100%) identical to the sequence set forth in SEQ ID NO: 451 or SEQ ID NO: 455, where the LCV domain includes a histidine at any of the specific combinations of one or more amino acid positions in SEQ ID NO: 451 or SEQ ID NO: 455 listed in Table 2.

TABLE 2
Exemplary Combinations of Amino Acid Positions in SEQ ID NO: 451 or SEQ ID NO: 455 that can be Substituted with Histidine

| Column | 1 | 2 | 3 | 4 | 5 | 6 | 7 | 8 | 9 | 10 |
|---|---|---|---|---|---|---|---|---|---|---|
| SEQ ID NO: 451 or SEQ ID | 29 | 30 | 32 | 34 | 50 | 92 | 93 | 95 | 96 | 97 |
| NO: 455 Position | | | | | | | | | | |

| | |
|---|---|
| A | Each and every unique 2-position combination of X and Y, wherein X and are each |
| | selected from a SEQ ID NO: 451 or SEQ ID NO: 455 amino acid position recited in |
| | one of columns 1 to 10, without replacement (i.e., X + Y). Expressed another way, the |
| | combinations of positions recited in the following combinations of columns: 1 + 2, |
| | 1 + 3, 1 + 4, 1 + 5, 1 + 6, 1 + 7, 1 + 8, 1 + 9, 1 + 10, 2 + 3, 2 + 4, 2 + 5, 2 + 6, 2 + 7, 2 + 8, 2 + 9, 2 + 10, |
| | 3 + 4, 3 + 5, 3 + 6, 3 + 7, 3 + 8, 3 + 9, 3 + 10, 4 + 5, 4 + 6, 4 + 7, 4 + 8, 4 + 9, 4 + 10, 5 + 6, 5 + 7, 5 + 8, |
| | 5 + 9, 5 + 10, 6 + 7, 6 + 8, 6 + 9, 6 + 10, 7 + 8, 7 + 9, 7 + 10, 8 + 9, 8 + 10, and 9 + 10. |
| B | Each and every unique 3-position combination of X, Y, and Z, wherein X and Y are |
| | together one of the 45 2-position combinations recited in “A” (above), and Z is |
| | selected from a SEQ ID NO: 451 or SEQ ID NO: 455 amino acid position recited in |
| | one of columns 1 to 10, without replacement (i.e. XYZ). In other words, 1 + 2 + 3, |
| | 1 + 2 + 4, 1 + 2 + 5, 1 + 2 + 6, 1 + 2 + 7, 1 + 2 + 8, 1 + 2 + 9, 1 + 2 + 10, 1 + 3 + 4, 1 + 3 + 5, 1 + 3 + 6, 1 + 3 + 7, |
| | 1 + 3 + 8, 1 + 3 + 9, 1 + 3 + 10, 1 + 4 + 5, 1 + 4 + 6, 1 + 4 + 7, 1 + 4 + 8, 1 + 4 + 9, 1 + 4 + 10, 1 + 5 + 6, |
| | 1 + 5 + 7, 1 + 5 + 8, 1 + 5 + 9, 1 + 5 + 10, 1 + 6 + 7, 1 + 6 + 8, 1 + 6 + 9, 1 + 6 + 10, 1 + 7 + 8, 1 + 7 + 9, |
| | 1 + 7 + 10, 1 + 8 + 9, 1 + 8 + 10, 1 + 9 + 10, 2 + 3 + 4, 2 + 3 + 5, 2 + 3 + 6, 2 + 3 + 7, 2 + 3 + 8, 2 + 3 + 9, |
| | 2 + 3 + 10, 2 + 4 + 5, 2 + 4 + 6, 2 + 4 + 7, 2 + 4 + 8, 2 + 4 + 9, 2 + 4 + 10, 2 + 5 + 6, 2 + 5 + 7, 2 + 5 + 8, |
| | 2 + 5 + 9, 2 + 5 + 10, 2 + 6 + 7, 2 + 6 + 8, 2 + 6 + 9, 2 + 6 + 10, 2 + 7 + 8, 2 + 7 + 9, 2 + 7 + 10, 2 + 8 + 9, |
| | 2 + 8 + 10, 2 + 9 + 10, 3 + 4 + 5, 3 + 4 + 6, 3 + 4 + 7, 3 + 4 + 8, 3 + 4 + 9, 3 + 4 + 10, 3 + 5 + 6, 3 + 5 + 7, |
| | 3 + 5 + 8, 3 + 5 + 9, 3 + 5 + 10, 3 + 6 + 7, 3 + 6 + 8, 3 + 6 + 9, 3 + 6 + 10, 3 + 7 + 8, 3 + 7 + 9, 3 + 7 + 10, |
| | 3 + 8 + 9, 3 + 8 + 10, 3 + 9 + 10, 4 + 5 + 6, 4 + 5 + 7, 4 + 5 + 8, 4 + 5 + 9, 4 + 5 + 10, 4 + 6 + 7, 4 + 6 + 8, |
| | 4 + 6 + 9, 4 + 6 + 10, 4 + 7 + 8, 4 + 7 + 9, 4 + 7 + 10, 4 + 8 + 9, 4 + 8 + 10, 4 + 9 + 10, 5 + 6 + 7, 5 + 6 + 8, |
| | 5 + 6 + 9, 5 + 6 + 10, 5 + 7 + 8, 5 + 7 + 9, 5 + 7 + 10, 5 + 8 + 9, 5 + 8 + 10, 5 + 9 + 10, 6 + 7 + 8, 6 + 7 + 9, |
| | 6 + 7 + 10, 6 + 8 + 9, 6 + 8 + 10, 6 + 9 + 10, 7 + 8 + 9, 7 + 8 + 10, 7 + 9 + 10, and 8 + 9 + 10. |

In some examples, the first ABD includes an HCV domain that is ≥90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1; and an LCV domain that that is ≥90% (e.g., ≥92%, ≥94%, ≥96%, ≥98%, ≥99%, or 100%) identical to the sequence set forth in SEQ ID NO: 451 or SEQ ID NO: 455, where the LCV domain includes a histidine at any of the specific combinations of one or more amino acid positions in SEQ ID NO: 451 or SEQ ID NO: 455 listed in Table 2.

In some examples, the first ABD comprises an LCV domain comprising the sequence set forth in SEQ ID NO: 451 or SEQ ID NO: 455 and an HCV domain that is ≥90% identical (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more (e.g., 2, 3, 4, 5, 6, 7, 8, 9, or 10) amino acid positions in SEQ ID NO: 378 OR 382 listed in Table 1.

In some examples, the first ABD comprises an LCV domain that is ≥90% identical to the sequence set forth in SEQ ID NO: 451 or SEQ ID NO: 455, wherein the LCV domain includes a histidine at position 29 in SEQ ID NO: 451 or SEQ ID NO: 455; and an HCV domain that is ≥90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is ≥90% identical to the sequence set forth in SEQ ID NO: 451 or SEQ ID NO: 455, wherein the LCV domain includes a histidine at position 30 in SEQ ID NO: 451 or SEQ ID NO: 455; and an HCV domain that is ≥90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 32 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 34 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 50 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 92 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 93 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 95 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 96 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an LCV domain that is at least 90% identical to the sequence set forth in SEQ ID NO: 451 or SEQ ID NO: 455, wherein the LCV domain includes a histidine at position 97 in SEQ ID NO: 451 or SEQ ID NO: 455; and an HCV domain that is at least 90% (e.g., ≥92%, ≥94%, ≥96%, ≥98%, ≥99%, or 100%) identical to the sequence set forth in SEQ ID NO: 378 OR 382, where the HCV domain includes a histidine at any of the specific combinations of one or more amino acid positions in SEQ ID NO: 378 OR 382 listed in Table 1.

In some examples, the first ABD comprises an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 378, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 382, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 383, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 384, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 385, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 386, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 387, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 388, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 389, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 390, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 391, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 392, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 393, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 394, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 395, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 396, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 397, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 398, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 399, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 400, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 401, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 402, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 403, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 404, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 405, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 406, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 407, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 408, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 409, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 410, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 411, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 412, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 413, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 414, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 415, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 416, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 417, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 418, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 419, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 420, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 421, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 422, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 423, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 424, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 425, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 426, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 427, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 428, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 429, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 430, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 431, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 432, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 433, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 434, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 435, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 436, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 437, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 438, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 439, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 440, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 441, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 442, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 443, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 444, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 445, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 446, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 447, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 448, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 449, and an LCV domain comprising the sequence set forth in SEQ ID NO: 451, SEQ ID NO: 455, or one of SEQ ID NOs: 456-511.

In some examples, the first ABD includes an HCV domain comprising the sequence set forth in SEQ ID NO: 450, and an LCV domain comprising the sequence set forth in SEQ ID NO: 451, SEQ ID NO: 455, or one of SEQ ID NOs: 456-511.

In some examples, the first ABD includes an LCV domain comprising the sequence set forth in SEQ ID NO: 451, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 456, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 457, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 458, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 459, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 460, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 461, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 462, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 463, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 464, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 465, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 466, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 467, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 468, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 469, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 470, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 471, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 472, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 473, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 474, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 475, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 476, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 477, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 478, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 479, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 480, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 481, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 482, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 483, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 484, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 485, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 486, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 487, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 488, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 489, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 490, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 491, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 492, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 493, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 494, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 495, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 496, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 497, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 498, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 499, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 500, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 501, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 502, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 503, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 504, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 505, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 506, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 507, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 508, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 509, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 510, and an HCV domain comprising the sequence set forth in SEQ ID NO: 378, SEQ ID NO: 382, or one of SEQ ID NOs: 383-450.

In some examples, the first ABD includes an LCV domain comprising the sequence set forth in SEQ ID NO: 511, and an HCV domain comprising the sequence set forth in SEQ ID NO: 378, SEQ ID NO: 382, or one of SEQ ID NOs: 383-450.

In some examples, the first ABD includes an HCV domain of a 24D9 optionally with one or more (e.g., 1, 2, 3, 4, 5, 6, 7, 8, 9, or 10) amino acids substituted with a histidine. In some examples, the first ABD includes an LCV domain of 24D9 optionally with one or more (e.g., 1, 2, 3, 4, 5, 6, 7, 8, 9, or 10) amino acids substituted with a histidine. In some examples, the first ABD includes an HCV domain of 24D9 optionally with one or more (e.g., 1, 2, 3, 4, 5, 6, 7, 8, 9, or 10) amino acids substituted with a histidine; and an LCV domain of 24D9 optionally with one or more (e.g., 1, 2, 3, 4, 5, 6, 7, 8, 9, or 10) amino acids substituted with a histidine. In some examples, the HCV domain of 24D9 comprises the amino acid sequence set forth in SEQ ID NO: 512 or SEQ ID NO: 516. In some examples, the LCV domain of 24D9 comprises the amino acid sequence set forth in SEQ ID NO: 518 or SEQ ID NO: 522.

In some examples, the first ABD comprises an HCV domain comprising a CDR1, a CDR2, and a CDR3 of SEQ ID NO: 513, 514, and 515, respectively, optionally with collectively a total of one or more (e.g., 1, 2, 3, 4, 5, 6, 7, 8, 9, or 10) amino acid positions in SEQ ID NOs: 513-515 substituted with a histidine. In some examples, the first ABD comprises an LCV domain comprising a CDR1, a CDR2, and a CDR3 of SEQ ID NO: 519, 520, and 521, respectively, optionally with collectively a total of one or more (e.g., 1, 2, 3, 4, 5, 6, 7, 8, 9, or 10) amino acid positions in SEQ ID NOs: 519-521 substituted with a histidine. In some examples, the first ABD includes: an HCV domain comprising a CDR1, a CDR2, and a CDR3 of SEQ ID NO: 513, 514, and 515, respectively, optionally with collectively a total of one or more (e.g., 1, 2, 3, 4, 5, 6, 7, 8, 9, or 10) amino acid positions in SEQ ID NOs: 513-515 substituted with a histidine; and an LCV domain comprising a CDR1, a CDR2, and a CDR3 of SEQ ID NO: 519, 520, and 521, respectively, optionally with collectively a total of one or more (e.g., 1, 2, 3, 4, 5, 6, 7, 8, 9, or 10) amino acid positions in SEQ ID NOs: 519-521 substituted with a histidine.

In some examples, the first ABD includes an HCV domain that is at least 90% (e.g., ≥92%, ≥94%, ≥96%, ≥98%, ≥99%, or 100%) identical to the sequence set forth in SEQ ID NO: 512 or SEQ ID NO: 516, optionally where the HCV domain includes a histidine at one or more (e.g., 1, 2, 3, 4, 5, 6, 7, 8, 9, or 10) amino acid positions in SEQ ID NO: 512 or SEQ ID NO: 516 selected from the group consisting of: 30, 31, 32, 52, 53, 58, 59, 60, 98, 105, 106, and 110. In some examples, the first ABD includes an LCV domain that is at least 90% (e.g., ≥92%, ≥94%, ≥96%, ≥98%, ≥99%, or 100%) identical to the sequence set forth in SEQ ID NO: 518 or SEQ ID NO: 522, optionally where the LCV domain includes a histidine at one or more (e.g., 1, 2, 3, 4, 5, 6, 7, 8, 9, or 10) amino acid positions in SEQ ID NO: 518 or SEQ ID NO: 522 selected from the group of: 35 and 97. In some examples the first ABD includes: an HCV domain that is at least 90% (e.g., ≥92%, ≥94%, ≥96%, ≥98%, ≥99%, or 100%) identical to the sequence set forth in SEQ ID NO: 512, optionally where the HCV domain includes a histidine at one or more (e.g., 1, 2, 3, 4, 5, 6, 7, 8, 9, or 10) amino acid positions in SEQ ID NO: 512 or SEQ ID NO: 516 selected from the group consisting of: 30, 31, 32, 52, 53, 58, 59, 60, 98, 105, 106, and 110; and an LCV domain that is at least 90% (e.g., ≥92%, ≥94%, ≥96%, ≥98%, ≥99%, or 100%) identical to the sequence set forth in SEQ ID NO: 518 or SEQ ID NO: 522, optionally where the LCV domain includes a histidine at one or more (e.g., 1, 2, 3, 4, 5, 6, 7, 8, 9, or 10) amino acid positions in SEQ ID NO: 518 or SEQ ID NO: 522 selected from the group consisting of: 35 and 97.

In some examples, an HCV domain includes an HCV domain that is at least 90% (e.g., ≥92%, ≥94%, ≥96%, ≥98%, ≥99%, or 100%) identical to the sequence set forth in SEQ ID NO: 512 or SEQ ID NO: 516, where the HCV domain includes a histidine at any of the specific combinations of one or more amino acid positions in SEQ ID NO: 512 or SEQ ID NO: 516 listed in Table 3.

TABLE 3
Exemplary Combinations of Amino Acid Positions in SEQ ID NO: 512 or SEQ ID NO: 516 that can be Substituted with Histidine

| Column | 1 | 2 | 3 | 4 | 5 | 6 | 7 | 8 | 9 | 10 | 11 | 12 |
|---|---|---|---|---|---|---|---|---|---|---|---|---|
| SEQ ID NO: 512 or SEQ ID | 30 | 31 | 32 | 52 | 53 | 58 | 59 | 60 | 98 | 105 | 106 | 110 |
| NO: 516 Position | | | | | | | | | | | | |

| | |
|---|---|
| A | Each and every unique 2-position SEQ combination of X and Y, wherein X and are each |
| | selected from a SEQ ID NO: 512 or ID NO: 516 amino acid position recited in |
| | one of columns 1 to 12, without replacement (i.e., XY). Expressed another way, the |
| | combinations of positions recited in the following combinations of columns: 1 + 2, |
| | 1 + 3, 1 + 4, 1 + 5, 1 + 6, 1 + 7, 1 + 8, 1 + 9, 1 + 10, 1 + 11, 1 + 12, 2 + 3, 2 + 4, 2 + 5, 2 + 6, 2 + 7, 2 + 8, |
| | 2 + 9, 2 + 10, 2 + 11, 2 + 12, 3 + 4, 3 + 5, 3 + 6, 3 + 7, 3 + 8, 3 + 9, 3 + 10, 3 + 11, 3 + 12, 4 + 5, 4 + 6, |
| | 4 + 7, 4 + 8, 4 + 9, 4 + 10, 4 + 11, 4 + 12, 5 + 6, 5 + 7, 5 + 8, 5 + 9, 5 + 10, 5 + 11, 5 + 12, 6 + 7, 6 + 8, |
| | 6 + 9, 6 + 10, 6 + 11, 6 + 12, 7 + 8, 7 + 9, 7 + 10, 7 + 11, 7 + 12, 8 + 9, 8 + 10, 8 + 11, 8 + 12, 9 + 10, |
| | 9 + 11, 9 + 12, 10 + 11, 10 + 12, 11 + 12. |
| B | Each and every unique 3-position combination of X, Y, and Z, wherein X and Y are |
| | together one of the 66 2-position combinations recited in “A” (above), and Z is |
| | selected from a SEQ ID NO: 512 or SEQ ID NO: 516 amino acid position recited in |
| | one of columns 1 to 12, without replacement (i.e. X + Y#Z). In other words, 1 + 2 + 3, |
| | 1 + 2 + 4, 1 + 2 + 5, 1 + 2 + 6, 1 + 2 + 7, 1 + 2 + 8, 1 + 2 + 9, 1 + 2 + 10, 1 + 2 + 11, 1 + 2 + 12, 1 + 3 + 4, |
| | 1 + 3 + 5, 1 + 3 + 6, 1 + 3 + 7, 1 + 3 + 8, 1 + 3 + 9, 1 + 3 + 10, 1 + 3 + 11, 1 + 3 + 12, 1 + 4 + 5, 1 + 4 + 6, |
| | 1 + 4 + 7, 1 + 4 + 8, 1 + 4 + 9, 1 + 4 + 10, 1 + 4 + 11, 1 + 4 + 12, 1 + 5 + 6, 1 + 5 + 7, 1 + 5 + 8, 1 + 5 + 9, |
| | 1 + 5 + 10, 1 + 5 + 11, 1 + 5 + 12, 1 + 6 + 7, 1 + 6 + 8, 1 + 6 + 9, 1 + 6 + 10, 1 + 6 + 11, 1 + 6 + 12, 1 + 7 + 8, |
| | 1 + 7 + 9, 1 + 7 + 10, 1 + 7 + 11, 1 + 7 + 12, 1 + 8 + 9, 1 + 8 + 10, 1 + 8 + 11, 1 + 8 + 12, 1 + 9 + 10, |
| | 1 + 9 + 11, 1 + 9 + 12, 1 + 10 + 11, 1 + 10 + 12, 1 + 11 + 12, 2 + 3 + 4, 2 + 3 + 5, 2 + 3 + 6, 2 + 3 + 7, |
| | 2 + 3 + 8, 2 + 3 + 9, 2 + 3 + 10, 2 + 3 + 11, 2 + 3 + 12, 2 + 4 + 5, 2 + 4 + 6, 2 + 4 + 7, 2 + 4 + 8, 2 + 4 + 9, |
| | 2 + 4 + 10, 2 + 4 + 11, 2 + 4 + 12, 2 + 5 + 6, 2 + 5 + 7, 2 + 5 + 8, 2 + 5 + 9, 2 + 5 + 10, 2 + 5 + 11, 2 + 5 + 12, |
| | 2 + 6 + 7, 2 + 6 + 8, 2 + 6 + 9, 2 + 6 + 10, 2 + 6 + 11, 2 + 6 + 12, 2 + 7 + 8, 2 + 7 + 9, 2 + 7 + 10, 2 + 7 + 11, |
| | 2 + 7 + 12, 2 + 8 + 9, 2 + 8 + 10, 2 + 8 + 11, 2 + 8 + 12, 2 + 9 + 10, 2 + 9 + 11, 2 + 9 + 12, 2 + 10 + 11, |
| | 2 + 10 + 12, 2 + 11 + 12, 3 + 4 + 5, 3 + 4 + 6, 3 + 4 + 7, 3 + 4 + 8, 3 + 4 + 9, 3 + 4 + 10, 3 + 4 + 11, |
| | 3 + 4 + 12, 3 + 5 + 6, 3 + 5 + 7, 3 + 5 + 8, 3 + 5 + 9, 3 + 5 + 10, 3 + 5 + 11, 3 + 5 + 12, 3 + 6 + 7, 3 + 6 + 8, |
| | 3 + 6 + 9, 3 + 6 + 10, 3 + 6 + 11, 3 + 6 + 12, 3 + 7 + 8, 3 + 7 + 9, 3 + 7 + 10, 3 + 7 + 11, 3 + 7 + 12, 3 + 8 + 9, |
| | 3 + 8 + 10, 3 + 8 + 11, 3 + 8 + 12, 3 + 9 + 10, 3 + 9 + 11, 3 + 9 + 12, 3 + 10 + 11, 3 + 10 + 12, 3 + 11 + 12, |
| | 4 + 5 + 6, 4 + 5 + 7, 4 + 5 + 8, 4 + 5 + 9, 4 + 5 + 10, 4 + 5 + 11, 4 + 5 + 12, 4 + 6 + 7, 4 + 6 + 8, 4 + 6 + 9, |
| | 4 + 6 + 10, 4 + 6 + 11, 4 + 6 + 12, 4 + 7 + 8, 4 + 7 + 9, 4 + 7 + 10, 4 + 7 + 11, 4 + 7 + 12, 4 + 8 + 9, |
| | 4 + 8 + 10, 4 + 8 + 11, 4 + 8 + 12, 4 + 9 + 10, 4 + 9 + 11, 4 + 9 + 12, 4 + 10 + 11, 4 + 10 + 12, 4 + 11 + 12, |
| | 5 + 6 + 7, 5 + 6 + 8, 5 + 6 + 9, 5 + 6 + 10, 5 + 6 + 11, 5 + 6 + 12, 5 + 7 + 8, 5 + 7 + 9, 5 + 7 + 10, 5 + 7 + 11, |
| | 5 + 7 + 12, 5 + 8 + 9, 5 + 8 + 10, 5 + 8 + 11, 5 + 8 + 12, 5 + 9 + 10, 5 + 9 + 11, 5 + 9 + 12, 5 + 10 + 11, |
| | 5 + 10 + 12, 5 + 11 + 12, 6 + 7 + 8, 6 + 7 + 9, 6 + 7 + 10, 6 + 7 + 11, 6 + 7 + 12, 6 + 8 + 9, 6 + 8 + 10, |
| | 6 + 8 + 11, 6 + 8 + 12, 6 + 9 + 10, 6 + 9 + 11, 6 + 9 + 12, 6 + 10 + 11, 6 + 10 + 12, 6 + 11 + 12, 7 + 8 + 9, |
| | 7 + 8 + 10, 7 + 8 + 11, 7 + 8 + 12, 7 + 9 + 10, 7 + 9 + 11, 7 + 9 + 12, 7 + 10 + 11, 7 + 10 + 12, 7 + 11 + 12, |
| | 8 + 9 + 10, 8 + 9 + 11, 8 + 9 + 12, 8 + 10 + 11, 8 + 10 + 12, 8 + 11 + 12, 9 + 10 + 11, 9 + 10 + 12, |
| | 9 + 11 + 12, and 10 + 11 + 12. |

In some examples, an LCV domain includes an LCV domain that is at least 90% (e.g., ≥92%, ≥94%, ≥96%, ≥98%, ≥99%, or 100%) identical to the sequence set forth in SEQ ID NO: 518 or SEQ ID NO: 522, where the LCV domain includes a histidine at any of the specific combinations of one or more amino acid positions in SEQ ID NO: 518 or SEQ ID NO: 522 listed in Table 4.

TABLE 4
Exemplary Combinations of Amino Acid Positions in SEQ ID NO:
518 or SEQ ID NO: 522 that can be Substituted with Histidine

Positions 35 and 97.

In some examples, the first ABD comprises an HCV domain comprising the sequence of SEQ ID NO: 512, SEQ ID NO: 516, or one of SEQ ID NOs: 523-571; and/or the first ABD includes an LCV domain comprising the sequence set forth in SEQ ID NO: 518 or SEQ ID NO: 522.

In some examples, the first ABD includes an LCV domain comprising the sequence set forth in SEQ ID NO: 518 or SEQ ID NO: 522, and an HCV domain comprising the sequence set forth in SEQ ID NO: 512 or SEQ ID NO: 516 or one of SEQ ID NOs: 523-567.

In some examples, the first ABD includes an LCV domain comprising the sequence set forth in SEQ ID NO: 518 or SEQ ID NO: 522, and an HCV domain comprising the sequence set forth in SEQ ID NO: 516 or one of SEQ ID NOs: 568-571.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 512.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 516.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 523.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 524.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 525.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 526.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 527.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 528.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 529.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 530.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 531.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 532.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 533.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 534.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 535.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 536.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 537.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 538.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 539.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 540.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 541.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 542.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 543.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 544.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 545.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 546.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 547.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 548.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 549.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 550.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 551.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 552.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 553.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 554.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 555.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 556.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 557.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 558.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 559.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 560.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 561.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 562.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 563.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 564.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 565.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 566.

In some examples, the first ABD includes an LCV domain comprising the sequence set forth in SEQ ID NO: 518 or SEQ ID NO: 518 or SEQ ID NO: 522 and an HCV domain comprising the sequence of SEQ ID NO: 567.

In some examples, the first ABD includes an LCV domain comprising the sequence set forth in SEQ ID NO: 518 or SEQ ID NO: 522 and an HCV domain comprising SEQ ID NO: 512.

In some examples, the first ABD includes an LCV domain comprising the sequence set forth in SEQ ID NO: 518 or SEQ ID NO: 522 and an HCV domain comprising SEQ ID NO: 516.

In some examples, the first ABD includes an LCV domain comprising the sequence set forth in SEQ ID NO: 518 or SEQ ID NO: 522 and an HCV domain comprising SEQ ID NO: 568.

In some examples, the first ABD includes an LCV domain comprising the sequence set forth in SEQ ID NO: 518 or SEQ ID NO: 522 and an HCV domain comprising SEQ ID NO: 569.

In some examples, the first ABD includes an LCV domain comprising the sequence set forth in SEQ ID NO: 518 or SEQ ID NO: 522 and an HCV domain comprising SEQ ID NO: 570.

In some examples, the first ABD includes an LCV domain comprising the sequence set forth in SEQ ID NO: 518 or SEQ ID NO: 522 and an HCV domain comprising SEQ ID NO: 571.

In some examples, the first ABD includes an HCV domain of 29F1 optionally with one or more (e.g., 1, 2, 3, 4, 5, 6, 7, 8, 9, or 10) amino acids substituted with a histidine, an aspartate, or a glutamate.

In some examples, the first ABD includes an LCV domain of 29F1 optionally with one or more (e.g., 1, 2, 3, 4, 5, 6, 7, 8, 9, or 10) amino acids substituted with a histidine, an aspartate, or a glutamate.

In some examples, the first ABD includes an HCV domain of 29F1 optionally with one or more (e.g., 1, 2, 3, 4, 5, 6, 7, 8, 9, or 10) amino acids substituted with a histidine, an aspartate, or a glutamate; and an LCV domain of 29F1 optionally with one or more (e.g., 1, 2, 3, 4, 5, 6, 7, 8, 9, or 10) amino acids substituted with a histidine, an aspartate, or a glutamate.

In some examples, the HCV domain of 29F1 comprises SEQ ID NO: 572 or SEQ ID NO: 572.

In some examples, the LCV domain of 29F1 comprises SEQ ID NO: 577 or SEQ ID NO: 581.

In some examples, the first ABD comprises an HCV domain comprising a CDR1, a CDR2, and a CDR3 of SEQ ID NO: 154, 155, and 156, respectively, optionally with collectively a total of one or more (e.g., 1, 2, 3, 4, 5, 6, 7, 8, 9, or 10) amino acid positions in SEQ ID NOs: 573-575 substituted with a histidine, an aspartate, or a glutamate.

In some examples, the first ABD comprises an LCV domain comprising a CDR1, a CDR2, and a CDR3 of SEQ ID NO: 157, 158, and 159, respectively, optionally with collectively a total of one or more (e.g., 1, 2, 3, 4, 5, 6, 7, 8, 9, or 10) amino acid positions in SEQ ID NOs: 578-580 substituted with a histidine, an aspartate, or a glutamate.

In some examples, the first ABD includes: an HCV domain comprising a CDR1, a CDR2, and a CDR3 of SEQ ID NO: 154, 155, and 156, respectively, optionally with collectively a total of one or more (e.g., 1, 2, 3, 4, 5, 6, 7, 8, 9, or 10) amino acid positions in SEQ ID NOs: 573-575 substituted with a histidine, an aspartate, or a glutamate; and an LCV domain comprising a CDR1, a CDR2, and a CDR3 of SEQ ID NO: 157, 158, and 159, respectively, optionally with collectively a total of one or more (e.g., 1, 2, 3, 4, 5, 6, 7, 8, 9, or 10) amino acid positions in SEQ ID NOs: 578-580 substituted with a histidine, an aspartate, or a glutamate.

In some examples, the first ABD includes an HCV domain that is at least 90% (e.g., ≥92%, ≥94%, ≥96%, ≥98%, ≥99%, or 100%) identical to the sequence set forth in SEQ ID NO: 572 or SEQ ID NO: 576, optionally where the HCV domain includes a histidine, an aspartate, or a glutamate at one or more (e.g., 1, 2, 3, 4, 5, 6, 7, 8, 9, or 10) amino acid positions in SEQ ID NO: 572 or SEQ ID NO: 576 selected from the group of: D positions selected from 31, 56 and 99; and an E position selected from 59. In some examples, the first ABD includes an LCV domain that is at least 90% (e.g., ≥92%, ≥94%, ≥96%, ≥98%, ≥99%, or 100%) identical to the sequence set forth in SEQ ID NO: 577 or SEQ ID NO: 581, optionally where the LCV domain includes a histidine, an aspartate (D), or a glutamate (E) at one or more (e.g., 1, 2, 3, 4, 5, 6, 7, 8, 9, or 10) amino acid positions in SEQ ID NO: 577 or SEQ ID NO: 581 selected from the group of: D positions selected from 27, 28, 31, 52 and 56; or E position selected from 51 and 56. In some examples the first ABD includes: an HCV domain that is at least 90% (e.g., ≥92%, ≥94%, ≥96%, ≥98%, ≥99%, or 100%) identical to the sequence set forth in SEQ ID NO: 572 or SEQ ID NO: 576, optionally where the HCV domain includes a histidine, an aspartate, or a glutamate at one or more (e.g., 1, 2, 3, 4, 5, 6, 7, 8, 9, or 10) amino acid positions in SEQ ID NO: 572 or SEQ ID NO: 576 selected from the group consisting of: 27, 32, 33, 34, 35, 51, 54, 56, 58, and 100, and an LCV domain that is at least 90% (e.g., ≥92%, ≥94%, ≥96%, ≥98%, ≥99%, or 100%) identical to the sequence set forth in SEQ ID NO: 577 or SEQ ID NO: 581, optionally where the LCV domain includes a histidine, an aspartate (D), or a glutamate (E) at one or more (e.g., 1, 2, 3, 4, 5, 6, 7, 8, 9, or 10) amino acid positions in SEQ ID NO: 577 or SEQ ID NO: 581 selected from the group consisting of: D positions selected from 27, 28, 31, 52 and 56; and E positions selected from 51 and 56.

In some examples, an HCV domain includes an HCV domain that is at least 90% (e.g., ≥92%, ≥94%, ≥96%, ≥98%, ≥99%, or 100%) identical to the sequence set forth in SEQ ID NO: 572 or SEQ ID NO: 576, where the HCV domain includes a histidine at any of the specific combinations of one or more amino acid positions in SEQ ID NO: 572 or SEQ ID NO: 576 listed in Table 5.

TABLE 5
Exemplary Combinations of Amino Acid Positions in SEQ ID NO: 572 or SEQ ID
NO: 576 that can be Substituted with Histidine, Aspartate, or Glutamate

Each and every 2, 3, or 4-way combination of the following positions: 31, 56, 59, and 99. For
example, 31 + 56; 31 + 59; 31 + 99; 56 + 59; 56 + 99; 59 + 99; 31 + 56 + 59; 31 + 56 + 99; 31 + 59 + 99;
56 + 59 + 99; 31 + 56 + 59 + 99.

In some examples, an LCV domain includes an LCV domain that is at least 90% (e.g., ≥92%, ≥94%, ≥96%, ≥98%, ≥99%, or 100%) identical to the sequence set forth in SEQ ID NO: 577 or SEQ ID NO: 581, where the LCV domain includes a histidine at any of the specific combinations of one or more amino acid positions in SEQ ID NO: 577 or SEQ ID NO: 581 listed in Table 6.

TABLE 6
Exemplary Combinations of Amino Acid Positions in SEQ ID NO: 577 or SEQ ID
NO: 581 that can be Substituted with Histidine, Aspartate, or Glutamate

| Column | 1 | 2 | 3 | 4 | 5 | 6 |
|---|---|---|---|---|---|---|
| SEQ ID NO: 577 or SEQ ID | 27 | 28 | 31 | 51 | 52 | 56 |
| NO: 581 Position | | | | | | |

| | |
|---|---|
| A | Each and every unique 2-position combination of X and Y, wherein X and are each |
| | selected from a SEQ ID NO: 577 or SEQ ID NO: 581 amino acid position recited in |
| | one of columns 1 to 6, without replacement (i.e., X/Y). Expressed another way, the |
| | combinations of positions recited in the following combinations of columns: 1 + 2, |
| | 1 + 3, 1 + 4, 1 + 5, 1 + 6, 2 + 3, 2 + 4, 2 + 5, 2 + 6, 3 + 4, 3 + 5, 3 + 6, 4 + 5, 4 + 6, and 5 + 6. |
| B | Each and every unique 3-position combination of X, Y, and Z, wherein X and Y are |
| | together one of the 15 2-position combinations recited in “A” (above), and Z is |
| | selected from a SEQ ID NO: 577 or SEQ ID NO: 382 amino acid position recited in |
| | one of columns 1 to 6, without replacement (i.e. X + Y + Z). In other words, 1 + 2 + 3, |
| | 1 + 2 + 4, 1 + 2 + 5, 1 + 2 + 6, 1 + 2 + 7, 1 + 2 + 8, 1 + 2 + 9, 1 + 3 + 4, 1 + 3 + 5, 1 + 3 + 6, 1 + 3 + 7, 1 + 3 + 8, |
| | 1 + 3 + 9, 1 + 4 + 5, 1 + 4 + 6, 1 + 4 + 7, 1 + 4 + 8, 1 + 4 + 9, 1 + 5 + 6, 1 + 5 + 7, 1 + 5 + 8, 1 + 5 + 9, 1 + 6 + 7, |
| | 1 + 6 + 8, 1 + 6 + 9, 1 + 7 + 8, 1 + 7 + 9, 1 + 8 + 9, 2 + 3 + 4, 2 + 3 + 5, 2 + 3 + 6, 2 + 3 + 7, 2 + 3 + 8, 2 + 3 + 9, |
| | 2 + 4 + 5, 2 + 4 + 6, 2 + 4 + 7, 2 + 4 + 8, 2 + 4 + 9, 2 + 5 + 6, 2 + 5 + 7, 2 + 5 + 8, 2 + 5 + 9, 2 + 6 + 7, 2 + 6 + 8, |
| | 2 + 6 + 9, 2 + 7 + 8, 2 + 7 + 9, 2 + 8 + 9, 3 + 4 + 5, 3 + 4 + 6, 3 + 4 + 7, 3 + 4 + 8, 3 + 4 + 9, 3 + 5 + 6, 3 + 5 + 7, |
| | 3 + 5 + 8, 3 + 5 + 9, 3 + 6 + 7, 3 + 6 + 8, 3 + 6 + 9, 3 + 7 + 8, 3 + 7 + 9, 3 + 8 + 9, 4 + 5 + 6, 4 + 5 + 7, 4 + 5 + 8, |
| | 4 + 5 + 9, 4 + 6 + 7, 4 + 6 + 8, 4 + 6 + 9, 4 + 7 + 8, 4 + 7 + 9, 4 + 8 + 9, 5 + 6 + 7, 5 + 6 + 8, 5 + 6 + 9, 5 + 7 + 8, |
| | 5 + 7 + 9, 5 + 8 + 9, 6 + 7 + 8, 6 + 7 + 9, 6 + 8 + 9, and 7 + 8 + 9. |

In some examples, the first ABD comprises an HCV domain comprising the sequence of SEQ ID NO: 572, SEQ ID NO: 576, one of SEQ ID NOs 582-624, or one of SEQ ID NOs: 653-695, and/or the first ABD comprises an LCV domain comprising the sequence of SEQ ID NO: 577, SEQ ID NO: 581, one of SEQ ID NOs: 625-652, or one of SEQ ID NOs: 696-723.

In some examples, the first ABD comprises an LCV domain comprising the sequence of SEQ ID NO: 577, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 581, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 625, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 626, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 627, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 628, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 629, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 630, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 631, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 632, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 633, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 634, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 635, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 636, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 637, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 638, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 639, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 640, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 641, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 642, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 643, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 644, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 645, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 646, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 647, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 648, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 649, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 650, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 651, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 652, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 696, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 697, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 698, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 699, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 700, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 701, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 702, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 703, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 704, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 705, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 706, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 707, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 708, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 709, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 710, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 711, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 712, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 713, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 714, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 715, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 716, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 717, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 718, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 719, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 720, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 721, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 722, and an HCV domain comprising the sequence of SEQ ID NO: 572, SEQ ID NO: 576, one of SEQ ID NOs: 582-624, or one of SEQ ID NOs: 653-695.

In some examples, the first ABD includes an LCV domain comprising the sequence of SEQ ID NO: 723, and an HCV domain comprising the sequence of SEQ ID NO: 572, SEQ ID NO: 576, one of SEQ ID NOs: 582-624, or one of SEQ ID NOs: 653-695.

Also provided herein are pharmaceutical compositions including any of the ABPCs described herein.

Also provided herein are methods of treating a subject in need thereof that include administering a therapeutically effective amount of any of the ABPCs described herein to the subject.

In some examples, a composition including the ABPC (e.g., any of the ABPCs described herein) can provide for an increase (e.g., a detectable increase) (e.g., any of the percents increase or ranges of percents increase recited in WO 2021/022039 at pages 389-404 (or recited in the corresponding U.S. Patent Application No. 2022/0281984), which are incorporated by reference herein in their entirety) in toxin liberation in the target mammalian cell (e.g., any of the target mammalian cells described herein) as compared to a composition including the same amount of a control ABPC (e.g., any of the exemplary control ABPCs described herein).

In some examples, a composition including the ABPC (e.g., any of the ABPCs described herein) can provide for an increase (e.g., a detectable increase) (e.g., any of the folds increase or ranges of folds increase recited in WO 2021/022039 at pages 404-420 (or recited in the corresponding U.S. Patent Application No. 2022/0281984), which are incorporated by reference herein in their entirety) in toxin liberation in the target mammalian cell (e.g., any of the target mammalian cells described herein) as compared to a composition including the same amount of a control ABPC (e.g., any of the exemplary control ABPCs described herein).

In some examples, a composition including the ABPC (e.g., any of the ABPCs described herein) can provide for an increase (e.g., a detectable increase) (e.g., ≥a 1%, 2%, 5%, 10%, 15%, 20%, 25%, 30%, 35%, 40%, 45%, 50%, 55%, 60%, 65%, 70%, 75%, 80%, 85%, 90%, 95%, 100%, 120%, 140%, 160%, 180%, 200%, 250%, 300%, 350%, 400%, 450%, 500%, 1,000%, 2,000%, 3,000%, 4,000%, 5,000%, 6,000%, 7,000%, 8,000%, 9,000%, or 10,000%, or a 1% to a 10,000% increase) in target mammalian cell killing as compared to a composition including the same amount of a control ABPC.

In some examples, a composition including the ABPC (e.g., any of the ABPCs described herein) can provide for an increase (e.g., a detectable increase) (e.g., at least a 0.1-, 0.2-, 0.3-, 0.4-, 0.5-, 0.6-, 0.7-, 0.8-, 0.9-, 1.0-, 1.2-, 1.4-, 1.5-, 1.6-, 1.8-, 2.0-, 2.2-, 2.4-, 2.5-, 2.6-, 2.8-, 3.0-, 3.5-, 4.0-, 4.5-, 5.0-, 5.5-, 6.0-, 6.5-, 7.0-, 7.5-, 8.0-, 8.5-, 9.0-, 9.5-, 10-, 15-, 20-, 25-, 30-, 35-, 40-, 40-, 45-, 50-, 55-, 60-, 65-, 70-, 80-, 85-, 90-, 95-fold increase, or at least a 100-fold increase, or a 0.1-100-fold increase in target mammalian cell killing as compared to a composition including the same amount of a control ABPC.

In some examples, a composition including any of the ABPCs described herein (e.g., upon contacting target mammalian cells presenting LRRC15 on their surface) results in decreased (e.g., ≥a 1%, 5%, 10%, 15%, 20%, 25%, 30%, 35%, 40%, 45%, 50%, 55%, 60%, 65%, 70%, 75%, 80%, 85%, 90%, 95% decrease, or at least a 99% decrease, about a 1%-99% decrease, or any of the subranges of this range described herein) IC50 (for target mammalian cell killing) as compared to the IC50 for a composition including the same amount of a control ABPC.

In some examples, a composition including any of the ABPCs described herein (e.g., upon contacting target mammalian cells presenting LRRC15 on their surface) can provide for an increase (e.g., at least a 0.1-, 0.2-, 0.4-, 0.6-, 0.8-, 1-, 2-, 5-, 10-, 15-, 20-, 25-, 30-, 35-, 40-, 45-, 50-, 55-, 60-, 65-, 70-, 75-, 80-, 85-, 90-, 95-fold increase, or at least a 100-fold increase, or about a 0.1-500-fold increase (or any of the subranges of this range described herein) in the ratio of K_{D} on target mammalian cells presenting LRRC15 on their surface at a neutral pH to IC50 at the neutral pH on the same target cells, e.g., as compared to a control ABPC. In some examples, a composition including the ABPC (e.g., any of the ABPCs described herein) can provide for an increase (e.g., a detectable increase) (e.g., at least a 1%, 2%, 5%, 10%, 15%, 20%, 25%, 30%, 35%, 40%, 45%, 50%, 55%, 60%, 65%, 70%, 75%, 80%, 85%, 90%, 95%, 100%, 120%, 140%, 160%, 180%, 200%, 250%, 300%, 350%, 400%, 450%, 500%, 1,000%, 2,000%, 3,000%, 4,000%, 5,000%, 6,000%, 7,000%, 8,000%, 9,000% increase, or at least a 10,000% increase, or about a 1%-10,000% increase (e.g., or any of the subranges of this range described herein)) in endolysosomal delivery in the target mammalian cell as compared to a composition including the same amount of a control ABPC. In some examples, a composition including the ABPC can provide for an increase (e.g., ≥a 0.1-, 0.2-, 0.3-, 0.4-, 0.5-, 0.6-, 0.7-, 0.8-, 0.9-, 1.0-, 1.2-, 1.4-, 1.5-, 1.6-, 1.8-, 2.0-, 2.2-, 2.4-, 2.5-, 2.6-, 2.8-, 3.0-, 3.5-, 4.0-, 4.5-, 5.0-, 5.5-, 6.0-, 6.5-, 7.0-, 7.5-, 8.0-, 8.5-, 9.0-, 9.5-, 10-, 15-, 20-, 25-, 30-, 35-, 40-, 45-, 50-, 55-, 60-, 65-, 70-, 75-, 80-, 85-, 90-, 95-fold increase, or at least a 100-fold increase, or about a 0.1-100-fold increase in endolysosomal delivery in the target mammalian cell as compared to a composition including the same amount of a control ABPC. In examples of any of the ABPCs described herein, the target mammalian cell does not express an FcRn receptor, or expresses a lower (e.g., a detectably lower) level (e.g., at least a 1% decreased, at least a 2%, 5%, 10% decrease, at least a 15%, 20%, 25%, 30%, 35%, 40%, 45%, 50%, 55%, 60%, 65%, 70%, 75%, 80%, 85%, 90%, 95% decreased, or at least a 99% decreased level) of FcRn receptor as compared to a FcRn expressing control cell (e.g., HUVEC-ThermoFisher #C0035C). In some examples, the target mammalian cell is a cancer cell. In some examples, the ABPC is cytotoxic or cytostatic to the target mammalian cell. In some examples, a composition including any of the ABPCs described herein (e.g., upon administration to a subject) results in less (e.g., a 1% decrease to about a 99% decrease, or any of the subranges of this range described herein) of a reduction in the level of LRRC15 presented on the surface of the target cell as compared to a composition including the same amount of a control ABPC (e.g., any of the control ABPCs described herein). In some examples, the composition does not result in a detectable reduction in the level of the LRRC15 presented on the surface of the target mammalian cell. In some examples, the ABPC is cross-reactive with a non-human primate LRRC15 and a human LRRC15. In some examples, the ABPC is cross-reactive with a non-human primate LRRC15, a human LRRC15, and one or both of rat LRRC15 and a mouse LRRC15. In some examples, the ABPC is cross-reactive with a non-human primate LRRC15, a human LRRC15, a rat LRRC15, and a mouse LRRC15. In some examples, the ABPC is cross-reactive with mouse LRRC15 and rat LRRC15. In some examples, the ABD binds to an epitope of LRRC15 that is present on the surface of cells from an Old World Monkey. Some examples of any of the ABPCs described herein can further include a second ABD (e.g., any of the exemplary ABDs described herein). Non-limiting aspects of these methods are described below and can be used in any combination without limitation. Additional aspects of these methods are known in the art.

### LRRC15 or Epitope of LRRC15

Multiple LRRC15-binding monoclonal antibodies have been described in the literature and can be used as a template for engineering pH-dependent binding. The amino acid sequence of the mature Human LRRC15 can be found in SEQ ID NO: 9. The amino acid sequence of the extracellular domain of LRRC15 can be found in SEQ ID NO: 11.

### Antigen-Binding Protein Constructs

Any of the antigen-binding protein constructs (ABPCs) described herein can be a single polypeptide, or can include 2, 3, 4, 5, 6, 7, 8, 9, or 10 (the same or different) polypeptides. In some embodiments where the ABPC is a single polypeptide, the ABPC can include a single ABD or two ABDs. In some embodiments where the ABPC is a single polypeptide and includes two ABDs, the first and second ABDs can be identical or different from each other (and can specifically bind to the same or different antigens or epitopes).

In some embodiments where the ABPC is a single polypeptide, the first ABD and the second ABD (if present) can each be independently selected from the group of: a VH domain, a VHH domain, a VNAR domain, and a scFv. In some embodiments where the ABPC is a single polypeptide, the antigen-binding protein construct can be a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, a DEP conjugate, a PROTAB, and a PROTAC.

In some embodiments, the ABPC or ABD includes a proteolysis-targeting antibody (PROTAB) (as described, e.g., in Marei et al, “Antibody targeting of E3 ubiquitin ligases for receptor degradation. Nature, 6 Oct. 2022). In some embodiments, a PROTAB or PROTAC may tether cell-surface E3 ubiquitin ligases to transmembrane proteins, resulting in target degradation both in vitro and in vivo.

Additional examples of ABDs that can be used when the ABPC is a single polypeptide are known in the art.

A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the ABPC is a single polypeptide and includes two ABDs, the first ABD and the second ABD can both be VHH domains, or at least one ABD can be a VHH domain. In some embodiments where the ABPC is a single polypeptide and includes two ABDs, the first ABD and the second ABD are both VNAR domains, or at least one ABD is a VNAR domain. In some embodiments where the ABPC is a single polypeptide, the first ABD is a scFv domain. In some embodiments where the ABPC is a single polypeptide and includes two ABDs, the first ABD and the second ABD can both be scFv domains, or at least one ABD can be a scFv domain.

In some embodiments, the ABPC can include two or more polypeptides (e.g., 2, 3, 4, 5, 6, 7, 8, 9, or 10 polypeptides). In some embodiments where the ABPC includes two or more polypeptides, two, three, four, five or six of the polypeptides of the two or more polypeptides can be identical.

In some embodiments where the ABPC includes two or more polypeptides (e.g., 2, 3, 4, 5, 6, 7, 8, 9, or 10 polypeptides), two or more of the polypeptides of the ABPC can assemble (e.g., non-covalently assemble) to form one or more ABDs, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements.

Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment includes a non-covalently-linked dimer of one HCV domain and one LCV domain.

A “Fab” fragment includes the constant domain of the light chain and the first constant domain (C_{H1}) of the heavy chain, in addition to the heavy and LCV domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Additional aspects of ABPCs are known in the art.

### Antigen Binding Domains

In some embodiments of any of the antigen-binding protein constructs (ABPCs) described herein, the dissociation rate of the first ABD (and optionally the second ABD, if present) at a pH of ˜4.0 to ˜6.5 (e.g., ˜4.0 to ˜6.4, or any of the ranges of pH recited in WO 2021/022039 at pages 428-430 (or recited in the corresponding U.S. Patent Application No. 2022/0281984), which are incorporated by reference herein in their entirety) is faster (e.g., at least 5% faster, or any of the % faster or ranges of % faster recited in WO 2021/022039 at pages 430-447 (or recited in the corresponding U.S. Patent Application No. 2022/0281984), which are incorporated by reference herein in their entirety) than the dissociation rate at a pH of ˜7.0 to ˜8.0 (e.g., ˜7.0-7.9, 7.0-7.8, 7.0-7.7, 7.0-7.6, 7.0-7.5, 7.0-7.4, 7.0-7.3, 7.0-7.2, 7.0-7.1, 7.1-8.0, 7.1-7.9, 7.1-7.8, 7.1-7.7, 7.1-7.6, 7.1-7.5, 7.1-7.4, 7.1-7.3, 7.1-7.2, 7.2-8.0, 7.2-7.9, 7.2-7.8, 7.2-7.7, 7.2-7.6, 7.2-7.5, 7.2-7.4, 7.2-7.3, 7.3-8.0, 7.3-7.9, 7.3-7.8, 7.3-7.7, 7.3-7.6, 7.3-7.5, 7.3-7.4, 7.4-8.0, 7.4-7.9, 7.4-7.8, 7.4-7.7, 7.4-7.6, 7.4-7.5, 7.5-8.0, 7.5-7.9, 7.5-7.8, 7.5-7.7, 7.5-7.6, 7.379-381.0, 7.6-7.9, 7.6-7.8, 7.6-7.7, 7.7-8.0, 7.7-7.9, 7.7-7.8, 7.8-8.0, 7.8-7.9, or ˜7.9-8.0).

In some embodiments of any of the antigen-binding protein constructs (ABPCs) described herein, the dissociation constant (K_{D}) of the first ABD (and optionally the second ABD, if present) at a pH of about 4.0 to about 6.5 (e.g., any of the subranges of this range described herein) is greater (e.g., detectably greater) (e.g., at least about 5, 10, 15, 20, 25, 30, 35, 40, 45, 50, 55, 60, 65, 70, 80, 85, 90, 95, 100, 120, 140, 160, 180, 200, 220, 240, 260, 280, 300, 320, 340, 360, 380, 400, 420, 440, 460, 480, 500, 1,000, 2,000, 3,000, 4,000, 5,000, 6,000, 7,000, 8,000, 9,000, or at least about 10,000% greater, or about 5% to about 10,000% greater, or any of the ranges of percents greater K_{D} recited in WO 2021/022039 at pages 448-466 (or recited in the corresponding U.S. Patent Application No. 2022/0281984), which are incorporated by reference herein in their entirety) than the K_{D} at a pH of about 7.0 to about 8.0 (e.g., any of the subranges of this range described herein).

In some embodiments of any of the antigen-binding protein constructs (ABPCs) described herein, the dissociation rate of the first ABD (and optionally the second ABD, if present) at a pH of about 4.0 to about 6.5 (e.g., any of the subranges of this range described herein) is faster (e.g., at least 0.2-fold faster, or any of the folds faster or ranges of folds faster recited in WO 2021/022039 at pages 466-468 (or recited in the corresponding U.S. Patent Application No. 2022/0281984), which are which are incorporated by reference herein in their entirety) than the dissociation rate at a pH of about 7.0 to about 8.0 (e.g., or any of the subranges of this range described herein).

In some embodiments of any of the antigen-binding protein constructs (ABPCs) described herein, the dissociation constant (K_{D}) of the first ABD (and optionally the second ABD, if present) at a pH of about 4.0 to about 6.5 (e.g., any of the subranges of this range described herein) is greater (e.g., detectably greater) (e.g., at least 0.2-fold greater, or any of the fold greater or ranges of fold greater recited in WO 2021/022039 at pages 468-470 (or recited in the corresponding U.S. Patent Application No. 2022/0281984), which are which are incorporated by reference herein in their entirety), than the K_{D} at a pH of about 7.0 to about 8.0 (e.g., any of the subranges of this range described herein).

In some embodiments of the ABPCs that include a first ABD and a second ABD, the first and second ABDs are identical or are at least 80% identical (e.g., ≥82%, ≥84%, ≥86%, ≥88%, ≥90%, ≥91%, ≥92%, ≥93%, ≥94%, ≥95%, ≥96%, ≥97%, ≥98%, or ≥99% identical) in amino acid sequence to each other. In some embodiments, the ABPCs that include a first ABD and a second ABD, the first ABD and the second ABD have a sequence that is less than 80% identical (e.g., <75%, <70%, <65%, <60%, <55%, <50%, <45%, <40%, <35%, <30%, <25%, <20%, <15%, <10%, or <5% identical) to each other. In some embodiments of ABPCs that include a first and a second ABD, the first and second ABD binds two different epitopes (e.g., two different epitopes on LRRC15 or the first ABD binding specifically to LRRC15 and the second ABD binding to an antigen other than LRRC15).

In some embodiments of any of the ABPCs described herein, the K_{D} of the first ABD (and optionally, the second ABD if present) at a pH of about 7.0-8.0 (e.g., any of the subranges of this range described herein) is between about 1 pM-5 μM (e.g., 1 pM-2 μM, or any of the ranges of K_{D} recited in WO 2021/022039 at pages 471-492 (or recited in the corresponding U.S. Patent Application No. 2022/0281984), which are herein incorporated by reference in their entirety).

In some embodiments of any of the ABPCs described herein, the K_{D} of the first ABD (and optionally, the second ABD, if present) at a pH of about 4.0-6.5 (e.g., any of the subranges of this range described herein) can be greater than 1 nM (e.g., between about 1 nM-1 mM, or any of the ranges of K_{D} recited in WO 2021/022039 at pages 492-496 (or recited in the corresponding U.S. Patent Application No. 2022/0281984), which are herein incorporated by reference in their entirety).

A variety of different methods known in the art can be used to determine the K_{D} values of any of the antigen-binding protein constructs described herein (e.g., an electrophoretic mobility shift assay, a filter binding assay, surface plasmon resonance, a biomolecular binding kinetics assay, in vitro binding assay on antigen-expressing cells, etc.).

In some examples, the half-life of the ABPC in vivo is decreased (e.g., a detectable decrease) (e.g., at least a 1% decrease, or any of the percents decrease or ranges of percents decrease recited in WO 2021/022039 at pages 496-500 (or recited in the corresponding U.S. Patent Application No. 2022/0281984), which are herein incorporated by reference in their entirety) as compared to the half-life of a control ABPC (e.g., any of the exemplary control ABPCs described herein). Conversely, in some examples, the half-life of the ABPC in vivo may be increased as compared to the half-life of a control ABPC.

### Conjugation

In some embodiments, the ABPCs provided herein can be conjugated to a drug (e.g., a chemotherapeutic drug, a small molecule), a toxin, or a radioisotope. Non-limiting examples of drugs, toxins, and radioisotopes are known in the art.

In some embodiments, at least one polypeptide of any of the ABPCs described herein is conjugated to the toxin, the radioisotope, or the drug via a cleavable linker. In some embodiments, the cleavable linker includes a protease cleavage site. In some embodiments, the cleavable linker is cleaved on the ABPC once it is transported to the lysosome or late endosome by the target mammalian cell. In some embodiments, cleavage of the linker functionally activates the drug or toxin.

In some embodiments, at least one polypeptide of any of the ABPCs described herein is conjugated to the toxin, the radioisotope, or the drug via a non-cleavable linker. In some embodiments, the conjugated toxin, radioisotope, or drug is released during lysosomal and/or late endosomal degradation of the ABPC.

Non-limiting examples of cleavable linkers include: hydrazone linkers, peptide linkers, disulfide linkers, and thioether linkers. See, e.g., Carter et al., Cancer J. 14 (3): 154-169, 2008; Sanderson et al., Clin. Cancer Res. 11 (2 Pt1): 843-852, 2005; Chari et al., Acc. Chem. Res. 41 (1): 98-107, 2008; Oflazoglu et al., Clin. Cancer Res. 14 (19): 6171-6180, 2008; and Lu et al., Int. J. Mol. Sci. 17 (4): 561, 2016.

Non-limiting examples of non-cleavable linkers include: maleimide alkane-linkers and maleimide cyclohexane linker (MMC) (see, e.g., those described in McCombs et al., AAPS J. 17 (2): 339-351, 2015).

In some embodiments, any of the ABPCs described herein is cytotoxic or cytostatic to the target mammalian cell.

In some embodiments, the antibodies provided herein can comprise one or more amino acid substitutions to provide a conjugation site (e.g., conjugated to a drug, a toxin, a radioisotope). In some embodiments, the antibodies provided herein can have one conjugation site. In some embodiments, the antibodies described herein can have two conjugation sites. In some embodiments, the antibodies provided herein can have three or more conjugation sites. A non-limiting example of an amino acid substitution to produce a conjugation site (e.g., “a triple hinge” conjugation site) is described in U.S. Patent Application No. 2017/0348429, which is incorporated herein by reference in its entirety. For example, a lysine to cysteine substitution at amino acid position 105 and deletion of a threonine at amino acid positions 106 and 108 of SEQ ID NO: 351 or SEQ ID NO: 352 can provide a “triple hinge” conjugation site in any of the antibodies described herein. In some embodiments, an alanine to a cysteine substitution at amino acid position 1 of SEQ ID NO: 351 or 352 can provide a conjugation site for any of the antibodies described herein. In some embodiments, a Val to Cys substitution at position 98 of SEQ ID NO: 353 can provide a conjugation site for any of the antibodies described herein.

Naturally-occurring cysteine amino acids can also provide a conjugation (e.g., conjugated to a drug, a toxin, a radioisotope). In some embodiments, the antibodies provided herein can have a drug, a toxin, or a radioisotope conjugated at one or more (e.g., one, two, three, or four) naturally-occurring conjugation sites. In some embodiments, the cysteine at amino acid position 103 of SEQ ID NO: 351 or 352 is a naturally occurring conjugation site. In some embodiments, the cysteine at amino acid position 109 of SEQ ID NO: 351 or 352 is a naturally occurring conjugation site. In some embodiments, the cysteine at amino acid position 112 of SEQ ID NO: 5 or 6 is a naturally-occurring conjugation site. In some embodiments, the cysteine at amino acid position 107 of SEQ ID NO: 353 is a naturally-occurring conjugation site.

In some embodiments, the antibodies provided herein can have a drug, a toxin, or a radioisotope conjugated at one or more (e.g., two, three, or four) naturally occurring conjugation sites, e.g., the cysteine at amino acid position 103, the cysteine at cysteine at amino acid position 109, and/or the cysteine at amino acid position 112 of SEQ ID NO: 351 or 352, and/or the cysteine at amino acid position 107 of SEQ ID NO: 353. In some embodiments, the antibodies provided herein can have a drug, a toxin, or a radioisotope conjugated at one or more naturally occurring conjugation sites and one or more engineered conjugation sites.

Conjugation through engineered cysteines is achieved by methods known in the art. Briefly, engineered cysteine-containing antibody is prepared for conjugation by treatment with a reducing agent, for example, tris (2-carboxyethyl) phosphine (TCEP), Dithiothreitol (DTT), or 2-Mercaptoethanol (BME). In the reduction reaction the reducing reagent with disulfide bonds in the antibody, breaking interchain disulfides and removing disulfide caps from the engineered cysteines. An optional reoxidation step, achieved by exposure of the solution to air, or an oxidizing agent such as dehydroascorbic acid, allows reformation of the interchain disulfide bonds, leaving the engineered cysteines with a thiolate reactive group. Conjugation with a maleimide functionality on the linker-payload, maleimide-vc-MMAE, is achieved by reaction with the payload in buffered solution, containing cosolvent such as ethanol, dimethylacetamide (DMA), or dimethyl sulfoxide (DMSO). The crude conjugated antibody solution is purified by size exclusion chromatography, or selective filtration methods, such as tangential flow filtration. In this step, residual unreacted payload, reducing agent and oxidizing agents are removed from the reaction mixture, and the conjugated ADC product may be transferred into a desirable formulation buffer.

Conjugation through hinge cysteines is achieved by similar methods, using antibodies with, or without, additional engineered cysteine conjugation sites. Briefly, the antibody is prepared for conjugation by treatment with a reducing agent, for example, tris (2-carboxyethyl) phosphine (TCEP) or Dithiothreitol (DTT). The reducing strength and concentration of the reducing agent are selected such that some or all of the interchain disulfide bonds are reduced leaving free cysteines for conjugation. The solution may be directly conjugated in the presence of excess reducing agent. Conjugation with a maleimide functionality on the linker-payload, maleimide-vc-MMAE, is achieved by reaction with the payload in buffered solution, containing cosolvent such as ethanol, dimethylacetamide (DMA), or dimethyl sulfoxide (DMSO). Unreacted linker-payload may be rendered non-reactive by addition of a sacrificial thiolate molecule such as acetyl-cysteine. The crude conjugated antibody solution may be further purified by methods known in the art, including hydrophobic interaction chromatography, ion-exchange chromatography, or mixed-mode chromatography such as ceramic hydroxyapatite chromatography. Isolation of chromatography fractions allows selection of the desired antibody to payload ratio and removal of unreacted antibody, protein aggregates and fragments, and payload-related reaction side products. The purified antibody drug conjugate may be further purified by size exclusion chromatography, or selective filtration methods, such as TFF. In this step the conjugated ADC product may also be transferred into a desirable formulation buffer.

In some examples, an antibody conjugate can be made comprising an antibody linked to monomethyl auristatin E (MMAE) via a valine-citrulline (vc) linker (hereafter, LRRC15-IgG-DC). Conjugation of the antibody with vcMMAE begins with a partial reduction of the LRRC15-IgG followed by reaction with maleimidocaproyl-Val-Cit-PABC-MMAE (vcMMAE). The LRRC15-IgG (10 mg/mL) is partially reduced by addition of TCEP (molar equivalents of TCEP:mAb is 2:1) followed by incubation at 4° C. overnight. The reduction reaction is then warmed to 25° C. To conjugate all of the thiols, vcMMAE is added to a final vcMMAE:reduced Cys molar ratio of 1:10. The conjugation reaction is carried out in the presence of 10% v/v of Dimethylacetamide (DMA) and allowed to proceed at 25° C. for 60 minutes.

In some examples, an antibody conjugate (ADC) is made comprising the LRRC15-binding IgG (hereafter, LRRC15-IgG) described herein linked to monomethyl auristatin E (MMAE) via a valine-citrulline (vc) linker (hereafter, LRRC15-IgG-DC). Conjugation of the antibody with vcMMAE begins with a partial reduction of the LRRC15-IgG followed by reaction with maleimidocaproyl-Val-Cit-PABC-MMAE (vcMMAE). The LRRC15-IgG (10 mg/mL) is reduced by addition of DTT (molar equivalents of DTT:mAb is 100:1) followed by incubation at 25° C. overnight. The reduced LRRC15-IgG (10 mg/mL) is then re-oxidized by exposure to DHAA (molar equivalents of DHAA:mAb is 10:1) followed by incubation at 25° C. for 2 hours. To conjugate all of the thiols, vcMMAE is added to a final vcMMAE:mAb molar ratio of 4:1. The conjugation reaction is carried out in the presence of 10% v/v of DMA and allowed to proceed at 25° C. for 3 hours.

### Expression of an Antigen-Binding Protein Construct in a Cell

Also provided herein are methods of generating a recombinant cell that expresses an ABPC (e.g., any of the ABPCs described herein) that include: introducing into a cell a nucleic acid encoding the ABPC to produce a recombinant cell; and culturing the recombinant cell under conditions sufficient for the expression of the ABPC. In some embodiments, the introducing step includes introducing into a cell an expression vector including a nucleic acid encoding the ABPC to produce a recombinant cell.

Any of the ABPCs described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. As used herein, the term “prokaryotic cell” refers to a cell that does not have a distinct, membrane-bound nucleus. In some embodiments, the prokaryotic cell is a bacterial cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

Provided herein are methods that further include isolation of the ABPCs from a cell (e.g., a eukaryotic cell) using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

### Avidity

Antibodies and antigen binding fragments thereof are multivalent, and thus comprise more than one binding site. Generally, the measure of total binding strength of an antibody at its binding site is termed avidity. Generally, the terms “fold avidity” and “selectivity” can refer to the fold-difference between the affinity of an antibody and the avidity of an antibody, for example as seen when measuring the total binding strength of an antibody on a cell line with high target expression (avidity; e.g., a cancer cell, e.g., SAOS-2 cells) as compared to the total binding strength of an antibody on a cell line with low target expression (affinity, e.g., a non-cancer cell, e.g., G-292 cells). Generally, avidity is determined by four factors: the binding affinity (e.g., the strength of the binding at an individual binding site); valency (e.g., the total number of binding sites); structural arrangement (e.g., the structure of the antigen and antibody); and antigen density (e.g., the number of antigens per cell).

Provided herein are methods of decreasing the risk of developing a metastasis or decreasing the risk of developing an additional metastasis in a subject having a cancer, where the cancer is characterized by having a population of cancer cells that have LRRC15 or an epitope of LRRC15 presented on their surface the method comprising, administering a therapeutically effective amount of any of the antibodies described herein or any of the pharmaceutical compositions described herein to a subject identified as having a cancer characterized by having the population of cancer cells. In some embodiments, the antibodies described herein can have an at least 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, or 400%, increase in selectivity for a cancer cell as compared to a non-cancer cell. In some embodiments, the cancer is a sarcoma, for example, a leiomyosarcoma, an osteosarcoma, or a chondrosarcoma.

### Methods of Treatment

Provided herein are methods of treating a cancer characterized by having a population of cancer cells that have LRRC15 or an epitope of LRRC15 presented on their surface, that include: administering a therapeutically effective amount of any of the pharmaceutical compositions described herein or any of the ABPCs described herein to a subject identified as having a cancer characterized by having the population of cancer cells.

Also provided herein are methods of reducing the volume of a tumor in a subject, wherein the tumor is characterized by having a population of cancer cells that have LRRC15 or an epitope of LRRC15 presented on their surface, that include: administering a therapeutically effective amount of any of the pharmaceutical compositions described herein or any of the ABPCs described herein to a subject identified as having a cancer characterized by having the population of cancer cells. In some embodiments of any of the methods described herein, the volume of at least one tumor or tumor location is reduced by ≥1, 2, 3, 4, 5, 6, 8, 10, 12, 14, 16, 18, 20, 22, 24, 26, 28, 30, 35, 40, 45, 50, 55, 60, 65, 70, 75, 80, 85, 90, 95, or 99% reduced as compared to the size of the at least one tumor before administration of the ABPC.

Also provided herein are methods of inducing cell death in a cancer cell in a subject, wherein the cancer cell has LRRC15 or an epitope of LRRC15 presented on its surface, that include: administering a therapeutically effective amount of any of the pharmaceutical compositions of described herein or any of the ABPCs described herein to a subject identified as having a cancer characterized as having the population of cancer cells. In some embodiments, the cell death that is induced is necrosis or apoptosis.

In some embodiments of any of the methods described herein, the cancer is a primary tumor. In some embodiments of any of the methods described herein, the cancer is a metastasis. In some embodiments of any of the methods described herein, the cancer is a non-T-cell-infiltrating tumor. In some embodiments of any of the methods described herein, the cancer is a T-cell-infiltrating tumor. In some embodiments of any of the methods described herein, the cellular compartment is part of the endosomal/lysosomal pathway. In some embodiments of any of the methods described herein, the cellular compartment is an endosome. In some embodiments, the cancer is a sarcoma, for example, a leiomyosarcoma, an osteosarcoma, or a chondrosarcoma.

Provided herein are methods of decreasing the risk of developing a metastasis or decreasing the risk of developing an additional metastasis in a subject having a cancer, wherein the cancer is characterized by having a population of cancer cells that have LRRC15 or an epitope of LRRC15 presented on their surface, that include: administering a therapeutically effective amount of any of the pharmaceutical compositions of described herein or any of the ABPCs described herein to a subject identified as having a cancer characterized as having the population of cancer cells. In some embodiments, the risk of developing a metastasis or the risk of developing an additional metastasis is decreased by ≥1, 2, 3, 4, 5, 6, 8, 10, 12, 14, 16, 18, 20, 25, 30, 35, 40, 45, 50, 55, 60, 65, 70, 75, 80, 85, 90, 95, or 99% in the subject as compared to the risk of a subject having a similar cancer, but administered no treatment or a treatment that does not include the administration of any of the ABPCs described herein.

The term “subject” refers to any mammal. In some embodiments, the subject or “subject suitable for treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject suitable for treatment” may be a non-human mammal, especially mammals that are conventionally used as models for demonstrating therapeutic efficacy in humans (e.g., murine, lapine, porcine, canine or primate animals) may be employed.

As used herein, treating includes reducing the number, frequency, or severity of one or more (e.g., two, three, four, or five) signs or symptoms of a cancer in a patient having a cancer (e.g., any of the cancers described herein). For example, treatment can reduce cancer progression, reduce the severity of a cancer, or reduce the risk of re-occurrence of a cancer in a subject having the cancer.

Provided herein are methods of inhibiting the growth of a solid tumor in a subject (e.g., any of the subjects described herein) that include administering to the subject a therapeutically effective amount of any of the ABPCs described herein or any of the pharmaceutical compositions described herein (e.g., as compared to the growth of the solid tumor in the subject prior to treatment or the growth of a similar solid tumor in a different subject receiving a different treatment or receiving no treatment).

In some embodiments of any of the methods described herein, the growth of a solid tumor is primary growth of a solid tumor. In some embodiments of any of the methods described herein, the growth of a solid tumor is recurrent growth of a solid tumor. In some embodiments of any of the methods described herein, the growth of a solid tumor is metastatic growth of a solid tumor. In some embodiments, treatment results in about a 1% decrease to about 99% decrease (or any of the subranges of this range described herein) in the growth of a solid tumor in the subject (e.g., as compared to the growth of the solid tumor in the subject prior to treatment or the growth of a similar solid tumor in a different subject receiving a different treatment or receiving no treatment). The growth of a solid tumor in a subject can be assessed by a variety of different imaging methods, e.g., positron emission tomography, X-ray computed tomography, computed axial tomography, and magnetic resonance imaging.

Also provided herein are methods of decreasing the risk of developing a metastasis or developing an additional metastasis over a period of time in a subject identified as having a cancer (e.g., any of the exemplary cancers described herein) that include administering to the subject a therapeutically effective amount of any of the proteins described herein or any of the pharmaceutical compositions described herein (e.g., as compared to a subject having a similar cancer and receiving a different treatment or receiving no treatment). In some embodiments of any of the methods described herein, the metastasis or additional metastasis is one or more to a bone, lymph nodes, brain, lung, liver, skin, chest wall including bone, cartilage and soft tissue, abdominal cavity, contralateral breast, soft tissue, muscle, bone marrow, ovaries, adrenal glands, and pancreas.

In some embodiments of any of the methods described herein, the period of time is about 1 month to about 3 years (e.g., about 1-30, 1-24, 2-18, 1-12, 1-10, 1-8, 1-6, 1-5, 1-4, 1-3, 1-2, 2-36, 2-30, 2-24, 2-18, 2-12, 2-10, 2-8, 2-6, 2-5, 2-4, 2-3, 3-36, 3-30, 3-24, 3-18, 3-12, 3-10, 3-8, 3-6, 379-381, 3-4, 4-36, 4-30, 4-24, 4-18, 4-12, 4-10, 4-8, 4-6, 4-5, 5-36, 5-30, 5-24, 5-18, 5-12, 5-10, 5-8, 5-6, 6-36, 6-30, 6-24, 6-18, 6-12, 6-10, 379-381, 8-36, 8-30, 8-24, 8-18, 8-12, 8-10, 10-36, 10-30, 10-24, 10-18, 10-12, 12-36, 12-30, 12-24, 12-18, 18-36, 18-30, 18-24, 24-36, 24-30, or about 30-36 months).

In some embodiments, the risk of developing a metastasis or developing an additional metastasis over a period of time in a subject identified as having a cancer is decreased by about 1% to about 99% (e.g., or any of the subranges of this range described herein), e.g., as compared to the risk in a subject having a similar cancer receiving a different treatment or receiving no treatment.

Non-limiting examples of cancer include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor. Additional examples of cancer are known in the art.

In some embodiments, the patient is further administered one or more additional therapeutic agents (e.g., one or more of a chemotherapeutic agent, a recombinant cytokine or interleukin protein, a kinase inhibitor, and a checkpoint inhibitor). In some embodiments, the one or more additional therapeutic agents is administered to the patient at approximately the same time as any of the ABPCs described herein are administered to the patient. In some embodiments, the one or more additional therapeutic agents are administered to the patient after the administration of any of the ABPCs described herein to the patient. In some embodiments, the one or more additional therapeutic agents are administered to the patient before the administration of any of the ABPCs described herein to the patient.

In some embodiments of any of the methods described herein, the cancer is a solid cancer (e.g., breast cancer, prostate cancer, or non-small cell lung cancer). In some embodiments, the cancer is a sarcoma, for example, a leiomyosarcoma, an osteosarcoma, or a chondrosarcoma.

### Compositions

Also provided herein are compositions (e.g., pharmaceutical compositions) that include at least one of any of the ABPCs described herein. In some embodiments, the compositions (e.g., pharmaceutical compositions) can be disposed in a sterile vial or a pre-loaded syringe.

In some embodiments, the compositions (e.g., pharmaceutical compositions) are formulated for different routes of administration (e.g., intravenous, subcutaneous, intramuscular, or intratumoral). In some embodiments, the compositions (e.g., pharmaceutical compositions) can include a pharmaceutically acceptable carrier (e.g., phosphate buffered saline). Single or multiple administrations of any of the pharmaceutical compositions described herein can be given to a subject depending on, for example: the dosage and frequency as required and tolerated by the patient. A dosage of the pharmaceutical composition should provide a sufficient quantity of the ABPC to effectively treat or ameliorate conditions, diseases, or symptoms.

Also provided herein are methods of treating a subject having a cancer (e.g., any of the cancers described herein) that include administering a therapeutically effective amount of at least one of any of the compositions or pharmaceutical compositions provided herein.

### Kits

Also provided herein are kits that include any of the ABPCs described herein, any of the compositions described herein, or any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any pharmaceutical composition as described herein.

### Protein Constructs

Also provided are protein constructs (PCs) that include: a first ABD that is capable of specifically binding LRRC15 or an epitope of LRRC15 presented on the surface of a target mammalian cell, where: (a) the dissociation rate of the first ABD at a pH of ˜7.0 to ˜8.0 (or any of the subranges of this range described herein) is faster than the dissociation rate at a pH of ˜4.0 to ˜6.5 (or any of the subranges of this range described herein); and/or (b) the dissociation constant (K_{D}) of the first ABD at a pH of ˜7.0 to ˜8.0 (or any of the subranges of this range) is greater than the K_{D} at a pH of ˜4.0 to ˜6.5.

Also provided herein are pharmaceutical compositions including any of the PCs described herein. Also provided herein are methods of treating a subject in need thereof that include administering a therapeutically effective amount of any PC described herein.

### Methods of Improving pH Dependence of an Antigen-Binding Protein Construct

Also provided herein are methods of improving pH dependence of an antigen-binding protein construct, the method comprises providing a starting antigen-binding protein construct comprising an ABD and introducing one or more histidine amino acid substitutions into one or more CDRs of the ABD in the starting antigen-binding protein construct, wherein the method results in the generation of an antigen-binding protein construct having one or both of: (a) an increased (e.g., at least a 0.1-fold increase to about a 100-fold increase, or any of the subranges of this range described herein) ratio of the dissociation rate of the ABD at a pH of about 4.0 to about 6.5 to the dissociation rate at a pH of about 7.0 to about 8.0, as compared to the starting antigen-binding protein construct, and (b) an increased (e.g., at least a 0.1-fold increase to about a 100-fold increase, or any of the subranges of this range described herein) ratio the dissociation constant (K_{D}) of the ABD at a pH of about 4.0 to about 6.5 to the K_{D} at a pH of about 7.0 to about 8.0, as compared to the starting antigen-binding protein construct.

The invention is further described in the following examples, which do not limit the scope of the invention described in the claims.

### EXAMPLES

### Example 1. Generation of LRRC15 Binders and Engineering of pH Binding Dependence

pH-engineered ABPCs specific for LRRC15 are generated using multiple methods. In a first approach, published or de novo-identified monoclonal antibodies against LRRC15 may be used as a starting template for introduction of mutations that allow engineering of pH-dependent binding to LRRC15 and i) enhanced endolysosomal accumulation of a conjugated toxin, as well as ii) enhanced LRRC15 recycling to the cell surface. A second approach involves discovery of de novo ABPCs specific for LRRC15 via antibody display methods from naive libraries or libraries with defined CDR compositions and screening under conditions designed for selection of pH-engineered ABPCs specific for LRRC15. In each case, histidine residues play an important role in engineering pH-dependent binding proteins.

Histidine residues are at least partially protonated at a pH below 6.5 owing to its pKa of 6.0. Therefore, if a histidine side chain in an antigen-binding domain participates in an electrostatic binding interaction with its antigen it will start to turn positively charged at a pH at or below 6.5. This could either weaken or enhance the binding affinity of the interaction at a pH below 6.5, based on the corresponding charge of and interactions with the antigen epitope. Thus, systematic introduction of histidines into antibody complementarity determining regions (CDRs) in an antibody or other binder library (e.g., an scFv library) can be used to identify substitutions that will affect an antigen-binding domain's interaction with an antigen at lower pH values. The first approach therefore involves histidine-scanning of variable domain sequences of published monoclonal antibodies to identify pH-dependent variants.

Multiple LRRC15-binding monoclonal antibodies have been described in the literature and can be used as a template for engineering pH-dependent binding Purcell et. al., LRRC15 Is a Novel Mesenchymal Protein and Stromal Target for Antibody-Drug Conjugates, Cancer Res. 78 (14): 4059-4072 (2018). Briefly, for a subset of the antibody sequences, CDRs in each chain are identified using the methods described by Kabat et al (Kabat et al. (1992) Sequences of Proteins of Immunological Interest (DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions are called as CDR residues. To engineer pH-dependent sequence variants, individual amino acid residues within the heavy chain and/or light chain CDRs are systematically substituted with a histidine, one at a time. In cases where the starting CDR residue is a histidine, it is mutated to an alanine. Antibody variants with only one histidine or alanine mutation in a heavy/light chain CDR are generated by co-transfection of Expi293 cells with a) one heavy chain or light chain sequence variant, and b) the corresponding starting ABPC (e.g., the starting LRRC15-binding monoclonal antibody) light chain or heavy chain, respectively, using methods known to the art.

After allowing for a period of protein expression, cell culture supernatants are collected, quantified, and the pH dependence of the variant is evaluated using biolayer interferometry (BLI) or other methods known to the art. Briefly, cell culture supernatants are normalized to an antibody expression level of 50 μg/mL, and captured on an anti-human Fc sensor (Forte Bio). A baseline is established using 1× kinetics buffer (Forte Bio), and the sensor is associated with 100 nM of LRRC15 in 1×PBS at pH 7.4 for 300 sec to generate an association curve. In the dissociation phase, the antibody-antigen complex on the sensor is exposed to 1×PBS at either pH 5.5 or pH 7.4 for 300-500 sec. Association and dissociation phase curves are examined for the starting ABPC antibody and each corresponding antibody variant at pH 5.5 and pH 7.4 to inform on two criteria: a) enhanced dissociation (i.e., higher koff values) at pH 5.5 due to histidine or alanine substitution compared to the starting ABPC, and b) reduced dissociation at pH 7.4 (i.e., lower koff values) compared to pH 5.5 in the antibody variant itself and with the starting ABPC. Variants that show either enhanced dissociation at pH 5.5 or reduced dissociation at pH 7.4 or both are selected for further analysis. It is also noted that while some histidine and alanine mutations obliterate LRRC15 binding, others are tolerated with little (e.g., less than 1-fold change in K_{D} or dissociation rate) or no change in LRRC15 binding kinetics. Especially because histidine is a large, positively charged amino acid, these histidine variants and alanine variants with no change are noted as positions that may tolerate a wide range of mutations and lead to antibodies with different sequence but similar binding properties, a designation that is not otherwise apparent. The variants selected for further analysis are expressed at a larger scale and purified using Protein A affinity chromatography. Binding kinetics (kon and koff) of the purified starting ABPC and variant antibodies are measured at pH 5.5 and pH 7.4 using Biacore (GE Healthcare). The ratio of the antibody's rate of dissociation (koff at pH 7.4 divided by koff at pH 5.5) is also used as a quantitative assessment of pH-dependent binding; similarly, the dissociation constant K_{D} is calculated at both pH 5.5 and pH 7.4 as koff divided by kon and the ratio of the antibody's dissociation constant (K_{D} at pH 7.4 divided by K_{D} at pH 5.5) is also used as a quantitative assessment of pH-dependent binding.

Antibodies with a rate of dissociation ratio less than that of the starting ABPC and/or a dissociation constant ratio less than that of the starting ABPC are selected for further assessment of combinatorial substitutions. Favorable histidine and/or alanine amino acid positions can also be combined to enhance pH dependence; this can be done by, e.g., combinatorially or rationally combining histidine and/or alanine substitutions on a given heavy or light chain that individually improve pH dependence, by, e.g., combinatorially or rationally combining modified heavy and light chains such that histidine and/or alanine substitutions are present on both chains, or combinations thereof. Such combinatorial variants are generated and tested/analyzed for differential pH dependence using the methods and protocols described herein, or others known to the art. Antibody variants that have the lowest rate of dissociation ratios and/or dissociation constant ratios are selected as candidates for further analysis (hereafter referred to as “pH-engineered ABPCs specific for LRRC15”).

The second method for selection of pH-engineered ABPCs specific for LRRC15 involves either screening libraries to identify de novo pH-dependent ABPCs specific for LRRC15 or ABPCs that could serve as templates for engineering pH-dependent binding as described herein. Two types of libraries can be used for these selections: naive phage/yeast display antibody libraries (e.g., Fab, scFv, VHH, VL, or others known to the art) or phage/yeast display libraries where CDRs have been mutated to express a subset of amino acid residues. Libraries are screened against soluble recombinant LRRC15 extracellular domains using methods known to the art with positive selection for variants that bind weakly (e.g., are eluted from beads) at pH 5.0 and bind strongly (e.g., are bound to beads) at pH 7.4. Three rounds of selections are performed. The final round of binders are screened using ELISA for binding to human LRRC15 and cyno LRRC15 and mouse LRRC15 or via mean fluorescence intensity in flow cytometric analysis. If more binders with cyno or murine cross-reactivity are desired, the final selection round can instead be performed on cyno LRRC15 or murine LRRC15. Selected binding proteins are subcloned into mammalian expression vectors and expressed as either full IgG proteins or Fc fusions in Expi293 cells. BLI analysis is performed as described herein for selection of pH-dependent binder variants and confirmed using Biacore.

### Production of the 15G7, 24D9, 29F1 Anti-LRRC15 Monoclonal Antibodies

Applicant inoculated rabbits with the extracellular domain of human LRRC15 protein to produce 250 rabbit mAbs (RabMAbs). After the initial production of b-cell clones, cell supernatants were evaluated in a secondary cytotoxicity assay. Each Ab that killed the cells was sequenced, procured as a chimeric antibody (i.e., rabbit variable domains with human constant regions), expressed, and then re-assayed for cytotoxicity, internalization, and other properties. Top candidates were humanized and, in parallel, histidine-, aspartate-, glutamate-, and other amino acid-scan variants of the chimeric antibodies were produced as described in U.S. Patent Application No. US 2022/0281984, which is incorporated by reference herein in its entirety.

Once promising RabMAb mutations and humanized frameworks were identified, the mutations were transferred to the humanized frameworks, and the resulting humanized mutant antibodies were re-tested for activity and developability. The most promising humanized mutant Abs were then conjugated with toxic payload (see, e.g., above paragraphs [0495-0496]) to produce candidate anti-LRRC15 ADCs, which were then evaluated for stability, activity and developability. Higher-performing candidates are listed in Table 7 and additional details are presented in Example 23 (below).

TABLE 7
Selected anti-LRRC15 antibodies exhibiting enhanced dissociation
at acidic pH, enhanced internalization, and increased cytotoxicity.
VH=heavy chain variable domain; VL=light chain variable domain

| | | | VH | | VL |
|---|---|---|---|---|---|
| | Parent | HC | Seq | LC | Seq |
| MYT# | Molecule | Descriptor | ID | Descriptor | ID |
| 2737 | sam | sam HC | 1 | LC His Var | 64 |
| 3315 | hu139.10 | hu139.10 HC | 84 | E68H | 154 |
| 8391 | hu15G7 | V106H | 430 | hu 15G7 LC | 455 |
| 8415 | hu15G7 | hu15G7 HC | 382 | Y108H | 489 |
| 8417 | hu15G7 | hu15G7 HC | 382 | G112H | 491 |
| 8483 | hu24D9 | hu24D9 HC | 516 | hu24D9 LC | 522 |
| 8489 | hu24D9 | hu24D9 HC Dev | 569 | hu24D9 LC | 522 |
| 8485 | hu24D9 | hu24D9 HC Dev | 568 | hu24D9 LC | 522 |
| 8500 | hu24D9 | hu24D9 HC Dev | 570 | hu24D9 LC | 522 |
| 9609 | hu24D9 | hu24D9 HC | 516 | FR4 Hum | 522 |
| 9776 | hu24D9 | HC S32H, N53H, | 571 | LC EIK | 517 |
| | | L62H | | | |
| 8094 | hu29F1 | hu29F1 HC | 576 | hu29F1 LC | 581 |
| 9521 | hu29F1 | hu29F1 HC | 576 | T66D | 639 |
| 9731 | hu29F1 | hu29F1 HC | 576 | N28E | 700 |
| 8416 | hu15G7 | hu15G7 HC | 382 | W109H | 490 |
| 8500 | hu24D9 | HC L57H | 570 | hu24D9 LC | 522 |
| 9523 | hu29F1 | hu29F1 HC | 576 | A68D | 641 |
| 4174 | hu139.10 | hu139.10 HC | 84 | W56H, E68H, | 177 |
| | | | | L115H | |
| 9507 | hu29F1 | hu29F1 HC | 576 | Q24D | 625 |

Tables 8-13 provide details for constructs of particular utility during the humanization process. And while the development candidates disclosed in Table 7 (above) are among the most advanced, many useful intermediate forms were produced during applicant's efforts to improve the developability of the binding proteins disclosed herein. Developability modifications include those that tend to improve Ab solubility, reduce aggregation, and optimize in vivo half-life. Now that applicant has fully disclosed the various “developability modifications” that are tolerated and/or beneficial, the skilled person may routinely produce similar binding proteins having desirable, drug-like properties. The Tables immediately below showcase some of these developability constructs and properties associated therewith.

TABLE 8
Selected humanized anti-LRRC15 binding proteins

| | | | | | HC Seq | LC Seq |
|---|---|---|---|---|---|---|
| MYT# | Parent | HC Seq | LC Seq | MYT# | ID | ID |
| (in IgG1) | Molecule | ID | ID | (w/ F405L) | (F405L) | (F405L) |
| MYT8385 | 15G7 | 382 | 455 | MYT6910 | 382 F405L | 455 |
| MYT8483 | 24D9 | 516 | 522 | MYT6929 | 516 F405L | 522 |
| MYT8094 | 29F1 | 576 | 581 | N/A | | |

TABLE 9
Selected histidine substituted anti-LRRC15 chimeric RabMAbs

| MYT# | Parent Molecule | HC Seq ID | LC Seq ID |
|---|---|---|---|
| MYT6116 | 15G7 | 419 | 451 |
| MYT6143 | 15G7 | 378 | 477 |
| MYT6147 | 15G7 | 378 | 481 |
| MYT7887 | 24D9 | 529 | 518 |
| MYT7894 | 24D9 | 536 | 518 |
| MYT7898 | 24D9 | 540 | 518 |

TABLE 10
Selected chimeric anti-LRRC15 RabMAbs

| MYT# | Parent Molecule | HC Seq ID | LC Seq ID |
|---|---|---|---|
| MYT5712 | 15G7 | 378 | 451 |
| MYT5742 | 24D9 | 512 | 518 |
| MYT8088 | 29F1 | 572 | 577 |

TABLE 11
Internalization of Abs in U87-MG at 10 nM at 24 hr or Cytotoxicity at 20 nM
(FOS = Fold over samrotamab; Cytotox = % Killing at 20 nM)

| MYT# of | Parent | | | |
|---|---|---|---|---|
| mAb | Molecule | Molecule Descriptor | FOS | Cytotox |
| | | Non-Binding ADC Control | −0.06 | 3.59 |
| MYT5438 | | Sam. ADC, Hinge Conjugated, DAR 2.0 | | 16.44 |
| MYT2737 | Samrotamab | Sam. LC His Variant | 1.61 | 19.22 |
| MYT3315 | hu139.10 | hu139.10 LC His Variant | 2.01 | 16.22 |
| MYT8391 | hu15G7 | hu 15G7 HC his scan variant | 2.13 | 23.65 |
| MYT8415 | hu15G7 | hu 15G7 LC his scan variant | 1.84 | 38.73 |
| MYT8416 | hu15G7 | hu 15G7 LC his scan variant | 1.84 | 23.22 |
| MYT8417 | hu15G7 | hu 15G7 LC his scan variant | 2.06 | 42.46 |
| MYT8483 | hu24D9 | Parental | 2 | 29.43 |
| MYT8500 | hu24D9 | hu 24D9 HC Hydrophobicity Variant | 1.86 | 23.62 |
| MYT9776 | hu24D9 | hu24D9 HC Dev Combos, + LC FR4 EIK | 1.51 | 66.26 |
| MYT8094 | hu29F1 | hu29F1 Parental | 1.93 | 27.36 |
| MYT9521 | hu29F1 | hu29F1 LC Asp Var | 2.1 | 45.38 |
| MYT9523 | hu29F1 | hu29F1 LC Asp Var | 2.01 | 40.11 |
| MYT9731 | hu29F1 | hu29F1 LC Glu Var | 2.05 | 41.16 |

TABLE 12
Affinity of selected binding proteins for LRRC15 positive cell line SAOS2, G292

| MYT# of | | SAOS2 | SAOS2 | G292 | G292 |
|---|---|---|---|---|---|
| mAb | Molecule Descriptor | pH 7.4 | pH 6.4 | pH 7.4 | pH 6.4 |
| | Non-Binding ADC Control | | | | |
| MYT5438 | Sam. ADC, Hinge (DAR 2.0) | 0.3105 | 0.3364 | 0.1654 | 0.1873 |
| MYT2737 | Sam. LC His Variant | 0.8397 | 0.9198 | 0.5277 | 0.6762 |
| MYT3315 | hu139.10 LC His Variant | 0.2957 | 0.3978 | 0.1548 | 0.2505 |
| MYT8391 | hu 15G7 HC his variant | 0.4834 | N/A | 0.3361 | N/A |
| MYT8415 | hu 15G7 LC his variant | 0.7543 | N/A | 1.112 | N/A |
| MYT8416 | hu 15G7 LC his variant | 1.148 | N/A | 1.374 | N/A |
| MYT8417 | hu 15G7 LC his variant | 0.5545 | N/A | 0.7605 | N/A |
| MYT8483 | Parental | 1.645 | N/A | 2.072 | N/A |
| MYT8500 | hu 24D9 HC Hydrophobicity Variant | 1.218 | N/A | 2.213 | N/A |
| MYT9776 | hu24D9 HC Dev Combos, +LC FR4 EIK | 7.323 | N/A | N/A | N/A |
| MYT8094 | hu29F1 Parental | 1.709 | N/A | N/A | N/A |
| MYT9521 | hu29F1 LC Asp Var | 1.577 | N/A | N/A | N/A |
| MYT9523 | hu29F1 LC Asp Var | 0.8168 | N/A | N/A | N/A |
| MYT9731 | hu29F1 LC Glu Var | 0.3518 | N/A | N/A | N/A |

TABLE 13
Binding of selected proteins to LRRC15 neg. cell line Ramos (measured by MFI)

| MYT# of mAb | Molecule Descriptor | Average MFI |
|---|---|---|
| | Non-Binding ADC Control | 474 |
| MYT5438 | samrotamab ADC, Hinge (DAR 2.0) | 474 |
| MYT2737 | samrotamab LC His Var | 521 |
| MYT3315 | hu139.10 LC His Var | 532 |
| MYT8391 | hu 15G7 HC His Var | 412 |
| MYT8415 | hu 15G7 LC His Var | 474 |
| MYT8416 | hu 15G7 LC His Var | 463 |
| MYT8417 | hu 15G7 LC His Var | 608.5 |
| MYT8483 | Parental | 456 |
| MYT8500 | hu 24D9 HC Hydrophobicity Variant | 481.5 |
| MYT9776 | hu24D9 HC Dev Combos, +LC FR4 EIK | 481 |
| MYT8094 | hu29F1 Parental | 481 |
| MYT9521 | hu29F1 LC Asp Var | 481.5 |
| MYT9523 | hu29F1 LC Asp Var | 470.5 |
| MYT9731 | hu29F1 LC Glu Var | 524.5 |

| Controls | | |
|---|---|---|
| Cells Only | 398 | 394.5 |
| Secondary Only | 456 | 445 |
| Positive Control | 13973 | 14537.5 |

TABLE 14
Shows selected antibodies (by MYT number)
along with component parts (by SEQ ID NO).

| MYT | VH | VL | MYT | VH | VL | MYT | VH | VL |
|---|---|---|---|---|---|---|---|---|
| 5712 | 378 | 451 | 8397 | 436 | 455 | 8420 | 382 | 494 |
| | 382 | 455 | 8398 | 437 | 455 | 8421 | 382 | 495 |
| 6080 | 383 | 451 | 8399 | 438 | 455 | 8422 | 382 | 496 |
| 6081 | 384 | 451 | 8400 | 439 | 455 | 8423 | 382 | 497 |
| 6082 | 385 | 451 | 8401 | 440 | 455 | 8424 | 382 | 498 |
| 6083 | 386 | 451 | 8402 | 441 | 455 | 8425 | 382 | 499 |
| 6084 | 387 | 451 | 8403 | 442 | 455 | 8426 | 382 | 500 |
| 6085 | 388 | 451 | 8404 | 443 | 455 | 8427 | 382 | 501 |
| 6086 | 389 | 451 | 8405 | 444 | 455 | 8428 | 382 | 502 |
| 6087 | 390 | 451 | 8406 | 445 | 455 | 8429 | 382 | 503 |
| 6088 | 391 | 451 | 8407 | 446 | 455 | 8430 | 382 | 504 |
| 6089 | 392 | 451 | 8408 | 447 | 455 | 8431 | 382 | 505 |
| 6090 | 393 | 451 | 8409 | 448 | 455 | 8432 | 382 | 506 |
| 6091 | 394 | 451 | 8410 | 449 | 455 | 8433 | 382 | 507 |
| 6092 | 395 | 451 | 8411 | 450 | 455 | 8434 | 382 | 508 |
| 6093 | 396 | 451 | 5712 | 378 | 451 | 8435 | 382 | 509 |
| 6094 | 397 | 451 | 5712 | 382 | 455 | 8436 | 382 | 510 |
| 6095 | 398 | 451 | 6122 | 378 | 456 | 8437 | 382 | 511 |
| 6096 | 399 | 451 | 6123 | 378 | 457 | 8483 | 516 | 522 |
| 6097 | 400 | 451 | 6124 | 378 | 458 | 9776 | 516 | 517 |
| 6098 | 401 | 451 | 6125 | 378 | 459 | 8483 | 516 | 522 |
| 6099 | 402 | 451 | 6126 | 378 | 460 | 7881 | 523 | 518 |
| 6100 | 403 | 451 | 6127 | 378 | 461 | 7882 | 524 | 518 |
| 6101 | 404 | 451 | 6128 | 378 | 462 | 7883 | 525 | 518 |
| 6102 | 405 | 451 | 6129 | 378 | 463 | 7884 | 526 | 518 |
| 6103 | 406 | 451 | 6130 | 378 | 464 | 7885 | 527 | 518 |
| 6104 | 407 | 451 | 6131 | 378 | 465 | 7886 | 528 | 518 |
| 6105 | 408 | 451 | 6132 | 378 | 466 | 7887 | 529 | 518 |
| 6106 | 409 | 451 | 6133 | 378 | 467 | 7888 | 530 | 518 |
| 6107 | 410 | 451 | 6134 | 378 | 468 | 7889 | 531 | 518 |
| 6108 | 411 | 451 | 6135 | 378 | 469 | 7890 | 532 | 518 |
| 6109 | 412 | 451 | 6136 | 378 | 470 | 7891 | 533 | 518 |
| 6110 | 413 | 451 | 6137 | 378 | 471 | 7892 | 534 | 518 |
| 6111 | 414 | 451 | 6138 | 378 | 472 | 7893 | 535 | 518 |
| 6112 | 415 | 451 | 6139 | 378 | 473 | 7894 | 536 | 518 |
| 6113 | 416 | 451 | 6140 | 378 | 474 | 7895 | 537 | 518 |
| 6114 | 417 | 451 | 6141 | 378 | 475 | 7896 | 538 | 518 |
| 6115 | 418 | 451 | 6142 | 378 | 476 | 7897 | 539 | 518 |
| 6116 | 419 | 451 | 6143 | 378 | 477 | 7898 | 540 | 518 |
| 6117 | 420 | 451 | 6144 | 378 | 478 | 7899 | 541 | 518 |
| 6118 | 421 | 451 | 6145 | 378 | 479 | 7900 | 542 | 518 |
| 6119 | 422 | 451 | 6146 | 378 | 480 | 7901 | 543 | 518 |
| 6120 | 423 | 451 | 6147 | 378 | 481 | 7902 | 544 | 518 |
| 6121 | 424 | 451 | 6148 | 378 | 482 | 7903 | 545 | 518 |
| 8386 | 425 | 455 | 6149 | 378 | 483 | 7904 | 546 | 518 |
| 8387 | 426 | 455 | 6150 | 378 | 484 | 7905 | 547 | 518 |
| 8388 | 427 | 455 | 6151 | 378 | 485 | 7906 | 548 | 518 |
| 8389 | 428 | 455 | 8412 | 382 | 486 | 7907 | 549 | 518 |
| 8390 | 429 | 455 | 8413 | 382 | 487 | 7908 | 550 | 518 |
| 8391 | 430 | 455 | 8414 | 382 | 488 | 7909 | 551 | 518 |
| 8392 | 431 | 455 | 8415 | 382 | 489 | 7910 | 552 | 518 |
| 8393 | 432 | 455 | 8416 | 382 | 490 | 7911 | 553 | 518 |
| 8394 | 433 | 455 | 8417 | 382 | 491 | 7912 | 554 | 518 |
| 8395 | 434 | 455 | 8418 | 382 | 492 | 7913 | 555 | 518 |
| 8396 | 435 | 455 | 8419 | 382 | 493 | 7914 | 556 | 518 |
| 7915 | 557 | 518 | 9500 | 618 | 581 | 9702 | 671 | 581 |
| 7916 | 558 | 518 | 9501 | 619 | 581 | 9703 | 672 | 581 |
| 7917 | 559 | 518 | 9502 | 620 | 581 | 9704 | 673 | 581 |
| 7918 | 560 | 518 | 9503 | 621 | 581 | 9705 | 674 | 581 |
| 7919 | 561 | 518 | 9504 | 622 | 581 | 9706 | 675 | 581 |
| 7920 | 562 | 518 | 9505 | 623 | 581 | 9707 | 676 | 581 |
| 7921 | 563 | 518 | 9506 | 624 | 581 | 9708 | 677 | 581 |
| 7922 | 564 | 518 | 9507 | 576 | 625 | 9709 | 678 | 581 |
| 7923 | 565 | 518 | 9508 | 576 | 626 | 9710 | 679 | 581 |
| 7924 | 566 | 518 | 9509 | 576 | 627 | 9711 | 680 | 581 |
| 7925 | 567 | 518 | 9510 | 576 | 628 | 9712 | 681 | 581 |
| 8485 | 568 | 522 | 9511 | 576 | 629 | 9713 | 682 | 58 |
| 8489 | 569 | 522 | 9512 | 576 | 630 | 9714 | 683 | 581 |
| 8500 | 570 | 522 | 9513 | 576 | 631 | 9715 | 684 | 581 |
| 9776 | 571 | 522 | 9514 | 576 | 632 | 9716 | 685 | 581 |
| 8094 | 576 | 581 | 9515 | 576 | 633 | 9717 | 686 | 581 |
| 8094 | 576 | 581 | 9516 | 576 | 634 | 9718 | 687 | 581 |
| 9464 | 582 | 581 | 9517 | 576 | 635 | 9719 | 688 | 581 |
| 9465 | 583 | 582 | 9518 | 576 | 636 | 9720 | 689 | 581 |
| 9466 | 584 | 583 | 9519 | 576 | 637 | 9721 | 690 | 581 |
| 9467 | 585 | 581 | 9520 | 576 | 638 | 9722 | 691 | 581 |
| 9468 | 586 | 581 | 9521 | 576 | 639 | 9723 | 692 | 581 |
| 9469 | 587 | 581 | 9522 | 576 | 640 | 9724 | 693 | 581 |
| 9470 | 588 | 581 | 9523 | 576 | 641 | 9725 | 694 | 581 |
| 9471 | 589 | 581 | 9524 | 576 | 642 | 9726 | 695 | 581 |
| 9472 | 590 | 581 | 9525 | 576 | 643 | 9727 | 576 | 696 |
| 9473 | 591 | 581 | 9526 | 576 | 644 | 9728 | 576 | 697 |
| 9474 | 592 | 581 | 9527 | 576 | 645 | 9729 | 576 | 698 |
| 9475 | 593 | 581 | 9528 | 576 | 646 | 9730 | 576 | 699 |
| 9476 | 594 | 581 | 9529 | 576 | 647 | 9731 | 576 | 700 |
| 9477 | 595 | 581 | 9530 | 576 | 648 | 9732 | 576 | 701 |
| 9478 | 596 | 581 | 9531 | 576 | 649 | 9733 | 576 | 702 |
| 9479 | 597 | 581 | 9532 | 576 | 650 | 9734 | 576 | 703 |
| 9480 | 598 | 581 | 9533 | 576 | 651 | 9735 | 576 | 704 |
| 9481 | 599 | 581 | 9534 | 576 | 652 | 9736 | 576 | 705 |
| 9482 | 600 | 581 | 9684 | 653 | 581 | 9737 | 576 | 706 |
| 9483 | 601 | 581 | 9685 | 654 | 581 | 9738 | 576 | 707 |
| 9484 | 602 | 581 | 9686 | 655 | 581 | 9739 | 576 | 708 |
| 9485 | 603 | 581 | 9687 | 656 | 581 | 9740 | 576 | 709 |
| 9486 | 604 | 581 | 9688 | 657 | 581 | 9741 | 576 | 710 |
| 9487 | 605 | 581 | 9689 | 658 | 581 | 9742 | 576 | 711 |
| 9488 | 606 | 581 | 9690 | 659 | 581 | 9743 | 576 | 712 |
| 9489 | 607 | 581 | 9691 | 660 | 581 | 9744 | 576 | 713 |
| 9490 | 608 | 581 | 9692 | 661 | 581 | 9745 | 576 | 714 |
| 9491 | 609 | 581 | 9693 | 662 | 581 | 9746 | 576 | 715 |
| 9492 | 610 | 581 | 9694 | 663 | 581 | 9747 | 576 | 716 |
| 9493 | 611 | 581 | 9695 | 664 | 581 | 9748 | 576 | 717 |
| 9494 | 612 | 581 | 9696 | 665 | 581 | 9749 | 576 | 718 |
| 9495 | 613 | 581 | 9697 | 666 | 581 | 9750 | 576 | 719 |
| 9496 | 614 | 581 | 9698 | 667 | 581 | 9751 | 576 | 720 |
| 9497 | 615 | 581 | 9699 | 668 | 581 | 9752 | 576 | 721 |
| 9498 | 616 | 581 | 9700 | 669 | 581 | 9753 | 576 | 722 |
| 9499 | 617 | 581 | 9701 | 670 | 581 | 9754 | 576 | 723 |

VH = heavy chain variable domain;
VL = light chain variable domain

### Example 2. In Vitro Demonstration of pH-Dependent Binding to LRRC15, pH-Dependent Release of LRRC15, Enhanced Endolysosomal Delivery in LRRC15+ Cells, and Increased LRRC15 Antigen Density in LRRC15+ Cells after Exposure to pH-Engineered ABPCs Specific for LRRC15 as Compared to Control ABPCs Specific for LRRC15

As discussed herein, pH-engineered ABPCs specific for LRRC15 exhibit the desirable property of decreased LRRC15 binding at acidic pH (e.g., about pH 5.0 to about pH 6.4), but enhanced binding at higher, for example, physiological pH (e.g., pH 7.4), which enhances their accumulation in endolysosomes under physiological conditions.

pH-Dependent Binding to LRRC15 on Cells

To demonstrate that pH-engineered ABPCs specific for LRRC15 binds cell surface LRRC15 at neutral pH, a cell surface binding assay is performed. A panel of human cells that are LRRC15+ is assembled. Methods of identifying and quantifying gene expression (e.g., LRRC15) for a given cell line are known to the art, and include, e.g., consulting the Cancer Cell Line Encyclopedia (CCLE; portals.broadinstitute.org/ccle) to ascertain the expression level and/or mutation status of a given gene in a tumor cell line), rtPCR, microarray, or RNA-Seq analysis, or cell staining with antibodies known in the art (e.g., (Recombinant Anti-LRRC15 antibody, Abcam Cat #ab150376 clone EPR8188 (2); Hi-Affi™ Recombinant Rabbit Anti-LRRC15 Monoclonal Antibody, Creative Biolabs Cat #MOR-2090 Clone #DS2090AB for LRRC15). Cells are seeded at approximately 5-10,000 per well in 150 μL of pH 7.4 culture medium and incubated at 37° C. for 5 minutes at several doses (e.g., a two-fold dilution series) from 1 pM to 1 μM with one of the following antibodies: a known, control ABPC specific for LRRC15 (e.g., an antibody, samrotamab, hu139.10, huAD208.4.1, huAD208.12.1, 1-13C3, or 1-19G12), the pH-engineered ABPC specific for LRRC15, and an appropriate negative isotype control mAb (e.g., Biolegend Purified Human IgG1 Isotype Control Recombinant Antibody, Cat #403501). Prior to the onset of the experiment, the binding properties of all antibodies are validated using methods known to the art. Following the 5 minute incubation, cells are fixed with 4% formaldehyde (20 min at room temperature) and incubated with an appropriate fluorophore-labeled secondary antibody (e.g., ThermoFisher Mouse anti-Human IgG1 Fc Secondary Antibody, Alexa Fluor 488, Cat #A-10631) for 60 minutes. Unbound reagents are washed with a series of PBS washes, and the cell panels are imaged using confocal microscopy. Upon analysis of the images, significant fluorescence can be observed on the surface of cells bound with the known, control ABPC specific for LRRC15 as well as the pH-engineered ABPC specific for LRRC15, but little surface binding can be observed for the isotype negative control. To isolate the effect of pH on surface binding, the same experiment is repeated twice, with the primary antibody incubation taking place at sequentially lower pH (e.g., pH 6.5, pH 5.5, and pH 5.0). Analysis of the resulting confocal microscopy images can show significant fluorescence on the surface of cells bound with all mAbs tested, excepting the isotype negative control, and that this fluorescence decreases for the pH-engineered ABPC specific for LRRC15 as the pH decreases. Alternatively, cells are analyzed for mean fluorescent intensity by flow cytometry using methods known in the art. A dissociation constant KD on cells at neutral pH of the antibodies analyzed is determined by nonlinear regression methods known in the art (e.g., a Scatchard plot). Taken together, the results can show that the pH-engineering process results in the creation of a pH-engineered ABPC specific for LRRC15 that is pH-dependent in its binding properties and that it more effectively binds at neutral pH as compared to more acidic pH. Other methods of assessing the pH dependence of the pH-engineered ABPCs specific for LRRC15 are known in the art and include, e.g., using flow cytometry to measure ABPC surface binding.

pH-Dependent Release of LRRC15 on Cells

To demonstrate that pH-engineered ABPCs specific for LRRC15 are capable of releasing LRRC15 at low pH after binding at a neutral pH, a variant of the cell surface binding assay described above is performed using methods known to the art (e.g., as generally described in Gera N. (2012) PLOS ONE 7 (11): e48928). Briefly, an appropriate LRRC15+ cell line (passage number less than 25) is harvested and 50,000 cells per well are plated in a U-Bottomed 96-well microplate. Three conditions are tested; binding and secondary staining at pH 7.4, binding and secondary staining at pH 5.0, and binding at pH 7.4 followed by release at pH 5.0 for 30 minutes and secondary staining at pH 7.4. Both pH-engineered ABPCs specific for LRRC15 as well as a control ABPC specific for LRRC15 are tested. The cells are washed two times with 200 μL of FACS buffer (1×PBS containing 3% Fetal Bovine Serum) at either pH 7.4 or 5.0 depending on the condition being tested. The purified protein samples are diluted into FACS buffer of the appropriate pH and added to the cells and allowed to bind for one hour on ice. After incubation with the primary antibodies the pH 7.4 and pH 5.0 conditions are washed twice as before, and then 100 μl of secondary rat anti-human Fc AF488 (BioLegend 410706) or other appropriate antibody, diluted 1:50, or anti Myc-Tag mouse mAb-AF488 (Cell Signaling Technologies 2279S) diluted 1:50 is added in FACS buffer of the appropriate pH, and incubated for 30 minutes on ice. The pH 5.0 release condition is washed twice with FACS buffer pH 7.4 and then resuspended in 100 μl of FACS buffer pH 5.0 and incubated on ice for 30 minutes, followed by secondary staining in FACS buffer pH 7.4 as described for the other conditions. The plates are washed twice as before and resuspended in 1% paraformaldehyde in the appropriate FACS buffer to fix them for flow cytometry analysis. All conditions are read on a flow cytometer (Accuri C6, BD Biosciences). Binding is observed as a shift in the FLI signal (as a mean fluorescence intensity) versus secondary alone. Upon analysis of the data, it can be determined that both the pH-engineered ABPC specific for LRRC15 as well as the control ABPC specific for LRRC15 effectively bind the surface of LRRC15+ cells at neutral pH, but the pH-engineered ABPC specific for LRRC15 binds poorly at pH 5.0; similarly, it can be determined that the pH-engineered ABPC specific for LRRC15 binds at pH 7.4, but then releases LRRC15 at pH 5.0.

Enhanced Endolysosomal Delivery in LRRC15+ Cells of pH-Engineered ABPCs Specific for LRRC15 as Compared to Control ABPCs Specific for LRRC15

To verify and demonstrate that ABPCs specific for LRRC15 achieve endolysosomal localization following cellular uptake, an internalization assay is performed using methods known to the art (e.g., Mahmutefendic et al., Int. J. Biochem. Cell Bio., 2011). Briefly, as described herein, a panel of human cells that express LRRC15 highly is assembled using methods known to the art. Cells are plated, washed three times with PBS, and incubated at 37° C. for 60 minutes in media at neutral pH, with added concentrations of 2 micrograms per milliliter of a known, control ABPC specific for LRRC15 (e.g., as described herein), the pH-engineered ABPC specific for LRRC15, and an appropriate negative isotype control mAb (e.g., as described herein). In a subset of cells, validation of antibody internalization and endosomal localization is performed using methods known to the art; e.g., cells are fixed in 4% formaldehyde as described herein, permeabilized using TWEEN 20 or other methods known to the art (Jamur M C et al (2010) Permeabilization of cell membranes, Methods Mol Biol. 588:63-6), additionally stained with an endosomal marker, e.g., a fluorescent RAB11 antibody (RAB11 Antibody, Alexa Fluor 488, 3H18L5, ABfinity™ Rabbit Monoclonal), stained with an appropriate fluorescently labeled anti-human secondary antibody (e.g., as described herein), and imaged using confocal fluorescence microscopy, as described herein. Analysis of the confocal images can be used to show that both the pH-engineered ABPC specific for LRRC15 as well as the control ABPC specific for LRRC15 are internalized and accumulate in the endolysosomes.

To demonstrate that pH-engineered ABPCs specific for LRRC15 achieve enhanced endolysosomal accumulation relative to a control ABPC specific for LRRC15, a pHrodo-based internalization assay is performed using both a known, control ABPC specific for LRRC15 (e.g., as described herein) as well as the pH-engineered ABPC specific for LRRC15. The assay makes use of pHrodo™ iFL (P36014, ThermoFisher), a dye whose fluorescence increases with decreasing pH, such that its level of fluorescence outside the cell at neutral pH is lower than its level of fluorescence inside the acidic pH environment of endolysosomes. Briefly, an appropriate LRRC15+ cell line (less than passage 25) is suspended in its recommended media (e.g., by cell banks or cell bank databases ATCC, DSMZ, or ExPASy Cellosaurus) and plated in a 24-well plate at a density of 2,000,000 cells/mL, 1 mL per well. While keeping the cells on ice, 1 mL of 2× pHrodo iFL-labeled antibody (prepared in accordance with the manufacturer's instructions) is added to each well, the well is pipetted/mixed five times, and the plate is incubated in a light-protected environment for 45 minutes, on ice. An identical but separate plate is also incubated on ice that is meant as a no-internalization negative control. Following this incubation, the experimental plate is moved to a 37 degree C. incubator, the negative control plate is kept on ice to slow or block internalization, and samples are taken at designated time points to create an internalization time course. Samples are placed into a U-bottom 96-well plate, and internalization is quenched via addition of 200 μL/well of ice-cold FACS buffer. The plates are spun down at 2000×g for 2 minutes, resuspended in 200 μL ice-cold FACS buffer, spun down again, and resuspended in FACS buffer a second time. Finally, the samples are loaded into a flow cytometer for read-out of cellular pHrodo fluorescence using excitation and emission wavelengths consistent with the excitation and emission maxima of the pHrodo iFL Red dye (566 nm and 590 nm, respectively). Upon completion of the flow cytometry experiment and analysis of the data, it can be observed that cells treated with the pH-engineered ABPC specific for LRRC15 have a higher pHrodo iFL signal relative to a known, control ABPC specific for LRRC15, indicating that pH-engineered ABPCs specific for LRRC15 achieve enhanced endolysosomal accumulation relative to a control ABPC specific for LRRC15.

Alternatively, to demonstrate that pH-engineered ABPCs specific for LRRC15 achieve enhanced endolysosomal accumulation relative to a control ABPC specific for LRRC15, a variation of the above-described experiment is performed. LRRC15+ cells are plated, washed three times with PBS, and incubated at 37° C. for 60 minutes in media at neutral pH with added concentrations of 2 μg/mL of either pH-engineered ABPC specific for LRRC15 or control ABPC specific for LRRC15. Following incubation, cells are washed three times with PBS, fixed and permeabilized, and stained with a panel of appropriately selected antibodies that bind late endosomal markers as well as lysosomes (e.g., RAB7, and LAMP1; Cell Signaling Technology, Endosomal Marker Antibody Sampler Kit #12666; AbCam, Anti-LAMP2 antibody [GL2A7], ab13524). After primary antibody staining, cells are stained with an appropriate mixture of fluorescently labeled secondary antibodies (e.g., Goat Anti-Human IgG (H&L) Secondary Antibody (Alexa Fluor 647) Cat #A-21445, and Abcam Goat Anti-Rabbit IgG H&L (Alexa Fluor 488), Cat #ab150077), imaged using confocal fluorescence microscopy, and regions of co-localization of signal from LRRC15-specific antibodies and endosomal markers are visualized and quantified. Upon analysis of the data, it can be revealed that there is increased co-localization of endolysosomal and LRRC15-specific antibody signal in wells treated with the pH-engineered ABPCs specific for LRRC15 as compared to wells treated with control ABPC specific for LRRC15, and can thereby demonstrate that pH-engineered ABPCs specific for LRRC15 achieve enhanced endolysosomal accumulation relative to control ABPC.

Increased LRRC15 Antigen Density in LRRC15+ Cells after Exposure to pH-Engineered ABPCs Specific for LRRC15 as Compared to Control ABPCs Specific for LRRC15

To demonstrate that treatment of cells with the pH-engineered ABPCs specific for LRRC15 does not result in a detectable reduction of the level of LRRC15 on the surface of cells exposed to the pH-engineered ABPCs specific for LRRC15, or that said treatment results in less of a reduction of the level of LRRC15 on the surface of cells exposed to the pH-engineered ABPC specific for LRRC15 versus a control ABPC specific for LRRC15, an antigen density study is performed using flow cytometry. Briefly, 4.0×10\hat{ }5 cells that express LRRC15 are plated per well in a 96-well plate in 100 μL media. Cells are treated with a titration from 1 pM to 1 μM of i) pH-engineered ABPCs specific for LRRC15, ii) a first control ABPC specific for LRRC15, iii) an appropriate isotype control, and iv) an untreated control. Cells are incubated for 2 hours at 37° C., at which point all cells are incubated with 200 nM of a fluorophore-labeled second control ABPC specific for LRRC15 (e.g., as described herein) which has a different epitope (as determined by, e.g., competitive binding studies on cells) than either the first control ABPC specific for LRRC15 or the pH-engineered ABPCs specific for LRRC15 for 30 minutes at 4° C. Following this 30-minute incubation, the mean fluorescence intensity (MFI) of all cells is read out using, e.g., flow cytometry, using methods known to one of ordinary skill in the art. In parallel, a quantitative standard curve that can be used to quantify the presence of LRRC15 on the surface of treated cells as a function of MFI is generated using a commercially available quantification kit (e.g., BD Biosciences PE Phycoerythrin Fluorescence Quantitation Kit, catalog #340495); the quantitative standard curve is created by following the manufacturer's instructions. Other methods of determining the absolute number of LRRC15 on the cell surface are known in the art and include, e.g., use of radioisotopically labeled reagents. Upon analysis of the data, it can be revealed that at least one antibody concentration, cells treated with a control ABPC specific for LRRC15 experience a reduction of the level of LRRC15 on their surface, whereas cells treated with pH-engineered ABPCs specific for LRRC15 experience a significantly smaller reduction or no reduction at all, both relative to the isotype and untreated controls.

### Example 3. Conjugation of pH-Engineered and Control ABPCs to Cytotoxic Drugs

An antigen-binding protein construct conjugate (ADC) is made comprising the LRRC15-binding IgG (hereafter, LRRC15-IgG) described herein linked to monomethyl auristatin E (MMAE) via a valine-citrulline (vc) linker (hereafter, LRRC15-IgG-DC). Conjugation of the antigen-binding protein construct with vcMMAE begins with a partial reduction of the LRRC15-IgG followed by reaction with maleimidocaproyl-Val-Cit-PABC-MMAE (vcMMAE). The LRRC15-IgG (20 mg/mL) is partially reduced by addition of TCEP (molar equivalents of TCEP:mAb is 2:1) followed by incubation at 0° C. overnight. The reduction reaction is then warmed to 20° C. To conjugate all of the thiols, vcMMAE is added to a final vcMMAE:reduced Cys molar ratio of 1:15. The conjugation reaction is carried out in the presence of 10% v/v of DMSO and allowed to proceed at 20° C. for 60 minutes.

After the conjugation reaction, excess free N (acetyl)-Cysteine (2 equivalents vs. vcMMAE charge) is added to quench unreacted vcMMAE to produce the Cys-Val-Cit-MMAE adduct. The Cys quenching reaction is allowed to proceed at 20° C. for approximately 30 minutes. The Cys-quenched reaction mixture is purified as per below. The above conjugation method can also be used to conjugate maleimidocaproyl monomethylauristatin F (mcMMAF) to an antigen-binding protein construct.

The LRRC15-IgG-DC is purified using a batch purification method. The reaction mixture is treated with the appropriate amount of water washed Bu-HIC resin (ToyoPearl; Tosoh Biosciences), i.e., seven weights of resin is added to the mixture. The resin/reaction mixture is stirred for the appropriate time and monitored by analytical hydrophobic interaction chromatography for removal of drug conjugate products, filtered through a coarse polypropylene filter, and washed by two bed volumes of a buffer (0.28 M sodium chloride, 7 mM potassium phosphate, pH 7). The combined filtrate and rinses are combined and analyzed for product profile by HIC HPLC. The combined filtrate and rinses are buffer exchanged by ultrafiltration/diafiltration (UF/DF) to 15 mM histidine, pH 6 with 10 diavolumes 15 nM histidine buffer.

A similar protocol can be used to conjugate DNA toxins such as SG3249 and SGD-1910 to LRRC15-IgG (see Tiberghien A C et al (2016) Design and Synthesis of Tesirine, a Clinical Antibody-Drug Conjugate Pyrrolobenzodiazepine Dimer Payload, ACS Med Chem Lett 7:983-987). Briefly, for SG3249, LRRC15-IgG (15 mg, 100 nanomoles) is diluted into 13.5 mL of a reduction buffer containing 10 mM sodium borate pH 8.4, 2.5 mM EDTA and a final antibody concentration of 1.11 mg/mL. A 10 mM solution of TCEP is added (1.5 molar equivalent/antibody, 150 nanomoles, 15 microliters) and the reduction mixture is heated at +37° C. for 1.5 hours in an incubator. After cooling down to room temperature, SG3249 is added as a DMSO solution (5 molar equivalent/antibody, 500 nanomoles, in 1.5 mL DMSO). The solution is mixed for 1.25 hours at room temperature, then the conjugation is quenched by addition of N-acetyl cysteine (1 micromole, 100 microliters at 10 mM), and injected into an AKTA™ Pure FPLC using a GE Healthcare HiLoad™ 26/600 column packed with Superdex 200 PG, and eluted with 2.6 mL/min of sterile-filtered phosphate-buffered saline (PBS). Fractions corresponding to the LRRC15-IgG-DC monomer peak are pooled, concentrated using a 15 mL Amicon Ultracell 50 KDa MWCO spin filter, analyzed and sterile-filtered. UHPLC analysis on a Shimadzu Prominence system using a Phenomenex Aeris 3.6u XB-C18 150×2.1 mm column eluting with a gradient of water and acetonitrile on a reduced sample of LRRC15-IgG-DC at 280 nm and 330 nm (SG3249 specific) can show a mixture of light and heavy chains attached to several molecules of SG3249, consistent with a drug-per-antibody ratio (DAR) of 1 to 4 molecules of SG3249 per antibody. UHPLC analysis on a Shimadzu Prominence system using a Phenomenex Yarra 3u SEC-3000 300 mm×4.60 mm column eluting with sterile-filtered SEC buffer containing 200 mM potassium phosphate pH 6.95, 250 mM potassium chloride and 10% isopropanol (v/v) on a sample of LRRC15-IgG-DC at 280 nm can show a monomer purity of over 90% with no impurity detected. UHPLC SEC analysis allows determination of final LRRC15-IgG-DC yield of greater than 30%.

Alternatively, methods to conjugate toxins to antibodies via lysine residues are known in the art (e.g., see Catcott K C et al (2016) Microscale screening of antibody libraries as maytansinoid antibody-drug conjugates, MAbs 8:513-23). In addition, similar methods to the above can be used to conjugate drugs and toxins to non-IgG formats with disulfide bonds, such as Vh-Fcs.

### Example 4. Demonstration of Enhanced Cytotoxicity of pH-Engineered ABPC ADCs Specific for LRRC15 in LRRC15+ Cells as Compared to a Control ABPC ADC Specific for LRRC15

The cytotoxic activity of both pH-engineered ADCs specific for LRRC15 (e.g., a pH-engineered LRRC15-IgG-DC) and control ABPC ADCs specific for LRRC15 (e.g., a control ABPC LRRC15-IgG-DC) are separately evaluated on a panel of LRRC15+ cell lines expressing a variety of antigen densities (e.g., as described herein) and a LRRC15-cell line, selected using the methods described herein, and, optionally, cells expressing transgenic LRRC15, e.g., HEK293 cells transfected with LRRC15 using methods known in the art (e.g., Expi293™ Expression System Kit ThermoFisher Catalog number: A14635). For purposes of validation, prior to use, all cell lines are tested for expression of LRRC15 using methods known to the art, e.g., qPCR, flow cytometry, mRNA RPKM, and antibody staining using anti-LRRC15 antibodies known to the art (e.g., as described herein) followed by visualization of the stain using fluorescence microscopy, immunohistochemistry, flow cytometry, ELISA, or other methods known to the art. To evaluate the cytotoxicity of compounds, cells are seeded at approximately 10-40,000 per well in 150 microliters of culture medium, then treated with graded doses of compounds from 1 pM to 1 μM in quadruplicates at the initiation of the assay. Cytotoxicity assays are carried out for 96 hours after addition of test compounds. Fifty microliters of resazurin dye are added to each well during the last 4 to 6 hours of the incubation to assess viable cells at the end of culture. Dye reduction is determined by fluorescence spectrometry using the excitation and emission wavelengths of 535 nm and 590 nm, respectively. For analysis, the extent of resazurin reduction by the treated cells is compared to that of untreated control cells, and percent cytotoxicity is determined. Alternatively, a WST-8 kit is used to measure cytotoxicity per the manufacturer's instructions (e.g., Dojindo Molecular Technologies Catalog #CCK-8). IC50, the concentration at which half-maximal killing is observed, is calculated using curve-fitting methods known in the art. Upon analysis of the data, it can be determined that pH-engineered and control ABPC ADCs specific for LRRC15 are substantially cytotoxic to one or more LRRC15+ cell line, but less toxic to LRRC15-cells. It also can be determined that pH-engineered ADCs specific for LRRC15 are more cytotoxic to one or more LRRC15+ cell lines than control ABPC ADCs specific for LRRC15 because: a) they show greater depth of killing at one or more concentrations or, b) they show lower IC50 or, c) they show a greater ratio of their dissociation constant KD on cells at neutral pH (as described herein) divided by their IC50 on those same cells.

Additionally, the cytotoxic activity of ABPCs specific for LRRC15 can be measured in a secondary ADC assay. Secondary ADC assays are known in the art (e.g., Moradec Cat #αHFc-NC-MMAF and Cat #αHFc-CL-MMAE, and associated manufacturer's instructions). Briefly, the assay is carried out as in the previous paragraph, except the ABPC specific for LRRC15 is substituted for the ADC specific for LRRC15, and to evaluate the cytotoxicity of compounds, cells are seeded at approximately 10-40,000 per well in 150 microliters of culture medium, then treated with graded doses of ABPC specific for LRRC15 from 1 pM to 1 μM (final concentration in culture medium, having been pre-mixed with 100 nM, final concentration in culture medium, of Moradec Cat #αHFc-NC-MMAF secondary ADC reagent and pre-incubated at 37° C. for 30 min before addition of the mixture to the culture medium) in quadruplicates at the initiation of the assay.

The cytotoxic activity of pH-engineered ADCs specific for LRRC15 and control ABPC ADCs specific for LRRC15 conjugates, as well as ABPCs specific for LRRC15 in a secondary ADC assay, are additionally measured by a cell proliferation assay employing the following protocol (Promega Corp. Technical Bulletin TB288; Mendoza et al., Cancer Res. 62:5485-5488, 2002):
1. An aliquot of 100 microliters of cell culture containing about 104 cells (e.g., LRRC15+ cells as described herein) in medium is deposited in each well of a 96-well, opaque-walled plate.
2. Control wells are prepared containing medium and without cells.
3. ADC specific for LRRC15 is added to the experimental wells at a range of concentrations from 1 pM-1 μM and incubated for 1-5 days. Alternatively, in a secondary ADC assay, 100 nM secondary ADC reagent (final concentration in culture medium, Moradec Cat #αHFc-NC-MMAF) and ABPC specific for LRRC15 at a range of concentrations from 1 pM-1 μM (final concentration in culture medium) are pre-mixed and pre-incubated at 37° C. for 30 min before addition of the mixture to the culture medium, and incubated for 1-5 days.
4. The plates are equilibrated to room temperature for approximately 30 minutes.
5. A volume of CellTiter-Glo Reagent equal to the volume of cell culture medium present in each well is added.
6. The contents are mixed for 2 minutes on an orbital shaker to induce cell lysis.
7. The plate is incubated at room temperature for 10 minutes to stabilize the luminescence signal.
8. Luminescence is recorded and reported in graphs as RLU=relative luminescence units.

### Example 5. Demonstration of Enhanced Toxin Liberation of pH-Engineered ABPC ADCs Specific for LRRC15 in LRRC15+ Cells as Compared to a Control ABPC ADC Specific for LRRC15

The pH-engineered ADCs specific for LRRC15 (e.g., a pH-engineered LRRC15-IgG-DC) can also demonstrate increased toxin liberation in LRRC15+ cells as compared to a control ABPC ADC specific for LRRC15 (e.g., a control ABPC LRRC15-IgG-DC). After treatment of LRRC15+ cells with pH-engineered and control ABPC ADCs specific for LRRC15 as described herein, an LC-MS/MS method is used to quantify unconjugated (i.e., liberated) MMAE in treated LRRC15+ cells (Singh, A. P. and Shah, D. K. Drug Metabolism and Disposition 45.11 (2017): 1120-1132.) An LC-MS/MS system with electrospray interphase and triple quadrupole mass spectrometer is used. For the detection of MMAE, a XBridge BEH Amide column (Waters, Milford, MA) is used with mobile phase A as water (with 5 mM ammonium formate and 0.1% formic acid) and mobile phase B as 95:5 acetonitrile/water (with 0.1% formic acid and 1 mM ammonium formate), using a gradient at a flow rate of 0.25 mL/min at 40° C. The total duration of the chromatographic run is 12 minutes, where two MRM scans (718.5/686.5 and 718.5/152.1 amu) are monitored. Deuterated (d8) MMAE (MCE MedChem Express, Monmouth Junction, NJ) is used as an internal standard. First, an equation for quantifying unconjugated MMAE in a biological sample is derived by dividing the peak area for each drug standard by the peak area obtained for the internal standard. The resultant peak area ratios are then plotted as a function of the standard concentrations, and data points are fitted to the curve using linear regression. Three QC samples are included in the low, middle, and upper ranges of the standard curve to assess the predictive capability of the developed standard curve. The standard curves obtained are then used to deduce the observed concentrations of MMAE in a biologic sample. For measurement of MMAE concentration, treated cell samples are pelleted and reconstituted in fresh media to a final concentration of 0.25 million cells/100 μL. Samples are spiked with d8-MMAE (1 ng/mL) before performing cell lysis by the addition of a 2-fold volume of ice-cold methanol followed by freeze-thaw cycle of 45 minutes at −20° C. The final cell lysate is obtained by centrifuging the samples at 13,000 rpm for 15 minutes at 4° C. followed by collection of supernatant. For the preparation of standards and QC samples, a fresh cell suspension (0.25 million/100 μl) is spiked with known concentrations of MMAE and internal standard (d8-MMAE) before a procedure similar to the cell lysis mentioned above. The resulting cell lysates are then evaporated and reconstituted in mobile phase B before injection into LC-MS/MS. The concentration of unconjugated MMAE in lysates of LRRC15+ cells treated with pH-engineered ADCs specific for LRRC15 is observed to be greater than that in LRRC15+ cells treated with control ABPC ADC specific for LRRC15.

For tubulin-inhibiting toxins, toxin liberation is also assessed by monitoring of cell viability and cell cycle phase. ˜2.0×10\hat{ }5 LRRC15+ cells are plated in a 96-well flat bottom plate and treated with pH-engineered and control ABPC ADCs specific for LRRC15 as described herein. After treatment, cells are transferred to a 96-round bottom plate, and the plate is centrifuged at 400 rcf for 2 min to decant supernatant. Decanted cells are stained with Live/Dead eFluor 660. Cells are then centrifuged and washed with FACS buffer (PBS with 2% FBS), after which cell cycle distribution is analyzed with a BD Cycletest™ Plus DNA Kit (cat #340242). Briefly, cells are re-suspended in 76 μl Solution A and incubated for 10 min at room temperature. 61 μL Solution B is then added, and cells are incubated for another 10 min at room temperature. Finally, 61 μL of cold Solution C is added, and cells are again incubated for 10 min at room temp. Immediately after the last incubation step, cells are analyzed by flow cytometry (without washing) at a flow rate of 10 μL/sec. Increased G2/M-phase arrest can be observed with exposure to pH-engineered ADCs specific for LRRC15 as compared to control ABPC ADC specific for LRRC15.

For DNA-damaging toxins (e.g., pyrrolobenzodiazepine or “PBD”), DNA damage is assessed by measuring the phosphorylated histone H2AX (γH2AX). H2AX is normally phosphorylated in response to double-strand breaks in DNA; however, increased levels γH2AX may also be observed as a result of treatment with DNA-cross-linking toxins such as PBD or cisplatin (Huang, X. et al. 2004, Cytometry Part A 58A, 99-110). LRRC15+ cells are treated with pH-engineered and control ABPC ADCs specific for LRRC15 as described herein. After treatment, cells are rinsed with PBS, and then fixed in suspension in 1% methanol-free formaldehyde (Polysciences, Warrington, PA) in PBS at 0° C. for 15 min. Cells are resuspended in 70% ethanol for at least 2 h at −20° C. Cells are then washed twice in PBS and suspended in 0.2% Triton X-100 (Sigma) in a 1% (w/v) solution of BSA (Sigma) in PBS for 30 min to suppress nonspecific Ab binding. Cells are centrifuged again (200 g, 5 min) and the cell pellet is suspended in 100 μL of 1% BSA containing 1:800 diluted anti-histone γH2AX polyclonal Ab (Trevigen, Gaithersburg, MD). The cells are then incubated overnight at 4° C., washed twice with PBS, and resuspended in 100 μL of 1:30 diluted FITC-conjugated F(ab′)₂ fragment of swine anti-rabbit immunoglobulin (DAKO, Carpinteria, CA) for 30 min at room temperature in the dark. The cells are then counterstained with 5 μg/mL of PI (Molecular Probes, Eugene, OR) dissolved in PBS containing 100 μg/mL of DNase-free RNase A (Sigma), for 20 min at room temperature. Cellular fluorescence of the FITC γH2AX signal and the PI counterstain are measured using flow cytometry using methods known in the art. When comparing cells within the same stage of the cell cycle (based on total DNA content), treated LRRC15+ cells can be observed to have an increased FITC γH2AX signal relative to untreated LRRC15+ cells (which serve as a baseline). Furthermore, LRRC15+ cells treated with pH-engineered ADCs specific for LRRC15 can be observed to have a greater increase in levels of γH2AX over baseline than cells treated with a control ABPC ADC specific for LRRC15. In addition to the γH2AX assay, DNA cross-linking can be more directly assessed with a Comet assay (Chandna, S. (2004) Cytometry 61A, 127-133).

In addition, as disclosed herein, pH-engineered and control ABPCs can be assayed using the methods in this example without direct conjugation by performing a secondary ADC assay instead of using primary conjugated ADCs.

### Example 6. Demonstration of Increased or Decreased Half-Life of pH-Engineered ABPCs Specific for LRRC15 as Compared to a Control ABPC Specific for LRRC15

One of the surprising aspects of the pH-engineered ABPCs specific for LRRC15 described by the invention can be their ability to facilitate increased dissociation of ABPCs from the LRRC15 within the endosome or lysosome resulting in a decreased or increased serum half-life relative to control ABPCs specific for LRRC15 or ABPCs that are not specific for LRRC15. To demonstrate these properties, a series of animal studies in mice and/or monkeys is performed using pH-engineered ABPC specific for LRRC15 and control ABPC specific for LRRC15 using methods known to the art (e.g., Gupta, P., et al. (2016), mAbs, 8:5, 991-997). Briefly, to conduct mouse studies, a single intravenous bolus (e.g., 5 mg/kg) of either pH-engineered ABPC specific for LRRC15 or control ABPC specific for LRRC15 is administered via tail vein to two groups of NOD SCID mice (e.g., Jackson Labs NOD.CB17-Prkdescid/J Stock No: 001303) xenografted with a LRRC15+ cell line (e.g., as described herein). Xenografted mice are prepared by growing 1-5 million LRRC15+ cells in vitro and inoculating subcutaneously into the right flank of the mouse. Tumors are size matched at 300 mm³. Measurements of the length (L) and width (W) of the tumors are taken via electronic caliper and the volume is calculated according to the following equation: V=L×W\hat{ }2/2. Blood samples are collected via retro-orbital bleeds from each group at each of the following time points: 15 m, 30 m, 1 h, 8 h, 24 h, and 3 d, 7 d, 10 d, 14 d, 17 d, 21 d, and 28 d. Samples are processed to collect serum, and antibody concentrations are quantified using ELISA or other methods known to the art (e.g., PAC assay or MAC assay; Fischer, S. K. et al. (2012), mAbs, 4:5, 623-631, utilizing, e.g., anti-human Fc antibody Jackson ImmunoResearch Labs, Cat #109-006-006). Antibody concentrations of pH-engineered ABPC specific for LRRC15 and control ABPC specific for LRRC15 are plotted as a function of time. Upon analysis of the data, it can be observed that the pH-engineered ABPC specific for LRRC15 has a significantly longer or shorter serum half-life relative to control ABPC specific for LRRC15. If the pH-engineered and control ABPCs specific for LRRC15 are cross-reactive with the mouse homolog of LRRC15, a similar experiment can be repeated with non-xenografted mice.

Optionally, if the pH-engineered and control ABPCs specific for LRRC15 are cross-reactive with the cynomolgus monkey homolog of LRRC15, a similar experiment can be performed on monkeys (e.g., cynomolgus monkeys). An equal number of male and female monkeys (e.g., n=1-2 each) are administered a bolus of either pH-engineered ABPC specific for LRRC15 or control ABPC specific for LRRC15 at a dose of, e.g., 1 mg/kg via saphenous vein injection. Alternatively, several different doses of LRRC15-binding protein are administered across a group of several monkeys. Blood samples are collected via the peripheral vein or femoral vein at intervals similar to those described above and analyzed for the presence of either pH-engineered ABPC specific for LRRC15 or control ABPC specific for LRRC15 using methods known to the art (e.g., ELISA). Upon analysis of the data, it can be observed that pH-engineered ABPC specific for LRRC15 has a significantly longer or shorter serum half-life relative to control ABPC specific for LRRC15. In some cases, this effect is observed only in certain doses, whereas in others it is observed across doses. In particular cases, increased half-life is desirable, especially where less frequent patient dosing is advantageous.

In addition, the half-life of pH-engineered and control ABPC ADCs specific for LRRC15 can be assessed using the above methods by substituting pH-engineered and control ABPC ADCs specific for LRRC15 for the pH-engineered and control ABPCs specific for LRRC15 (i.e., studying the ABPCs after conjugation to a drug or toxin, as described herein).

### Example 7. Increased Potency of pH-Engineered ADCs Specific for LRRC15 vs. A Control ABPC ADC Specific for LRRC15 in Mouse Xenograft Models

The enhanced anti-tumor activity of the pH-engineered ADCs specific for LRRC15 against LRRC15+ tumors can be demonstrated in a subcutaneous xenograft model of LRRC15+ cells. For the experiments, 1-5 million LRRC15+ cells are grown in vitro and inoculated subcutaneously per mouse into the right flank of female immunodeficient (e.g., SCID-Beige or NOD scid) mice. Tumors are size matched at 100-200 mm³ and dosed intraperitoneally (IP) (1 dose given every ˜4-7 days for a total of ˜2-6 doses). Measurements of the length (L) and width (W) of the tumors are taken via electronic caliper and the volume is calculated according to the following equation: V=L×W\hat{ }2/2. A bolus (e.g., 5 mg/kg) of either pH-engineered ADC specific for LRRC15 or control ABPC ADC specific for LRRC15 is administered via tail vein. Tumor growth inhibition (TGI) and tumor growth delay (TGD) and survival are significantly improved with administration of pH-engineered ADC specific for LRRC15 compared to administration of control ABPC ADC specific for LRRC15 at the same regimen.

Optionally, spread of tumor cells into the various tissues is determined in sacrificed animals. Metastasis is measured according to Schneider, T., et al., Clin. Exp. Metas. 19 (2002) 571-582. Briefly, tissues are harvested and human Alu sequences are quantified by real-time PCR. Higher human DNA levels, quantified by real-time PCR, correspond to higher levels of metastasis. Levels of human Alu sequences (correlating to invasion of tumor cells into secondary tissue) are significantly lower in animals treated with pH-engineered ADC specific for LRRC15, corresponding to reduced metastasis, compared to mice treated with control ABPC ADC specific for LRRC15 at the same regimen. Alternatively, the enhanced anti-tumor activity of the pH-engineered ADC specific for LRRC15 can be shown in LRRC15+ patient-derived xenograft models (e.g., available from Charles River Laboratories).

### Example 8. Creation of a pH-Engineered Bispecific LRRC15 Bispecific ABPC and Demonstration of Exemplary Properties as Compared to a Control Bispecific ABPC

To create a pH-engineered ABPC specific for LRRC15 with modified toxicity and internalization properties, a bispecific antibody that binds two different epitopes on LRRC15 is constructed. It is known in the art that biparatopic antibodies can show increased antigen-dependent internalization, and are therefore useful for applications such as antibody-drug conjugates (e.g., see Li et al (2016) A Biparatopic HER2-Targeting Antibody-Drug Conjugate Induces Tumor Regression in Primary Models Refractory to or Ineligible for HER2-Targeted Therapy, Cancer Cell 29:117-29). Briefly, a pH-engineered LRRC15×LRRC15 bispecific, biparatopic ABPC specific for LRRC15 is assembled using light chain/heavy chain pairs from two different pH-engineered ABPCs specific for LRRC15, each of which binds a distinct epitope on LRRC15 that does not overlap with the other epitope. A set of pH-engineered ABPCs specific for LRRC15 that bind non-overlapping epitopes are discovered, e.g., using the methods described herein, or others known to one of ordinary skill in the art. Briefly, two binders are selected on the basis that they bind substantially different epitopes on LRRC15, as determined by, e.g., a binding competition assay as in Abdiche Y N et al (2009) Exploring blocking assays using Octet, ProteOn, and Biacore biosensors, Anal Biochem 386:172-80. Alternatively, briefly, as described herein, cell culture supernatants of cells transfected with a first ABPC specific for LRRC15 are normalized to an antibody expression level of 50 μg/mL, and captured on an anti-human Fc sensor (Forte Bio). A baseline is established using 1× kinetics buffer (Forte Bio), and the sensor is associated with 50 nM of LRRC15 in 1×PBS (that has been mixed and pre-incubated for 30 min at 37° C. with a second ABPC specific for LRRC15 transfection supernatant or the first ABPC specific for LRRC15 transfection supernatant, both normalized to 50 ug/mL) at pH 7.4 for 300 sec to generate an association curve. If the association rate in the presence of the second ABPC specific for LRRC15 is significantly faster (as calculated by the instrument software, or as seen by an elevated level of association over time) than the association rate in the presence of the first ABPC specific for LRRC15, then the second ABPC specific for LRRC15 is deemed to bind a non-overlapping epitope of LRRC15. Optionally, each antibody is screened for its internalization properties when bound to its epitope on a cell expressing LRRC15, and well-internalizing antibodies are selected. Assays for determining the internalization rate of a molecule present on the surface of a cell are known to the art. See, e.g., Wiley et al. (1991) J. Biol. Chem. 266:11083-11094; and Sorkin and Duex (2010) Curr. Protoc. Cell Biol. Chapter, Unit-15.14; Vainshtein et al. (2015) Pharm Res. 32:286-299. Once selected, heavy and light chain constructs with engineered mutations for heavy and light chain pairing (Spiess et al., “Alternative molecular formats and therapeutic applications of bispecific antibodies,” 2015) are synthesized for both arms. Bispecific ABPCs specific for LRRC15 are produced by co-expression of corresponding heavy and light chain plasmids in, e.g., Expi293 cells. Cell culture supernatants are harvested and subjected to Protein A purification. Heterodimeric ABPCs specific for LRRC15 are separated from homodimeric species via additional purification steps such as ion exchange chromatography, hydrophobic interaction chromatography, and mixed mode chromatography. The purified pH-engineered LRRC15×LRRC15 bispecific, biparatopic ABPCs specific for LRRC15 are characterized via mass spectrometry to confirm the purity and absence of homodimeric species and size exclusion chromatography to confirm the presence of monomeric antigen-binding protein construct species. For the product antibody, binding to the LRRC15 is confirmed via Biacore analysis. Other methods of bispecific antibody production are known to the art and could also be used to create a bispecific antibody, e.g., the LRRC15×LRRC15 bispecific, biparatopic ABPCs specific for LRRC15 described herein (e.g., Labrijn et al (2014) “Controlled Fab-arm exchange for the generation of stable bispecific IgG1” Nature Protocols 9:2450-2463, accessed at http://www.nature.com/nprot/journal/v9/n10/abs/nprot.2014.169.html), as would be apparent to one of ordinary skill in the art. Alternatively, instead of a LRRC15×LRRC15 ABPC specific for LRRC15, a pH-engineered LRRC15×BINDER ABPC specific for LRRC15 can be constructed using similar methods apparent to one skilled in the art, where BINDER is any antibody that has been published in the art or discovered using methods like those herein or those known in the art (e.g., display-based or immunization-based methods).

Next, exemplary properties of pH-engineered LRRC15×LRRC15 ABPCs specific for LRRC15 can be demonstrated using the methods described herein, with the appropriate control being a control ABPC monospecific or bispecific ABPC specific for LRRC15. Briefly, it can be shown that, as compared to a control, the pH-engineered LRRC15×LRRC15 ABPCs specific for LRRC15: a) bind in a pH-dependent manner to cells, e.g., bind at a neutral pH but not an acidic pH and b) release from cells in a pH-dependent manner, e.g., bind at a neutral pH and release at an acidic pH and c) show enhanced endolysosomal accumulation in LRRC15+ cells and d) show increased LRRC15 antigen density after exposure to LRRC15+ cells and e) when conjugated to a toxin, show increased cytotoxicity to LRRC15+ cells and f) when conjugated to a toxin, show increased toxin liberation when incubated with LRRC15+ cells and g) show decreased half-life when exposed to LRRC15 antigen in a relevant animal model and h) when conjugated to a toxin, show increased efficacy in a mouse xenograft model of LRRC15+ cells. Similarly, the exemplary properties of pH-engineered LRRC15×BINDER ABPCs specific for LRRC15 can be demonstrated using the methods described herein, with the appropriate control being a control ABPC LRRC15×BINDER bispecific ABPC specific for LRRC15.

### Example 9. Construction and Screening of pH-Engineered LRRC15 ABPCs

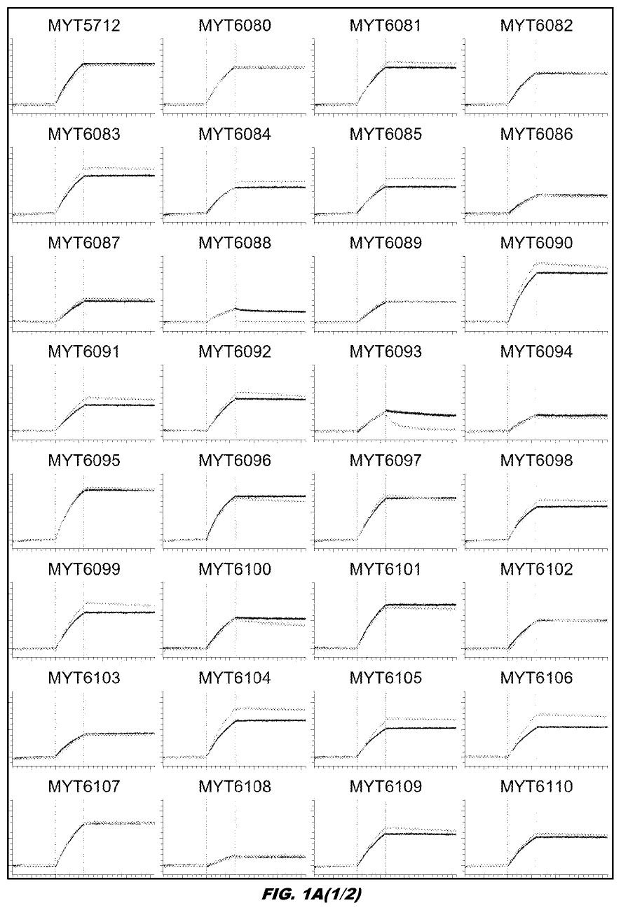

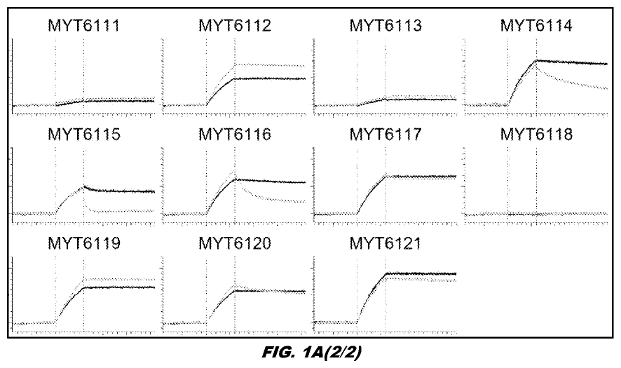

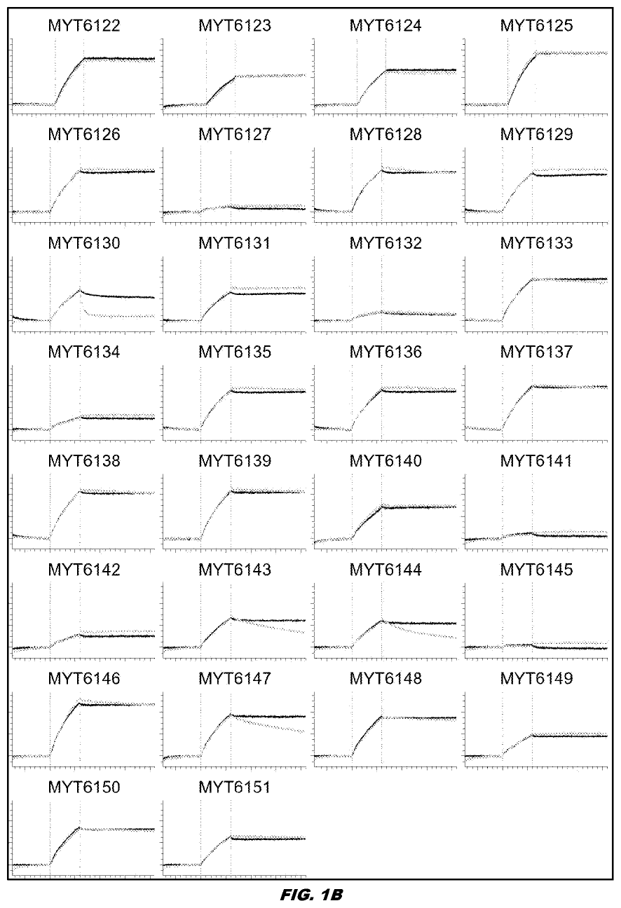

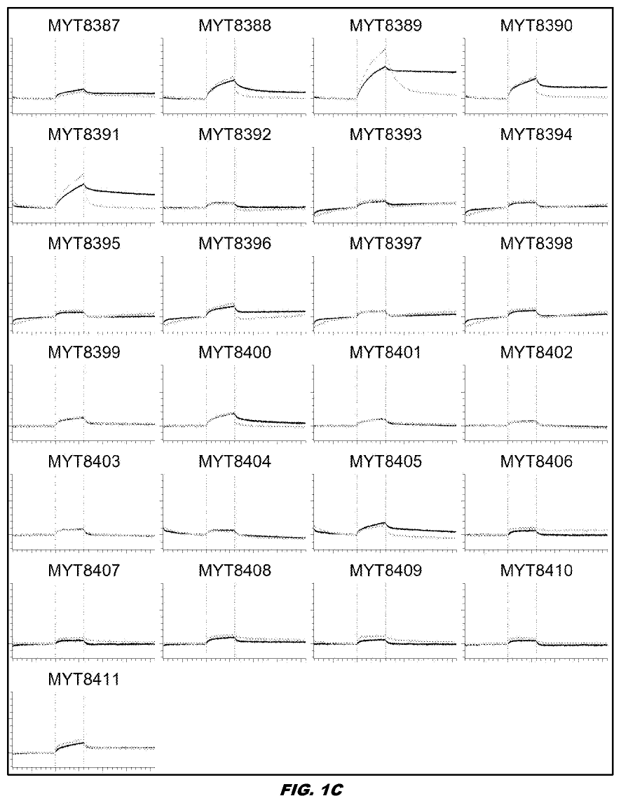

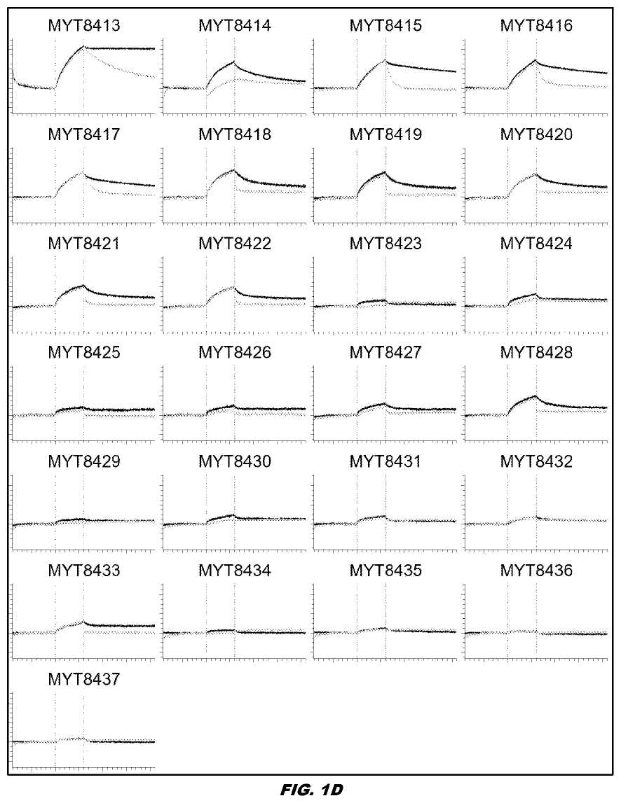

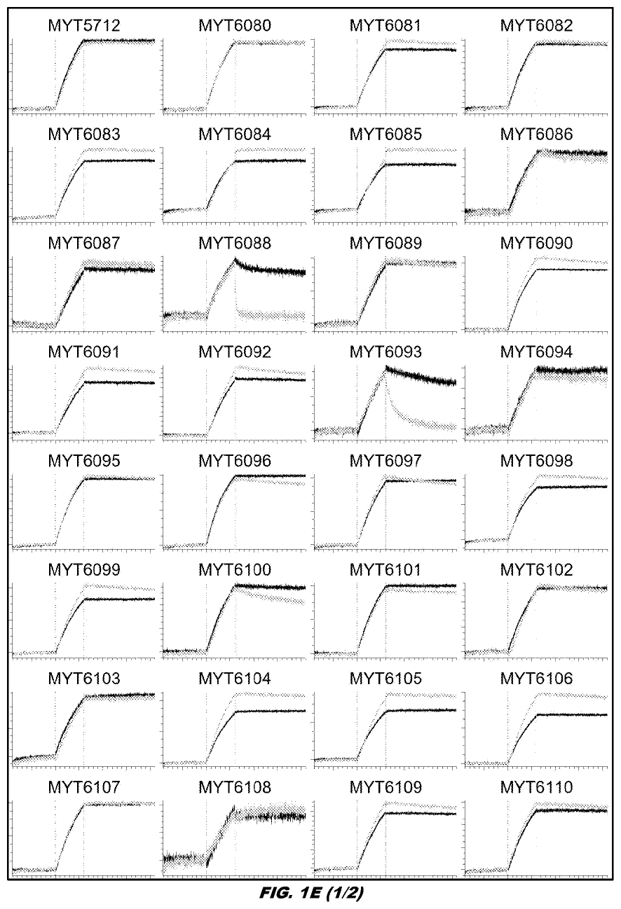

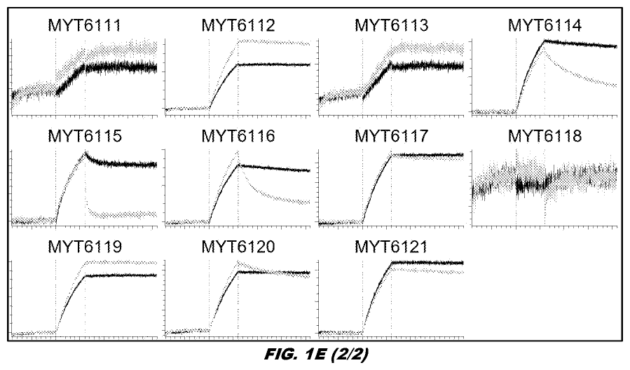

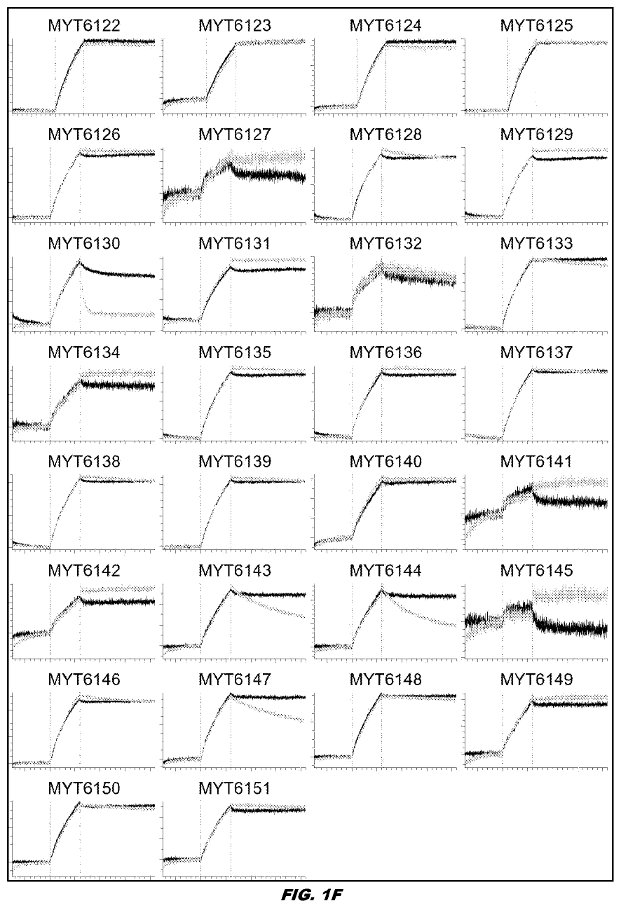

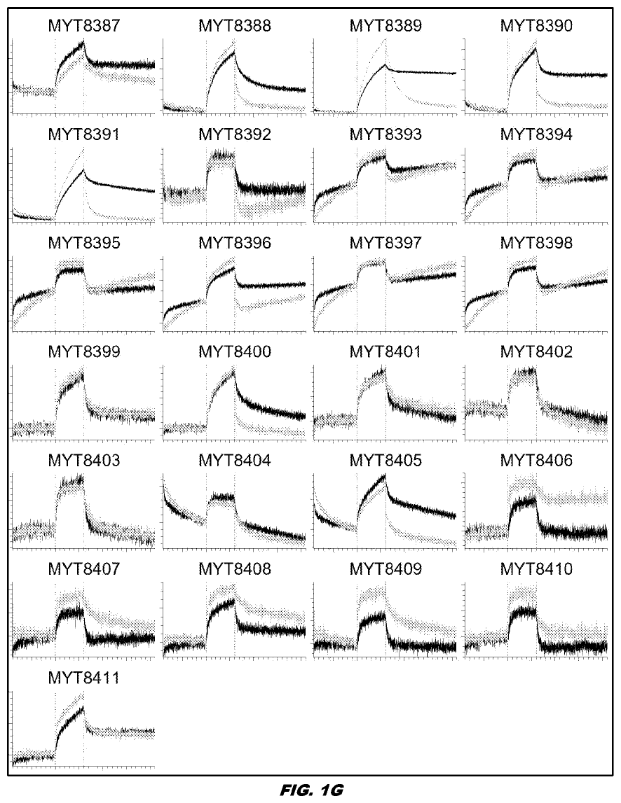

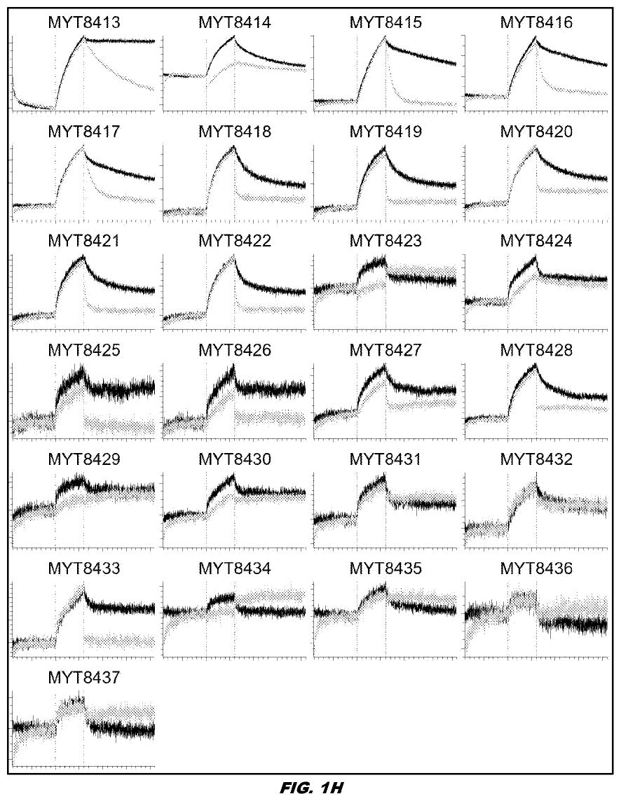

Multiple LRRC15-binding monoclonal antibodies have been described in the literature and can be used as a template for engineering pH-dependent binding. We selected discovery RabMAb clone 15G7 (rabbit VH SEQ ID NO: 378, rabbit VL SEQ ID NO: 451; humanized VH SEQ ID NO: 382, humanized VL SEQ ID NO: 455) as a LRRC15-binding monoclonal antibody for pH-engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine (or alanine if H was already present in a given position; e.g., MYT6099), one at a time (rabbit, MYT6080-6121). Desirable mutations were transferred to a humanized framework (humanized, MYT 8387-8411). After allowing for four days of protein expression, cell culture supernatants were collected, quantified by SDS-PAGE analysis, and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. The resulting diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sen1qsor was associated with 50 nM of LRRC15 (Human LRRC15 Protein, His Tag, Acro Biosystems) in 1×PBST pH 7.4 for 120 sec to generate an association curve. In the dissociation phase, the antibody-antigen complex on the sensor was exposed to 1×PBST pH 7.4 or pH 5.4, for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine substitution compared to the starting ABPC, (with no substitutions), and b) reduced dissociation at pH 7.4 (i.e., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to starting ABPC) as shown in FIGS. 1A & 1C and Tables 15 & 16 were selected for further analysis.

TABLE 15
Rabbit 15G7 VH mutations and binding characteristics

| | VH | VL | | | | Binding Characteristics | | |
|---|---|---|---|---|---|---|---|---|
| | Seq | Seq | VH Seq | | AA | Better @ | Similar | |
| MYT# | ID | ID | Position | WT AA | Mutation | pH 7.4 | to WT | Obliterated |
| 6080 | 383 | 451 | 26 | G | H | | X | |
| 6081 | 384 | 451 | 27 | L | H | | X | |
| 6082 | 385 | 451 | 28 | D | H | | X | |
| 6083 | 386 | 451 | 29 | F | H | | X | |
| 6084 | 387 | 451 | 30 | R | H | | X | |
| 6085 | 388 | 451 | 31 | R | H | | X | |
| 6086 | 389 | 451 | 32 | T | H | X | | |
| 6087 | 390 | 451 | 33 | N | H | | X | |
| 6088 | 391 | 451 | 34 | Y | H | X | | |
| 6089 | 392 | 451 | 35 | I | H | | X | |
| 6090 | 393 | 451 | 36 | C | H | | X | |
| 6091 | 394 | 451 | 51 | C | H | | X | |
| 6092 | 395 | 451 | 52 | I | H | | X | |
| 6093 | 396 | 451 | 53 | Y | H | X | | |
| 6094 | 397 | 451 | 54 | I | H | | X | |
| 6095 | 398 | 451 | 55 | A | H | | X | |
| 6096 | 399 | 451 | 56 | D | H | | X | |
| 6097 | 400 | 451 | 57 | V | H | | X | |
| 6098 | 401 | 451 | 58 | V | H | | X | |
| 6099 | 402 | 451 | 59 | H | A | | X | |
| 6100 | 403 | 451 | 60 | Y | H | X | | |
| 6101 | 404 | 451 | 61 | Y | H | | X | |
| 6102 | 405 | 451 | 62 | A | H | | X | |
| 6103 | 406 | 451 | 63 | S | H | | X | |
| 6104 | 407 | 451 | 64 | W | H | | X | |
| 6105 | 408 | 451 | 65 | A | H | | X | |
| 6106 | 409 | 451 | 66 | K | H | | X | |
| 6107 | 410 | 451 | 67 | G | H | | X | |
| 6108 | 411 | 451 | 97 | A | H | | X | |
| 6109 | 412 | 451 | 98 | R | H | | X | |
| 6110 | 413 | 451 | 99 | G | H | | X | |
| 6111 | 414 | 451 | 100 | A | H | | X | |
| 6112 | 415 | 451 | 101 | D | H | | X | |
| 6113 | 416 | 451 | 102 | G | H | | X | |
| 6114 | 417 | 451 | 103 | G | H | X | | |
| 6115 | 418 | 451 | 104 | F | H | X | | |
| 6116 | 419 | 451 | 105 | V | H | X | | |
| 6117 | 420 | 451 | 106 | Y | H | | X | |
| 6118 | 421 | 451 | 107 | G | H | | | X |
| 6119 | 422 | 451 | 108 | M | H | | X | |
| 6120 | 423 | 451 | 109 | D | H | X | | |
| 6121 | 424 | 451 | 110 | L | H | X | | |

TABLE 16
Humanized 15G7 VH mutations and binding characteristics

| | | | | | | Second | | | | | | Binding | | |
|---|---|---|---|---|---|---|---|---|---|---|---|---|---|---|
| | | | First Mutation | | | Mutation | | | Third Mutation | | | Characteristic | | |
| | VH | VL | VH | | | VH | | | VH | | | Better | | |
| | Seq | Seq | Seq | WT | Mut | Seq | WT | Mut | Seq | WT | Mut | @ pH | | |
| MYT# | ID | ID | Pos | AA | AA | Pos | AA | AA | Pos | AA | AA | 7.4 | WT | Oblit. |
| 8387 | 426 | 455 | 34 | Y | H | | | | | | | X | | |
| 8388 | 427 | 455 | 53 | Y | H | | | | | | | X | | |
| 8389 | 428 | 455 | 104 | G | H | | | | | | | X | | |
| 8390 | 429 | 455 | 105 | F | H | | | | | | | X | | |
| 8391 | 430 | 455 | 106 | V | H | | | | | | | X | | |
| 8392 | 431 | 455 | 34 | Y | H | 53 | Y | H | | | | | | X |
| 8393 | 432 | 455 | 34 | Y | H | 104 | G | H | | | | | | X |
| 8394 | 433 | 455 | 34 | Y | H | 105 | F | H | | | | | | X |
| 8395 | 434 | 455 | 34 | Y | H | 106 | V | H | | | | | | X |
| 8396 | 435 | 455 | 53 | Y | H | 104 | G | H | | | | | | X |
| 8397 | 436 | 455 | 53 | Y | H | 105 | F | H | | | | | | X |
| 8398 | 437 | 455 | 53 | Y | H | 106 | V | H | | | | | | X |
| 8399 | 438 | 455 | 104 | G | H | 105 | F | H | | | | X | | |
| 8400 | 439 | 455 | 104 | G | H | 106 | V | H | | | | X | | |
| 8401 | 440 | 455 | 105 | F | H | 106 | V | H | | | | X | | |
| 8402 | 441 | 455 | 34 | Y | H | 53 | Y | H | 104 | G | H | | | X |
| 8403 | 442 | 455 | 34 | Y | H | 53 | Y | H | 105 | F | H | | | X |
| 8404 | 443 | 455 | 34 | Y | H | 53 | Y | H | 106 | V | H | | | X |
| 8405 | 444 | 455 | 34 | Y | H | 104 | G | H | 105 | F | H | X | | |
| 8406 | 445 | 455 | 34 | Y | H | 104 | G | H | 106 | V | H | | | X |
| 8407 | 446 | 455 | 34 | Y | H | 105 | F | H | 106 | V | H | | | X |
| 8408 | 447 | 455 | 53 | Y | H | 104 | G | H | 105 | F | H | | | X |
| 8409 | 448 | 455 | 53 | Y | H | 104 | G | H | 106 | V | H | | | X |
| 8410 | 449 | 455 | 53 | Y | H | 105 | F | H | 106 | V | H | | | X |
| 8411 | 450 | 455 | 104 | G | H | 105 | F | H | 106 | V | H | X | | |

### Example 10. Construction and Screening of pH-Engineered LRRC15 ABPCs

Briefly, CDRs in the 15G7 light chain were identified using the methods described in Example 9, and for each CDR, residues falling under either or both Kabat and IMGT CDR definitions were called as CDR residues. To generate pH-dependent sequence variants, individual amino acid residues within the light chain CDRs were systematically substituted with a histidine, one at a time (rabbit, MYT6122-6151). Desirable mutations were transferred to a humanized framework MYT8413-8487). After allowing for four days of protein expression, cell culture supernatants were collected, quantified by SDS-PAGE analysis, and the pH dependence of the variant was evaluated using biolayer interferometry (BLI) on an Octet RED 96e instrument (as described in Example 9). Light chain variants that showed either enhanced dissociation at pH 5.4 or reduced dissociation at pH 7.4 or both (as compared to the starting ABPC), as shown in FIGS. 1B & ID and Tables 17 & 18 were selected for further analysis.

TABLE 17
Rabbit 15G7 VL mutations and binding characteristics

| | VH Seq | VL Seq | VL Seq | WT | Mut | Better @ | | |
|---|---|---|---|---|---|---|---|---|
| MYT# | ID | ID | Position | AA | AA | pH 7.4 | WT | Obliterated |
| 6122 | 378 | 456 | 24 | Q | H | | X | |
| 6123 | 378 | 457 | 25 | A | H | | X | |
| 6124 | 378 | 458 | 26 | S | H | | X | |
| 6125 | 378 | 459 | 27 | Q | H | | X | |
| 6126 | 378 | 460 | 28 | S | H | | X | |
| 6127 | 378 | 461 | 29 | I | H | X | | |
| 6128 | 378 | 462 | 30 | G | H | X | | |
| 6129 | 378 | 463 | 31 | N | H | | X | |
| 6130 | 378 | 464 | 32 | Y | H | X | | |
| 6131 | 378 | 465 | 33 | L | H | | X | |
| 6132 | 378 | 466 | 34 | S | H | X | | |
| 6133 | 378 | 467 | 50 | S | H | X | | |
| 6134 | 378 | 468 | 51 | A | H | | X | |
| 6135 | 378 | 469 | 52 | S | H | | X | |
| 6136 | 378 | 470 | 53 | T | H | | X | |
| 6137 | 378 | 471 | 54 | L | H | | X | |
| 6138 | 378 | 472 | 55 | P | H | | X | |
| 6139 | 378 | 473 | 56 | S | H | | X | |
| 6140 | 378 | 474 | 89 | Q | H | | X | |
| 6141 | 378 | 475 | 90 | N | H | | | X |
| 6142 | 378 | 476 | 91 | N | H | | X | |
| 6143 | 378 | 477 | 92 | Y | H | X | | |
| 6144 | 378 | 478 | 93 | W | H | X | | |
| 6145 | 378 | 479 | 94 | L | H | | | X |
| 6146 | 378 | 480 | 95 | A | H | X | | |
| 6147 | 378 | 481 | 96 | G | H | X | | |
| 6148 | 378 | 482 | 97 | Y | H | X | | |
| 6149 | 378 | 483 | 98 | G | H | | X | |
| 6150 | 378 | 484 | 99 | H | A | | X | |
| 6151 | 378 | 485 | 100 | P | H | | X | |

TABLE 18
Humanized 15G7 VL mutations and binding characteristics

| | | | | | | | | | | | | Binding | | |
|---|---|---|---|---|---|---|---|---|---|---|---|---|---|---|
| | | | 1^{st} Mutation | | | 2^{nd} Mutation | | | 3^{rd} Mutation | | | Characteristics | | |
| | VH | VL | VL | | | VL | | | VL | | | Better | | Better |
| | Seq | Seq | Seq | WT | Mut | Seq | WT | Mut | Seq | WT | Mut | @ pH | | @ pH |
| MYT# | ID | ID | Pos | AA | AA | Pos | AA | AA | Pos | AA | AA | 7.4 | Oblit. | 5.4 |
| 8413 | 382 | 487 | 30 | G | H | | | | | | | X | | |
| 8414 | 382 | 488 | 32 | Y | H | | | | | | | X | | |
| 8415 | 382 | 489 | 92 | Y | H | | | | | | | X | | |
| 8416 | 382 | 490 | 93 | W | H | | | | | | | X | | |
| 8417 | 382 | 491 | 96 | G | H | | | | | | | X | | |
| 8418 | 382 | 492 | 30 | G | H | 32 | Y | H | | | | X | | |
| 8419 | 382 | 493 | 30 | G | H | 92 | Y | H | | | | X | | |
| 8420 | 382 | 494 | 30 | G | H | 93 | W | H | | | | X | | |
| 8421 | 382 | 495 | 30 | G | H | 96 | G | H | | | | X | | |
| 8422 | 382 | 496 | 32 | Y | H | 92 | Y | H | | | | X | | |
| 8423 | 382 | 497 | 32 | Y | H | 93 | W | H | | | | | X | |
| 8424 | 382 | 498 | 32 | Y | H | 96 | G | H | | | | | | X |
| 8425 | 382 | 499 | 92 | Y | H | 93 | W | H | | | | X | | |
| 8426 | 382 | 500 | 92 | Y | H | 96 | G | H | | | | X | | |
| 8427 | 382 | 501 | 93 | W | H | 96 | G | H | | | | X | | |
| 8428 | 382 | 502 | 30 | G | H | 32 | Y | H | 92 | Y | H | X | | |
| 8429 | 382 | 503 | 30 | G | H | 32 | Y | H | 93 | W | H | | X | |
| 8430 | 382 | 504 | 30 | G | H | 32 | Y | H | 96 | G | H | | | X |
| 8431 | 382 | 505 | 30 | G | H | 92 | Y | H | 93 | W | H | X | | |
| 8432 | 382 | 506 | 30 | G | H | 92 | Y | H | 96 | G | H | X | | |
| 8433 | 382 | 507 | 30 | G | H | 93 | W | H | 96 | G | H | X | | |
| 8434 | 382 | 508 | 32 | Y | H | 92 | Y | H | 93 | W | H | | X | |
| 8435 | 382 | 509 | 32 | Y | H | 92 | Y | H | 96 | G | H | | X | |
| 8436 | 382 | 510 | 32 | Y | H | 93 | W | H | 96 | G | H | | X | |
| 8437 | 382 | 511 | 92 | Y | H | 93 | W | H | 96 | G | H | | X | |

### Example 11. Construction and Screening of pH-Engineered LRRC15 ABPCs

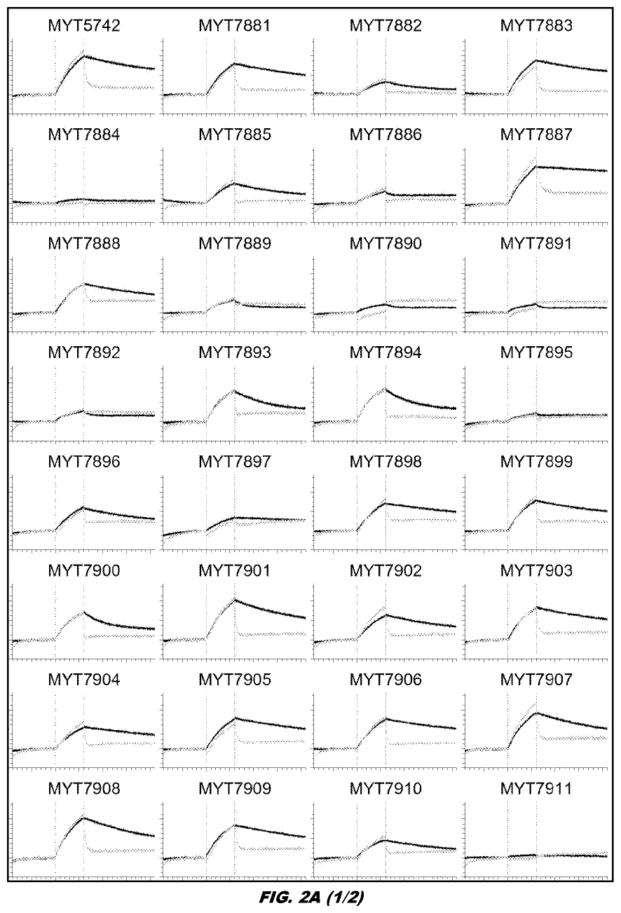

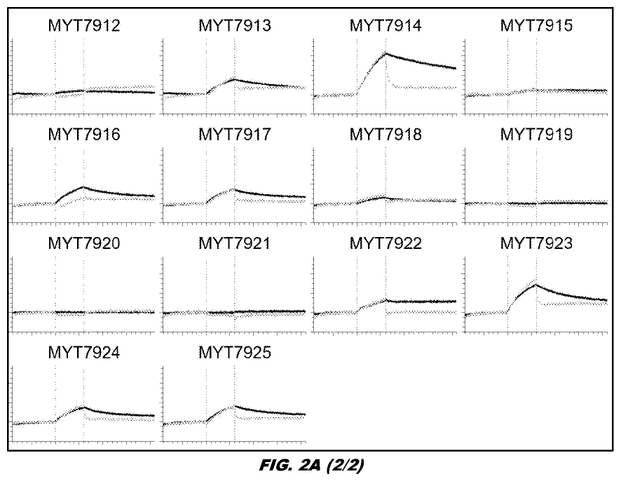

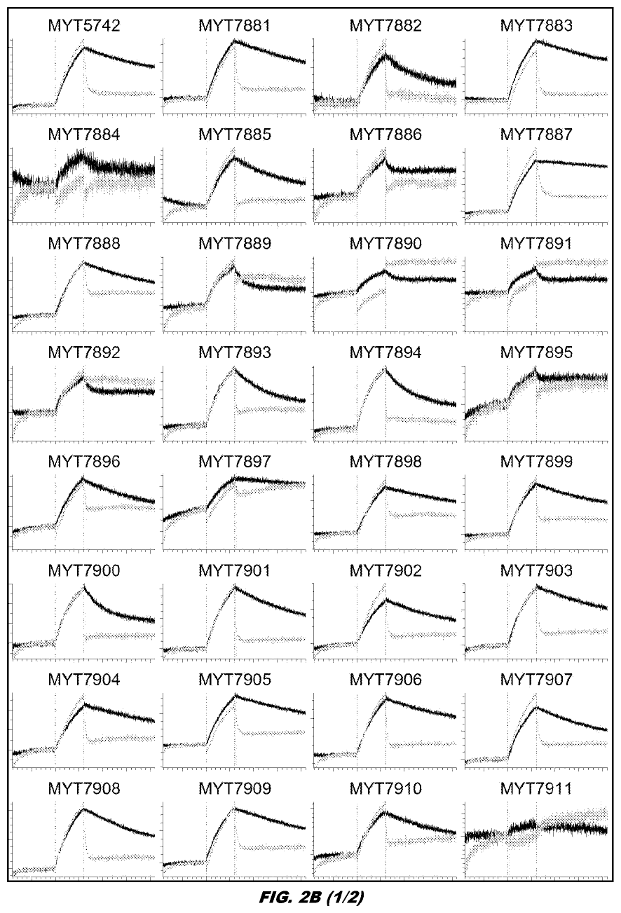

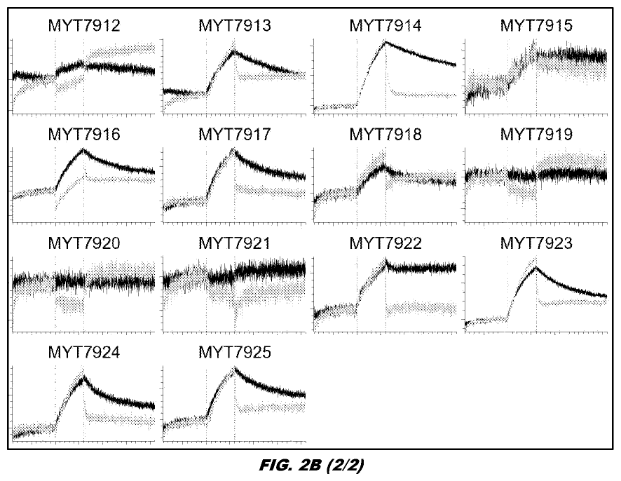

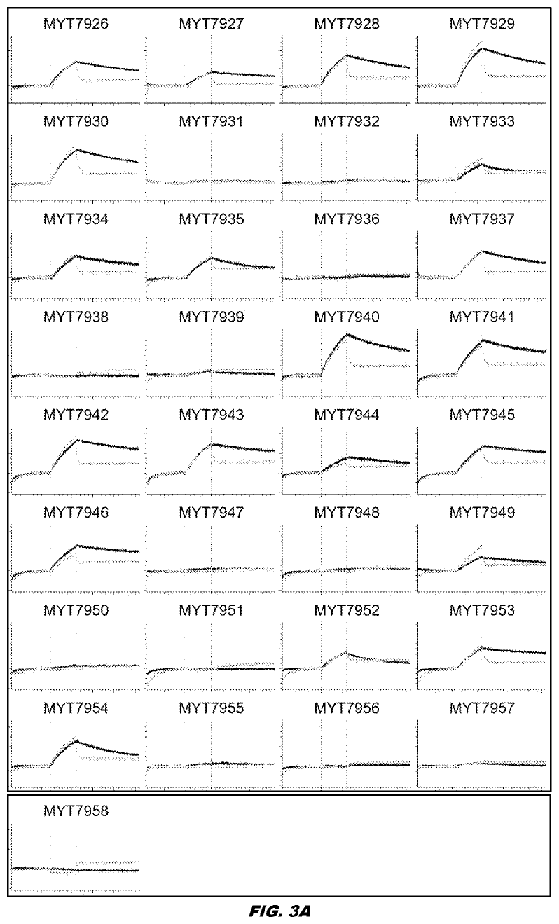

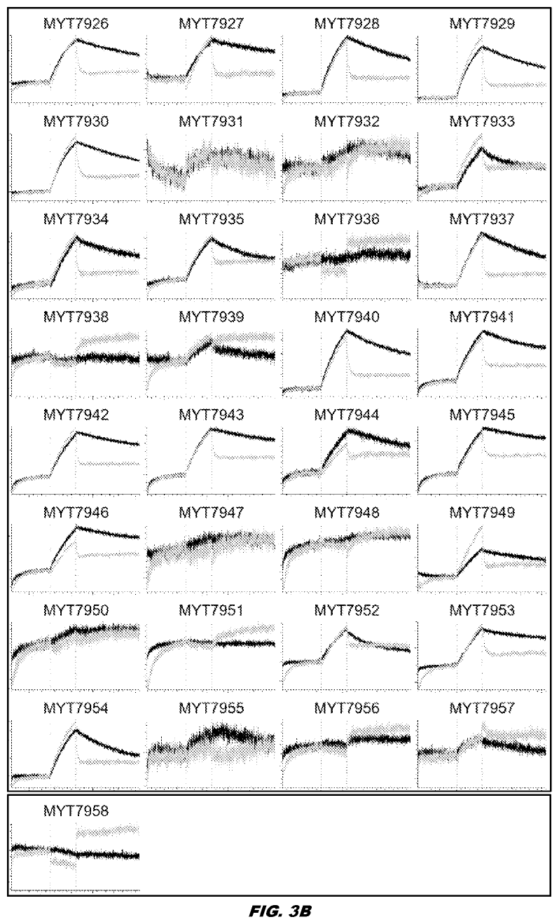

The methods of Examples 9-10 were applied to 24D9 (rabbit VH SEQ ID NO: 512 and VL SEQ ID NO: 518; and humanized VH SEQ ID NO: 516 and VL SEQ ID NO: 522). Variants that showed either enhanced dissociation at pH 5.4 or reduced dissociation at pH 7.4 or both were selected for further analysis (FIGS. 2 & 3 and Tables 19-21).

TABLE 19
Rabbit 24D9 VH mutations and binding characteristics

| | VH | VL | VH | | | Better | | | Better |
|---|---|---|---|---|---|---|---|---|---|
| | Seq | Seq | Seq | WT | | @ pH | | | @ pH |
| MYT# | ID | ID | Pos | AA | Mut | 7.4 | WT | Obliterated | 5.4 |
| 7881 | 523 | 518 | 26 | G | H | | X | | |
| 7882 | 524 | 518 | 27 | F | H | | X | | |
| 7883 | 525 | 518 | 28 | S | H | | X | | |
| 7884 | 526 | 518 | 29 | F | H | | | X | |
| 7885 | 527 | 518 | 30 | S | H | X | | | |
| 7886 | 528 | 518 | 31 | G | H | X | | | |
| 7887 | 529 | 518 | 32 | S | H | X | | | |
| 7888 | 530 | 518 | 33 | Y | H | | X | | |
| 7889 | 531 | 518 | 34 | W | H | | | | X |
| 7890 | 532 | 518 | 35 | I | H | | | | X |
| 7891 | 533 | 518 | 36 | C | H | | | | X |
| 7892 | 534 | 518 | 51 | C | H | | | | X |
| 7893 | 535 | 518 | 52 | I | H | X | | | |
| 7894 | 536 | 518 | 53 | N | H | X | | | |
| 7895 | 537 | 518 | 54 | G | H | | | | X |
| 7896 | 538 | 518 | 55 | G | H | | X | | |
| 7897 | 539 | 518 | 56 | S | H | | | | X |
| 7898 | 540 | 518 | 57 | L | H | | X | | |
| 7899 | 541 | 518 | 58 | D | H | X | | | |
| 7900 | 542 | 518 | 59 | I | H | X | | | |
| 7901 | 543 | 518 | 60 | T | H | X | | | |
| 7902 | 544 | 518 | 61 | Y | H | | X | | |
| 7903 | 545 | 518 | 62 | Y | H | | X | | |
| 7904 | 546 | 518 | 63 | A | H | | X | | |
| 7905 | 547 | 518 | 64 | S | H | | X | | |
| 7906 | 548 | 518 | 65 | W | H | | X | | |
| 7907 | 549 | 518 | 66 | A | H | | X | | |
| 7908 | 550 | 518 | 67 | K | H | | X | | |
| 7909 | 551 | 518 | 68 | G | H | | X | | |
| 7910 | 552 | 518 | 98 | A | H | X | | | |
| 7911 | 553 | 518 | 99 | S | H | | | X | |
| 7912 | 554 | 518 | 100 | D | H | | | X | |
| 7913 | 555 | 518 | 101 | T | H | | X | | |
| 7914 | 556 | 518 | 102 | S | H | | X | | |
| 7915 | 557 | 518 | 103 | Y | H | | | X | |
| 7916 | 558 | 518 | 104 | T | H | | X | | |
| 7917 | 559 | 518 | 105 | A | H | X | | | |
| 7918 | 560 | 518 | 106 | Y | H | X | | | |
| 7919 | 561 | 518 | 107 | G | H | | | X | |
| 7920 | 562 | 518 | 108 | N | H | | | X | |
| 7921 | 563 | 518 | 109 | F | H | | | X | |
| 7922 | 564 | 518 | 110 | Y | H | X | | | |
| 7923 | 565 | 518 | 111 | F | H | | X | | |
| 7924 | 566 | 518 | 112 | N | H | | X | | |
| 7925 | 567 | 518 | 113 | L | H | | X | | |

TABLE 20
Rabbit 24D9 VL mutations and binding characteristics

| | | | | | | | | | | Loss |
|---|---|---|---|---|---|---|---|---|---|---|
| | | | | | | Better | | | Better | @ |
| | VH | | VL | WT | | @ pH | | | @ pH | pH |
| MYT# | Seq | VL Seq | Pos | AA | Mut | 7.4 | WT | Obliterated | 5.4 | 7.4 |
| 7926 | 512 | 518 Q24H | 24 | Q | H | | X | | | |
| 7927 | 512 | 518 S25H | 25 | S | H | | X | | | |
| 7928 | 512 | 518 S26H | 26 | S | H | | X | | | |
| 7929 | 512 | 518 Q27H | 27 | Q | H | | X | | | |
| 7930 | 512 | 518 S28H | 28 | S | H | | X | | | |
| 7931 | 512 | 518 V29H | 29 | V | H | | | X | | |
| 7932 | 512 | 518 L30H | 30 | L | H | | | X | | |
| 7933 | 512 | 518 N31H | 31 | N | H | | | | | X |
| 7934 | 512 | 518 N32H | 32 | N | H | | X | | | |
| 7935 | 512 | 518 N33H | 33 | N | H | | | | | X |
| 7936 | 512 | 518 W34H | 34 | W | H | | | X | | |
| 7937 | 512 | 518 L35H | 35 | L | H | X | | | | |
| 7938 | 512 | 518 A36H | 36 | A | H | | | X | | |
| 7939 | 512 | 518 D52H | 52 | D | H | | | X | | |
| 7940 | 512 | 518 A53H | 53 | A | H | | X | | | |
| 7941 | 512 | 518 S54H | 54 | S | H | | X | | | |
| 7942 | 512 | 518 S55H | 55 | S | H | | X | | | |
| 7943 | 512 | 518 L56H | 56 | L | H | | X | | | |
| 7944 | 512 | 518 A57H | 57 | A | H | | | | X | |
| 7945 | 512 | 518 S58H | 58 | S | H | | X | | | |
| 7946 | 512 | 518 Q91H | 91 | Q | H | | X | | | |
| 7947 | 512 | 518 G92H | 92 | G | H | | | X | | |
| 7948 | 512 | 518 E93H | 93 | E | H | | | X | | |
| 7949 | 512 | 518 F94H | 94 | F | H | | X | | | |
| 7950 | 512 | 518 S95H | 95 | S | H | | | X | | |
| 7951 | 512 | 518 C96H | 96 | C | H | | | X | | |
| 7952 | 512 | 518 G97H | 97 | G | H | X | | | | |
| 7953 | 512 | 518 S98H | 98 | S | H | | X | | | |
| 7954 | 512 | 518 A99H | 99 | A | H | | | | | X |
| 7955 | 512 | 518 D100H | 100 | D | H | | | X | | |
| 7956 | 512 | 518 C96S | 96 | C | S | | | X | | |
| 7957 | 512 | 518 N102H | 102 | N | H | | | X | | |
| 7958 | 512 | 518 A103H | 103 | A | H | | | X | | |

TABLE 21
Rabbit 24D9 VH combo mutations and binding characteristics

| | | First Mutation | | | Second Mutation | | | Third Mutation | | | | |
|---|---|---|---|---|---|---|---|---|---|---|---|---|
| | | VH | | | VH | | | VH | | | Binding | |
| | | Pos | | | Pos | | | Pos | | | Characteristic | |
| | VL | (Seq | WT | | (Seq | WT | | (Seq | WT | | Better @ | |
| MYT# | Seq | 512) | AA | Mut | 512) | AA | Mut | 512) | AA | Mut | pH 7.4 | Obliterated |
| 8509 | 518 | 27 | F | H | 28 | S | H | 112 | F | H | | X |
| 8510 | 518 | 52 | I | H | 57 | L | H | 59 | I | H | X | |
| 8511 | 518 | 27 | F | H | 28 | S | H | 52 | I | H | | X |
| 9761 | 518 | 32 | S | H | 57 | L | H | | | | | |
| 9762 | 518 | 32 | S | H | 57 | L | R | | | | | |
| 9763 | 518 | 32 | S | H | 59 | I | Q | | | | | |
| 9764 | 518 | 53 | N | H | 57 | L | H | | | | | |
| 9765 | 518 | 53 | N | H | 57 | L | R | | | | X | |
| 9766 | 518 | 53 | N | H | 59 | I | Q | | | | | |
| 9767 | 518 | 32 | S | H | 53 | N | H | 57 | L | H | X | |
| 9768 | 518 | 32 | S | H | 53 | N | H | 57 | L | R | X | |
| 9769 | 518 | 32 | S | H | 53 | N | H | 59 | I | Q | X | |

### Example 12. Construction and Screening of pH-Engineered LRRC15 ABPCs

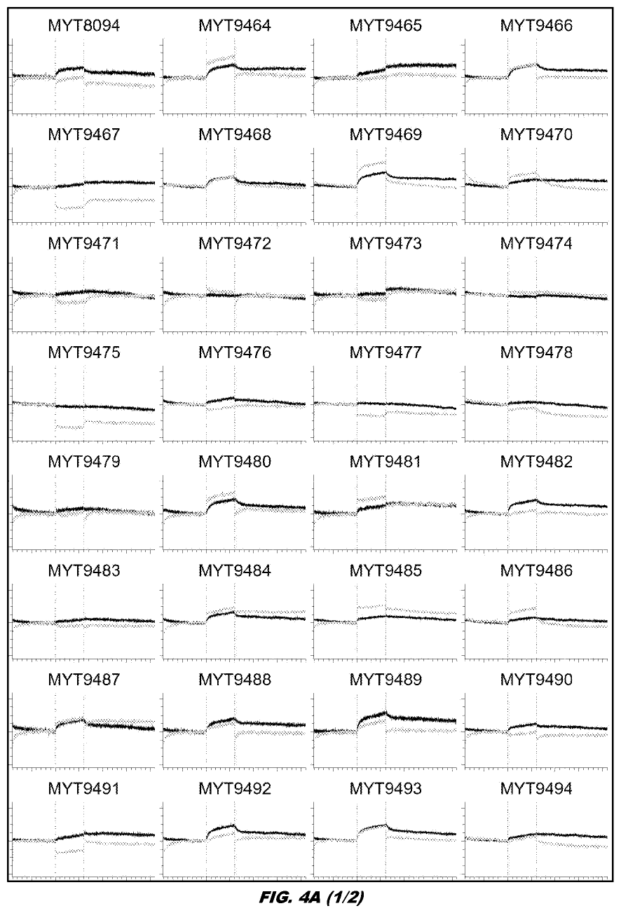

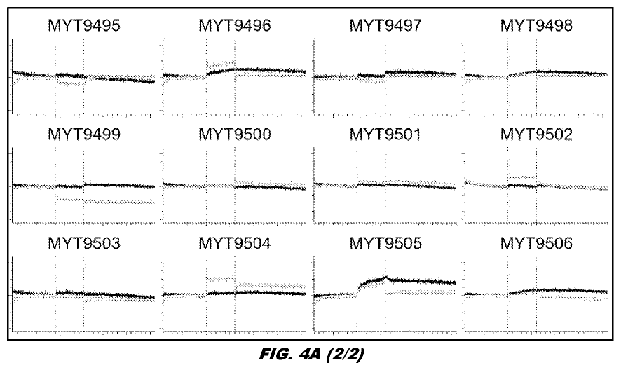

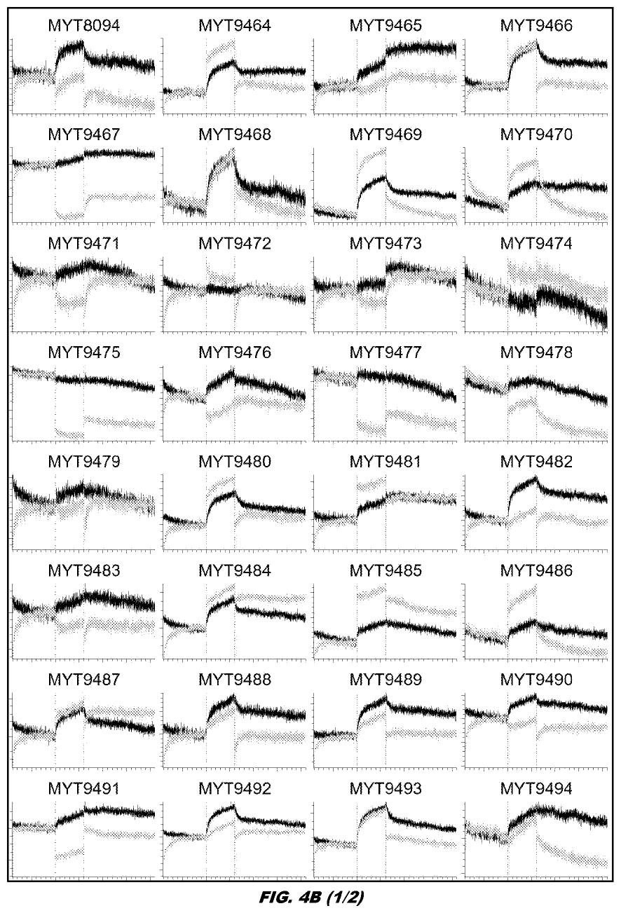

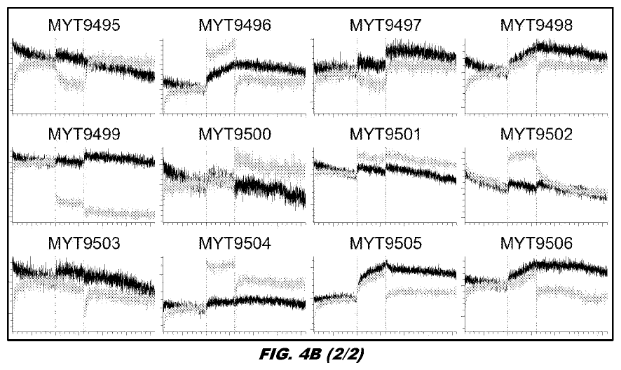

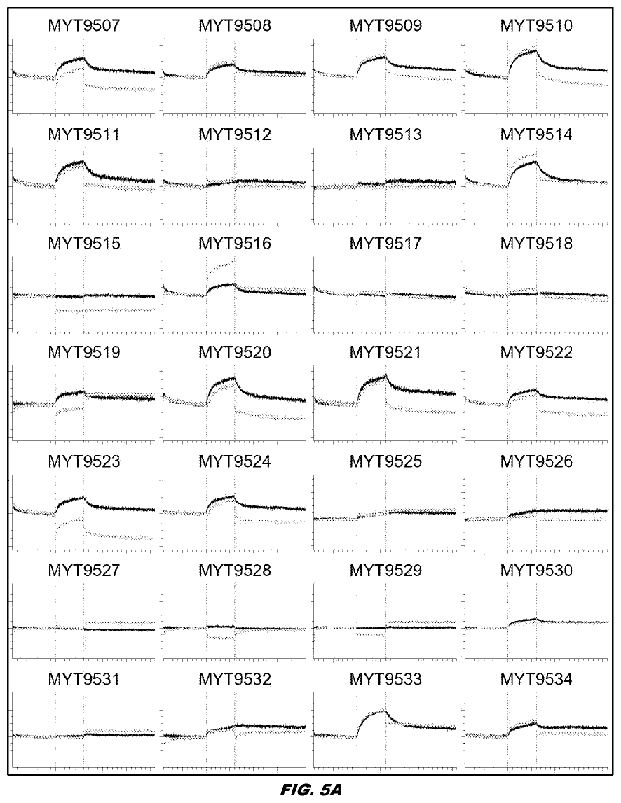

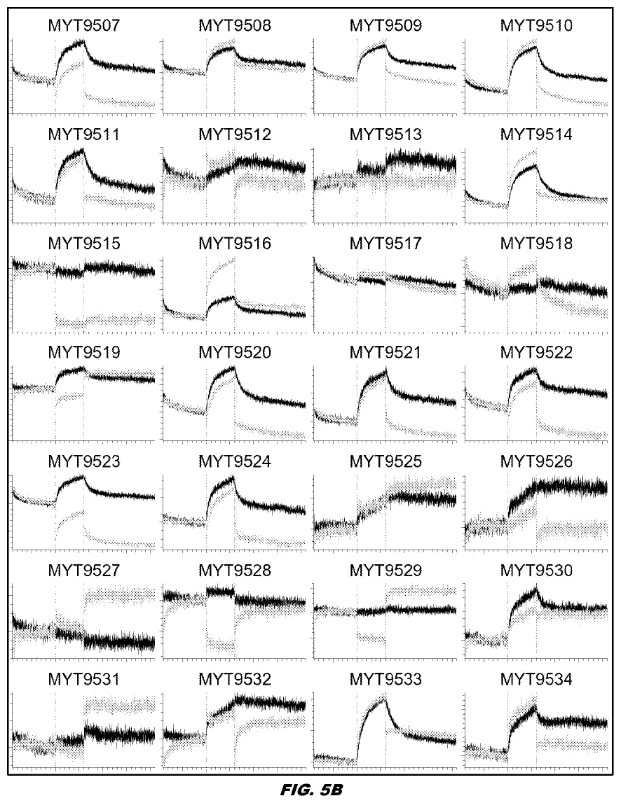

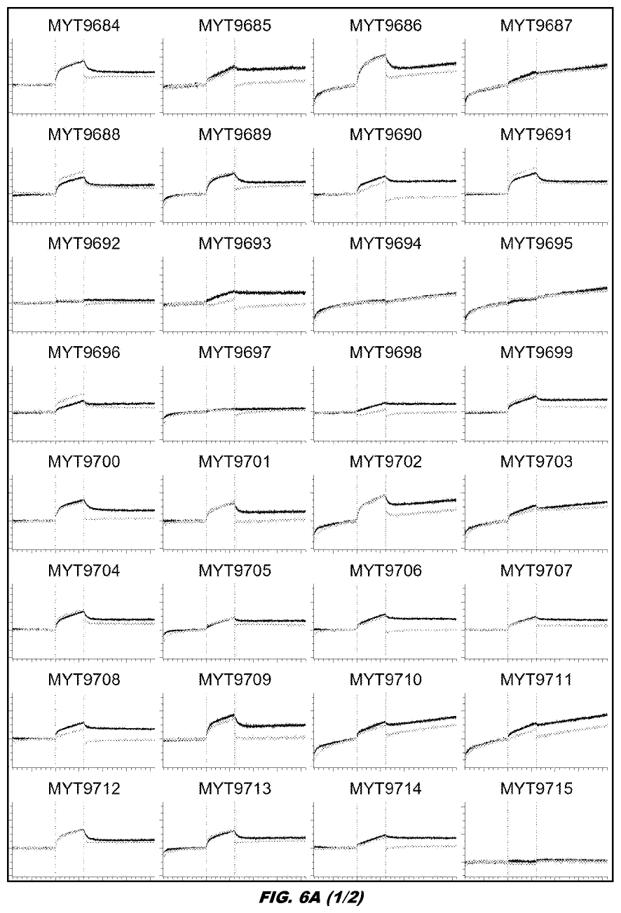

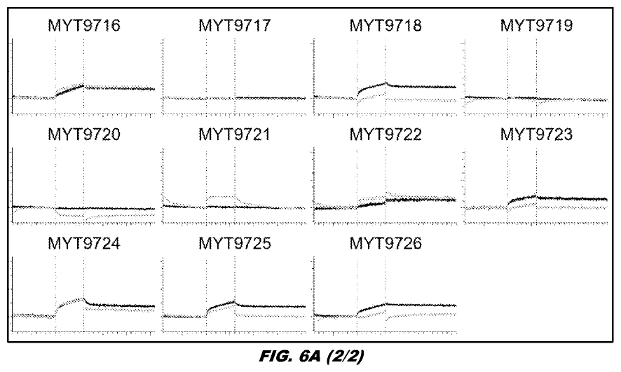

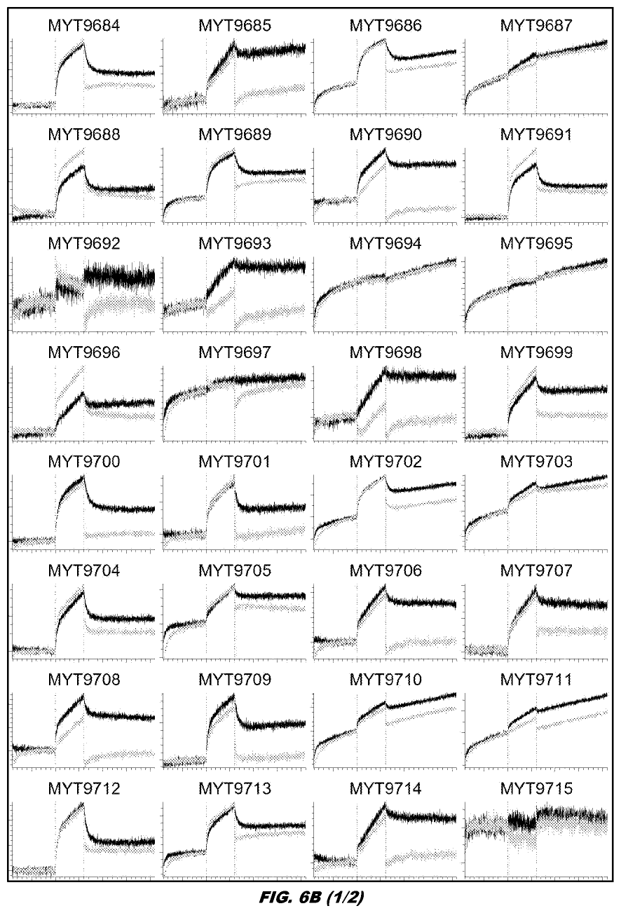

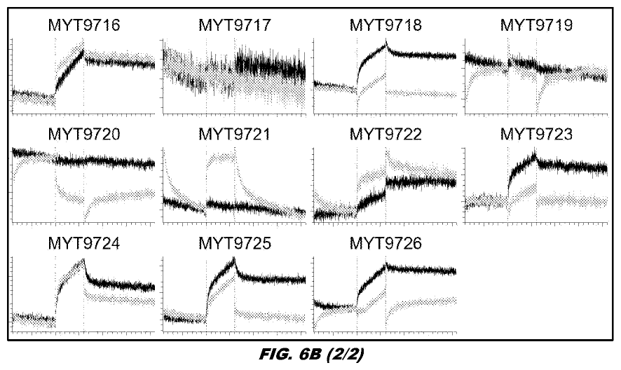

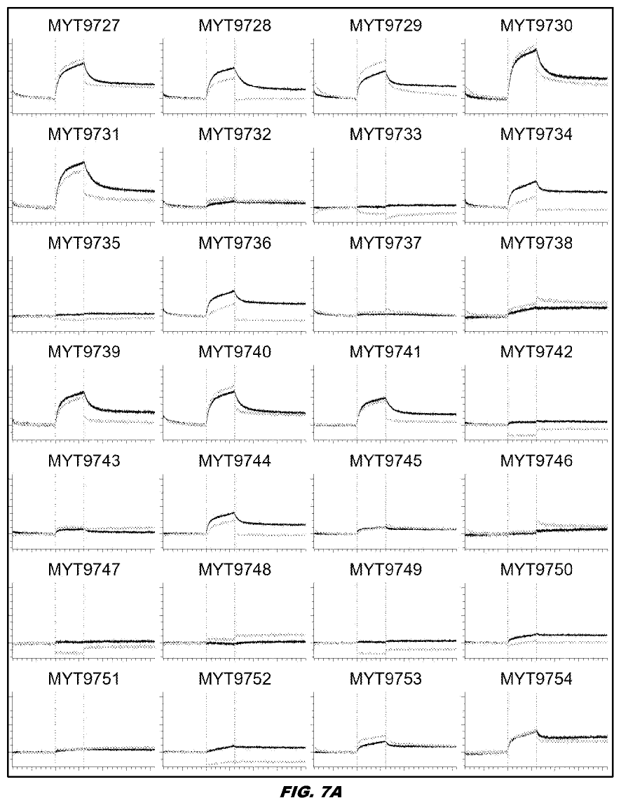

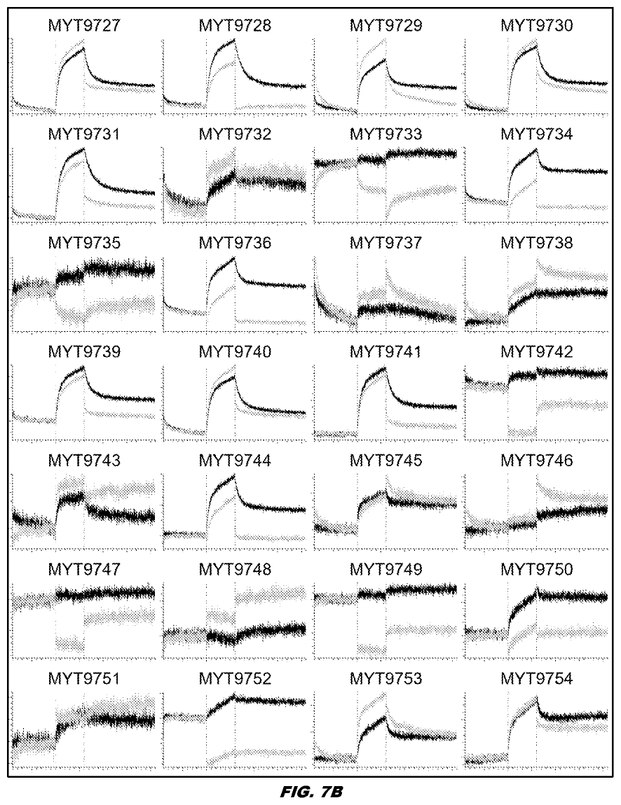

The methods of Examples 9-10 were applied to RabMAb clone 29F1 (rabbit VH SEQ ID NO: 572 and VL SEQ ID NO: 578; and humanized VH SEQ ID NO: 576 and VL SEQ ID NO: 582). Variants that showed either enhanced dissociation at pH 5.4 or reduced dissociation at pH 7.4 or both were selected for further analysis (FIGS. 4-7 and Tables 22-25).

TABLE 22
Humanized 29F1 VH aspartate mutations
and binding characteristics

| | | | VH | | | | |
|---|---|---|---|---|---|---|---|
| | VH | VL | Seq | WT | | | |
| MYT# | Seq ID | Seq ID | Pos | AA | Mut | Octet | SMAC RT |
| MYT9464 | 582 | 581 | 26 | G | D | | |
| MYT9465 | 583 | 581 | 27 | F | D | | |
| MYT9466 | 584 | 581 | 28 | S | D | | |
| MYT9467 | 585 | 581 | 29 | F | D | | |
| MYT9468 | 586 | 581 | 30 | S | D | | |
| MYT9469 | 587 | 581 | 31 | S | D | Good | 9.694 |
| MYT9470 | 588 | 581 | 32 | S | D | | |
| MYT9471 | 589 | 581 | 33 | H | D | | |
| MYT9472 | 590 | 581 | 34 | W | D | | |
| MYT9473 | 591 | 581 | 35 | I | D | | |
| MYT9474 | 592 | 581 | 36 | C | D | | |
| MYT9475 | 593 | 581 | 51 | C | D | | |
| MYT9476 | 594 | 581 | 52 | I | D | | |
| MYT9477 | 595 | 581 | 53 | Y | D | | |
| MYT9478 | 596 | 581 | 54 | A | D | | |
| MYT9479 | 597 | 581 | 55 | G | D | | |
| MYT9480 | 598 | 581 | 56 | S | D | Good | 9.591 |
| MYT9481 | 599 | 581 | 57 | I | D | | |
| MYT9482 | 600 | 581 | 58 | G | D | | |
| MYT9483 | 601 | 581 | 59 | S | D | | |
| MYT9484 | 602 | 581 | 60 | T | D | | |
| MYT9485 | 603 | 581 | 61 | Y | D | | |
| MYT9486 | 604 | 581 | 62 | Y | D | | |
| MYT9487 | 605 | 581 | 63 | A | D | | |
| MYT9488 | 606 | 581 | 64 | S | D | | |
| MYT9489 | 607 | 581 | 65 | W | D | | |
| MYT9490 | 608 | 581 | 66 | A | D | | |
| MYT9491 | 609 | 581 | 67 | K | D | | |
| MYT9492 | 610 | 581 | 68 | G | D | | |
| MYT9493 | 611 | 581 | 99 | A | D | Good | 9.687 |
| MYT9494 | 612 | 581 | 100 | R | D | | |
| MYT9495 | 613 | 581 | 101 | A | D | | |
| MYT9496 | 614 | 581 | 102 | P | D | | |
| MYT9497 | 615 | 581 | 103 | N | D | | |
| MYT9498 | 616 | 581 | 104 | D | A | | |
| MYT9499 | 617 | 581 | 105 | I | D | | |
| MYT9500 | 618 | 581 | 106 | Y | D | | |
| MYT9501 | 619 | 581 | 107 | N | D | | |
| MYT9502 | 620 | 581 | 108 | Y | D | | |
| MYT9503 | 621 | 581 | 109 | Y | D | | |
| MYT9504 | 622 | 581 | 110 | F | D | | |
| MYT9505 | 623 | 581 | 111 | N | D | | |
| MYT9506 | 624 | 581 | 112 | L | D | | |
| MYT9684 | 653 | 581 | 26 | G | E | | |
| MYT9685 | 654 | 581 | 27 | F | E | | |
| MYT9686 | 655 | 581 | 28 | S | E | | |
| MYT9687 | 656 | 581 | 29 | F | E | | |
| MYT9688 | 657 | 581 | 30 | S | E | | |
| MYT9689 | 658 | 581 | 31 | S | E | | |
| MYT9690 | 659 | 581 | 32 | S | E | | |
| MYT9691 | 660 | 581 | 33 | H | E | | |
| MYT9692 | 661 | 581 | 34 | W | E | | |
| MYT9693 | 662 | 581 | 35 | I | E | | |
| MYT9694 | 663 | 581 | 36 | C | E | | |
| MYT9695 | 664 | 581 | 51 | C | E | | |
| MYT9696 | 665 | 581 | 52 | I | E | | |
| MYT9697 | 666 | 581 | 53 | Y | E | | |
| MYT9698 | 667 | 581 | 54 | A | E | | |
| MYT9699 | 668 | 581 | 55 | G | E | | |
| MYT9700 | 669 | 581 | 56 | S | E | | |
| MYT9701 | 670 | 581 | 57 | I | E | | |
| MYT9702 | 671 | 581 | 58 | G | E | | |
| MYT9703 | 672 | 581 | 59 | S | E | Good | 9.545 |
| MYT9704 | 673 | 581 | 60 | T | E | | |
| MYT9705 | 674 | 581 | 61 | Y | E | | |
| MYT9706 | 675 | 581 | 62 | Y | E | | |
| MYT9707 | 676 | 581 | 63 | A | E | | |
| MYT9708 | 677 | 581 | 64 | S | E | | |
| MYT9709 | 678 | 581 | 65 | W | E | | |
| MYT9710 | 679 | 581 | 66 | A | E | | |
| MYT9711 | 680 | 581 | 67 | K | E | | |
| MYT9712 | 681 | 581 | 68 | G | E | | |
| MYT9713 | 682 | 581 | 99 | A | E | | |
| MYT9714 | 683 | 581 | 100 | R | E | | |
| MYT9715 | 684 | 581 | 101 | A | E | | |
| MYT9716 | 685 | 581 | 102 | P | E | | |
| MYT9717 | 686 | 581 | 103 | N | E | | |
| MYT9718 | 687 | 581 | 104 | D | E | | |
| MYT9719 | 688 | 581 | 105 | I | E | | |
| MYT9720 | 689 | 581 | 106 | Y | E | | |
| MYT9721 | 690 | 581 | 107 | N | E | | |
| MYT9722 | 691 | 581 | 108 | Y | E | | |
| MYT9723 | 692 | 581 | 109 | Y | F | | |
| MYT9724 | 693 | 581 | 110 | F | E | | |
| MYT9725 | 694 | 581 | 111 | N | E | | |
| MYT9726 | 695 | 581 | 112 | L | E | | |

TABLE 23
Humanized 29F1 VH glutamate mutations and binding characteristics

| | VH Seq | VL Seq | VH | WT | Mut | | SMAC |
|---|---|---|---|---|---|---|---|
| MYT# | ID | ID | Position | AA | AA | Octet | RT |
| MYT9684 | 653 | 581 | 26 | G | E | | |
| MYT9685 | 654 | 581 | 27 | F | E | | |
| MYT9686 | 655 | 581 | 28 | S | E | | |
| MYT9687 | 656 | 581 | 29 | F | E | | |
| MYT9688 | 657 | 581 | 30 | S | E | | |
| MYT9689 | 658 | 581 | 31 | S | E | | |
| MYT9690 | 659 | 581 | 32 | S | E | | |
| MYT9691 | 660 | 581 | 33 | H | E | | |
| MYT9692 | 661 | 581 | 34 | W | E | | |
| MYT9693 | 662 | 581 | 35 | I | E | | |
| MYT9694 | 663 | 581 | 36 | C | E | | |
| MYT9695 | 664 | 581 | 51 | C | E | | |
| MYT9696 | 665 | 581 | 52 | I | E | | |
| MYT9697 | 666 | 581 | 53 | Y | E | | |
| MYT9698 | 667 | 581 | 54 | A | E | | |
| MYT9699 | 668 | 581 | 55 | G | E | | |
| MYT9700 | 669 | 581 | 56 | S | E | | |
| MYT9701 | 670 | 581 | 57 | I | E | | |
| MYT9702 | 671 | 581 | 58 | G | E | | |
| MYT9703 | 672 | 581 | 59 | S | E | Good | 9.545 |
| MYT9704 | 673 | 581 | 60 | T | E | | |
| MYT9705 | 674 | 581 | 61 | Y | E | | |
| MYT9706 | 675 | 581 | 62 | Y | E | | |
| MYT9707 | 676 | 581 | 63 | A | E | | |
| MYT9708 | 677 | 581 | 64 | S | E | | |
| MYT9709 | 678 | 581 | 65 | W | E | | |
| MYT9710 | 679 | 581 | 66 | A | E | | |
| MYT9711 | 680 | 581 | 67 | K | E | | |
| MYT9712 | 681 | 581 | 68 | G | E | | |
| MYT9713 | 682 | 581 | 99 | A | E | | |
| MYT9714 | 683 | 581 | 100 | R | E | | |
| MYT9715 | 684 | 581 | 101 | A | E | | |
| MYT9716 | 685 | 581 | 102 | P | E | | |
| MYT9717 | 686 | 581 | 103 | N | E | | |
| MYT9718 | 687 | 581 | 104 | D | E | | |
| MYT9719 | 688 | 581 | 105 | I | E | | |
| MYT9720 | 689 | 581 | 106 | Y | E | | |
| MYT9721 | 690 | 581 | 107 | N | E | | |
| MYT9722 | 691 | 581 | 108 | Y | E | | |
| MYT9723 | 692 | 581 | 109 | Y | E | | |
| MYT9724 | 693 | 581 | 110 | F | E | | |
| MYT9725 | 694 | 581 | 111 | N | E | | |
| MYT9726 | 695 | 581 | 112 | L | E | | |

TABLE 24
Humanized 29F1 VL aspartate mutations
and binding characteristics

| | VH Seq | VL Seq | | | Mut | | SMAC |
|---|---|---|---|---|---|---|---|
| MYT# | ID | ID | VL Pos | WT AA | AA | Octet | RT |
| MYT9507 | 576 | 625 | 24 | Q | D | 0 | 9.659 |
| MYT9508 | 576 | 626 | 25 | A | D | | |
| MYT9509 | 576 | 627 | 26 | S | D | 0 | 9.664 |
| MYT9510 | 576 | 628 | 27 | E | D | Good | 9.627 |
| MYT9511 | 576 | 629 | 28 | N | D | Good | 9.52 |
| MYT9512 | 576 | 630 | 29 | I | D | | |
| MYT9513 | 576 | 631 | 30 | Y | D | | |
| MYT9514 | 576 | 632 | 31 | N | D | Good | 9.461 |
| MYT9515 | 576 | 633 | 32 | Y | D | | |
| MYT9516 | 576 | 634 | 33 | L | D | | |
| MYT9517 | 576 | 635 | 34 | A | D | | |
| MYT9518 | 576 | 636 | 50 | Y | D | | |
| MYT9519 | 576 | 637 | 51 | A | D | | |
| MYT9520 | 576 | 638 | 52 | S | D | Good | 9.524 |
| MYT9521 | 576 | 639 | 53 | T | D | 0 | 9.519 |
| MYT9522 | 576 | 640 | 54 | L | D | 0 | 9.67 |
| MYT9523 | 576 | 641 | 55 | A | D | 0 | 9.686 |
| MYT9524 | 576 | 642 | 56 | S | D | Good | 9.677 |
| MYT9525 | 576 | 643 | 89 | Q | D | | |
| MYT9526 | 576 | 644 | 90 | S | D | | |
| MYT9527 | 576 | 645 | 91 | G | D | | |
| MYT9528 | 576 | 646 | 92 | Y | D | | |
| MYT9529 | 576 | 647 | 93 | V | D | | |
| MYT9530 | 576 | 648 | 94 | S | D | Bad | 9.656 |
| MYT9531 | 576 | 649 | 95 | K | D | | |
| MYT9532 | 576 | 650 | 96 | S | D | | |
| MYT9533 | 576 | 651 | 97 | S | D | | |
| MYT9534 | 576 | 652 | 98 | T | D | | |

TABLE 25
Humanized 29F1 VL glutamate mutations
and binding characteristics

| | VH Seq | VL Seq | VL | WT | Mut | | |
|---|---|---|---|---|---|---|---|
| MYT# | ID | ID | Position | AA | AA | Octet | SMAC RT |
| MYT9727 | 576 | 696 | 24 | Q | E | 0 | 9.661 |
| MYT9728 | 576 | 697 | 25 | A | E | | |
| MYT9729 | 576 | 698 | 26 | S | E | | |
| MYT9730 | 576 | 699 | 27 | E | A | 0 | 9.69 |
| MYT9731 | 576 | 700 | 28 | N | E | 0 | 9.548 |
| MYT9732 | 576 | 701 | 29 | I | E | | |
| MYT9733 | 576 | 702 | 30 | Y | E | | |
| MYT9734 | 576 | 703 | 31 | N | E | | |
| MYT9735 | 576 | 704 | 32 | Y | E | | |
| MYT9736 | 576 | 705 | 33 | L | E | | |
| MYT9737 | 576 | 706 | 34 | A | E | | |
| MYT9738 | 576 | 707 | 50 | Y | E | Bad | 9.429 |
| MYT9739 | 576 | 708 | 51 | A | E | Good | 9.533 |
| MYT9740 | 576 | 709 | 52 | S | E | 0 | 9.541 |
| MYT9741 | 576 | 710 | 53 | T | E | 0 | 9.607 |
| MYT9742 | 576 | 711 | 54 | L | E | | |
| MYT9743 | 576 | 712 | 55 | A | E | | |
| MYT9744 | 576 | 713 | 56 | S | E | Good | 9.698 |
| MYT9745 | 576 | 714 | 89 | Q | E | | |
| MYT9746 | 576 | 715 | 90 | S | E | | |
| MYT9747 | 576 | 716 | 91 | G | E | | |
| MYT9748 | 576 | 717 | 92 | Y | E | | |
| MYT9749 | 576 | 718 | 93 | V | E | | |
| MYT9750 | 576 | 719 | 94 | S | E | | |
| MYT9751 | 576 | 720 | 95 | K | E | | |
| MYT9752 | 576 | 721 | 96 | S | E | | |
| MYT9753 | 576 | 722 | 97 | S | E | | |
| MYT9754 | 576 | 723 | 98 | T | E | | |

### Example 13. Construction and Screening of pH-Engineered LRRC15 ABPCs

Using the methods detailed in Examples 9-12, desirable mutations may be systematically combined two, three, or more at a time. Heavy and light chain combination variants showing either enhanced dissociation at pH 5.4 or reduced dissociation at pH 7.4 or both, may be selected for further analysis. For example, the mutation positions of MYT8414 and MYT8415 were combined to yield MYT8422. Other double, triple, or even quadruple combinations of desirable mutations may now be routinely combined, and the resulting doubly, triply, and quadruply mutated sequences may be combined and evaluated as disclosed herein.

### Example 14. Characterization of Binding Affinity for Anti-LRRC15 mAbs

Binding affinity of selected LRRC15 pH-engineered antibody variants from Examples 9-12 were measured on U-87 MG (LRRC15+) cells. Briefly, 100,000 U-87 MG cells (ATCC HTB-14) were seeded per well in a 96 well deep well plate. Samrotamab and a pH-engineered antibody variant were serially diluted at a 1:3 dilution in ice cold FC buffer (phosphate buffered saline (PBS), pH 7.4+2 mM ethylenediaminetetraacetic acid (EDTA)+2% (v/v) HI FBS). The plates were spun at 2,000 RPM for 2 minutes, the supernatant was removed, and 100 μL of diluted antibody was added to each well with final concentration ranging from 60 nM to 1 pM and incubated for 2 hours at 4° C. Post incubation, the cells were spun at 2000 rpm for 2 min and supernatant was discarded. Cells were washed twice with 500 μL of ice-cold FC buffer and resuspended with 100 μL of FC buffer. The cells were then transferred from the deep well plate to 96 well round bottom plate, spun at 2000 rpm for 2 min and incubated with 100 L of 10 μg/mL Alexa Fluor 488 conjugated goat anti-human IgG secondary antibody (ThermoFisher Scientific, A11013) for 30 min. Post incubation, cells were washed with FC buffer and resuspended in 100 μL of FC buffer to read on a BD Accuri C6 flow cytometer. Mean fluorescence intensity at each concentration per sample was background subtracted. Binding affinity was measured as a dissociation constant K_{D} by GraphPad Prism assuming Michaelis-Menten binding kinetics. Variants with dissociation constants K_{D} less than 100 nM were selected for further analysis.

### Example 15. Characterization of Cellular Internalization and Endolysosomal Delivery of pH-Engineered Anti-LRRC15 ABPCs

Selected anti-LRRC15 pH-engineered antibody variants from Examples 9-12 were analyzed for internalization and endolysosomal delivery to LRRC15+ SAOS-2 or U87MG cells. Cell counts were determined using trypan blue staining and the Countess II FL Automated Cell Counter (ThermoFisher; AMQAF1000). Cells were then diluted to 75,000 cells/mL and 100 μl/well were seeded into 96-well flat bottom cell culture plates (Genesee Scientific; 25-109) for overnight incubation at 37° C. and 5% CO². The following day, anti-LRRC15 pH-engineered antibody variants, starting ABPC antibodies, control IgG1 isotype control (BP0297, Bioxcell), and vehicle control were diluted in native culture media, and then mixed 1:1 with a 3× molar ratio Zenon pHrodo iFL Human IgG Labeling Reagent (ThermoFisher; Z25611). The mixture was incubated for 30 minutes at room temperature, the existing media was removed from the plated cells and the mixture was added. The mixture of cells, anti-LRRC15 antibody variants, and Zenon pHrodo iFL Human IgG Labeling Reagent was incubated at 37° C., 5% CO2 for 24 hours. Following incubation, the media was removed, and cells are washed with 100 μL of room temperature culture medium. Following the wash 50 μL of Trypsin solution (ThermoFisher Scientific; 12605036) was added and the plate incubated at 37° C. until most of the cells were detached, followed by addition of 50 μL of culture medium. Cells were transferred to a 96-well U-bottom plate (Genesee Scientific; 25-221) and spun down at 2000 RPM for 2 minutes. Cells were washed 2× with 200 μL of ice-cold FACS buffer (phosphate buffered saline (PBS), pH 7.4+2 mM ethylenediaminetetraacetic acid (EDTA)+2% (v/v) HI FBS) before being resuspended in 150 μL of FACS buffer. Cells (Median green fluorescence intensity was detected using an Attune NXT flow cytometer (ThermoFisher Scientific). Data is analyzed using Flowjo analysis software. pHrodo green is a pH sensitive dye that fluoresces in the low pH environment of the endosomes and lysosomes and therefore can be used to quantify antibody internalization and endolysosomal delivery. Internalization and endolysosomal delivery of anti-LRRC15 starting ABPC's and variants at 25 nM in LRRC15+ cells is measured by pHrodo green median fluorescence intensity. Several pH-engineered anti-LRRC15 antibody variants showed increased median fluorescence intensity relative to their corresponding starting ABPC antibodies demonstrating that increased dissociation at lower pH leads to enhanced internalization and endolysosomal delivery inside cells as shown by increased fluorescence or increased fluorescence as compared to IgG1 isotype control. Increased endolysosomal delivery was quantitated for each pH-engineered anti-LRRC15 antibody variant as a ratio of: the variant's median fluorescence intensity minus the median fluorescence intensity of the IgG control, then all divided by the variant's corresponding starting ABPC's median fluorescence intensity minus the median fluorescence intensity of the IgG control.

TABLE 26
Internalization of selected anti-LRRC15 candidates
in SAOS-2 Cells (10 nM, 24 hours). FOS = fold
over samrotamab; FOB = fold over background (IgG control)

| MYT | Sample | FOS | FOB |
|---|---|---|---|
| MYT2737 | Samrotamab LC | 0.62 | 2.01 |
| MYT8391 | hu 15G7 HC his scan variant | 1.20 | 2.94 |
| MYT8416 | hu 15G7 LC his scan variant | 1.05 | 2.71 |
| MYT8417 | hu 15G7 LC his scan variant | 1.04 | 2.68 |
| MYT8483 | hu24D9 Parental | 1.00 | 2.62 |
| MYT8500 | hu 24D9 HC Hydrophobicity Variant | 1.03 | 2.68 |
| MYT8094 | hu29F1 Parental | 1.32 | 3.15 |
| MYT9507 | hu29F1 LC Asp Var | 1.33 | 3.16 |
| MYT9521 | hu29F1 LC Asp Var | 1.24 | 3.02 |
| MYT9523 | hu29F1 LC Asp Var | 1.37 | 3.23 |

TABLE 27
Internalization of selected anti-LRRC15 candidates
in U87MG Cells (10 nM, 24 hours). FOS = fold
over samrotamab; FOB = fold over background (IgG control)

| MYT | Sample | FOS | FOB |
|---|---|---|---|
| MYT2737 | Samrotamab LC | 1.24 | 2.07 |
| MYT3315 | hu139.10 LC | 1.83 | 2.57 |
| MYT8391 | hu 15G7 HC his scan variant | 2.49 | 3.13 |
| MYT8415 | hu 15G7 LC his scan variant | 1.79 | 2.53 |
| MYT8416 | hu 15G7 LC his scan variant | 2.39 | 3.05 |
| MYT8417 | hu 15G7 LC his scan variant | 2.06 | 2.76 |
| MYT8483 | hu24D9 Parental | 1.45 | 2.25 |
| MYT8500 | hu 24D9 HC Hydrophobicity | 1.93 | 2.65 |
| MYT9776 | hu24D9 HC Dev Combos, + LC | 1.97 | 2.69 |
| MYT8094 | hu29F1 Parental | 2.94 | 3.52 |
| MYT9507 | hu29F1 LC Asp Var | 3.01 | 3.58 |
| MYT9521 | hu29F1 LC Asp Var | 2.82 | 3.42 |
| MYT9523 | hu29F1 LC Asp Var | 2.40 | 3.05 |

### Example 16. Thermal Stability of Anti-LRRC15 mAbs

Protein melting temperature (Tm) was measured through the use of Differential Scanning Fluorimetry (DSF). DSF visualizes protein unfolding by measuring the fluorescent signal from the molecule Sypro Orange (Thermo Scientific cat. no. S6650) as a protein unfolds due to heating. As a protein unfolds it exposes more hydrophilic regions to the Sypro Orange dye, which in turns binds to these hydrophilic regions resulting in increase in signal. The Tm for a protein is calculated as the half-maximal of the unfolding transition and can be visualized by plotting the first derivative of the Sypro Orange signal and finding a local maximum of this derivative plot. 20 μL of protein samples in 1×PBS, pH 7.4, was mixed with 5 μL of 25× Sypro Orange master mix, yielding a final concentration of 5× Sypro Orange. The samples were added to 96-well PCR plates (Thermo Scientific Cat. No. AB-2400/W) and sealed with optical covers (Thermo Scientific Cat. No. 4360954). The PCR plate was inserted into a real-time PCR machine (Thermo Scientific Quant Studio 3) and the plate temperature was stabilized for 3 minutes at 25° C. before ramping to 95° C. by 0.2° C. increments, stabilizing for 1 second before the Sypro Orange signal was measured.

### Example 17. Improvement in Selective Binding Affinity of pH Engineered Antibodies

Measurement of the affinity of pH-engineered antibodies specific for LRRC15 is performed by FACS analysis on cell lines with ranges of LRRC15 expression; SAOS-2, U118-MG, G-292. Cell lines selected for this study are obtained from commercial sources and cultured using manufacturer recommended conditions. All cell lines are cultured upon receipt, expanded and cryopreserved at a similar passage number for use in the affinity experiments. Briefly, 1.0×10\hat{ }5 cells that express LRRC15 are plated per well in a 96-well plate in 100 μL media. The cells are washed two times with 200 μL of FACS buffer (1×PBS containing 3% Fetal Bovine Serum) at pH 7.4. The purified protein samples are diluted into FACS buffer at pH 7.4, for a titration from 100 nM to 1 pM, and added to the cells and allowed to bind for 2 hours on ice. After incubation with the primary antibodies, cells are washed twice as before, and then 100 μl of secondary goat anti-Human AF488 (IgG Cross-Adsorbed polyclonal secondary antibody), diluted 1:200, is added in FACS buffer pH 7.4, and incubated for 1 hour on ice. The plates are washed twice. Binding is read on a flow cytometer (Accuri C6, BD Biosciences). Binding is observed as a shift in the FLI signal (as a mean fluorescence intensity) versus secondary alone.

Parent antibodies binding to SAOS-2 cells, expressing high levels of LRRC15, and G-292 cells, expressing lower levels of LRRC15 (e.g., lower levels than SAOS-2 cells), showed similar affinity. Select pH-engineered antibodies specific for LRRC15 may bind more strongly to target cells with higher LRRC15 expression levels, SAOS-2, than to target cells with lower LRRC15 expression, G-292 cells. The pH-engineered antibody constructs may therefore be differentiated from the parent compounds in their avidity, which may be expected to translate to increased selectivity to high expressing target cells in the treatment of LRRC15 overexpressing malignancies, with reduced binding to normal tissues for the selected antibodies.

### Example 18. Increased Half-Life of pH-Engineered ADCs Specific for LRRC15 as Compared to a Control ADCs Specific for LRRC15 in Non-Tumor Bearing Animals

Another aspect of the pH-engineered ADCs specific for LRRC15 described herein can be their ability to facilitate increased serum half-life relative to control antibody ADCs specific for LRRC15 in non-tumor bearing animals. To demonstrate these properties, a series of animal studies in cynomolgus monkeys is performed using pH-engineered ADC specific for LRRC15 and control antibody ADC specific for LRRC15 using methods known to the art (e.g., Gupta, P., et al. (2016), mAbs, 8:5, 991-997). Female monkeys (3 per test article) are administered a bolus of either pH-engineered ADC specific for LRRC15 or control antibody ADC specific for LRRC15 at a dose of 2 to 5 mg/kg via saphenous vein injection. Alternatively, several different doses of LRRC15-binding protein are administered across a group of several monkeys. Blood samples are collected via the peripheral vein or femoral vein at the following time points: pre-dose, 15 minutes, 12 hours, 2 days, 3 days, 4 days, 7 days, 10 days, 14 days, 17 days, 21 days and 28 days post-dose. Blood samples are analyzed for the presence of either pH-engineered ADC specific for LRRC15 or control antibody ADC specific for LRRC15 using methods known to the art (e.g., ELISA).

Antibody concentrations of pH-engineered ADC specific for LRRC15 and control antibody ADC specific for LRRC15 are plotted as a function of time. Upon analysis of the data, it can be observed that the pH-engineered ADC specific for LRRC15 has a significantly longer serum half-life relative to control antibody ADC specific for LRRC15, thereby demonstrating the improved serum stability and exposure profile.

### Example 19. Rabbit mAb Cytotoxicity from Supernatants Assay

The cytotoxicity of rabbit monoclonal antibodies from supernatants was evaluated on an LRRC15 expressing cell line, U118-MG. The expression of LRRC15 was characterized by measuring receptor density using flow cytometry. Cells were seeded at ˜2,500 per well in 100 μL of culture medium (DMEM with 10% FBS) in 96 well assay plates then incubated at 37° C. overnight to allow for cell attachment. After approximately 24 hours, cells were treated with B cell supernatant derived from rabbit immunization. The supernatant samples were diluted at a ratio of 1:10, 1:100, and 1:1000 using culture medium. Diluted supernatant samples were combined with a secondary non-cleavable MMAF (Monomethylauristatin F) toxin at a 1:1 ratio and incubated for 20 minutes at room temperature to form a complex. After 20 minutes, media from the assay plate is replaced with 100 μL of the supernatant-toxin complex and incubated for 4 days. At the end of the culture period, viability was assessed using CCK-8, a viability reagent used to generate absorbance generated by the activities of dehydrogenases in cells directly proportional to the number of live cells. Absorbance was read using a plate reader.

### Example 20. Anti-LRRC15 Cytotoxicity in SAOS-2 Cells

The cytotoxicity of various anti-LRRC15 candidates was evaluated by complexing them to toxin-conjugated secondary antibodies, and then exposing them to SAOS-2 cells. Briefly, cells were plated at 2.5E3 cells/well in 384 well plates and incubated overnight. The mAbs were prepared beginning at a 100 nm titrate (1:5), 11-point curve. Secondary antibody conjugated with A-MMAF was added to form a complex (25 nM), and 5 μL was added to each well. Cells were incubated for 5 days and a CTG assay was performed.

TABLE 28
Cytotoxicity of candidates against SAOS-2 cells

| | | Fold over |
|---|---|---|
| MYT # | Description | MYT5438 |
| MYT5438 | Samrotamab WT | 1.00 |
| MYT8500 | hu24D9 HC Hydrophobicity Variant | 1.06 |
| MYT8391 | hu15G7 HC his scan variants | 1.14 |
| MYT8416 | hu15G7 LC his scan variants | 1.13 |
| MYT8094 | hu29F1 Parent | 1.45 |
| MYT9521 | hu29F1 LC Asp Var | 1.49 |
| MYT9731 | hu29F1 LC Glu Var | 1.05 |
| IgG | Non-Binding Control | -1.16 |

TABLE 29
Cytotoxicity of candidates against SAOS-2 cells

| Index | Description | 20 nM Efficacy |
|---|---|---|
| 123 | Samrotamab ADC LC Variant | 20.20 |
| 124 | hu139.10 ADC LC Variant | 22.04 |
| 071 | Non-Binding ADC Control | 3.59 |
| 076 | Samrotamab V205C ADC | 16.44 |
| 127 | hu15G7 ADC HC Variant | 63.78 |
| 128 | hu15G7 ADC LC Variant | 38.73 |
| 129 | hu15G7 ADC LC Variant | 23.22 |
| 130 | hu15G7 ADC LC Variant | 42.46 |
| 071 | Non-Binding ADC Control | 3.59 |
| 076 | Samrotamab V205C ADC | 16.44 |
| 131 | hu24D9 ADC | 29.43 |
| 132 | hu24D9 ADC HC Variant | 26.36 |
| 133 | hu24D9 ADC HC/LC Variant | 66.26 |
| 071 | Non-Binding ADC Control | 3.59 |
| 076 | Samrotamab V205C ADC | 16.44 |

### Example 21. Increased Potency of pH-Engineered ADCs Specific for LRRC15 vs. A Control ADC Specific for LRRC15 in Mouse Xenograft Models

The enhanced anti-tumor activity of the pH-engineered ADCs specific for LRRC15 against LRRC15+ tumors was demonstrated in a subcutaneous xenograft model of LRRC15+ cells. For the experiments, LRRC15+SA4033 cells (leiomyosarcoma; IHC 2+) were grown in vitro, propagated in vivo, and inoculated subcutaneously into the right flank of female immunodeficient (SCID-Beige, NOD-SCID) mice. When the tumor reached 150-250 mm³, the mice were dosed intraperitoneally (IP) with either vehicle, samrotamab ADC, or the test article indicated in Table 30. Briefly, the test articles were produced substantially as described herein (see, e.g., paragraphs [0495-0496] above), by conjugating vcMMAE to the cysteine at position 205 of the light chain of each antibody. Measurements of the length (L) and width (W) of the tumors were taken via an electronic caliper. The volume was calculated using the following equation: V=(L×W\hat{ }2)/2. A bolus (e.g., 2-6 mg/kg) of either pH-engineered ADC specific for LRRC15 or control ADC specific for LRRC15 was administered via tail vein. Tumor growth inhibition (TGI), tumor growth delay (TGD), and survival were significantly improved with administration of pH-engineered ADC specific for LRRC15 compared to administration of non-pH-engineered control ADC specific for LRRC15 at the same regimen. As indicated in FIG. 8, all candidates outperformed samrotamab ADC save for one of the 24D9-based ADCs.

Optionally, the spread of tumor cells into various tissues is determined in sacrificed animals. Metastasis is measured according to Schneider, T. et al., Clin. Exp. Metas. 19 (2002) 571-582. Briefly, tissues are harvested, and human Alu sequences are quantified by real-time PCR. Higher human DNA levels, quantified by real-time PCR, correspond to higher levels of metastasis. Levels of human Alu sequences (correlating to the invasion of tumor cells into secondary tissue) are significantly lower in animals treated with pH-engineered ADC specific for LRRC15, corresponding to reduced metastasis, compared to mice treated with control ABPC ADC specific for LRRC15 at the same regimen. Alternatively, the enhanced anti-tumor activity of the pH-engineered ADC specific for LRRC15 can be shown in LRRC15+ PDX models (e.g., available from Charles River Laboratories).

TABLE 30
Candidate anti-LRRC15 ADCs administered to mice harboring SA4033 cells

| MYT # | Index # | HC Plasmid | HC Seq ID | LC Plasmid | LC Seq ID |
|---|---|---|---|---|---|
| MYT2737-c-ADC | 123 | pNGH0761 | 724 | pNGL1019 | 725 |
| MYT3315-c-ADC | 124 | pNGH1909 | 726 | pNGL1322 | 727 |
| MYT8391-c-ADC | 127 | pNGH4960 | 728 | pNGL2802 | 729 |
| MYT8415-c-ADC | 128 | pNGH5088 | 730 | pNGL3334 | 731 |
| MYT8417-c-ADC | 130 | pNGH5088 | 730 | pNGL3336 | 732 |
| MYT8483-c-ADC | 131 | pNGH5090 | 733 | pNGL2828 | 734 |
| MYT9776-c-ADC | 133 | pNGH6020 | 735 | pNGL4035 | 736 |
| MYT9521-c-ADC | 135 | pNGH4765 | 737 | pNGL3940 | 738 |
| MYT9731-c-ADC | 137 | pNGH4765 | 737 | pNGL3958 | 739 |
| MYT8094-c-ADC | 115 | pNGH4765 | 737 | pNGL3191 | 740 |

### Example 22. Increased Exposure of pH-Engineered ADCs Specific for LRRC15 vs. A Control ABPC ADC Specific for LRRC15 in Mouse & Monkey Models

PK analysis was conducted to evaluate the exposure of pH-engineered LRRC15 ADC. PK was determined in non-tumor bearing 6-8-week SCID female mice weighing 18-22 gm. Each animal received a single tail vein injection of the indicated LRRC15 ADC diluted in PBS. A control ABPC ADC specific for LRRC15 was also administered as a reference control.

Adverse events were not found during the study, including any weight loss due to ADC infusion. Blood was collected via submandibular bleed and plasma was separated in different groups of mice at the following time points: 1 h, 6 h, 1 d, 3 d, 5 d, 7 d, 10 d, 14 d, 21 d, 28 d. The collected plasma was stored in −80° C. Validated assays were used to determine observed plasma concentration (Cmax) and the time taken to reach the peak (Tmax) for each pH-engineered ABPC-ADC and control ABPC-ADC. The area under the curve (AUC) and half-life (T1/2) was then calculated using non-compartmental analysis. PK analysis was also conducted in primates, and the results are summarized in Table 31.

TABLE 31
Anti-LRRC15 ADC candidates exhibited longer half-
lives vs. samrotamab in non-human primates

| MYT # | Index | T ½ |
|---|---|---|
| MYT5438-ADC | Sam replicate 1 | 1.228 |
| MYT5438-ADC | Sam replicate 2 | 1.818 |
| MYT5438-ADC | Sam replicate 3 | 1.528 |
| MYT2737-c-ADC | 123 | 2.464 |
| MYT3315-c-ADC | 124 | 3.785 |
| MYT8391-c-ADC | 127 | 3.524 |
| MYT8415-c-ADC | 128 | 5.212 |
| MYT8417-c-ADC | 130 | 3.536 |
| MYT8483-c-ADC | 131 | 3.142 |
| MYT9776-c-ADC | 133 | 2.679 |
| MYT9521-c-ADC | 135 | 3.954 |
| MYT9731-c-ADC | 137 | 3.721 |
| MYT8094-c-ADC | 115 | 4.115 |

### Example 23. Engineering of Developable, Humanized Versions of 15G7, 24D9, and 29F1

The term “developability” as used herein encompasses the feasibility of antigen-binding proteins (ABPs) to successfully progress from discovery to development via evaluation and engineering of their physicochemical properties. These properties include but are not limited to the tendency for aggregation and self-interaction, colloidal stability, thermal stability, etc. Ultimately, selection of candidate therapeutic antibodies and ADCs depends upon a rigorous evaluation of the biological function, efficacy, safety, and developability of each starting or parental ABP.

Highly developable, humanized versions of the newly discovered 15G7, 24D9, and 29F1 RabMAb clones were generated through non-routine antibody engineering techniques, especially as evidenced by the unpredictability of producing acceptable variants. For example, even if a given mutation provides a benefit for one of the developability parameters, it can negatively impact the other parameters. However, now that Applicant has disclosed the tolerated/favorable developability mutations (e.g., as embodied, for example, by the candidates listed in Table 7), the skilled person may generate alternative versions by applying routine techniques. For example, if a favorable developability mutation comprises a substitution of a wildtype (WT) amino acid residue to an aspartate, it would be reasonable to expect that a neutral substitution (e.g., a glutamate substitution) would likewise function well (see Table 32, grouping amino acids by class). A “neutral substitution” may include the substitution of one amino with a different amino acid from the same class.

TABLE 32
Proteinogenic amino acids grouped by class

| Class | Amino acids | 1-letter code |
|---|---|---|
| Aliphatic | Glycine, Alanine, Valine, Leucine, Isoleucine | G, A, V, L, I |
| Hydroxyl | Serine, Cysteine, Selenocysteine, Threonine, | S, C, U, T, M |
| or sulfur/selenium- | Methionine | |
| containing | | |
| Cyclic | Proline | P |
| Aromatic | Phenylalanine, Tyrosine, Tryptophan | F, Y, W |
| Basic | Histidine, Lysine, Arginine | H, K, R |
| Acidic and their amides | Aspartate, Glutamate, Asparagine, Glutamine | D, E, N, Q |

Applicant conducted extensive mutagenesis of the 15G7, 24D9, and 29F1 RabMAbs to produce humanized antibodies having desirable developability characteristics. In addition to the histidine, aspartate, and glutamate scanning described above, further scanning was performed to overcome certain “developability flags”. Such red flags include but are not limited to a hydrophobic interaction chromatography (HIC) retention time (RT) of ≥16.6 minutes; protein expression of less than ˜10 mg/L; presence of less than ˜85% monomer (measured by size exclusion chromatography, SEC), or a baculovirus viral particle assay value of greater than ˜30.

TABLE 33
Summary of selected 15G7 and 24D9 useful developability mutations

| Clone | Useful Developability Mutations |
|---|---|
| 15G7 | L46R in the light chain variable domain (LCVD) |
| 15G7 | V49I in the heavy chain variable domain (HCVD) + L46R in the LCVD |
| 15G7 | I71F in the HCVD + L46R in the LCVD |
| 15G7 | I71F, R73K, D74T, N78T, Y81T, N85T, R88T, and Y96F in the HCVD + |
| | L46R in the LCVD |
| 24D9 | M85K, N86T in the HCVD |
| 24D9 | M85K, N86T in the HCVD + T1OP in the LCVD |
| 24D9 | T1OP in the LCVD |
| 24D9 | M85K, N86T in the HCVD |

The developability efforts yielded multiple promising antibodies, including MYT8391-c (comprising SEQ ID NOs: 728 & 729); MYT8415-c (comprising SEQ ID NOs: 730 & 731); MYT8417-c (comprising SEQ ID NOs: 730 & 732); MYT8483-c (comprising SEQ ID NOs: 733 & 734); MYT9776-c (comprising SEQ ID NOs: 735 & 736); MYT9521-c (comprising SEQ ID NOs: 737 & 738); MYT9731-c (comprising SEQ ID NOs: 737 & 739); and MYT8094-c (comprising 737 & 740). Each of these antibodies has been produced with and without a cysteine at light chain position 205.

### Example 24. Increased Potency of pH-Engineered ADCs Specific for LRRC15 vs. A Control ABPC ADC Specific for LRRC15 in Mouse Patient-Derived Xenograft (PDX) Models of Sarcoma

Anti-LRRC15 ABPCs of the disclosure demonstrated significant antitumor activity in multiple LRRC15 cancer-positive models, including a PDX model of osteosarcoma (CTG-0241) with high LRRC15 cancer and stromal expression (3+ IHC). Briefly, 5×5×5 mm diameter tumor fragments were implanted unilaterally on the left flank with tumor fragments harvested from stock animals. Measurements of the tumors' length (L) and width (W) were taken via an electronic caliper. The volume was calculated using the following equation: Tumor volume (TV)=width*width*length*0.52. When tumors reached an average tumor volume of 200-400 mm³, animals were matched by tumor volume into treatment or control groups for dosing. On Day 1, a bolus (6 mg/kg) of either pH-engineered ADC specific for LRRC15 or control ADC specific for LRRC15 was administered via the tail vein. Body weight and TVs were measured bi-weekly (FIG. 9).

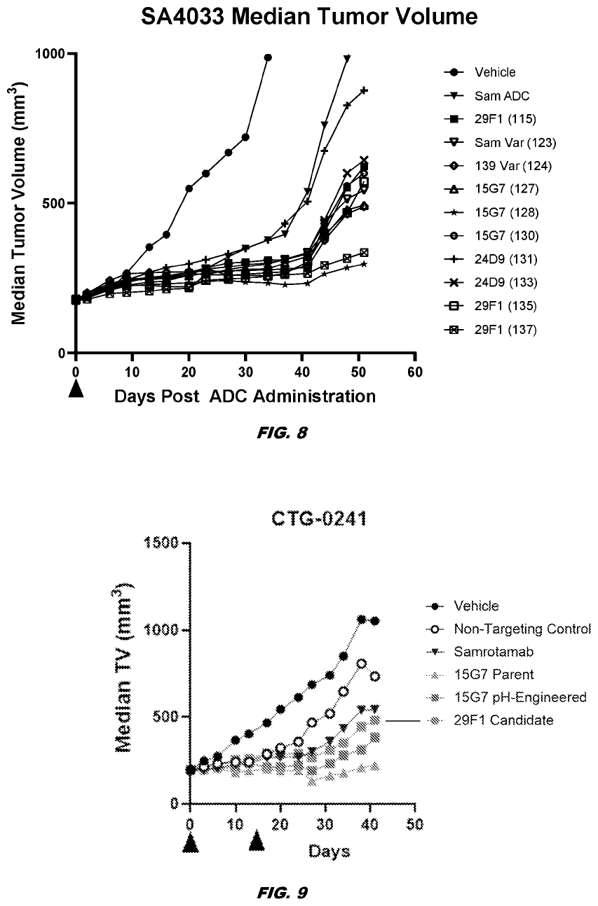

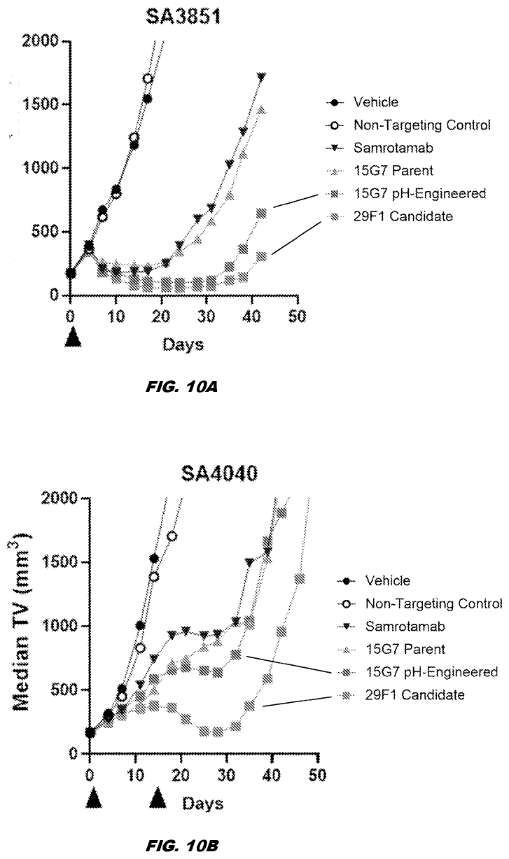

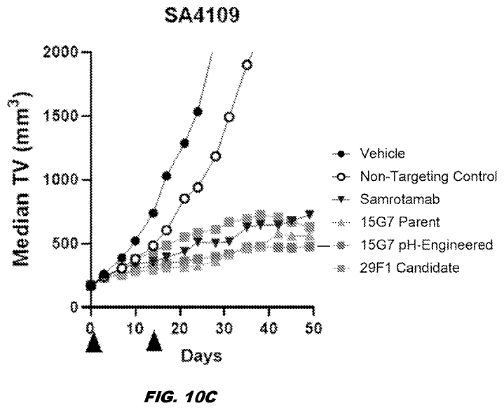

Cells from LRRC15+SA3851 (osteosarcoma; IHC 3+), SA4040 (osteosarcoma; IHC 3+), SA4109 (chondrosarcoma; IHC 2+), PDX models were propagated in vivo in NOD-SCID mice. Approximately 2-3 mm diameter tumor fragments from stock mice were harvested and inoculated subcutaneously in the right upper flank of each mouse. Measurements of the tumors' length (L) and width (W) were taken via an electronic caliper. The volume was calculated using the following equation: TV=(L×W\hat{ }2)/2. When the tumor reached 150-200 mm³, the mice were randomized into six groups. Each group of mice received an intravenous (IV) dose of huIgG1, with treatment being initiated on day 0. On Day 1, a bolus (6 mg/kg) of either pH-engineered ADC specific for LRRC15 or control ADC specific for LRRC15 was administered via the tail vein. Body weight and TVs were measured bi-weekly. The studies were terminated as follows: SA3851 (day 63), SA4040 (day 59); SA4109 (day 70), and the TVs were graphed as shown in FIGS. 10A, 10B, and 10C, respectively.

Other aspects, advantages, and modifications are within the scope of the following numbered Embodiments.

Embodiment 1. A pharmaceutical composition comprising an effective amount of an antigen-binding protein construct (ABPC) comprising: a first antigen-binding domain (ABD) that is capable of specifically binding LRRC15 or an epitope of LRRC15 presented on the surface of a target mammalian cell, optionally wherein the dissociation rate of the first ABD at a pH of ˜4.0-6.5 is faster than the dissociation rate at a pH of ˜7.0-8.0 or the KD of the first ABD at a pH of ˜4.0-6.5 is greater than the KD at a pH of ˜7.0-8.0. When subsequent embodiments refer to a control ABPC, it is intended (unless otherwise specified) that the subject ABPC contains amino acid substitution(s) with respect to its control, which render(s) the subject ABPC pH-dependent (for specific target binding) relative to its respective control ABPC.

Embodiment 2. The pharmaceutical composition of embodiment 1, wherein the first ABD comprises a heavy chain variable domain (HCVD) of one of: an HCVD of (a) 15G7; (b) 24D9; and (c) 29F1, each HCVD optionally with one or more amino acids substituted with a histidine, and optionally wherein the 29F1 HCVD has one or more amino acids substituted with an aspartate or a glutamate.

Embodiment 3. The pharmaceutical composition of embodiment 1, wherein the first ABD comprises a light chain variable domain (LCVD) of one of: an LCVD of (a) 15G7; (b) 24D9; and (c) 29F1, each LCVD optionally with one or more amino acids substituted with a histidine, optionally wherein the 29F1 LCVD has one or more amino acids substituted with an aspartate or a glutamate.

Embodiment 4. The pharmaceutical composition of embodiment 1, wherein the ABD comprises (a) an HCVD of 15G7; and/or an LCVD of 15G7; (b) an HCVD of 24D9; and/or an LCVD of 24D9; or (c) an HCVD of 29F1; and/or an LCVD of 29F1; optionally wherein each HCVD and/or LCVD has one or more amino acid substituted with a histidine, an aspartate, or a glutamate; optionally, wherein the ABD is selected from MYT2737 (HCV=SEQ ID NO: 1, LCV=SEQ ID NO: 64), MYT3315 (HCV=SEQ ID NO: 84, LCV=SEQ ID NO: 154), MYT8391 (HCV=SEQ ID NO: 430, LCV=SEQ ID NO: 455), MYT8415 (HCV=SEQ ID NO: 382, LCV=SEQ ID NO: 489), MYT8417 (HCV=SEQ ID NO: 382, LCV=SEQ ID NO: 491), MYT8483 (HCV=SEQ ID NO: 516, LCV=SEQ ID NO: 522), MYT9776 (HCV=SEQ ID NO: 571, LCV=SEQ ID NO: 517), MYT8094 (HCV=SEQ ID NO: 576, LCV=SEQ ID NO: 581), MYT9521 (HCV=SEQ ID NO: 576, LCV=SEQ ID NO: 639), MYT9731 (HCV=SEQ ID NO: 576, LCV-SEQ ID NO: 700), MYT8416 (HCV=SEQ ID NO: 382, LCV=SEQ ID NO: 490), MYT8500 (HCV=SEQ ID NO: 570, LCV=SEQ ID NO: 522), MYT9523 (HCV=SEQ ID NO: 576, LCV=SEQ ID NO: 641), MYT4174 (HCV=SEQ ID NO: 84, LCV=SEQ ID NO: 177), and MYT9507 (HCV=SEQ ID NO: 576, LCV=SEQ ID NO: 625); or optionally, wherein the ABD is selected from an antibody comprising heavy and light chain polypeptide sequence pairs that are at least 95% identical to the pairs of sequences set forth in SEQ ID NOs: 724 and 725; 726 and 727; 728 and 729; 730 and 731; 730 and 732; 733 and 734; 735 and 736; 737 and 738; 737 and 739; and 737 and 740. In some embodiments, the LCV sequence does not comprise a cysteine at position 205. In some embodiments, any LCV sequence recited in Embodiment 4 may comprise a C205V, a C205I, or a C205L substitution.

Embodiment 5. The pharmaceutical composition of embodiment 2 or 4, wherein the HCVD comprises one of: (a) an HCVD of 15G7 comprising a sequence comprising SEQ ID NO: 378 or SEQ ID NO: 382; (b) an HCVD of 24D9 comprising a sequence comprising SEQ ID NO: 512 or SEQ ID NO: 516; and (c) an HCVD of 29F1 comprising a sequence comprising SEQ ID NO: 572 or SEQ ID NO: 576.

Embodiment 6. The pharmaceutical composition of embodiment 3 or 4, wherein the LCVD comprises one of: (a) an LCVD of 15G7 comprising a sequence comprising SEQ ID NO: 451 or SEQ ID NO: 455; (b) an LCVD of 24D9 comprising a sequence comprising SEQ ID NO: 518 or SEQ ID NO: 522; and (c) an LCVD of 29F1 comprising a sequence comprising SEQ ID NO: 577 or SEQ ID NO: 581.

Embodiment 7. The pharmaceutical composition of embodiment 1, wherein the first ABD comprises an HCVD comprising the CDR1, 2, and 3 of one of: (a) SEQ ID NOs: 379-381; (b) SEQ ID NOs: 513-515; and (c) SEQ ID NOs: 573-575, optionally wherein each set of three CDRs has collectively one or more amino acid position substituted with a histidine, an aspartate, or a glutamate.

Embodiment 8. The pharmaceutical composition of embodiment 1, wherein the first ABD comprises an LCVD comprising the CDR1, 2, and 3 of one of: (a) SEQ ID NOs: 452-454; (b) SEQ ID NOs: 519-521; and (c) SEQ ID NOs: 578-580, optionally wherein each set of three CDRs has collectively one or more amino acid position substituted with a histidine, an aspartate, or a glutamate.

Embodiment 9. The pharmaceutical composition of embodiment 1, wherein the first ABD comprises an HCVD of one of: (a) an HCVD comprising the CDR1, 2, and 3 of SEQ ID NOs: 379-381; and/or an LCVD comprising the CDR1, 2, and 3 of one of SEQ ID NOs: 452-454; (b) an HCVD comprising the CDR1, 2, and 3 of one of SEQ ID NOs: 513-515; and/or an LCVD comprising the CDR1, 2, and 3 of SEQ ID NOs: 519-521; and (c) an HCVD comprising the CDR1, 2, and 3 of SEQ ID NOs: 573-575; and/or an LCVD comprising the CDR1, 2, and 3 of SEQ ID NOs: 578-580; optionally wherein each set of three HCVD and/or LCVD CDRs has collectively one or more amino acid position substituted with a histidine, an aspartate, or a glutamate.

Embodiment 10. The pharmaceutical composition of embodiment 1, 2, or 7, wherein the first ABD comprises an HCVD of one of: (a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; (b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and (c) an HCVD comprising a sequence that is at least 90% identical to the sequence set forth in SEQ ID NO: 572 or SEQ ID NO: 576, wherein the HCVD includes a histidine, an aspartate, or a glutamate at one or more positions in SEQ ID NO: 572 or SEQ ID NO: 576 selected from the group consisting of: D positions selected from 31, 56 and 99; and an E position selected from 59.

Embodiment 11. The pharmaceutical composition of embodiment 1, 3, or 8, wherein the first ABD comprises an LCVD of one of: (a) an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and (c) an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

Embodiment 12. The pharmaceutical composition of embodiment 1, 2, or 7, wherein the first ABD comprises an HCVD of one of: (a) an HCVD comprising a sequence that is at least 90% identical to the sequence set forth in SEQ ID NO: 378 or SEQ ID NO: 382, wherein the HCVD includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53,106; (b) an HCVD comprising a sequence that is at least 90% identical to the sequence set forth in SEQ ID NO: 512 or SEQ ID NO: 516, wherein the HCVD includes a histidine at two or more positions in SEQ ID NO: 512 or SEQ ID NO: 516; and (c) an HCVD comprising a sequence that is at least 90% identical to the sequence set forth in SEQ ID NO: 572 or SEQ ID NO: 576, wherein the HCVD includes a histidine, an aspartate, or a glutamate at two or more positions in SEQ ID NO: 572 or SEQ ID NO: 576.

Embodiment 13. The pharmaceutical composition of embodiment 1, 3, or 8, wherein the first ABD comprises an LCVD of one of: (a) an LCVD comprising a sequence that is at least 90% identical to the sequence set forth in SEQ ID NO: 451 or SEQ ID NO: 455, wherein the LCVD includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96 and 93, 96; and (b) an LCVD comprising a sequence that is at least 90% identical to the sequence set forth in SEQ ID NO: 518 or SEQ ID NO: 522, wherein the LCVD includes a histidine at two or more positions in SEQ ID NO: 518 or SEQ ID NO: 522; and (c) an LCVD comprising a sequence that is at least 90% identical to the sequence set forth in SEQ ID NO: 577 or SEQ ID NO: 581, wherein the LCVD includes a histidine, an aspartate, or a glutamate at two or more positions in SEQ ID NO: 577 or SEQ ID NO: 581.

Embodiment 14. The pharmaceutical composition of embodiment 1, 4, or 9, wherein the first ABD comprises one of: (a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; and/or an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and (c) an HCVD comprising a sequence that is at least 90% identical to the sequence set forth in SEQ ID NO: 572 or SEQ ID NO: 576, wherein the HCVD includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

Embodiment 15. The pharmaceutical composition of embodiment 1, wherein the first ABD comprises an HCVD comprising the sequence set forth in SEQ ID NO: 378, SEQ ID NO: 382, one of SEQ ID NOs: 383-424, one of SEQ ID NOs 425-450, SEQ ID NO: 512, SEQ ID NO: 516, one of SEQ ID NOs: 523-567, one of SEQ ID NOs: 568-571, SEQ ID NO: 572, SEQ ID NO: 576, one of SEQ ID NOs 582-624, or one of SEQ ID NOs: 653-695.

Embodiment 16. The pharmaceutical composition of embodiment 1 or 15, wherein the first ABD comprises an LCVD comprising the sequence set forth in SEQ ID NO: 451, SEQ ID NO: 455, one of SEQ ID NOs: 456-485, SEQ ID NOs: 486-511, SEQ ID NO: 518, SEQ ID NO: 522, SEQ ID NO: 577, SEQ ID NO: 581, one of SEQ ID NOs: 625-652, or one of SEQ ID NOs: 696-723.

Embodiment 17. The pharmaceutical composition of embodiment 1, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO: 451, SEQ ID NO: 455, one of SEQ ID NOs: 456-485, or one of SEQ ID NOs: 486-511, wherein the first ABD does not comprise (a) an HCVD comprising the sequence set forth in SEQ ID NO: 378 or SEQ ID NO: 382 and an LCVD that does not comprise the sequence set forth in SEQ ID NO: 461, SEQ ID NO: 462, SEQ ID NO: 464, SEQ ID NO: 466, SEQ ID NO: 467, one of SEQ ID NOs: 477-478, or one of SEQ ID NOs: 480-482; or (b) an LCVD of SEQ ID NO: 451 or SEQ ID NO: 455 and an HCVD that does not comprise the sequence set forth in SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, or SEQ ID NO: 450.

Embodiment 18. The pharmaceutical composition of any one of embodiments 1-17, wherein the ABPC is degraded in the target mammalian cell following internalization of the ABPC by the target mammalian cell.

Embodiment 19. The pharmaceutical composition of any one of embodiments 1-18, wherein the ABPC further comprises a conjugated toxin, radioisotope, drug, or small molecule.

Embodiment 20. The pharmaceutical composition of embodiment 19, wherein the composition provides for an increase in toxin liberation in the target mammalian cell as compared to a composition comprising the same amount of a control ABPC.

Embodiment 21. The pharmaceutical composition of embodiment 20, wherein the composition provides for at least a 20%, 50%, 2-fold, or 5-fold increase in toxin liberation in the target mammalian cell as compared to a composition comprising the same amount of a control ABPC.

Embodiment 22. The pharmaceutical composition of any one of embodiments 19-21, wherein the composition provides for an increase in target mammalian cell killing as compared to a composition comprising the same amount of a control ABPC.

Embodiment 23. The pharmaceutical composition of embodiment 22, wherein the composition provides for at least a 20%, 50%, 2-fold, or 5-fold increase in target mammalian cell killing as compared to a composition comprising the same amount of a control ABPC.

Embodiment 24. The pharmaceutical composition of any one of embodiments 1-23, wherein the composition provides for an increase in endolysosomal delivery in the target mammalian cell as compared to a composition comprising the same amount of a control ABPC.

Embodiment 25. The pharmaceutical composition of embodiment 24, wherein the composition provides for at least a 20%, 50%, 2-fold, or 5-fold increase in endolysosomal delivery in the target mammalian cell as compared to a composition comprising the same amount of a control ABPC.

Embodiment 26. The pharmaceutical composition of any one of embodiments 1-25, wherein the composition results in a less or no detectable reduction in the level of LRRC15 presented on the surface of the target mammalian cell as compared to a composition comprising the same amount of a control ABPC.

Embodiment 27. A pharmaceutical composition comprising an effective amount of an antigen-binding protein construct (ABPC) comprising: a first ABD that is capable of specifically binding LRRC15 or an epitope of LRRC15 presented on the surface of a target mammalian cell; and a conjugated toxin, radioisotope, drug, or small molecule, optionally wherein: (a) the dissociation rate of the first ABD at a pH of ˜4.0-6.5 is faster than the dissociation rate at a pH of ˜7.0-8.0; or the dissociation constant (KD) of the first ABD at a pH of ˜4.0-6.5 is greater than the KD at a pH of ˜7.0-8.0; and (b) the composition provides for one or more of: (i) an increase in toxin liberation in the target mammalian cell as compared to a composition comprising the same amount of a control ABPC; (ii) an increase in target mammalian cell killing as compared to a composition comprising the same amount of a control ABPC; and (iii) an increase in endolysosomal delivery in the target mammalian cell as compared to a composition comprising the same amount of a control ABPC.

Embodiment 28. The pharmaceutical composition of embodiment 27, wherein the first ABD comprises a heavy chain variable domain (HCVD) of one of: an HCVD of (a) 15G7; (b) 24D9; and (c) 29F1, optionally wherein the HCVD has one or more amino acid position substituted with a histidine, an aspartate, or a glutamate.

Embodiment 29. The pharmaceutical composition of embodiment 27, wherein the first ABD comprises a light chain variable domain (LCVD) of one of: an LCVD of (a) 15G7; (b) 24D9; and (c) 29F1, optionally wherein the LCVD has one or more amino acid position substituted with a histidine, an aspartate, or a glutamate.

Embodiment 30. The pharmaceutical composition of embodiment 27, wherein the ABD comprises (a) an HCVD of 15G7; and/or an LCVD of 15G7; (b) an HCVD of 24D9; and/or an LCVD of 24D9; or (c) an HCVD of 29F1; and/or an LCVD of 29F1; optionally wherein each HCVD and/or LCVD has one or more amino acid substituted with a histidine, an aspartate, or a glutamate.

Embodiment 31. The pharmaceutical composition of embodiment 28 or 30, wherein the HCVD comprises one of: (a) an HCVD of 15G7 comprising a sequence comprising SEQ ID NO: 378 or SEQ ID NO: 382; (b) an HCVD of 24D9 comprising a sequence comprising SEQ ID NO: 512 or SEQ ID NO: 516; and (c) an HCVD of 29F1 comprising a sequence comprising SEQ ID NO: 572 or SEQ ID NO: 576.

Embodiment 32. The pharmaceutical composition of embodiment 29 or 30, wherein the LCVD comprises one of: (a) an LCVD of 15G7 comprising a sequence comprising SEQ ID NO: 451 or SEQ ID NO: 455; (b) an LCVD of 24D9 comprising a sequence comprising SEQ ID NO: 518 or SEQ ID NO: 522; and (c) an LCVD of 29F1 comprising a sequence comprising SEQ ID NO: 577 or SEQ ID NO: 581.

Embodiment 33. The pharmaceutical composition of embodiment 27, wherein the first ABD comprises an HCVD comprising the CDR1, 2, and 3 of one of: (a) SEQ ID NOs: 379-381; (b) SEQ ID NOs: 513-515; and (c) SEQ ID NOs: 573-575, optionally wherein each set of three CDRs has collectively one or more amino acid position substituted with a histidine, an aspartate, or a glutamate.

Embodiment 34. The pharmaceutical composition of embodiment 27, wherein the first ABD comprises an LCVD comprising the CDR1, 2, and 3 of one of: (a) SEQ ID NOs: 452-454; (b) SEQ ID NOs: 519-521; and (c) SEQ ID NOs: 578-580, optionally wherein each set of three CDRs has collectively one or more amino acid position substituted with a histidine, an aspartate, or a glutamate.

Embodiment 35. The pharmaceutical composition of embodiment 27, wherein the first ABD comprises one of: (a) an HCVD comprising the CDR1, 2, and 3 of SEQ ID NOs: 379-381; and/or an LCVD comprising the CDR1, 2, and 3 of one of SEQ ID NOs: 452-454; (b) an HCVD comprising the CDR1, 2, and 3 of one of SEQ ID NOs: 513-515; and/or an LCVD comprising the CDR1, 2, and 3 of SEQ ID NOs: 519-521; or (c) an HCVD comprising the CDR1, 2, and 3 of SEQ ID NOs: 573-575; and/or an LCVD comprising the CDR1, 2, and 3 of SEQ ID NOs: 578-580; optionally wherein each set of three CDRs has collectively one or more amino acid position substituted with a histidine, an aspartate, or a glutamate.

Embodiment 36. The pharmaceutical composition of embodiment 27, 28, or 33, wherein the first ABD comprises an LCVD of one of: (a) an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and (c) an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

Embodiment 37. The pharmaceutical composition of embodiment 27, 29, or 34, wherein the first ABD comprises an HCVD of one of: (a) an HCVD comprising a sequence that is at least 90% identical to the sequence set forth in SEQ ID NO: 378 or SEQ ID NO: 382, wherein the HCVD includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53,106; and (b) an HCVD comprising a sequence that is at least 90% identical to the sequence set forth in SEQ ID NO: 512 or SEQ ID NO: 516, wherein the HCVD includes a histidine at two or more positions in SEQ ID NO: 512 or SEQ ID NO: 516.

Embodiment 38. The pharmaceutical composition of embodiment 27, 28, or 33, wherein the first ABD comprises an LCVD of one of: (a) an LCVD comprising a sequence that is at least 90% identical to the sequence set forth in SEQ ID NO: 451 or SEQ ID NO: 455, wherein the LCVD includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96 and 93, 96; and (b) an LCVD comprising a sequence that is at least 90% identical to the sequence set forth in SEQ ID NO: 518 or SEQ ID NO: 522, wherein the LCVD includes a histidine at two or more positions in SEQ ID NO: 518 or SEQ ID NO: 522.

Embodiment 39. The pharmaceutical composition of embodiment 27, 29, 34, wherein the first ABD comprises one of: (a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; and/or an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and (c) an HCVD comprising a sequence that is at least 90% identical to the sequence set forth in SEQ ID NO: 572 or SEQ ID NO: 576, wherein the HCVD includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

Embodiment 40. The pharmaceutical composition of embodiment 27, 30, or 35, wherein the first ABD comprises an HCVD comprising the sequence set forth in SEQ ID NO: 378, SEQ ID NO: 382, one of SEQ ID NOs: 383-424, one of SEQ ID NOs 425-450, SEQ ID NO: 512, SEQ ID NO: 516, one of SEQ ID NOs: 523-567, one of SEQ ID NOs: 568-571, SEQ ID NO: 572, SEQ ID NO: 576, one of SEQ ID NOs 582-624, or one of SEQ ID NOs: 653-695.

Embodiment 41. The pharmaceutical composition of embodiment 27, wherein the first ABD comprises an LCVD comprising the sequence set forth in SEQ ID NO: 451, SEQ ID NO: 455, one of SEQ ID NOs: 456-485, SEQ ID NOs: 486-511, SEQ ID NO: 518, SEQ ID NO: 522, SEQ ID NO: 577, SEQ ID NO: 581, one of SEQ ID NOs: 625-652, or one of SEQ ID NOs: 696-723.

Embodiment 42. The pharmaceutical composition of embodiment 27 or 41, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO: 451, SEQ ID NO: 455, one of SEQ ID NOs: 456-485, or one of SEQ ID NOs: 486-511, wherein the first ABD does not comprise (a) an HCVD comprising the sequence set forth in SEQ ID NO: 378 or SEQ ID NO: 382 and an LCVD that does not comprise the sequence set forth in SEQ ID NO: 461, SEQ ID NO: 462, SEQ ID NO: 464, SEQ ID NO: 466, SEQ ID NO: 467, one of SEQ ID NOs: 477-478, or one of SEQ ID NOs: 480-482; or (b) an LCVD of SEQ ID NO: 451 or SEQ ID NO: 455 and an HCVD that does not comprise the sequence set forth in SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, or SEQ ID NO: 450.

Embodiment 43. The pharmaceutical composition of embodiment 27, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO: 451, SEQ ID NO: 455, one of SEQ ID NOs: 456-485, or one of SEQ ID NOs: 486-511, wherein the first ABD does not comprise (a) SEQ ID NOs: 378 and 451; (b) an HCVD comprising the sequence set forth in SEQ ID NO: 378 or SEQ ID NO: 382 and an LCVD that does not comprise the sequence of SEQ ID NOs: 389, 391, 396, 403, 417-419, 423-424, 426-430, 438-440, 444, and 450; or (c) an LCVD of SEQ ID NO: 451 or SEQ ID NO: 455 and an HCVD that is not one of SEQ ID NO: 461, SEQ ID NO: 462, SEQ ID NO: 464, SEQ ID NO: 466, SEQ ID NO: 467, one of SEQ ID NOs: 477-478, and one of SEQ ID NOs: 480-482.

Embodiment 44. The pharmaceutical composition of any one of embodiments 27-43, wherein the ABPC is degraded in the target mammalian cell following internalization of the ABPC by the target mammalian cell.

Embodiment 45. The pharmaceutical composition of any one of embodiments 27-44, wherein the ABPC further comprises a conjugated toxin, radioisotope, drug, or small molecule.

Embodiment 46. The pharmaceutical composition of embodiment 45, wherein the composition provides for an increase in toxin liberation in the target mammalian cell as compared to a composition comprising the same amount of a control ABPC.

Embodiment 47. The pharmaceutical composition of embodiment 46, wherein the composition provides for at least a 20%, 50%, 2-fold, or 5-fold increase in toxin liberation in the target mammalian cell as compared to a composition comprising the same amount of a control ABPC.

Embodiment 48. The pharmaceutical composition of any one of embodiments 45-47, wherein the composition provides for an increase in target mammalian cell killing as compared to a composition comprising the same amount of a control ABPC.

Embodiment 49. The pharmaceutical composition of embodiment 48, wherein the composition provides for at least a 20%, 50%, 2-fold, or 5-fold increase in target mammalian cell killing as compared to a composition comprising the same amount of a control ABPC.

Embodiment 50. The pharmaceutical composition of any one of embodiments 27-49, wherein the composition provides for an increase in endolysosomal delivery in the target mammalian cell as compared to a composition comprising the same amount of a control ABPC.

Embodiment 51. The pharmaceutical composition of embodiment 50, wherein the composition provides for at least a 20%, 50%, 2-fold, or 5-fold increase in endolysosomal delivery in the target mammalian cell as compared to a composition comprising the same amount of a control ABPC.

Embodiment 52. The pharmaceutical composition of any one of embodiments 27-51, wherein the composition results in a less or no detectable reduction in the level of LRRC15 presented on the surface of the target mammalian cell as compared to a composition comprising the same amount of a control ABPC.

Embodiment 53. The pharmaceutical composition of any one of embodiments 1-52, wherein the target mammalian cell is a cancer cell, optionally wherein the cancer is a sarcoma.

Embodiment 54. The pharmaceutical composition of any one of embodiments 1-53, wherein the dissociation rate of the ABD at a pH of ˜4.0-6.5 is at least 10% faster than the dissociation rate of the ABD at a pH of ˜7.0-8.0.

Embodiment 55. The pharmaceutical composition of any one of embodiments 1-53, wherein the dissociation rate of the ABD at a pH of ˜4.0-6.5 is at least 3-fold faster than the dissociation rate of the ABD at a pH of ˜7.0-8.0.

Embodiment 56. The pharmaceutical composition of any one of embodiments 1-53, wherein the dissociation rate of the ABD at a pH of ˜4.0-6.5 is at least 10-fold faster than the dissociation rate of the ABD at a pH of ˜7.0-8.0.

Embodiment 57. The pharmaceutical composition of any one of embodiments 1-56, wherein the KD of the ABD at a pH of ˜4.0-6.5 is at least 10% greater than the KD of the ABD at a pH of ˜7.0-8.0.

Embodiment 58. The pharmaceutical composition of any one of embodiments 1-56, wherein the KD of the ABD at a pH of ˜4.0-6.5 is at least 3-fold greater than the KD of the ABD at a pH of ˜7.0-8.0.

Embodiment 59. The pharmaceutical composition of any one of embodiments 1-56, wherein the KD of the ABD at a pH of ˜4.0-6.5 is at least 10-fold greater than the KD of the ABD at a pH of ˜7.0-8.0.

Embodiment 60. The pharmaceutical composition of any one of embodiments 1-59, wherein the ABPC is cytotoxic or cytostatic to the target mammalian cell.

Embodiment 61. The pharmaceutical composition of any one of embodiments 1-60, wherein the ABPC is cross-reactive with a non-human primate LRRC15 and human LRRC15.

Embodiment 62. The pharmaceutical composition of any one of embodiments 1-60, wherein the ABPC is cross-reactive with a non-human primate LRRC15, a human LRRC15, and one or both of rat LRRC15 and a mouse LRRC15.

Embodiment 63. The pharmaceutical composition of embodiment 62, wherein the ABPC is cross-reactive with a non-human primate LRRC15, a human LRRC15, a rat LRRC15, and a mouse LRRC15.

Embodiment 64. The pharmaceutical composition of any one of embodiments 1-63, wherein the ABD binds to an epitope of LRRC15 that is present on the surface of cells from an Old World Monkey.

Embodiment 65. The pharmaceutical composition of any one of embodiments 1-64, wherein the ABPC comprises a single polypeptide, optionally wherein the ABD is selected from the group consisting of: a VH domain, a VHH domain, a VNAR domain, and a scFv.

Embodiment 66. The pharmaceutical composition of embodiment 64 or 65, wherein the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

Embodiment 67. The pharmaceutical composition of any one of embodiments 1-64, wherein the ABPC comprises two or more polypeptides.

Embodiment 68. The pharmaceutical composition of embodiment 67, wherein the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAC, and a PROTAB.

Embodiment 69. The pharmaceutical composition of any one of embodiments 19-68, wherein at least one polypeptide of the ABPC is conjugated to the toxin, the radioisotope, the drug, or the small molecule via a cleavable linker.

Embodiment 70. The pharmaceutical composition of any one of embodiments 19-68, wherein at least one polypeptide of the ABPC is conjugated to the toxin, the radioisotope, the drug, or the small molecule via a non-cleavable linker.

Embodiment 71. The pharmaceutical composition of any of embodiments 1-70, wherein the half-life of the ABPC in vivo is increased as compared to the half-life of a control ABPC in vivo, optionally wherein the increase is ˜5%-95%, ˜10%-95%, ˜30%-95%, ˜50%-95%, or ˜70%-95% as compared to the half-life of a control ABPC in vivo.

Embodiment 72. The pharmaceutical composition of any one of embodiments 20-71, wherein the control ABPC is capable of specifically binding to LRRC15 or an epitope of LRRC15 presented on the surface of a target mammalian cell, wherein: (a) the control ABPC comprises a first ABD; (b) the dissociation rate of the first ABD of the control ABPC at a pH of ˜4.0-6.5 is no more than 3-fold faster than the dissociation rate at a pH of ˜7.0-8.0; and (c) the dissociation constant (KD) of the first ABD of the control ABPC at a pH of ˜4.0-6.5 is no more than 3-fold greater than the KD at a pH of ˜7.0-8.0.

Embodiment 73. The pharmaceutical composition of any one of embodiments 20-72, wherein the control ABPC is capable of specifically binding to LRRC15 or an epitope of LRRC15 presented on the surface of a target mammalian cell, wherein: (a) the control ABPC comprises a first ABD; (b) the dissociation rate of the first ABD of the control ABPC at a pH of ˜4.0-6.5 is no more than 2-fold faster than the dissociation rate at a pH of ˜7.0-8.0; and (c) the dissociation constant (KD) of the first ABD of the control ABPC at a pH of ˜4.0-6.5 is no more than 2-fold greater than the KD at a pH of ˜7.0-8.0.

Embodiment 74. The pharmaceutical composition of any one of embodiments 20-72, wherein the control ABPC is capable of specifically binding to LRRC15 or an epitope of LRRC15 presented on the surface of a target mammalian cell, wherein: (a) the control ABPC comprises a first ABD; (b) the dissociation rate of the first ABD of the control ABPC at a pH of ˜4.0-6.5 is no more than 1-fold faster than the dissociation rate at a pH of ˜7.0-8.0; and (c) the dissociation constant (KD) of the first ABD of the control ABPC at a pH of ˜4.0-6.5 is no more than 1-fold greater than the KD at a pH of ˜7.0-8.0.

Embodiment 75. The pharmaceutical composition of any one of embodiments 20-72, wherein the control ABPC is 15G7, hu15G7, 24D9, hu24D9, 29F1, or hu29F1.

Embodiment 76. The pharmaceutical composition of any one of embodiments 1-75, wherein the ABPC further comprises a second ABD.

Embodiment 77. A kit comprising at least one dose of the pharmaceutical composition of any one of embodiments 1-76.

Embodiment 78. An antigen-binding protein construct (ABPC) comprising: a first antigen-binding domain (ABD) that is capable of specifically binding LRRC15 or an epitope of LRRC15 presented on the surface of a target mammalian cell, wherein: the dissociation rate of the first ABD at a pH of ˜4.0-6.5 is faster than the dissociation rate at a pH of ˜7.0-8.0; or the KD of the first ABD at a pH of ˜4.0-6.5 is greater than the KD at a pH of ˜7.0-8.0.

Embodiment 79. The ABPC of embodiment 78, wherein the first ABD comprises an HCVD of one of an HCVD of (a) 15G7; (b) 24D9; and (c) 29F1, optionally wherein each HCVD has one or more amino acids substituted with a histidine, an aspartate, or a glutamate.

Embodiment 80. The ABPC of embodiment 78, wherein the first ABD comprises an LCVD of one of an LCVD of (a) 15G7; (b) 24D9; and (c) 29F1, optionally wherein each LCVD has one or more amino acids substituted with a histidine, an aspartate, or a glutamate.

Embodiment 81. The ABPC of embodiment 78, wherein the ABD comprises (a) an HCVD of 15G7; and/or an LCVD of 15G7; (b) an HCVD of 24D9; and/or an LCVD of 24D9; or (c) an HCVD of 29F1; and/or an LCVD of 29F1; optionally wherein each HCVD and/or LCVD has one or more amino acid substituted with a histidine, an aspartate, or a glutamate.

Embodiment 82. The ABPC of embodiment 79 or 81, wherein the HCVD comprises one of: (a) an HCVD of 15G7 comprising a sequence comprising SEQ ID NO: 378 or SEQ ID NO: 382; (b) an HCVD of 24D9 comprising a sequence comprising SEQ ID NO: 512 or SEQ ID NO: 516; and (c) an HCVD of 29F1 comprising a sequence comprising SEQ ID NO: 572 or SEQ ID NO: 576.

Embodiment 83. The ABPC of embodiment 80 or 81, wherein the LCVD comprises one of: (a) an LCVD of 15G7 comprising a sequence comprising SEQ ID NO: 451 or SEQ ID NO: 455; (b) an LCVD of 24D9 comprising a sequence comprising SEQ ID NO: 518 or SEQ ID NO: 522; and (c) an LCVD of 29F1 comprising a sequence comprising SEQ ID NO: 577 or SEQ ID NO: 581.

Embodiment 84. The ABPC of embodiment 78, wherein the first ABD comprises an HCVD comprising the CDR1, 2, and 3 of one of: (a) SEQ ID NOs: 379-381; (b) SEQ ID NOs: 513-515; and (c) SEQ ID NOs: 573-575, optionally wherein each set of three CDRs has collectively one or more amino acid position substituted with a histidine, an aspartate, or a glutamate.

Embodiment 85. The ABPC of embodiment 78, wherein the first ABD comprises an LCVD comprising the CDR1, 2, and 3 of one of: (a) SEQ ID NOs: 452-454; (b) SEQ ID NOs: 519-521; and (c) SEQ ID NOs: 578-580, optionally wherein each set of three CDRs has collectively one or more amino acid position substituted with a histidine, an aspartate, or a glutamate.

Embodiment 86. The ABPC of embodiment 78, wherein the first ABD comprises one of: (a) an HCVD comprising the CDR1, 2, and 3 of SEQ ID NOs: 379-381; and/or an LCVD comprising the CDR1, 2, and 3 of one of SEQ ID NOs: 452-454; (b) an HCVD comprising the CDR1, 2, and 3 of one of SEQ ID NOs: 513-515; and/or an LCVD comprising the CDR1, 2, and 3 of SEQ ID NOs: 519-521; or (c) an HCVD comprising the CDR1, 2, and 3 of SEQ ID NOs: 573-575; and/or an LCVD comprising the CDR1, 2, and 3 of SEQ ID NOs: 578-580; optionally wherein each set of three CDRs has collectively one or more amino acid position substituted with a histidine, an aspartate, or a glutamate.

Embodiment 87. The ABPC of embodiment 78, 79, or 84, wherein the first ABD comprises an HCVD of one of: (a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; (b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and (c) an HCVD comprising a sequence that is at least 90% identical to the sequence set forth in SEQ ID NO: 572 or SEQ ID NO: 576, wherein the HCVD includes an aspartate (D) or a glutamate (E) at one or more positions in SEQ ID NO: 572 or SEQ ID NO: 576 selected from the group consisting of: D positions selected from 31, 56, and 99; and an E position selected from 59.

Embodiment 88. The ABPC of embodiment 78, 80, or 85, wherein the first ABD comprises an LCVD of one of: (a) an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and (c) an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

Embodiment 89. The ABPC of embodiment 78, 79, or 84, wherein the first ABD comprises an HCVD of one of: (a) an HCVD comprising a sequence that is at least 90% identical to the sequence set forth in SEQ ID NO: 378 or SEQ ID NO: 382, wherein the HCVD includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53,106; and (b) an HCVD comprising a sequence that is at least 90% identical to the sequence set forth in SEQ ID NO: 512 or SEQ ID NO: 516, wherein the HCVD includes a histidine at two or more positions in SEQ ID NO: 512 or SEQ ID NO: 516.

Embodiment 90. The ABPC of embodiment 78, 80, or 85, wherein the first ABD comprises an LCVD of one of: (a) an LCVD comprising a sequence that is at least 90% identical to the sequence set forth in SEQ ID NO: 451 or SEQ ID NO: 455, wherein the LCVD includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96 and 93, 96; and (b) an LCVD comprising a sequence that is at least 90% identical to the sequence set forth in SEQ ID NO: 518 or SEQ ID NO: 522, wherein the LCVD includes a histidine at two or more positions in SEQ ID NO: 518 or SEQ ID NO: 522.

Embodiment 91. The ABPC of embodiment 78, 81, or 86, wherein the first ABD comprises one of: (a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; and/or an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and (c) an HCVD comprising a sequence that is at least 90% identical to the sequence set forth in SEQ ID NO: 572 or SEQ ID NO: 576, wherein the HCVD includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

Embodiment 92. The ABPC of embodiment 78, wherein the first ABD comprises an HCVD comprising the sequence set forth in SEQ ID NO: 378, SEQ ID NO: 382, one of SEQ ID NOs: 383-424, one of SEQ ID NOs 425-450, SEQ ID NO: 512, SEQ ID NO: 516, one of SEQ ID NOs: 523-567, one of SEQ ID NOs: 568-571, SEQ ID NO: 572, SEQ ID NO: 576, one of SEQ ID NOs 582-624, or one of SEQ ID NOs: 653-695.

Embodiment 93. The ABPC of embodiment 78 or 92, wherein the first ABD comprises an LCVD comprising the sequence set forth in SEQ ID NO: 451, SEQ ID NO: 455, one of SEQ ID NOs: 456-485, SEQ ID NOs: 486-511, SEQ ID NO: 518, SEQ ID NO: 522, SEQ ID NO: 577, SEQ ID NO: 581, one of SEQ ID NOs: 625-652, or one of SEQ ID NOs: 696-723.

Embodiment 94. The ABPC of embodiment 78, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO: 451, SEQ ID NO: 455, one of SEQ ID NOs: 456-485, or one of SEQ ID NOs: 486-511, wherein the first ABD does not comprise (a) SEQ ID NOs: 378 and 451 or SEQ ID NOs: 382 and 455; (b) an HCVD comprising a sequence of SEQ ID NO: 378 or SEQ ID NO: 382 and an LCVD that does not comprise the sequence set forth in SEQ ID NO: 461, SEQ ID NO: 462, SEQ ID NO: 464, SEQ ID NO: 466, SEQ ID NO: 467, one of SEQ ID NOs: 477-478, or one of SEQ ID NOs: 480-482; or (c) an LCVD of SEQ ID NO: 451 or SEQ ID NO: 455 and an HCVD that does not comprise the sequence set forth in SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, or SEQ ID NO: 450.

Embodiment 95. The ABPC of any one of embodiments 78-94, wherein the ABPC is degraded in the target mammalian cell following internalization of the ABPC by the target mammalian cell.

Embodiment 96. The ABPC of any one of embodiments 78-95, wherein the ABPC further comprises a conjugated toxin, radioisotope, drug, or small molecule.

Embodiment 97. The conjugated ABPC of embodiment 96, which provides for an increase in toxin liberation in the target mammalian cell as compared to the same amount of a control ABPC.

Embodiment 98. The conjugated ABPC of embodiment 97, which provides for at least a 20%, 50%, 2-fold, or 5-fold increase in toxin liberation in the target mammalian cell as compared to a composition comprising the same amount of a control ABPC.

Embodiment 99. The ABPC of any one of embodiments 96-98, wherein the composition provides for an increase in target mammalian cell killing as compared to the same amount of a control ABPC.

Embodiment 100. The ABPC of embodiment 99, which provides for at least a 20%, 50%, 2-fold, or 5-fold increase in target mammalian cell killing as compared to the same amount of a control ABPC.

Embodiment 101. The ABPC of any one of embodiments 78-100, which provides for an increase in endolysosomal delivery in the target mammalian cell as compared to the same amount of a control ABPC.

Embodiment 102. The ABPC of embodiment 101, which provides for at least a 20%, 50%, 2-fold, or 5-fold increase in endolysosomal delivery in the target mammalian cell as compared to the same amount of a control ABPC.

Embodiment 103. The ABPC of any one of embodiments 78-102, which results in a less or no detectable reduction in the level of LRRC15 presented on the surface of the target mammalian cell as compared to the same amount of a control ABPC.

Embodiment 104. An antigen-binding protein construct (ABPC) comprising: a first ABD that is capable of specifically binding LRRC15 or an epitope of LRRC15 presented on the surface of a target mammalian cell; and a conjugated toxin, radioisotope, drug, or small molecule, optionally wherein: (a) the dissociation rate of the first ABD at a pH of ˜4.0-6.5 is faster than the dissociation rate at a pH of ˜7.0-8.0; or the dissociation constant (KD) of the first ABD at a pH of ˜4.0-6.5 is greater than the KD at a pH of ˜7.0-8.0; and (b) the composition provides for one or more of: (i) an increase in toxin liberation in the target mammalian cell; (ii) an increase in target mammalian cell killing; and (iii) an increase in endolysosomal delivery in the target mammalian cell, each (i-iii) as compared to the same amount of a control ABPC.

Embodiment 105. The ABPC of embodiment 104, wherein the first ABD comprises an HCVD of one of: an HCVD of (a) 15G7; (b) 24D9; and (c) 29F1, each HCVD optionally with one or more amino acids substituted with a histidine, an aspartate, or a glutamate.

Embodiment 106. The ABPC of embodiment 105, wherein the first ABD comprises an LCVD of one of: an LCVD of (a) 15G7; (b) 24D9; and (c) 29F1, each LCVD optionally with one or more amino acids substituted with a histidine, an aspartate, or a glutamate.

Embodiment 107. The ABPC of embodiment 104, wherein the ABD comprises one of: (a) an HCVD and/or an LCVD of 15G7; (b) an HCVD and/or an LCVD of 24D9; or (c) an HCVD and/or an LCVD of 29F1, each optionally with one or more amino acids substituted with a histidine, an aspartate, or a glutamate.

Embodiment 108. The ABPC of embodiment 105 or 107, wherein the HCVD comprises one of (a) an HCVD of 15G7 comprising a sequence comprising SEQ ID NO: 378 or

SEQ ID NO: 382; (b) an HCVD of 24D9 comprising a sequence comprising SEQ ID NO: 512 or SEQ ID NO: 516; and (c) an HCVD of 29F1 comprising a sequence comprising SEQ ID NO: 572 or SEQ ID NO: 576.

Embodiment 109. The ABPC of embodiment 106 or 107, wherein the LCVD comprises one of: (a) an LCVD of 15G7 comprising a sequence comprising SEQ ID NO: 451 or SEQ ID NO: 455; (b) an LCVD of 24D9 comprising a sequence comprising SEQ ID NO: 518 or SEQ ID NO: 522; and (c) an LCVD of 29F1 comprising a sequence comprising SEQ ID NO: 577 or SEQ ID NO: 581.

Embodiment 110. The ABPC of embodiment 104, wherein the first ABD comprises an HCVD comprising the CDR1, 2, and 3 of one of: (a) SEQ ID NOs: 379-381; (b) SEQ ID NOs: 513-515; and (c) SEQ ID NOs: 573-575, each set of three HCVD and/or LCVD CDRs optionally having one or more amino acid position substituted with histidine, an aspartate, or a glutamate.

Embodiment 111. The ABPC of embodiment 104, wherein the first ABD comprises an LCVD comprising the CDR1, 2, and 3 of one of: (a) SEQ ID NOs: 452-454; (b) SEQ ID NOs: 519-521; and (c) SEQ ID NOs: 578-580, each set of three HCVD and/or LCVD CDRs optionally having one or more amino acid position substituted with histidine, an aspartate, or a glutamate.

Embodiment 112. The ABPC of embodiment 104, wherein the first ABD comprises one of: (a) an HCVD comprising the CDR1, 2, and 3 of SEQ ID NOs: 379-381; and/or an LCVD comprising the CDR1, 2, and 3 of one of SEQ ID NOs: 452-454; (b) an HCVD comprising the CDR1, 2, and 3 of one of SEQ ID NOs: 513-515; and/or an LCVD comprising the CDR1, 2, and 3 of SEQ ID NOs: 519-521; or (c) an HCVD comprising the CDR1, 2, and 3 of SEQ ID NOs: 573-575; and/or an LCVD comprising the CDR1, 2, and 3 of SEQ ID NOs: 578-580; each set of three HCVD and/or LCVD CDRs optionally having one or more amino acid position substituted with histidine, an aspartate, or a glutamate.

Embodiment 113. The ABPC of embodiment 104, 105, or 110, wherein the ABD comprises an LCVD of one of: (a) an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and (c) an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

Embodiment 114. The ABPC of embodiment 104, 106, or 111, wherein the ABD comprises an HCVD of one of: (a) an HCVD comprising a sequence that is at least 90% identical to the sequence set forth in SEQ ID NO: 378 or SEQ ID NO: 382, wherein the HCVD includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53,106; and (b) an HCVD comprising a sequence that is at least 90% identical to the sequence set forth in SEQ ID NO: 512 or SEQ ID NO: 516, wherein the HCVD includes a histidine at two or more positions in SEQ ID NO: 512 or SEQ ID NO: 516.

Embodiment 115. The ABPC of embodiment 104, 105, or 110, wherein the ABD comprises an LCVD of one of: (a) an LCVD comprising a sequence that is at least 90% identical to the sequence set forth in SEQ ID NO: 451 or SEQ ID NO: 455, wherein the LCVD includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96 and 93, 96; and (b) an LCVD comprising a sequence that is at least 90% identical to the sequence set forth in SEQ ID NO: 518 or SEQ ID NO: 522, wherein the LCVD includes a histidine at two or more positions in SEQ ID NO: 518 or SEQ ID NO: 522.

Embodiment 116. The ABPC of embodiment 104, 106, 111, wherein the first ABD comprises one of: (a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; and/or an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97; (b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from: 30, 31, 32, 52, 53, 58, 59, 60, 98, 105, 106, and 110; and/or an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from: 35 and 97; and (c) an HCVD comprising a sequence that is at least 90% identical to the sequence set forth in SEQ ID NO: 572 or SEQ ID NO: 576, wherein the HCVD includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCVD comprising a sequence that is at least 90% identical to the sequence set forth in SEQ ID NO: 577 or SEQ ID NO: 581, wherein the LCVD includes a histidine at one or more positions in SEQ ID NO: 577 or SEQ ID NO: 581 selected from: D positions selected from 27, 28, 31, 52 and 56; or E position selected from 51 and 56.

Embodiment 117. The ABPC of embodiment 104, 104, or 112, wherein the first ABD comprises an HCVD comprising the sequence set forth in SEQ ID NO: 378, SEQ ID NO: 382, one of SEQ ID NOs: 383-424, one of SEQ ID NOs 425-450, SEQ ID NO: 512, SEQ ID NO: 516, one of SEQ ID NOs: 523-567, one of SEQ ID NOs: 568-571, SEQ ID NO: 572, SEQ ID NO: 576, one of SEQ ID NOs 582-624, or one of SEQ ID NOs: 653-695.

Embodiment 118. The ABPC of embodiment 104, wherein the first ABD comprises an LCVD comprising the sequence set forth in SEQ ID NO: 451, SEQ ID NO: 455, one of SEQ ID NOs: 456-485, SEQ ID NOs: 486-511, SEQ ID NO: 518, SEQ ID NO: 522, SEQ ID NO: 577, SEQ ID NO: 581, one of SEQ ID NOs: 625-652, or one of SEQ ID NOs: 696-723.

Embodiment 119. The ABPC of embodiment 104 or 118, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO: 451, SEQ ID NO: 455, one of SEQ ID NOs: 456-485, or one of SEQ ID NOs: 486-511, wherein the first ABD does not comprise one of (a) SEQ ID NOs: 378 and 451 or SEQ ID NOs: 382 and 455; (b) an HCVD comprising the sequence set forth in SEQ ID NO: 378 or SEQ ID NO: 382 and an LCVD that does not comprise the sequence of SEQ ID NO: 461, SEQ ID NO: 462, SEQ ID NO: 464, SEQ ID NO: 466, SEQ ID NO: 467, one of SEQ ID NOs: 477-478, or one of SEQ ID NOs: 480-482; or (c) an LCVD of SEQ ID NO: 451 or SEQ ID NO: 455 and an HCVD that does not comprise the sequence set forth in SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, or SEQ ID NO: 450.

Embodiment 120. The ABPC of embodiment 104, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO: 451, SEQ ID NO: 455, one of SEQ ID NOs: 456-485, or one of SEQ ID NOs: 486-511, wherein the first ABD does not comprise (a) SEQ ID NOs: 378 and 451 or SEQ ID NOs: 382 and 455; (b) an HCVD comprising the sequence set forth in SEQ ID NO: 378 or SEQ ID NO: 382 and an LCVD that does not comprise the sequence of SEQ ID NO: 461, SEQ ID NO: 462, SEQ ID NO: 464, SEQ ID NO: 466, SEQ ID NO: 467, one of SEQ ID NOs: 477-478, or one of SEQ ID NOs: 480-482; or (c) an LCVD of SEQ ID NO: 451 or SEQ ID NO: 455 and an HCVD that does not comprise the sequence set forth in SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, or SEQ ID NO: 450.

Embodiment 121. The ABPC of any one of embodiments 104-120, wherein the ABPC is degraded in the target mammalian cell following internalization of the ABPC by the target mammalian cell.

Embodiment 122. The ABPC of any one of embodiments 104-121, wherein the increase in toxin liberation is at least a 20%, 50%, 2-fold, or 5-fold.

Embodiment 123. The ABPC of any one of embodiments 104-122, wherein the increase in cell killing is at least a 20%, 50%, 2-fold, or 5-fold.

Embodiment 124. The ABPC of any one of embodiments 104-123, wherein increase in endolysosomal delivery is at least a 20%, 50%, 2-fold, or 5-fold.

Embodiment 125. The ABPC of any one of embodiments 104-124, which results in a lower or no detectable reduction in the level of cell-surface LRRC15 as compared to the same amount of a control ABPC.

Embodiment 126. The ABPC of any one of embodiments 78-125, wherein the target mammalian cell is a cancer cell.

Embodiment 127. The ABPC of any one of embodiments 78-126, wherein the dissociation rate of the ABD at a pH of ˜4.0-6.5 is at least 10%, 3-fold, or 10-fold faster than the rate at a pH of ˜7.0-8.0.

Embodiment 128. The ABPC of any one of embodiments 78-126, wherein the KD of the ABD at a pH of ˜4.0-6.5 is at least 10%, 3-fold, or 10-fold greater than the KD at a pH of ˜7.0-8.0.

Embodiment 129. The ABPC of any one of embodiments 78-126, wherein the KD of the ABD at a pH of ˜4.0-6.5 is at least 3-fold greater than the KD of the ABD at a pH of ˜7.0-8.0.

Embodiment 130. The ABPC of any one of embodiments 78-126, wherein the KD of the ABD at a pH of ˜4.0-6.5 is at least 10-fold greater than the KD of the ABD at a pH of ˜7.0-8.0.

Embodiment 131. The ABPC of any one of embodiments 78-130, wherein the ABPC is cytotoxic or cytostatic to the target mammalian cell.

Embodiment 132. The ABPC of any one of embodiments 78-131, wherein the ABPC is cross-reactive with a non-human primate LRRC15 and human LRRC15.

Embodiment 133. The ABPC of embodiment 132, wherein the ABPC is cross-reactive with one or both of rat LRRC15 and a mouse LRRC15.

Embodiment 134. The ABPC of embodiment 133, cross-reactive with both the rat and mouse LRRC15.

Embodiment 135. The ABPC of any one of embodiments 78-134, wherein the ABD binds to an epitope of LRRC15 that is present on the surface of cells from an Old World Monkey.

Embodiment 136. The ABPC of any one of embodiments 78-135, wherein the ABPC comprises a single polypeptide.

Embodiment 137. The ABPC of embodiment 136, wherein the ABD is selected from: a VH domain, a VHH domain, a VNAR domain, and a scFv.

Embodiment 138. The ABPC of embodiment 136 or 137, wherein the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv; and/or wherein the ABPC comprises two or more polypeptides.

Embodiment 139. The ABPC of embodiment 138, wherein the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KA-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAB, and a PROTAC.

Embodiment 140. The ABPC of any one of embodiments 104-139, wherein at least one polypeptide of the ABPC is conjugated to the toxin, the radioisotope, the drug, or the small molecule via a cleavable linker.

Embodiment 141. The ABPC of any one of embodiments 104-139, wherein at least one polypeptide of the ABPC is conjugated to the toxin, the radioisotope, the drug, or the small molecule via a non-cleavable linker.

Embodiment 142. The ABPC of any one of embodiments 104-141, wherein the half-life of the ABPC in vivo is increased ˜5%-95%, ˜10%-95%, ˜30%-95%, ˜50%-95%, or ˜70%-95% as compared to the half-life of a control ABPC in vivo.

Embodiment 143. The ABPC of any one of embodiments 97-142, wherein the control ABPC is capable of specifically binding to LRRC15 or an epitope of LRRC15 presented on the surface of a target mammalian cell, wherein: (a) the control ABPC comprises a first ABD; (b) the dissociation rate of the first ABD of the control ABPC at a pH of ˜4.0-6.5 is no more than 3-fold faster than the dissociation rate at a pH of ˜7.0-8.0; and (c) the dissociation constant (KD) of the first ABD of the control ABPC at a pH of ˜4.0-6.5 is no more than 3-fold greater than the KD at a pH of ˜7.0-8.0.

Embodiment 144. The ABPC of embodiment 143, wherein the dissociation rate and the KD of the first ABD of the control ABPC are both no more than 2-fold greater at the pH of ˜4.0-6.5 as compared to the rate and the KD at the pH of ˜7.0-8.0.

Embodiment 145. The ABPC of embodiment 144, wherein both the rate and KD are no more than 1-fold greater at pH ˜4.0-6.5 as compared to at pH ˜7.0-8.0.

Embodiment 146. The ABPC of any one of embodiments 97-145, wherein the control ABPC is selected from one of: (a) 15G7; (b) 24D9; and (c) 29F1.

Embodiment 147. The ABPC of any one of embodiments 78-146, wherein the ABPC further comprises a second ABD.

Embodiment 148. A kit comprising at least one dose of the ABPC of any one of embodiments 78-147.

Embodiment 149. A method of treating a cancer characterized by having a population of cancer cells, CAFs, and/or stromal cells that have a predetermined level of LRRC15 or an epitope of LRRC15 presented on their surface, comprising administering a therapeutically effective amount of the pharmaceutical composition of any one of embodiments 1-76 or the ABPC of any one of embodiments 78-147 to a subject identified as having a cancer characterized by having the population of cancer cells, CAFs, and/or stromal cells.

Embodiment 150. A method of reducing the volume of a tumor in a subject, wherein the tumor is characterized by having a population of cancer cells, CAFs, and/or stromal cells that have a predetermined level of LRRC15 or an epitope of LRRC15 presented on their surface, comprising administering a therapeutically effective amount of the pharmaceutical composition of any one of embodiments 1-76 or the ABPC of any one of embodiments 78-147 to a subject identified as having a cancer characterized by having the population of cancer cells, CAFs, and/or stromal cells.

Embodiment 151. A method of inducing cell death in a cancer cell, CAF, and/or stromal cell in a subject, wherein the cancer cell, CAF, and/or stromal cell has a predetermined level of LRRC15 or an epitope of LRRC15 presented on its surface, comprising administering a therapeutically effective amount of the pharmaceutical composition of any one of embodiments 1-76 or the ABPC of any one of embodiments 78-147 to a subject identified as having a cancer characterized by having a population of cancer cells, CAFs, and/or stromal cells.

Embodiment 152. A method of decreasing the risk of developing a metastasis or decreasing the risk of developing an additional metastasis in a subject having a cancer, wherein the cancer is characterized by having a population of cancer cells, CAFs, and/or stromal cells that have a predetermined level of LRRC15 or an epitope of LRRC15 presented on their surface, comprising administering a therapeutically effective amount of the pharmaceutical composition of any one of embodiments 1-76 or the ABPC of any one of embodiments 78-147 to a subject identified as having a cancer characterized by having the population of cancer cells, CAFs, and/or stromal cells. In some embodiments, the cancer is a sarcoma, for example, a leiomyosarcoma, an osteosarcoma, or a chondrosarcoma.

Embodiment 153. The method of any one of embodiments 149-152, wherein the cancer is a primary tumor, a metastasis, a non-T-cell-infiltrating tumor, or a T-cell infiltrating tumor. In some embodiments, the cancer is a sarcoma, for example, a leiomyosarcoma, an osteosarcoma, or a chondrosarcoma. In some embodiments, the cancer is a sarcoma, for example, a leiomyosarcoma, an osteosarcoma, or a chondrosarcoma.

Embodiment 154. An anti-LRRC15 ABPC or antibody comprising the same heavy chain CDRs and light chain CDRs as contained in one of the following pairs of sequences: SEQ ID NOs: 1 and 64, SEQ ID NOs: 84 and 154, SEQ ID NOs: 430 and 455, SEQ ID NOs: 382 and 489, SEQ ID NOs: 382 and 491, SEQ ID NOs: 516 and 522, SEQ ID NOs: 571 and 517, SEQ ID NOs: 576 and 581, SEQ ID NOs: 576 and 639, SEQ ID NOs: 576 and 700, SEQ ID NOs: 382 and 490, SEQ ID NOs: 570 and 522, SEQ ID NOs: 576 and 641, SEQ ID NOs: 84 and 177, SEQ ID NOs: 576 and 625, SEQ ID NOs: 724 and 725, SEQ ID NOs: 726 and 727, SEQ ID NOs: 728 and 729, SEQ ID NOs: 730 and 731, SEQ ID NOs: 730 and 732, SEQ ID NOs: 733 and 734, SEQ ID NOs: 735 and 736, SEQ ID NOs: 737 and 738, SEQ ID NOs: 737 and 739, and SEQ ID NOs: 737 and 740.

Embodiment 155. The ABPC or antibody of embodiment 154, wherein the heavy chain variable domain and light chain variable domain have the same variable domains as contained in one of the following pairs of sequences: SEQ ID NOs: 1 and 64, SEQ ID NOs: 84 and 154, SEQ ID NOs: 430 and 455, SEQ ID NOs: 382 and 489, SEQ ID NOs: 382 and 491, SEQ ID NOs: 516 and 522, SEQ ID NOs: 571 and 517, SEQ ID NOs: 576 and 581, SEQ ID NOs: 576 and 639, SEQ ID NOs: 576 and 700, SEQ ID NOs: 382 and 490, SEQ ID NOs: 570 and 522, SEQ ID NOs: 576 and 641, SEQ ID NOs: 84 and 177, SEQ ID NOs: 576 and 625, SEQ ID NOs: 724 and 725, SEQ ID NOs: 726 and 727, SEQ ID NOs: 728 and 729, SEQ ID NOs: 730 and 731, SEQ ID NOs: 730 and 732, SEQ ID NOs: 733 and 734, SEQ ID NOs: 735 and 736, SEQ ID NOs: 737 and 738, SEQ ID NOs: 737 and 739, and SEQ ID NOs: 737 and 740.

Embodiment 156. The ABPC or antibody of embodiment 155, wherein the heavy chain and light chain are selected from any one of the polypeptide sequence pairs set forth in SEQ ID NOs: SEQ ID NOs: 724 and 725, SEQ ID NOs: 726 and 727, SEQ ID NOs: 728 and 729, SEQ ID NOs: 730 and 731, SEQ ID NOs: 730 and 732, SEQ ID NOs: 733 and 734, SEQ ID NOs: 735 and 736, SEQ ID NOs: 737 and 738, SEQ ID NOs: 737 and 739, and SEQ ID NOs: 737 and 740.

Embodiment 157. The ABPC or antibody of any one of embodiments 154-156, wherein position 205 of the light chain is an amino acid other than cysteine.

Embodiment 158. An antibody comprising a heavy chain variable domain and a light chain variable domain comprising polypeptide sequences that are at least 85%, 90%, 95%, 99% identical to, or are 100% identical to, and contain the same HCDRs and LCDRs as, the pairs of sequences set forth in SEQ ID NOs: 1 and 64, SEQ ID NOs: 84 and 154, SEQ ID NOs: 430 and 455, SEQ ID NOs: 382 and 489, SEQ ID NOs: 382 and 491, SEQ ID NOs: 516 and 522, SEQ ID NOs: 571 and 517, SEQ ID NOs: 576 and 581, SEQ ID NOs: 576 and 639, SEQ ID NOs: 576 and 700, SEQ ID NOs: 382 and 490, SEQ ID NOs: 570 and 522, SEQ ID NOs: 576 and 641, SEQ ID NOs: 84 and 177, SEQ ID NOs: 576 and 625, SEQ ID NOs: 724 and 725, SEQ ID NOs: 726 and 727, SEQ ID NOs: 728 and 729, SEQ ID NOs: 730 and 731, SEQ ID NOs: 730 and 732, SEQ ID NOs: 733 and 734, SEQ ID NOs: 735 and 736, SEQ ID NOs: 737 and 738, SEQ ID NOs: 737 and 739, and SEQ ID NOs: 737 and 740.

Embodiment 159. The antibody of embodiment 158, wherein the HCVD and/or LCVD comprise favorable developability mutations.

Embodiment 160. The ABPC, antibody, or composition of any of the preceding embodiments, wherein the cancer is a sarcoma, for example, a leiomyosarcoma, an osteosarcoma, or a chondrosarcoma.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antigen-binding protein construct (ABPC) comprising: a first antigen-binding domain (ABD) that is capable of specifically binding LRRC15 or an epitope of LRRC15 presented on the surface of a target mammalian cell;
wherein the first ABD comprises an ABD of 15G7, 24D9, or 29F1, optionally with one or more amino acids of the ABD substituted with a histidine, an aspartate, or a glutamate;
optionally wherein the dissociation rate of the first ABD at a pH of ˜4.0-6.5 is faster than the dissociation rate at a pH of ˜7.0-8.0 or the KD of the first ABD at a pH of ˜4.0-6.5 is greater than the KD at a pH of ˜7.0-8.0.

2. The ABPC of claim 1, wherein the first ABD comprises a heavy chain variable domain (HCVD) of one of an HCVD of (a) 15G7; (b) 24D9; and (c) 29F1, each HCVD optionally with one or more amino acids substituted with a histidine, and optionally wherein the 29F1 HCVD has one or more amino acids substituted with an aspartate or a glutamate; and/or wherein the first ABD comprises a light chain variable domain (LCVD) of one of an LCVD of (a) 15G7; (b) 24D9; and (c) 29F1, each LCVD optionally with one or more amino acids substituted with a histidine, optionally wherein the 29F1 LCVD has one or more amino acids substituted with an aspartate or a glutamate.

3. The ABPC of claim 2, wherein the ABD comprises:
(a) an HCVD of 15G7; and/or an LCVD of 15G7;
(b) an HCVD of 24D9; and/or an LCVD of 24D9; or
(c) an HCVD of 29F1; and/or an LCVD of 29F1;
optionally wherein each HCVD and/or LCVD has one or more amino acid substituted with a histidine, an aspartate, or a glutamate;
optionally wherein the ABD is selected from MYT2737 (HCV=SEQ ID NO: 1, LCV=SEQ ID NO: 64), MYT3315 (HCV=SEQ ID NO: 84, LCV=SEQ ID NO: 154), MYT8391 (HCV=SEQ ID NO: 430, LCV=SEQ ID NO: 455), MYT8415 (HCV=SEQ ID NO: 382, LCV=SEQ ID NO: 489), MYT8417 (HCV=SEQ ID NO: 382, LCV=SEQ ID NO: 491), MYT8483 (HCV=SEQ ID NO: 516, LCV=SEQ ID NO: 522), MYT9776 (HCV=SEQ ID NO: 571, LCV=SEQ ID NO: 517), MYT8094 (HCV=SEQ ID NO: 576, LCV=SEQ ID NO: 581), MYT9521 (HCV=SEQ ID NO: 576, LCV=SEQ ID NO: 639), MYT9731 (HCV=SEQ ID NO: 576, LCV=SEQ ID NO: 700), MYT8416 (HCV=SEQ ID NO: 382, LCV=SEQ ID NO: 490), MYT8500 (HCV=SEQ ID NO: 570, LCV=SEQ ID NO: 522), MYT9523 (HCV=SEQ ID NO: 576, LCV=SEQ ID NO: 641), MYT4174 (HCV=SEQ ID NO: 84, LCV=SEQ ID NO: 177), and MYT9507 (HCV=SEQ ID NO: 576, LCV=SEQ ID NO: 625); or
optionally wherein the ABD is selected from an antibody comprising heavy and light chain polypeptide sequence pairs that are at least 95% identical to the pairs of sequences set forth in SEQ ID NOs: 724 and 725; 726 and 727; 728 and 729; 730 and 731; 730 and 732; 733 and 734; 735 and 736; 737 and 738; 737 and 739; and 737 and 740.

4. The ABPC of claim 1, wherein the HCVD comprises one of:
(a) an HCVD of 15G7 comprising the sequence set forth in SEQ ID NO: 378 or SEQ ID NO: 382;
(b) an HCVD of 24D9 comprising the sequence set forth in SEQ ID NO: 512 or SEQ ID NO: 516; and
(c) an HCVD of 29F1 comprising the sequence set forth in SEQ ID NO: 572 or SEQ ID NO: 576; and/or
the LCVD comprises one of:
(a) an LCVD of 15G7 comprising the sequence set forth in SEQ ID NO: 451 or SEQ ID NO: 455;
(b) an LCVD of 24D9 comprising the sequence set forth in SEQ ID NO: 518 or SEQ ID NO: 522; and
(c) an LCVD of 29F1 comprising the sequence set forth in SEQ ID NO: 577 or SEQ ID NO: 581;
optionally wherein each HCVD and/or LCVD has one or more amino acid position substituted with a histidine, an aspartate, or a glutamate.

5. The ABPC of claim 1, wherein the first ABD comprises an HCVD comprising the CDR1, 2, and 3 of one of: (a) SEQ ID NOs: 379-381; (b) SEQ ID NOs: 513-515; and (c) SEQ ID NOs: 573-575, optionally wherein each set of three CDRs has collectively one or more amino acid position substituted with a histidine, an aspartate, or a glutamate; and/or
wherein the first ABD comprises an LCVD comprising the CDR1, 2, and 3 of one of: (a) SEQ ID NOs: 452-454; (b) SEQ ID NOs: 519-521; and (c) SEQ ID NOs: 578-580, optionally wherein each set of three CDRs has collectively one or more amino acid position substituted with a histidine, an aspartate, or a glutamate.

6. The ABPC of any one of claims 1 to 5, wherein the first ABD comprises an HCVD of one of:
(a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110;
(b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and
(c) an HCVD comprising a sequence that is at least 90% identical to the sequence set forth in SEQ ID NO: 572 or SEQ ID NO: 576, wherein the HCVD includes a histidine, an aspartate, or a glutamate at one or more positions in SEQ ID NO: 572 or SEQ ID NO: 576 selected from the group consisting of: D positions selected from 31, 56 and 99; and an E position selected from 59.

7. The ABPC of any one of claims 1 to 5, wherein the first ABD comprises an LCVD of one of:
(a) an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97;
(b) an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and
(c) an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

8. The ABPC of any one of claims 1 to 5, wherein the first ABD comprises an HCVD of one of:
(a) an HCVD comprising a sequence that is at least 90% identical to the sequence set forth in SEQ ID NO: 378 or SEQ ID NO: 382, wherein the HCVD includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53, 106;
(b) an HCVD comprising a sequence that is at least 90% identical to the sequence set forth in SEQ ID NO: 512 or SEQ ID NO: 516, wherein the HCVD includes a histidine at two or more positions in SEQ ID NO: 512 or SEQ ID NO: 516; and
(c) an HCVD comprising a sequence that is at least 90% identical to the sequence set forth in SEQ ID NO: 572 or SEQ ID NO: 576, wherein the HCVD includes a histidine, an aspartate, or a glutamate at two or more positions in SEQ ID NO: 572 or SEQ ID NO: 576; and/or
wherein the first ABD comprises an LCVD of one of: (a) an LCVD comprising a sequence that is at least 90% identical to the sequence set forth in SEQ ID NO: 451 or SEQ ID NO: 455, wherein the LCVD includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96 and 93, 96; and (b) an LCVD comprising a sequence that is at least 90% identical to the sequence set forth in SEQ ID NO: 518 or SEQ ID NO: 522, wherein the LCVD includes a histidine at two or more positions in SEQ ID NO: 518 or SEQ ID NO: 522; and (c) an LCVD comprising a sequence that is at least 90% identical to the sequence set forth in SEQ ID NO: 577 or SEQ ID NO: 581, wherein the LCVD includes a histidine, an aspartate, or a glutamate at two or more positions in SEQ ID NO: 577 or SEQ ID NO: 581.

9. The ABPC of any one of claims 1 to 5, wherein the first ABD comprises one of:
(a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; and/or an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97;
(b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and
(c) an HCVD comprising a sequence that is at least 90% identical to the sequence set forth in SEQ ID NO: 572 or SEQ ID NO: 576, wherein the HCVD includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

10. The ABPC of claim 1, wherein the first ABD comprises an HCVD comprising the sequence set forth in SEQ ID NO: 378, SEQ ID NO: 382, one of SEQ ID NOs: 383-424, one of SEQ ID NOs 425-450, SEQ ID NO: 512, SEQ ID NO: 516, one of SEQ ID NOs: 523-567, one of SEQ ID NOs: 568-571, SEQ ID NO: 572, SEQ ID NO: 576, one of SEQ ID NOs 582-624, or one of SEQ ID NOs: 653-695; and/or
wherein the first ABD comprises an LCVD comprising the sequence set forth in SEQ ID NO: 451, SEQ ID NO: 455, one of SEQ ID NOs: 456-485, SEQ ID NOs: 486-511, SEQ ID NO: 518, SEQ ID NO: 522, SEQ ID NO: 577, SEQ ID NO: 581, one of SEQ ID NOs: 625-652, or one of SEQ ID NOs: 696-723; or
wherein the first ABD comprises an HCVD and an LCVD comprising HCDRs and LCDRs, respectively, present in the HCVDs and LCVDs of one of 15G7, 24D9, and 29F1.

11. The ABPC of claim 1, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO: 451, SEQ ID NO: 455, one of SEQ ID NOs: 456-485, or one of SEQ ID NOs: 486-511, wherein the first ABD does not comprise (a) an HCVD comprising the sequence set forth in SEQ ID NO: 378 or SEQ ID NO: 382 and an LCVD that does not comprise the sequence set forth in SEQ ID NO: 461, SEQ ID NO: 462, SEQ ID NO: 464, SEQ ID NO: 466, SEQ ID NO: 467, one of SEQ ID NOs: 477-478, or one of SEQ ID NOs: 480-482; or (b) an LCVD of SEQ ID NO: 451 or SEQ ID NO: 455 and an HCVD that does not comprise the sequence set forth in SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, or SEQ ID NO: 450.

12. The ABPC of any one of claims 1-11, wherein the ABPC is degraded in the target mammalian cell following internalization of the ABPC by the target mammalian cell; optionally wherein the ABPC further comprises a conjugated toxin, radioisotope, drug, or small molecule.

13. The conjugated ABPC of claim 12, wherein the conjugated ABPC provides an increase in toxin liberation in the target mammalian cell as compared to the same amount of a control conjugated ABPC; optionally wherein the increased toxin liberation is at least a 20%, 50%, 2-fold, or 5-fold.

14. The ABPC of claim 12 or 13, wherein the ABPC provides an increase in target mammalian cell killing as compared to the same amount of a control ABPC; optionally wherein the increase cell killing is at least a 20%, 50%, 2-fold, or 5-fold.

15. The ABPC of any one of claims 1-14, wherein the ABPC provides an increase in endolysosomal delivery in the target mammalian cell as compared to the same amount of a control ABPC; optionally wherein the increase in delivery is at least a 20%, 50%, 2-fold, or 5-fold increase.

16. The ABPC of any one of claims 1-15, wherein the ABPC results in a less or no detectable reduction in the level of LRRC15 presented on the surface of the target mammalian cell as compared to the same amount of a control ABPC.

17. A composition, optionally a pharmaceutical composition, comprising an effective amount of the ABPC of any one of claims 1-16.

18. A composition comprising an effective amount of an antigen-binding protein construct (ABPC) comprising: a first ABD that is capable of specifically binding LRRC15 or an epitope of LRRC15 presented on the surface of a target mammalian cell; and a conjugated toxin, radioisotope, drug, or small molecule, optionally wherein: (a) the dissociation rate of the first ABD at a pH of ˜4.0-6.5 is faster than the dissociation rate at a pH of ˜7.0-8.0; or the dissociation constant (KD) of the first ABD at a pH of ˜4.0-6.5 is greater than the KD at a pH of ˜7.0-8.0; and (b) the composition provides for one or more of: (i) an increase in toxin liberation in the target mammalian cell as compared to a composition comprising the same amount of a control ABPC; (ii) an increase in target mammalian cell killing as compared to a composition comprising the same amount of a control ABPC; and (iii) an increase in endolysosomal delivery in the target mammalian cell as compared to a composition comprising the same amount of a control ABPC.

19. The composition of claim 18, wherein the first ABD comprises a heavy chain variable domain (HCVD) of one of an HCVD of (a) 15G7; (b) 24D9; and (c) 29F1, each HCVD optionally with one or more amino acids substituted with a histidine, and optionally wherein the 29F1 HCVD has one or more amino acids substituted with an aspartate or a glutamate; and/or wherein the first ABD comprises a light chain variable domain (LCVD) of one of an LCVD of (a) 15G7; (b) 24D9; and (c) 29F1, each LCVD optionally with one or more amino acids substituted with a histidine, optionally wherein the 29F1 LCVD has one or more amino acids substituted with an aspartate or a glutamate.

20. The composition of claim 19, wherein the ABD comprises
(a) an HCVD of 15G7; and/or an LCVD of 15G7;
(b) an HCVD of 24D9; and/or an LCVD of 24D9; or
(c) an HCVD of 29F1; and/or an LCVD of 29F1;
optionally wherein each HCVD and/or LCVD has one or more amino acid substituted with a histidine, an aspartate, or a glutamate;
optionally wherein the ABD is selected from MYT2737 (HCV=SEQ ID NO: 1, LCV=SEQ ID NO: 64), MYT3315 (HCV=SEQ ID NO: 84, LCV=SEQ ID NO: 154), MYT8391 (HCV=SEQ ID NO: 430, LCV=SEQ ID NO: 455), MYT8415 (HCV=SEQ ID NO: 382, LCV=SEQ ID NO: 489), MYT8417 (HCV=SEQ ID NO: 382, LCV=SEQ ID NO: 491), MYT8483 (HCV=SEQ ID NO: 516, LCV=SEQ ID NO: 522), MYT9776 (HCV=SEQ ID NO: 571, LCV=SEQ ID NO: 517), MYT8094 (HCV=SEQ ID NO: 576, LCV=SEQ ID NO: 581), MYT9521 (HCV=SEQ ID NO: 576, LCV=SEQ ID NO: 639), MYT9731 (HCV=SEQ ID NO: 576, LCV=SEQ ID NO: 700), MYT8416 (HCV=SEQ ID NO: 382, LCV=SEQ ID NO: 490), MYT8500 (HCV=SEQ ID NO: 570, LCV=SEQ ID NO: 522), MYT9523 (HCV=SEQ ID NO: 576, LCV=SEQ ID NO: 641), MYT4174 (HCV=SEQ ID NO: 84, LCV=SEQ ID NO: 177), and MYT9507 (HCV-SEQ ID NO: 576, LCV=SEQ ID NO: 625); or
optionally wherein the ABD is selected from an antibody comprising heavy and light chain polypeptide sequence pairs that are at least 95% identical to the pairs of sequences set forth in SEQ ID NOs: 724 and 725; 726 and 727; 728 and 729; 730 and 731; 730 and 732; 733 and 734; 735 and 736; 737 and 738; 737 and 739; and 737 and 740.

21. The composition of claim 18, wherein the HCVD comprises one of:
(a) an HCVD of 15G7 comprising the sequence set forth in SEQ ID NO: 378 or SEQ ID NO: 382;
(b) an HCVD of 24D9 comprising the sequence set forth in SEQ ID NO: 512 or SEQ ID NO: 516; and
(c) an HCVD of 29F1 comprising the sequence set forth in SEQ ID NO: 572 or SEQ ID NO: 576; and/or
the LCVD comprises one of:
(a) an LCVD of 15G7 comprising the sequence set forth in SEQ ID NO: 451 or SEQ ID NO: 455;
(b) an LCVD of 24D9 comprising the sequence set forth in SEQ ID NO: 518 or SEQ ID NO: 522; and
(c) an LCVD of 29F1 comprising the sequence set forth in SEQ ID NO: 577 or SEQ ID NO: 581;
optionally wherein each HCVD and/or LCVD has one or more amino acid position substituted with a histidine, an aspartate, or a glutamate.

22. The composition of claim 18, wherein the first ABD comprises an HCVD comprising the CDR1, 2, and 3 of one of: (a) SEQ ID NOs: 379-381; (b) SEQ ID NOs: 513-515; and (c) SEQ ID NOs: 573-575, optionally wherein each set of three CDRs has collectively one or more amino acid position substituted with a histidine, an aspartate, or a glutamate; and/or
wherein the first ABD comprises an LCVD comprising the CDR1, 2, and 3 of one of: (a) SEQ ID NOs: 452-454; (b) SEQ ID NOs: 519-521; and (c) SEQ ID NOs: 578-580, optionally wherein each set of three CDRs has collectively one or more amino acid position substituted with a histidine, an aspartate, or a glutamate.

23. The composition of any one of claims 18 to 22, wherein the first ABD comprises an HCVD of one of:
(a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110;
(b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and
(c) an HCVD comprising a sequence that is at least 90% identical to the sequence set forth in SEQ ID NO: 572 or SEQ ID NO: 576, wherein the HCVD includes a histidine, an aspartate, or a glutamate at one or more positions in SEQ ID NO: 572 or SEQ ID NO: 576 selected from the group consisting of: D positions selected from 31, 56 and 99; and an E position selected from 59.

24. The composition of any one of claims 18 to 22, wherein the first ABD comprises an LCVD of one of:
(a) an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97;
(b) an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and
(c) an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

25. The composition of any one of claims 18 to 22, wherein the first ABD comprises an HCVD of one of:
(a) an HCVD comprising a sequence that is at least 90% identical to the sequence set forth in SEQ ID NO: 378 or SEQ ID NO: 382, wherein the HCVD includes a histidine at two or more positions in SEQ ID NO: 378 or SEQ ID NO: 382 including one pair selected from the group consisting of: 34, 53; 34, 104; 34, 105; 34, 106; 53, 104; 53, 105 and 53, 106;
(b) an HCVD comprising a sequence that is at least 90% identical to the sequence set forth in SEQ ID NO: 512 or SEQ ID NO: 516, wherein the HCVD includes a histidine at two or more positions in SEQ ID NO: 512 or SEQ ID NO: 516; and
(c) an HCVD comprising a sequence that is at least 90% identical to the sequence set forth in SEQ ID NO: 572 or SEQ ID NO: 576, wherein the HCVD includes a histidine, an aspartate, or a glutamate at two or more positions in SEQ ID NO: 572 or SEQ ID NO: 576; and/or
wherein the first ABD comprises an LCVD of one of:
(a) an LCVD comprising a sequence that is at least 90% identical to the sequence set forth in SEQ ID NO: 451 or SEQ ID NO: 455, wherein the LCVD includes a histidine at two or more positions in SEQ ID NO: 451 or SEQ ID NO: 455 including one pair of positions selected from the group consisting of: 30, 32; 30, 92; 30, 93; 30, 96; 32, 92; 32, 93; 32, 96; 92, 93; 92, 96 and 93, 96;
(b) an LCVD comprising a sequence that is at least 90% identical to the sequence set forth in SEQ ID NO: 518 or SEQ ID NO: 522, wherein the LCVD includes a histidine at two or more positions in SEQ ID NO: 518 or SEQ ID NO: 522; and
(c) an LCVD comprising a sequence that is at least 90% identical to the sequence set forth in SEQ ID NO: 577 or SEQ ID NO: 581, wherein the LCVD includes a histidine, an aspartate, or a glutamate at two or more positions in SEQ ID NO: 577 or SEQ ID NO: 581.

26. The composition of any one of claims 18 to 22, wherein the first ABD comprises one of:
(a) an HCVD comprising a sequence that is at least 90% identical to the sequence set forth in SEQ ID NO: 378 or SEQ ID NO: 382, wherein the HCVD includes a histidine at one or more positions in SEQ ID NO: 378 or SEQ ID NO: 382 selected from the group consisting of: 32, 34, 53, 60, 103, 104, 105, 109, and 110; and/or an LCVD comprising a sequence that is at least 90% identical to the sequence set forth in SEQ ID NO: 451 or SEQ ID NO: 455, wherein the LCVD includes a histidine at one or more positions in SEQ ID NO: 451 or SEQ ID NO: 455 selected from the group consisting of: 29, 30, 32, 34, 50, 92, 93, 95, 96, and 97;
(b) an HCVD comprising a sequence that is at least 90% identical to the sequence set forth in SEQ ID NO: 512 or SEQ ID NO: 516, wherein the HCVD includes a histidine at one or more positions in SEQ ID NO: 512 or SEQ ID NO: 516 selected from the group consisting of: 30, 31, 32, 52, 53, 58, 59, 60, 98, 105, 106, and 110; and/or an LCVD comprising a sequence that is at least 90% identical to the sequence set forth in SEQ ID NO: 518 or SEQ ID NO: 522, wherein the LCVD includes a histidine at one or more positions in SEQ ID NO: 518 or SEQ ID NO: 522 selected from the group consisting of: 35 and 97; and
(c) an HCVD comprising a sequence that is at least 90% identical to the sequence set forth in SEQ ID NO: 572 or SEQ ID NO: 576, wherein the HCVD includes an aspartate (D) or a glutamate (E) at one or more positions in SEQ ID NO: 572 or SEQ ID NO: 576 selected from the group consisting of: D positions selected from 31, 56 and 99; and an E position selected from 59; and/or an LCVD comprising a sequence that is at least 90% identical to the sequence set forth in SEQ ID NO: 577 or SEQ ID NO: 581, wherein the LCVD includes an aspartate (D) or glutamate (E) at one or more positions in SEQ ID NO: 577 or SEQ ID NO: 581 selected from the group consisting of: D positions selected from 27, 28, 31, 52 and 56; and E positions selected from 51 and 56.

27. The composition of claim 18, wherein the first ABD comprises an HCVD comprising the sequence set forth in SEQ ID NO: 378, SEQ ID NO: 382, one of SEQ ID NOs: 383-424, one of SEQ ID NOs 425-450, SEQ ID NO: 512, SEQ ID NO: 516, one of SEQ ID NOs: 523-567, one of SEQ ID NOs: 568-571, SEQ ID NO: 572, SEQ ID NO: 576, one of SEQ ID NOs 582-624, or one of SEQ ID NOs: 653-695; and/or
wherein the first ABD comprises an LCVD comprising the sequence set forth in SEQ ID NO: 451, SEQ ID NO: 455, one of SEQ ID NOs: 456-485, SEQ ID NOs: 486-511, SEQ ID NO: 518, SEQ ID NO: 522, SEQ ID NO: 577, SEQ ID NO: 581, one of SEQ ID NOs: 625-652, or one of SEQ ID NOs: 696-723; or
wherein the first ABD comprises an HCVD and an LCVD comprising HCDRs and LCDRs, respectively, present in the HCVDs and LCVDs of one of 15G7, 24D9, and 29F1.

28. The composition of claim 18, wherein the first ABD comprises an HCVD comprising the sequence set forth in SEQ ID NO: 378, SEQ ID NO: 382, one of SEQ ID NOs: 383-424, or one of SEQ ID NOs: 425-450, and/or an LCVD of comprising the sequence set forth in SEQ ID NO:
451, SEQ ID NO: 455, one of SEQ ID NOs: 456-485, or one of SEQ ID NOs: 486-511, wherein the first ABD does not comprise (a) an HCVD comprising the sequence set forth in SEQ ID NO: 378 or SEQ ID NO: 382 and an LCVD that does not comprise the sequence set forth in SEQ ID NO: 461, SEQ ID NO: 462, SEQ ID NO: 464, SEQ ID NO: 466, SEQ ID NO: 467, one of SEQ ID NOs: 477-478, or one of SEQ ID NOs: 480-482; or (b) an LCVD of SEQ ID NO: 451 or SEQ ID NO: 455 and an HCVD that does not comprise the sequence set forth in SEQ ID NO: 389, SEQ ID NO: 391, SEQ ID NO: 396, SEQ ID NO: 403, one of SEQ ID NOs: 417-419, one of SEQ ID NOs: 423-424, one of SEQ ID NOs: 426-430, one of SEQ ID NOs: 438-440, SEQ ID NO: 444, or SEQ ID NO: 450.

29. The composition of any one of claims 17-28, wherein the ABPC is degraded in the target mammalian cell following internalization of the ABPC by the target mammalian cell; optionally wherein the ABPC further comprises a conjugated toxin, radioisotope, drug, or small molecule.

30. The composition of claim 29, wherein the conjugated ABPC provides an increase in toxin liberation in the target mammalian cell as compared to the same amount of a control conjugated ABPC; optionally wherein the increased toxin liberation is at least a 20%, 50%, 2-fold, or 5-fold.

31. The composition of claim 29 or 30, wherein the ABPC provides an increase in target mammalian cell killing as compared to the same amount of a control ABPC; optionally wherein the increase cell killing is at least a 20%, 50%, 2-fold, or 5-fold.

32. The composition of any one of claims 17-31, wherein the composition provides an increase in endolysosomal delivery in the target mammalian cell as compared to a composition comprising the same amount of control ABPC; optionally wherein the increase in delivery is at least a 20%, 50%, 2-fold, or 5-fold increase.

33. The composition of any one of claims 17-32, wherein the ABPC results in a less or no detectable reduction in the level of LRRC15 presented on the surface of the target mammalian cell as compared to a composition comprising the same amount of a control ABPC.

34. The ABPC of any one of claims 1-16 or the composition of any one of claims 17-33, wherein the target mammalian cell is a cancer cell or a cancer-associated fibroblast (CAF);
optionally wherein the cancer cell is part of or from a sarcoma, optionally selected from a leiomyosarcoma, an osteosarcoma, and a chondrosarcoma; and/or
wherein the CAF is positive for LRRC15 expression.

35. The ABPC or composition of claim 34, wherein the dissociation rate of the ABD at a pH of ˜4.0-6.5 is at least 10%, 3-fold, or 10-fold faster than the dissociation rate of the ABD at a pH of ˜7.0-8.0; and/or
wherein the K_{D} of the ABD at a pH of ˜4.0-6.5 is at least 10%, 3-fold, or 10-fold greater than the K_{D} of the ABD at a pH of ˜7.0-8.0; and/or
wherein the ABPC is cytotoxic or cytostatic to the cancer cell or CAF.

36. The ABPC or composition of claim 34 or 35, wherein the ABPC is cross-reactive with a non-human primate LRRC15 and human LRRC15;
wherein the ABPC is cross-reactive with a non-human primate LRRC15, a human LRRC15, and one or both of rat LRRC15 and a mouse LRRC15;
wherein the ABPC is cross-reactive with a non-human primate LRRC15, a human LRRC15, a rat LRRC15, and a mouse LRRC15; and/or
wherein the wherein the ABD binds to an epitope of LRRC15 that is present on the surface of cells from an Old World Monkey.

37. The ABPC or composition of any one of claims 1-36, wherein the ABPC comprises a single polypeptide, optionally wherein the ABD is selected from the group consisting of: a VH domain, a VHH domain, a VNAR domain, and a scFv.

38. The ABPC or composition of any one of claims 1-36, wherein the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

39. The ABPC or composition of any one of claims 1-36, wherein the ABPC comprises two or more polypeptides.

40. The ABPC of claim 39, wherein the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAC, and a PROTAB.

41. The ABPC of any one of claims 12-16, and 38-40, or the composition of any one of claims 17-33, wherein at least one polypeptide of the ABPC is conjugated to the toxin, the radioisotope, the drug, or the small molecule via a cleavable or non-cleavable linker.

42. The ABPC or composition of any one of claims 1-41, wherein the half-life of the ABPC in vivo is increased as compared to the half-life of a control ABPC in vivo, optionally wherein the increase is ˜5%-95%, ˜10%-95%, ˜30%-95%, ˜50%-95%, or ˜70%-95% as compared to the half-life of a control ABPC in vivo.

43. The ABPC or composition of any one of claims 1-42, wherein the ABPC further comprises a second ABD.

44. A method of treating a cancer characterized by having a population of cancer cells and/or CAFs that have a predetermined level of LRRC15 or an epitope of LRRC15 presented on their surface, comprising administering a therapeutically effective amount of the ABPC or composition of any one of claims 1-43 to a subject identified as having a cancer characterized by having the population of cancer cells and/or CAFs, thereby treating the cancer.

45. A method of reducing the volume of a tumor in a subject, wherein the tumor is characterized by having a population of cancer cells and/or CAFs that have a predetermined level of LRRC15 or an epitope of LRRC15 presented on their surface, comprising administering a therapeutically effective amount of the ABPC or composition of any one of claims 1-43 to a subject identified as having a cancer characterized by having the population of cancer cells and/or CAFs, thereby reducing the volume of the tumor.

46. A method of inducing cell death in a cancer cell and/or CAF in a subject, wherein the cancer cell and/or CAF has a predetermined level of LRRC15 or an epitope of LRRC15 presented on its surface, comprising administering a therapeutically effective amount of the ABPC or composition of any one of claims 1-43 to a subject identified as having a cancer characterized by having a population of cancer cells and/or CAFs, thereby inducing the cell death in the cancer cell and/or CAF.

47. A method of decreasing the risk of developing a metastasis or decreasing the risk of developing an additional metastasis in a subject having a cancer, wherein the cancer is characterized by having a population of cancer cells, CAFs, and/or stromal cells that have a predetermined level of LRRC15 or an epitope of LRRC15 presented on their surface, comprising administering a therapeutically effective amount of the ABPC or composition of any one of claims 1-43 to a subject identified as having a cancer characterized by having the population of cancer cells, CAFs, and/or stromal cells thereby decreasing the risk.

48. An anti-LRRC15 antibody comprising:
(a) a heavy chain variable domain (HCVD) and a light chain variable domain (LCVD) comprising sequences that are 90% identical to and comprise the same heavy chain CDRs and light chain CDRs as the HCVD and LCVD sequences set forth in one of the following pairs of sequences: SEQ ID NOs: 1 and 64, SEQ ID NOs: 84 and 154, SEQ ID NOs: 430 and 455, SEQ ID NOs: 382 and 489, SEQ ID NOs: 382 and 491, SEQ ID NOs: 516 and 522, SEQ ID NOs: 571 and 517, SEQ ID NOs: 576 and 581, SEQ ID NOs: 576 and 639, SEQ ID NOs: 576 and 700, SEQ ID NOs: 382 and 490, SEQ ID NOs: 570 and 522, SEQ ID NOs: 576 and 641, SEQ ID NOs: 84 and 177, and SEQ ID NOs: 576 and 625; or
(b) an HCVD and an LCVD comprising sequences that are 95% identical to and comprise the same heavy chain CDRs and light chain CDRs as the HCVD and LCVD sequences set forth in one of the following pairs of sequences: SEQ ID NOs: 1 and 64, SEQ ID NOs: 84 and 154, SEQ ID NOs: 430 and 455, SEQ ID NOs: 382 and 489, SEQ ID NOs: 382 and 491, SEQ ID NOs: 516 and 522, SEQ ID NOs: 571 and 517, SEQ ID NOs: 576 and 581, SEQ ID NOs: 576 and 639, SEQ ID NOs: 576 and 700, SEQ ID NOs: 382 and 490, SEQ ID NOs: 570 and 522, SEQ ID NOs: 576 and 641, SEQ ID NOs: 84 and 177, and SEQ ID NOs: 576 and 625; or
(c) one of the following pairs of sequences: SEQ ID NOs: 1 and 64, SEQ ID NOs: 84 and 154, SEQ ID NOs: 430 and 455, SEQ ID NOs: 382 and 489, SEQ ID NOs: 382 and 491, SEQ ID NOs: 516 and 522, SEQ ID NOs: 571 and 517, SEQ ID NOs: 576 and 581, SEQ ID NOs: 576 and 639, SEQ ID NOs: 576 and 700, SEQ ID NOs: 382 and 490, SEQ ID NOs: 570 and 522, SEQ ID NOs: 576 and 641, SEQ ID NOs: 84 and 177, SEQ ID NOs: 576 and 625, SEQ ID NOs: 724 and 725, SEQ ID NOs: 726 and 727, SEQ ID NOs: 728 and 729, SEQ ID NOs: 730 and 731, SEQ ID NOs: 730 and 732, SEQ ID NOs: 733 and 734, SEQ ID NOs: 735 and 736, SEQ ID NOs: 737 and 738, SEQ ID NOs: 737 and 739, and SEQ ID NOs: 737 and 740, optionally wherein position 205 of the light chain sequence of the antibody comprises an amino acid other than cysteine.

49. The anti-LRRC15 antibody of claim 48, comprising heavy chain and light chain sequences having the sequences as set forth in one of the following pairs of sequences: SEQ ID NOs: 724 and 725, SEQ ID NOs: 726 and 727, SEQ ID NOs: 728 and 729, SEQ ID NOs: 730 and 731, SEQ ID NOs: 730 and 732, SEQ ID NOs: 733 and 734, SEQ ID NOs: 735 and 736, SEQ ID NOs: 737 and 738, SEQ ID NOs: 737 and 739, and SEQ ID NOs: 737 and 740, optionally wherein position 205 of the light chain sequence of the antibody comprises an amino acid other than cysteine.

50. The anti-LRRC15 antibody of claim 48 or 49, for use in treating a subject having an LRRC15+ tumor comprising LRRC15+ cancer cells and/or LRRC15+ CAFs.
